SEQUENCE LISTING (391 sequences)

SEQ ID NO: 1 - RNA, 98 nt; single-stranded, linear
  All C′s are 2′-NH2 cytosine
  All U′s are 2′-NH2 uracil
gggaaaagcgaaucauacacaagannnnnnnnnnnnnnnnnnnnnnnnnnnnnnnnnnnnnnnnnnnnnnnnnngcuccgccagagaccaaccgagaa

SEQ ID NO: 2 - RNA, 41 nt; single-stranded, linear
  All C′s are 2′-NH2 cytosine
  All U′s are 2′-NH2 uracil
uaauacgacucacuauagggaaaagcgaaucauacacaaga

SEQ ID NO: 3 - RNA, 24 nt; single-stranded, linear
  All C′s are 2′-NH2 cytosine
  All U′s are 2′-NH2 uracil
uucucgguuggucucuggcggagc

SEQ ID NO: 4 - RNA, 96 nt; single-stranded, linear
  All C′s are 2′-NH2 cytosine
  All U′s are 2′-NH2 uracil
gggaaaagcgaaucauacacaagaaugguuggccugggcgcaggcuucgaagacucggcgggaacgggaauggcuccgccagagaccaaccgagaa

SEQ ID NO: 5 - RNA, 98 nt; single-stranded, linear
  All C′s are 2′-NH2 cytosine
  All U′s are 2′-NH2 uracil
gggaaaagcgaaucauacacaagacaggcacugaaaacucggcgggaacgaaaguagugccgacucagacgcgugcuccgccagagaccaaccgagaa

SEQ ID NO: 6 - RNA, 91 nt; single-stranded, linear
  All C′s are 2′-NH2 cytosine
  All U′s are 2′-NH2 uracil
gggaaaagcgaaucauacacaagaagucuggccaaagacucggcgggaacguaaaacggccagaauugcuccgccagagaccaaccgagaa

SEQ ID NO: 7 - RNA, 94 nt; single-stranded, linear
  All C′s are 2′-NH2 cytosine
  All U′s are 2′-NH2 uracil
gggaaaagcgaaucauacacaagaguaggagguuccaucaccaggacucggcgggaacggaaggugaugsgcuccgccagagaccaaccgagaa

SEQ ID NO: 8 - RNA, 95 nt; single-stranded, linear
  All C′s are 2′-NH2 cytosine
  All U′s are 2′-NH2 uracil
gggaaaagcgaaucauacacaagaacaaggaucgauggcgagccggggagggcucggcgggaacgaaaucugcuccgccagagaccaaccgagaa

SEQ ID NO: 9 - RNA, 97 nt; single-stranded, linear
  All C′s are 2′-NH2 cytosine
  All U′s are 2′-NH2 uracil
gggaaaagcgaaucauacacaagauugggcaggcagagcgagaccgggggcucggcgggaacggaacaggaaugcuccgccagagaccaaccgagaa

SEQ ID NO: 10 - RNA, 97 nt; single-stranded, linear
  All C′s are 2′-NH2 cytosine
  All U′s are 2′-NH2 uracil
gggaaaagcgaaucauacacaagaaagggaugggauugggacgagcggccaagacucggcgggaacgaagggugcuccgccagagaccaaccgagaa

SEQ ID NO: 11 - RNA, 96 nt; single-stranded, linear
  All C′s are 2′-NH2 cytosine
  All U′s are 2′-NH2 uracil
gggaaaagcgaaucauacacaagacucggcgggaacgaaagugucaugguagcaaguccaaugguggacucugcuccgccagagaccaaccgagaa

SEQ ID NO: 12 - RNA, 98 nt; single-stranded, linear
  All C′s are 2′-NH2 cytosine
  All U′s are 2′-NH2 uracil
gggaaaagcgaaucauacacaagacucggcgggaacgugaaguggguagguagcugaagacggucugggcgccagcuccgccagagaccaaccgagaa

SEQ ID NO: 13 - RNA, 99 nt; single-stranded, linear
  All C′s are 2′-NH2 cytosine
  All U′s are 2′-NH2 uracil
gggaaaagcgaaucauacacaagaaagggaugggauugggacgagcggccaagacucggcgggaacgaaggguccgcuccgccagagaccaaccgagaa

SEQ ID NO: 14 - RNA, 98 nt; single-stranded, linear
  All C′s are 2′-NH2 cytosine
  All U′s are 2′-NH2 uracil
gggaaaagcgaaucauacacaagacucggcgggaacgaagugugugaguaacgaucacuugguacuaaaagcccgcuccgccagagaccaaccgagaa

SEQ ID NO: 15 - RNA, 100 nt; single-stranded, linear
  All C′s are 2′-NH2 cytosine
  All U′s are 2′-NH2 uracil
gggaaaagcgaaucauacacaagacucggcgggaaucgaaaguguacugaauuagaacggugggccugcucaucgugcuccgccagagaccaaccgagaa

SEQ ID NO: 16 - RNA, 103 nt; single-stranded, linear
  All C′s are 2′-NH2 cytosine
  All U′s are 2′-NH2 uracil
gggaaaagcgaaucauacacaagacucggcgggaaucguaauguggaugauagcacgauggcagyaguagucggaccgcgcuccgccagagaccaaccgagaa

SEQ ID NO: 17 - RNA, 98 nt; single-stranded, linear
  All C′s are 2′-NH2 cytosine
  All U′s are 2′-NH2 uracil
gggaaaagcgaaucauacacaagacagcggcggagucagugaaagcguggggggygcgggaggucuacccugacgcuccgccagagaccaaccgagaa

SEQ ID NO: 18 - RNA, 95 nt; single-stranded, linear
  All C′s are 2′-NH2 cytosine
  All U′s are 2′-NH2 uracil
gggaaaagcgaaucauacacaagacggcugugugugguagcgucauaguaggagucgucacgaaccaaggcgcuccgccagagaccaaccgagaa

SEQ ID NO: 19 - RNA, 98 nt; single-stranded, linear
  All C′s are 2′-NH2 cytosine
  All U′s are 2′-NH2 uracil
gggaaaagcgaaucauacacaagacggcugugugguguuggagcgucauaguaggagucgucacgaaccaaggcgcuccgccagagaccaaccgagaa

SEQ ID NO: 20 - RNA, 97 nt; single-stranded, linear
  All C′s are 2′-NH2 cytosine
  All U′s are 2′-NH2 uracil
gggaaaagcgaaucauacacaagacgaugcgaggcaagaaauggagucguuacgaacccucuugcagugcgcggcuccgccagagaccaaccgagaa

SEQ ID NO: 21 - RNA, 95 nt; single-stranded, linear
  All C′s are 2′-NH2 cytosine
  All U′s are 2′-NH2 uracil
gggaaaagcgaaucauacacaagacgugcggagcaaauaggggaucauggagucguacgaaccguuaucgcgcuccgccagagaccaaccgagaa

SEQ ID NO: 22 - RNA, 97 nt; single-stranded, linear
  All C′s are 2′-NH2 cytosine
  All U′s are 2′-NH2 uracil
gggaaaagcgaaucauacacaagacuggggagcaggauaugagaugugcggggcaauggagucgugacgaaccgcuccgccagagaccaaccgagaa

SEQ ID NO: 23 - RNA, 95 nt; single-stranded, linear
  All C′s are 2′-NH2 cytosine
  All U′s are 2′-NH2 uracil
gggaaaagcgaaucauacacaagaguccgcccccagggaugcaacgggguggcucuaaaaggcuuggcuaagcuccgccagagaccaaccgagaa

SEQ ID NO: 24 - RNA, 94 nt; single-stranded, linear
  All C′s are 2′-NH2 cytosine
  All U′s are 2′-NH2 uracil
gggaaaagcgaaucauacacaagagagaaugagcauggccggggcaggaaguggguggcaacggaggccagcuccgccagagaccaaccgagaa

SEQ ID NO: 25 - RNA, 95 nt; single-stranded, linear
  All C′s are 2′-NH2 cytosine
  All U′s are 2′-NH2 uracil
gggaaaagcgaaucauacacaagagauacagcgcgggucuaaagaccuugccccuaggaugcaacgggguggcuccgccagagaccaaccgagaa

SEQ ID NO: 26 - RNA, 97 nt; single-stranded, linear
  All C′s are 2′-NH2 cytosine
  All U′s are 2′-NH2 uracil
gggaaaagcgaaucauacacaagaugaagggugguaagagagagucugagcucguccuagggaugcaacggcagcuccgccagagaccaaccgagaa

SEQ ID NO: 27 - RNA, 99 nt; single-stranded, linear
  All C′s are 2′-NH2 cytosine
  All U′s are 2′-NH2 uracil
gggaaaagcgaaucauacacaagacaaaccugcagucgcgcggugaaaccuaggguugcaacgguacaucgcugugcuccgccagagaccaaccgagaa

SEQ ID NO: 28 - RNA, 97 nt; single-stranded, linear
  All C′s are 2′-NH2 cytosine
  All U′s are 2′-NH2 uracil
gggaaaagcgaaucauacacaagaguggacuggaaucuucgaggacaggaacguuccuagggaugcaacggacgcuccgccagagaccaaccgagaa

SEQ ID NO: 29 - RNA, 97 nt; single-stranded, linear
  All C′s are 2′-NH2 cytosine
  All U′s are 2′-NH2 uracil
gggaaaagcgaaucauacacaagaguguaccaauggaggcaaugcugcgggaauggaggccuagggaugcaacgcuccgccagagaccaaccgagaa

SEQ ID NO: 30 - RNA, 97 nt; single-stranded, linear
  All C′s are 2′-NH2 cytosine
  All U′s are 2′-NH2 uracil
gggaaaagcgaaucauacacaagagucccuagggaugcaacgggcagcauucgcauaggaguaaucggaggucgcuccgccagagaccaaccgagaa

SEQ ID NO: 31 - RNA, 97 nt; single-stranded, linear
  All C′s are 2′-NH2 cytosine
  All U′s are 2′-NH2 uracil
gggaaaagcgaaucauacacaagagccuagggaugcaacggcgaauggauagcgaugucguggacagccaggugcuccgccagagaccaaccgagaa

SEQ ID NO: 32 - RNA, 98 nt; single-stranded, linear
  All C′s are 2′-NH2 cytosine
  All U′s are 2′-NH2 uracil
gggaaaagcgaaucauacacaagaaucgaaccuagggaugcaacggugaagguugugaggauucgccauuaggcgcuccgccagagaccaaccgagaa

SEQ ID NO: 33 - RNA, 98 nt; single-stranded, linear
  All C′s are 2′-NH2 cytosine
  All U′s are 2′-NH2 uracil
gggaaaagcgaaucauacacaagagcuagggaugccgcagaauggucgcggauguaauaggugaagauuguugcgcuccgccagagaccaaccgagaa

SEQ ID NO: 34 - RNA, 98 nt; single-stranded, linear
  All C′s are 2′-NH2 cytosine
  All U′s are 2′-NH2 uracil
gggaaaagcgaaucauacacaagaggaccuagggaugcaacgguccgaccuugaugcgcggguguccaagcuacgcuccgccagagaccaaccgagaa

SEQ ID NO: 35 - RNA, 97 nt; single-stranded, linear
  All C′s are 2′-NH2 cytosine
  All U′s are 2′-NH2 uracil
gggaaaagcgaaucauacacaagaaagggaggagcuagagagggaaagguuacuacgcgccagaauaggaugugcuccgccagagaccaaccgagaa

SEQ ID NO: 36 - RNA, 97 nt; single-stranded, linear
  All C′s are 2′-NH2 cytosine
  All U′s are 2′-NH2 uracil
gggaaaagcgaaucauacacaagaccaacguacaucgcgagcugguggagaguucaugaggguguuacggggugcuccgccagagaccaaccgagaa

SEQ ID NO: 37 - RNA, 96 nt; single-stranded, linear
  All C′s are 2′-NH2 cytosine
  All U′s are 2′-NH2 uracil
gggaaaagcgaaucauacacaagacccaacgugucaucgcgagcuggcggagaguucaugaggguuacgggugcuccgccagagaccaaccgagaa

SEQ ID NO: 38 - RNA, 98 nt; single-stranded, linear
  All C′s are 2′-NH2 cytosine
  All U′s are 2′-NH2 uracil
gggaaaagcgaaucauacacaagaguuggugcgagcuggggcggcgagaagguaggcgguccgaguguucgaaugcuccgccagagaccaaccgagaa

SEQ ID NO: 39 - RNA, 98 nt; single-stranded, linear
  All C′s are 2′-NH2 cytosine
  All U′s are 2′-NH2 uracil
gggaaaagcgaaucauacacaagacuggcaagragugcgugaggguacguuagggguguuugggccgaucgcaugcuccgccagagaccaaccgagaa

SEQ ID NO: 40 - RNA, 97 nt; single-stranded, linear
  All C′s are 2′-NH2 cytosine
  All U′s are 2′-NH2 uracil
gggaaaagcgaaucauacacaagauuggucguacuggacagagccgugguagagggauugggacaaagugucagcuccgccagagaccaaccgagaa

SEQ ID NO: 41 - RNA, 99 nt; single-stranded, linear
  All C′s are 2′-NH2 cytosine
  All U′s are 2′-NH2 uracil
gggaaaagcgaaucauacacaagaugugagaaaguggccaacuuuaggacgucgguggacugygcggguaggcucgcuccgccagagaccaaccgagaa

SEQ ID NO: 42 - RNA, 98 nt; single-stranded, linear
  All C′s are 2′-NH2 cytosine
  All U′s are 2′-NH2 uracil
gggaaaagcgaaucauacacaagacaggcagaugugucugaguucgucggaguagacgucgguggacgcggaacgcuccgccagagaccaaccgagaa

SEQ ID NO: 43 - RNA, 97 nt; single-stranded, linear
  All C′s are 2′-NH2 cytosine
  All U′s are 2′-NH2 uracil
gggaaaagcgaaucauacacaagaugugauuaggcaguugcagccgccgugcggagacgugacucgaggauucgcuccgccagagaccaaccgagaa

SEQ ID NO: 44 - RNA, 96 nt; single-stranded, linear
  All C′s are 2′-NH2 cytosine
  All U′s are 2′-NH2 uracil
gggaaaagcgaaucauacacaagaugccgguggaaaggcggguaggugacccgaggauuccuaccaagccaugcuccgccagagaccaaccgagaa

SEQ ID NO: 45 - RNA, 93 nt; single-stranded, linear
  All C′s are 2′-NH2 cytosine
  All U′s are 2′-NH2 uracil
gggaaaagcgaaucauacacaagagaggugraugggagaguggagcccgggugacucgaggauucccgugcuccgccagagaccaaccgagaa

SEQ ID NO: 46 - RNA, 97 nt; single-stranded, linear
  All C′s are 2′-NH2 cytosine
  All U′s are 2′-NH2 uracil
gggaaaagcgaaucauacacaagagucaugcuguggcugaacauacuggugaaaguucaguaggguggauacagcuccgccagagaccaaccgagaa

SEQ ID NO: 47 - RNA, 96 nt; single-stranded, linear
  All C′s are 2′-NH2 cytosine
  All U′s are 2′-NH2 uracil
gggaaaagcgaaucauacacaagaccggggauggugagucgggcagugugaccgaacuggugcccgcugagagcuccgccagagaccaaccgagaa

SEQ ID NO: 48 - RNA, 97 nt; single-stranded, linear
  All C′s are 2′-NH2 cytosine
  All U′s are 2′-NH2 uracil
gggaaaagcgaaucauacacaagaacacuaaccaggucucugaacgcgggacggaggugugggcgagguggaagcuccgccagagaccaaccgagaa

SEQ ID NO: 49 - RNA, 99 nt; single-stranded, linear
  All C′s are 2′-NH2 cytosine
  All U′s are 2′-NH2 uracil
gggaaaagcgaaucauacacaagaccgucucccgagaaccaggcagaggacgugcugaaggagcugcaucuagaagcuccgccagagaccaaccgagaa

SEQ ID NO: 50 - RNA, 99 nt; single-stranded, linear
  All C′s are 2′-NH2 cytosine
  All U′s are 2′-NH2 uracil
gggaaaagcgaaucauacacaagaccgucuccgagaaccaggcagaggaggugcugaaggrgcuggcaucuacaagcuccgccagagaccaaccgagaa

SEQ ID NO: 51 - RNA, 96 nt; single-stranded, linear
  All C′s are 2′-NH2 cytosine
  All U′s are 2′-NH2 uracil
gggaaaagcgaaucauacacaagacccgcacauaauguagggaacaauguuauggcggaauugauaaccggugcuccgccagagaccaaccgagaa

SEQ ID NO: 52 - RNA, 98 nt; single-stranded, linear
  All C′s are 2′-NH2 cytosine
  All U′s are 2′-NH2 uracil
gggaaaagcgaaucauacacaagacgauguuagcgccuccgggagagguuagggucgugcggnaagagugaggugcuccgccagagaccaaccgagaa

SEQ ID NO: 53 - RNA, 99 nt; single-stranded, linear
  All C′s are 2′-NH2 cytosine
  All U′s are 2′-NH2 uracil
gggaaaagcgaaucauacacaagagguacgggcgagacgagauggacuuauaggucgaugaacggguagcagcucgcuccgccagagaccaaccgagaa

SEQ ID NO: 54 - RNA, 96 nt; single-stranded, linear
  All C′s are 2′-NH2 cytosine
  All U′s are 2′-NH2 uracil
gggaaaagcgaaucauacacaagacgguugcugaacagaacgugagucuuggugagucgcacagauuguccugcuccgccagagaccaaccgagaa

SEQ ID NO: 55 - RNA, 97 nt; single-stranded, linear
  All C′s are 2′-NH2 cytosine
  All U′s are 2′-NH2 uracil
gggaaaagcgaaucauacacaagaacugaguaaggucuggcguggcauuagguuagugggaggcuuggaguaggcuccgccagagaccaaccgagaa

SEQ ID NO: 56 - RNA, 20 nt; single-stranded, linear
  All C′s are 2′-NH2 cytosine
  All U′s are 2′-NH2 uracil
aagacucggcgggaacgaaa

SEQ ID NO: 57 - RNA, 16 nt; single-stranded, linear
  All C′s are 2′-NH2 cytosine
  All U′s are 2′-NH2 uracil
ggagucgugacgaacc

SEQ ID NO: 58 - RNA, 16 nt; single-stranded, linear
  All C′s are 2′-NH2 cytosine
  All U′s are 2′-NH2 uracil
ccuagggaugcaacgg

SEQ ID NO: 59 - RNA, 18 nt; single-stranded, linear
  All C′s are 2′-NH2 cytosine
  All U′s are 2′-NH2 uracil
rcugggagrguggguguu

SEQ ID NO: 60 - RNA, 42 nt; single-stranded, linear
  All C′s are 2′-NH2 cytosine
  All U′s are 2′-NH2 uracil
ugugnnnnagunnnnnnnnnuagacgucgguggacnnngcgg

SEQ ID NO: 61 - RNA, 21 nt; single-stranded, linear
  All C′s are 2′-NH2 cytosine
  All U′s are 2′-NH2 uracil
gggnnngugacycgrggayuc

SEQ ID NO: 62 - RNA, 23 nt; single-stranded, linear
  All C′s are 2′-NH2 cytosine
  All U′s are 2′-NH2 uracil
ugancnnacuggugnnngngnag

SEQ ID NO: 63 - RNA, 32 nt; single-stranded, linear
  All C′s are 2′-NH2 cytosine
  All U′s are 2′-NH2 uracil
gucucygaacnnggnaggangugnuggagnug

SEQ ID NO: 64 - RNA, 71 nt; single-stranded, linear
  All C′s are 2′-NH2 cytosine
  All U′s are 2′-NH2 uracil
gggaggacgaugcggnnnnnnnnnnnnnnnnnnnnnnnnnnnnnnnnnnnnnnnncagacgacucgcccga

SEQ ID NO: 65 - RNA, 32 nt; single-stranded, linear
  All C′s are 2′-NH2 cytosine
  All U′s are 2′-NH2 uracil
taatacgactcactatagggaggacgatgcgg

SEQ ID NO: 66 - RNA, 17 nt; single-stranded, linear
  All C′s are 2′-NH2 cytosine
  All U′s are 2′-NH2 uracil
tcgggcgagtcgtcctg

SEQ ID NO: 67 - RNA, 71 nt; single-stranded, linear
  All C′s are 2′-NH2 cytosine
  All U′s are 2′-NH2 uracil
gggaggacgaugcggcgcguaugugugaaagcgugugcacggaggcgucuacaaucagacgacucgcccga

SEQ ID NO: 68 - RNA, 71 nt; single-stranded, linear
  All C′s are 2′-NH2 cytosine
  All U′s are 2′-NH2 uracil
gggaggacgaugcggggcauugugugaauagcugaucccacagguaacaacagcacagacgacucgcccga

SEQ ID NO: 69 - RNA, 71 nt; single-stranded, linear
  All C′s are 2′-NH2 cytosine
  All U′s are 2′-NH2 uracil
gggaggacgaugcgguaaugugugaaucaagcagucugaauagauuagacaaaaucagacgacucgcccga

SEQ ID NO: 70 - RNA, 71 nt; single-stranded, linear
  All C′s are 2′-NH2 cytosine
  All U′s are 2′-NH2 uracil
gggaggacgaugcggaugugugaguagcugagcgcccgaguaugawaccugacuacagacgacucgcccga

SEQ ID NO: 71 - RNA, 71 nt; single-stranded, linear
  All C′s are 2′-NH2 cytosine
  All U′s are 2′-NH2 uracil
gggaggacgaugcggaaaccuugaugugugauagagcaucccccaggcgacguaccagacgacucgcccga

SEQ ID NO: 72 - RNA, 70 nt; single-stranded, linear
  All C′s are 2′-NH2 cytosine
  All U′s are 2′-NH2 uracil
gggaggacgaugcgguugagaugugugaguacaagcucaaaaucccguuggaggcagacgacucgcccga

SEQ ID NO: 73 - RNA, 71 nt; single-stranded, linear
  All C′s are 2′-NH2 cytosine
  All U′s are 2′-NH2 uracil
gggaggacgaugcgguagagguaguaugugugggagaugaaaauacuguggaaagcagacgacucgcccga

SEQ ID NO: 74 - RNA, 71 nt; single-stranded, linear
  All C′s are 2′-NH2 cytosine
  All U′s are 2′-NH2 uracil
gggaggacgaugcggaaaguuaugaguccguauaucaaggucgacaugugugaaucagacgacucgcccga

SEQ ID NO: 75 - RNA, 71 nt; single-stranded, linear
  All C′s are 2′-NH2 cytosine
  All U′s are 2′-NH2 uracil
gggaggacgaugcggcacgaaaaacccgaauugggucgcccauaaggaugugugacagacgacucgcccga

SEQ ID NO: 76 - RNA, 71 nt; single-stranded, linear
  All C′s are 2′-NH2 cytosine
  All U′s are 2′-NH2 uracil
gggaggacgaugcggguaaagagauccuaauggcucgcuagaugugaugugaaaccagacgacucgcccga

SEQ ID NO: 77 - RNA, 71 nt; single-stranded, linear
  All C′s are 2′-NH2 cytosine
  All U′s are 2′-NH2 uracil
gggaggacgaugcgguaacaacaaucaaggcggguucaccgccccaguaugagugcagacgacucgcccga

SEQ ID NO: 78 - RNA, 71 nt; single-stranded, linear
  All C′s are 2′-NH2 cytosine
  All U′s are 2′-NH2 uracil
gggaggacgaugcgguaacaacaaucaaggcggguuyaccgccccaguaugaguacagacgacucgcccga

SEQ ID NO: 79 - RNA, 71 nt; single-stranded, linear
  All C′s are 2′-NH2 cytosine
  All U′s are 2′-NH2 uracil
gggaggacgaugcgguaacaacaaucaaggcggguuyaccgcuccaguaugaguacagacgacucgcccga

SEQ ID NO: 80 - RNA, 71 nt; single-stranded, linear; identical to SEQ ID NO: 77
  All C′s are 2′-NH2 cytosine
  All U′s are 2′-NH2 uracil

SEQ ID NO: 81 - RNA, 71 nt; single-stranded, linear
  All C′s are 2′-NH2 cytosine
  All U′s are 2′-NH2 uracil
gggaggacgaugcggaccaagcaaucuauggucgaacgcuacacaugaaugacgucagacgacucgcccga

SEQ ID NO: 82 - RNA, 71 nt; single-stranded, linear
  All C′s are 2′-NH2 cytosine
  All U′s are 2′-NH2 uracil
gggaggacgaugcgggaacaugaaguaaucaaagucguaccaauauacaggaagccagacgacucgcccga

SEQ ID NO: 83 - RNA, 70 nt; single-stranded, linear
  All C′s are 2′-NH2 cytosine
  All U′s are 2′-NH2 uracil
gggaggacgaugcgggacaugaaguaagaccgucacaauucgaaugauugaauacagacgacucgcccga

SEQ ID NO: 84 - RNA, 72 nt; single-stranded, linear
  All C′s are 2′-NH2 cytosine
  All U′s are 2′-NH2 uracil
gggaggacgaugcgggaacaugaaguaaaaagucgacgaauuagcuguaaccaaaacagacgacucgcccga

SEQ ID NO: 85 - RNA, 71 nt; single-stranded, linear
  All C′s are 2′-NH2 cytosine
  All U′s are 2′-NH2 uracil
gggaggacgaugcgggaacaugaaguaaaagucugaguuaguaaauuacagugaucagacgacucgcccga

SEQ ID NO: 86 - RNA, 72 nt; single-stranded, linear
  All C′s are 2′-NH2 cytosine
  All U′s are 2′-NH2 uracil
gggaggacgaugcgggaacuugaaguugaanucgcuaagguuauggauucaagauucagacgacucgcccga

SEQ ID NO: 87 - RNA, 71 nt; single-stranded, linear
  All C′s are 2′-NH2 cytosine
  All U′s are 2′-NH2 uracil
gggaggacgaugcggaacaugaaguaauaagucgacguaauuagcuguaacuaaacagacgacucgcccga

SEQ ID NO: 88 - RNA, 70 nt; single-stranded, linear
  All C′s are 2′-NH2 cytosine
  All U′s are 2′-NH2 uracil
gggaggacgaugcggaacaugaaguaaaagucugaguuagaaauuacaagugaucagacgacucgcccga

SEQ ID NO: 89 - RNA, 71 nt; single-stranded, linear
  All C′s are 2′-NH2 cytosine
  All U′s are 2′-NH2 uracil
gggaggacgaugcgguaacauaaaguagcgcgucugugagaggaagugccuggaucagacgacucgcccga

SEQ ID NO: 90 - RNA, 71 nt; single-stranded, linear
  All C′s are 2′-NH2 cytosine
  All U′s are 2′-NH2 uracil
gggaggacgaugcggauagaaccgcaaggauaaccucgaccguggucaacugagacagacgacucgcccga

SEQ ID NO: 91 - RNA, 70 nt; single-stranded, linear
  All C′s are 2′-NH2 cytosine
  All U′s are 2′-NH2 uracil
gggaggacgaugcgguaagaaccgcuagcgcacgaucaaacaaagagaaacaaacagacgacucgcccga

SEQ ID NO: 92 - RNA, 71 nt; single-stranded, linear
  All C′s are 2′-NH2 cytosine
  All U′s are 2′-NH2 uracil
gggaggacgaugcgguucucuccaagaacygagcgaauaaacsaccggasucacacagacgacucgcccga

SEQ ID NO: 93 - RNA, 71 nt; single-stranded, linear
  All C′s are 2′-NH2 cytosine
  All U′s are 2′-NH2 uracil
gggaggacgaugcggugucucuccugacuuuuauucuuaguucgagcuguccuggcagacgacucgcccga

SEQ ID NO: 94 - RNA, 71 nt; single-stranded, linear
  All C′s are 2′-NH2 cytosine
  All U′s are 2′-NH2 uracil
gggaggacgaugcggccguacaugguaarccucgaaggauucccgggaugauccccagacgacucgcccga

SEQ ID NO: 95 - RNA, 71 nt; single-stranded, linear
  All C′s are 2′-NH2 cytosine
  All U′s are 2′-NH2 uracil
gggaggacgaugcggucccagagucccgugaugcgaagaauccauuaguaccagacagacgacucgcccga

SEQ ID NO: 96 - RNA, 70 nt; single-stranded, linear
  All C′s are 2′-NH2 cytosine
  All U′s are 2′-NH2 uracil
gggaggacgaugcgggauguaaaugacaaaugaaccucgaaagauugcacacuccagacgacucgcccga

SEQ ID NO: 97 - RNA, 72 nt; single-stranded, linear
  All C′s are 2′-NH2 cytosine
  All U′s are 2′-NH2 uracil
gggaggacgaugcggauguaaaucuaggcagaaacguagggcauccaccgcaacgacagacgacucgcccga

SEQ ID NO: 98 - RNA, 70 nt; single-stranded, linear
  All C′s are 2′-NH2 cytosine
  All U′s are 2′-NH2 uracil
gggaggacgaugcggauaacccaagcagcnucgagaaagagcuccauagaugaucagacgacucgcccga

SEQ ID NO: 99 - RNA, 71 nt; single-stranded, linear
  All C′s are 2′-NH2 cytosine
  All U′s are 2′-NH2 uracil
gggaggacgaugcggcaaagcacgcguauggcaugaaacuggcancccaaguaagcagacgacucgcccga

SEQ ID NO: 100 - RNA, 71 nt; single-stranded, linear
  All C′s are 2′-NH2 cytosine
  All U′s are 2′-NH2 uracil
gggaggacgaugcggcaaaagguugacguagcgaagcucucaaaauggucaugaccagacgacucgcccga

SEQ ID NO: 101 - RNA, 70 nt; single-stranded, linear
  All C′s are 2′-NH2 cytosine
  All U′s are 2′-NH2 uracil
gggaggacgaugcggaagugaagcuaaagcggagggccauucaguuucncaccacagacgacucgcccga

SEQ ID NO: 102 - RNA, 70 nt; single-stranded, linear
  All C′s are 2′-NH2 cytosine
  All U′s are 2′-NH2 uracil
gggaggacgaugcggaagugaagcuaaagsggagggccacucagaaacgcaccacagacgacucgcccga

SEQ ID NO: 103 - RNA, 71 nt; single-stranded, linear
  All C′s are 2′-NH2 cytosine
  All U′s are 2′-NH2 uracil
gggaggacgaugcggcaccgcuaagcaguggcauagcccaguaaccuguaagagacagacgacucgcccga

SEQ ID NO: 104 - RNA, 67 nt; single-stranded, linear
  All C′s are 2′-NH2 cytosine
  All U′s are 2′-NH2 uracil
gggaggacgaugcggcacgcuaagcaguggcauagcgwaaccuguaagagacagacgacucgcccga

SEQ ID NO: 105 - RNA, 71 nt; single-stranded, linear
  All C′s are 2′-NH2 cytosine
  All U′s are 2′-NH2 uracil
gggaggacgaugcggagauuaccauaaccgcguagucgaagacauauaguagcgacagacgacucgcccga

SEQ ID NO: 106 - RNA, 71 nt; single-stranded, linear
  All C′s are 2′-NH2 cytosine
  All U′s are 2′-NH2 uracil
gggaggacgaugcggacucggguagaacgcgacuugccaccacucccauaaagaccagacgacucgcccga

SEQ ID NO: 107 - RNA, 71 nt; single-stranded, linear
  All C′s are 2′-NH2 cytosine
  All U′s are 2′-NH2 uracil
gggaggacgaugcggucagaacucugccgcuguagacaaagaggagcuuagcgaacagacgacucgcccga

SEQ ID NO: 108 - RNA, 71 nt; single-stranded, linear
  All C′s are 2′-NH2 cytosine
  All U′s are 2′-NH2 uracil
gggaggacgaugcggaaugagcaucgagagagcgcgaacucaucgagcguacuaacagacgacucgcccga

SEQ ID NO: 109 - RNA, 71 nt; single-stranded, linear; identical to SEQ ID NO: 99
  All C′s are 2′-NH2 cytosine
  All U′s are 2′-NH2 uracil

SEQ ID NO: 110 - RNA, 71 nt; single-stranded, linear
  All C′s are 2′-NH2 cytosine
  All U′s are 2′-NH2 uracil
gggaggacgaugcgggaugcagcaaccugaaaacggcguccacagguaauaacagcagacgacucgcccga

SEQ ID NO: 111 - RNA, 71 nt; single-stranded, linear
  All C′s are 2′-NH2 cytosine
  All U′s are 2′-NH2 uracil
gggaggacgaugcggaaacucgcuacaaacacccaauccuagaacguuauggagacagacgacucgcccga

SEQ ID NO: 112 - RNA, 71 nt; single-stranded, linear
  All C′s are 2′-NH2 cytosine
  All U′s are 2′-NH2 uracil
gggaggacgaugcggcuagcauagccaccggaacagacagauacgagcacgaucacagacgacucgcccga

SEQ ID NO: 113 - RNA, 71 nt; single-stranded, linear
  All C′s are 2′-NH2 cytosine
  All U′s are 2′-NH2 uracil
gggaggacgaugcgggauucggaguacugaaaaacaacccucaaaagugcauaggcagacgacucgcccga

SEQ ID NO: 114 - RNA, 71 nt; single-stranded, linear
  All C′s are 2′-NH2 cytosine
  All U′s are 2′-NH2 uracil
gggaggacgaugcggguccaggacggaccgcagcugugauacaaucgacuuacaccagacgacucgcccga

SEQ ID NO: 115 - RNA, 71 nt; single-stranded, linear; identical to SEQ ID NO: 111
  All C′s are 2′-NH2 cytosine
  All U′s are 2′-NH2 uracil

SEQ ID NO: 116 - RNA, 70 nt; single-stranded, linear
  All C′s are 2′-NH2 cytosine
  All U′s are 2′-NH2 uracil
gggaggacgaugcggcggcccuuaucggaggucugcgccacuaauuacauccaccagacgacucgcccga

SEQ ID NO: 117 - RNA, 67 nt; single-stranded, linear
  All C′s are 2′-NH2 cytosine
  All U′s are 2′-NH2 uracil
gggaggacgaugcgguccagagcgugaagaucaacgucccggngucgaagacagacgacucgcccga

SEQ ID NO: 118 - RNA, 8 nt; single-stranded, linear
  All C′s are 2′-NH2 cytosine
  All U′s are 2′-NH2 uracil
auguguga

SEQ ID NO: 119 - RNA, 15 nt; single-stranded, linear
  All C′s are 2′-NH2 cytosine
  All U′s are 2′-NH2 uracil
caacaaucaugagur

SEQ ID NO: 120 - RNA, 21 nt; single-stranded, linear
  All C′s are 2′-NH2 cytosine
  All U′s are 2′-NH2 uracil
aacaugaaguaagucaruuag

SEQ ID NO: 121 - RNA, 11 nt; single-stranded, linear
  All C′s are 2′-NH2 cytosine
  All U′s are 2′-NH2 uracil
agaaccgcwag

SEQ ID NO: 122 - RNA, 7 nt; single-stranded, linear
  All C′s are 2′-NH2 cytosine
  All U′s are 2′-NH2 uracil
ucucucc

SEQ ID NO: 123 - RNA, 10 nt; single-stranded, linear
  All C′s are 2′-NH2 cytosine
  All U′s are 2′-NH2 uracil
cgaagaauyc

SEQ ID NO: 124 - RNA, 8 nt; single-stranded, linear
  All C′s are 2′-NH2 cytosine
  All U′s are 2′-NH2 uracil
auguaaau

SEQ ID NO: 125 - RNA, 8 nt; single-stranded, linear
  All C′s are 2′-NH2 cytosine
  All U′s are 2′-NH2 uracil
aacccaag

SEQ ID NO: 126 - DNA, 80 bp; single-stranded, linear
ctacctacgatctgactagcnnnnnnnnnnnnnnnnnnnnnnnnnnnnnnnnnnnnnnnngcttactctcatgtagttcc

SEQ ID NO: 127 - DNA, 20 bp; single-stranded, linear
ctacctacgatctgactagc

SEQ ID NO: 128 - DNA, 25 bp; single-stranded, linear
  N AT POSITION 2 AND 4 IS BIOTIN
ananaggaactacatgagagtaagc

SEQ ID NO: 129 - DNA, 80 bp; single-stranded, linear
ctacctacgatctgactagcggaacacgtgaggtttacaaggcactcgacgtaaacacttgcttactctcatgtagttcc

SEQ ID NO: 130 - DNA, 80 bp; single-stranded, linear
ctacctacgatctgactagcccccgaagaacattttacaaggtgctaaacgtaaaatcaggcttactctcatgtagttcc

SEQ ID NO: 131 - DNA, 80 bp; single-stranded, linear
ctacctacgatctgactagcggcatccctgagtcattacaaggttcttaacgtaatgtacgcttactctcatgtagttcc

SEQ ID NO: 132 - DNA, 80 bp; single-stranded, linear
ctacctacgatctgactagctgcacacctgagggttacaaggcgctagacgtaacctctcgcttactctcatgtagttcc

SEQ ID NO: 133 - DNA, 80 bp; single-stranded, linear
ctacctacgatctgactagccacgtttcaaggggttacacgaaacgattcactccttggcgcttactctcatgtagttcc

SEQ ID NO: 134 - DNA, 79 bp; single-stranded, linear
ctacctacgatctgactagccggacatgagcgttacaaggtgctaaacgtaacgtacttgcttactctcatgtagttcc

SEQ ID NO: 135 - DNA, 79 bp; single-stranded, linear
ctacctacgatctgactagccgcatccacatagttcaaggggctacacgaaatattgcagcttactctcatgtagttcc

SEQ ID NO: 136 - DNA, 79 bp; single-stranded, linear
ctacctacgatctgactagctaccccttgggcctcatagacaaggtcttaaacgttagcgcttactctcatgtagttcc

SEQ ID NO: 137 - DNA, 79 bp; single-stranded, linear
ctacctacgatctgactagccacatgcctgacgcggtacaaggcctggacgtaacgttggcttactctcatgtagttcc

SEQ ID NO: 138 - DNA, 80 bp; single-stranded, linear
ctacctacgatctgactagctagtgctccacgtattcaaggtgctaaacgaagacggcctgcttactctcatgtagttcc

SEQ ID NO: 139 - DNA, 79 bp; single-stranded, linear
ctacctacgatctgactagcagcgatgcaaggggctacacgcaacgatttagatgctctgcttactctcatgtagttcc

SEQ ID NO: 140 - DNA, 79 bp; single-stranded, linear
ctacctacgatctgactagcccaggagcacagtacaaggtgttaaacgtaatgtctggtgcttactctcatgtagttcc

SEQ ID NO: 141 - DNA, 79 bp; single-stranded, linear
ctacctacgatctgactagcaccacacctgggcggtacaaggagttatccgtaacgtgtgcttactctcatgtagttcc

SEQ ID NO: 142 - DNA, 79 bp; single-stranded, linear
ctacctacgatctgactagccaaggtaaccagtacaaggtgctaaacgtaatggcttcggcttactctcatgtagttcc

SEQ ID NO: 143 - DNA, 79 bp; single-stranded, linear
ctacctacgatctgactagcacccccgacccgagtacaaggcattcgacgtaatctggtgcttactctcatgtagttcc

SEQ ID NO: 144 - DNA, 79 bp; single-stranded, linear
ctacctacgatctgactagccagtacaaggtgttaaacgtaatgccgatcgagttgtatgcttactctcatgtagttcc

SEQ ID NO: 145 - DNA, 81 bp; single-stranded, linear
ctacctacgatctgactagcacaacgagtacaaggagatagacgtaatcggcgcaggtatcgcttactctcatgtagttcc

SEQ ID NO: 146 - DNA, 79 bp; single-stranded, linear
ctacctacgatctgactagccacgacagagaacaaggcgttagacgttatccgaccacggcttactctcatgtagttcc

SEQ ID NO: 147 - DNA, 80 bp; single-stranded, linear
ctacctacgatctgactagcagggagaacaaggtgctaaacgtttatctacacttcacctgcttactctcatgtagttcc

SEQ ID NO: 148 - DNA, 80 bp; single-stranded, linear
ctacctacgatctgactagcaggaccaaggtgttaaacggctcccctggctatgcctcttgcttactctcatgtagttcc

SEQ ID NO: 149 - DNA, 80 bp; single-stranded, linear
ctacctacgatctgactagctacacaaggtgctaaacgtagagccagatcggatctgagcgcttactctcatgtagttcc

SEQ ID NO: 150 - DNA, 80 bp; single-stranded, linear
ctacctacgatctgactagcggacaaggcactcgacgtagtttataactccctccgggccgcttactctcatgtagttcc

SEQ ID NO: 151 - DNA, 81 bp; single-stranded, linear
ctacctacgatctgactagctacacaaggggccaaacggagagccagacgcggatctgacagcttactctcatgtagttcc

SEQ ID NO: 152 - DNA, 79 bp; single-stranded, linear
ctacctacgatctgactagccggctatacnnggtgctaaacgcagagactcgatcaacagcttactctcatgtagttcc

SEQ ID NO: 153 - DNA, 80 bp; single-stranded, linear
ctacctacgatctgactagcgagtagccaaggcgttagacggagggggaatggaagcttggcttactctcatgtagttcc

SEQ ID NO: 154 - DNA, 73 bp; single-stranded, linear
ctacctacgatctgactagcgagtagccaaggcgttagacggagggggaatgggcttactctcatgtagttcc

SEQ ID NO: 155 - DNA, 80 bp; single-stranded, linear
ctacctacgatctgactagcgagtagccaaggcgttagacggagggggaatgtgagcacagcttactctcatgtagttcc

SEQ ID NO: 156 - DNA, 80 bp; single-stranded, linear
ctacctacgatctgactagctagctccacacacaasscgcrgcacataggggatatctgggcttactctcatgtagttcc

SEQ ID NO: 157 - DNA, 80 bp; single-stranded, linear
ctacctacgatctgactagccatcaaggactttgcccgaaaccctaggttcacgtgtggggcttactctcatgtagttcc

SEQ ID NO: 158 - DNA, 79 bp; single-stranded, linear
ctacctacgatctgactagccattcaccatggccccttcctacgtatgttctgcgggtggcttactctcatgtagttcc

SEQ ID NO: 159 - DNA, 80 bp; single-stranded, linear
ctacctacgatctgactagcgcaacgtggccccgtttagctcatttgaccgttccatccggcttactctcatgtagttcc

SEQ ID NO: 160 - DNA, 79 bp; single-stranded, linear
ctacctacgatctgactagcccacagacaatcgcagtccccgtgtagctctgggtgtctgcttactctcatgtagttcc

SEQ ID NO: 161 - DNA, 79 bp; single-stranded, linear
ctacctacgatctgactagcccaccgtgatgcacgatacatgagggtgtgtcagcgcatgcttactctcatgtagttcc

SEQ ID NO: 162 - DNA, 80 bp; single-stranded, linear
ctacctacgatctgactagccgaggtagtcgttatagggtrcrcacgacacaaarcrgtrgcttactctcatgtagttcc

SEQ ID NO: 163 - DNA, 79 bp; single-stranded, linear
ctacctacgatctgactagctggcggtacgggccgtgcacccacttacctgggaagtgagcttactctcatgtagttcc

SEQ ID NO: 164 - DNA, 81 bp; single-stranded, linear
ctacctacgatctgactagcctctgcttacctcatgtagttccaagcttggcgtaatcatggcttactctcatgtagttcc

SEQ ID NO: 165 - DNA, 79 bp; single-stranded, linear
ctacctacgatctgactagcagcgttgtacggggttacacacaacgatttagatgctctgcttactctcatgtagttcc

SEQ ID NO: 166 - DNA, 81 bp; single-stranded, linear
ctacctacgatctgactagctgatgcgactttagtcgaacgttactggggctcagaggacagcttactctcatgtagttcc

SEQ ID NO: 167 - DNA, 81 bp; single-stranded, linear
ctacctacgatctgactagccgaggatctgatacttattgaacatamccgcacncaggcttgcttactctcatgtagttcc

SEQ ID NO: 168 - DNA, 73 bp; single-stranded, linear
ctacctacgatctgactagccgatcgtgtgtcatgctacctacgatctgactagcttactctcatgtagttcc

SEQ ID NO: 169 - DNA, 80 bp; single-stranded, linear
ctacctacgatctgactagcgcacacaagtcaagcatgcgaccttcaaccatcgacccgagcttactctcatgtagttcc

SEQ ID NO: 170 - DNA, 80 bp; single-stranded, linear
ctacctacgatctgactagcatgccagtgcaggcttccatccatcagtctgacannnnnngcttactctcatgtagttcc

SEQ ID NO: 171 - DNA, 79 bp; single-stranded, linear
ctacctacgatctgactagccacttcggctctactccacctcggtcctccactccacaggcttactctcatgtagttcc

SEQ ID NO: 172 - DNA, 80 bp; single-stranded, linear
ctacctacgatctgactagccgctaactgaccctcgatccccccaagccatcctcatcgcgcttactctcatgtagttcc

SEQ ID NO: 173 - DNA, 90 bp; single-stranded, linear
ctacctacgatctgactagcatctgactagctcggcgagagtacccgctcatggcttcggcgaatgccctgcttactctcatgtagttcc

SEQ ID NO: 174 - DNA, 80 bp; single-stranded, linear
ctacctacgatctgactagctcctgagacgttacaataggctgcggtactgcaacgtggagcttactctcatgtagttcc

SEQ ID NO: 175 - DNA, 79 bp; single-stranded, linear
ctacctacgatctgactagccggcagggcactaacaaggtgttaaacgttacggatgccgcttactctcatgtagttcc

SEQ ID NO: 176 - DNA, 90 bp; single-stranded, linear
ctacctacgatctgactagctgcacaccggcccacccggacaaggcgctagacgaaatgactctgttctggcttactctcatgtagttcc

SEQ ID NO: 177 - DNA, 79 bp; single-stranded, linear
ctacctacgatctgactagcgacgaagaggccaaggtgataaccggagtttccgtccgcgcttactctcatgtagttcc

SEQ ID NO: 178 - DNA, 79 bp; single-stranded, linear
ctacctacgatctgactagcaaggacttagctatccaaggcactcgacgaagagcccgagcttactctcatgtagttcc

SEQ ID NO: 179 - DNA, 80 bp; single-stranded, linear
ctacctacgatctgactagcatgcccagttcaaggttctgaccgaaatgactctgttctggcttactctcatgtagttcc

SEQ ID NO: 180 - DNA, 80 bp; single-stranded, linear
ctacctacgatctgactagcgcagcgtggccctgtttagctcatttgaccgttccatccggcttactctcatgtagttcc

SEQ ID NO: 181 - DNA, 18 bp; single-stranded, linear
tacaaggygytavacgta

SEQ ID NO: 182 - DNA, 8 bp; single-stranded, linear
ggccccgt

SEQ ID NO: 183 - DNA, 10 bp; single-stranded, linear
rcacgayaca

SEQ ID NO: 184 - DNA, 7 bp; single-stranded, linear
cttacct

SEQ ID NO: 185 - DNA, 49 bp; single-stranded, linear
tagccaaggtaaccagtacaaggtgctaaacgtaatggcttcggcttac

SEQ ID NO: 186 - DNA, 41 bp; single-stranded, linear
gtaaccagtacaaggtgctaaacgtaatggcttcggcttac

SEQ ID NO: 187 - DNA, 26 bp; single-stranded, linear
ccagtacaaggtgctaaacgtaatgg

SEQ ID NO: 188 - DNA, 38 bp; single-stranded, linear
cgcggtaaccagtacaaggtgctaaacgtaatggcgcg

SEQ ID NO: 189 - DNA, 36 bp; single-stranded, linear
gcggtaaccagtacaaggtgctaaacgtaatggcgc

SEQ ID NO: 190 - DNA, 50 bp; single-stranded, linear
acatgagcgttacaaggtgctaaacgtaacgtacttgcttactctcatgt

SEQ ID NO: 191 - DNA, 44 bp; single-stranded, linear
cgcgcgttacaaggtgctaaacgtaacgtacttgcttactcgcg

SEQ ID NO: 192 - DNA, 26 bp; single-stranded, linear
gcgttacaaggtgctaaacgtaacgt

SEQ ID NO: 193 - DNA (<Unknown>), 52 bp; single-stranded, linear
  N at position 1 is an amino modifier C6 dT
  Nucleotide 51 is an inverted- orientation (3′3′ linkage) phosphoramidite
ntagccaaggtaaccagtacaaggtgctaaacgtaatggcttcggcttactt

SEQ ID NO: 194 - DNA, 48 bp; single-stranded, linear
tagccattcaccatggccccttcctacgtatgttctgcgggtggctta

SEQ ID NO: 195 - DNA, 47 bp; single-stranded, linear
agctggcggtacgggccgtgcacccacttacctgggaagtgagctta

SEQ ID NO: 196 - DNA, 29 bp; single-stranded, linear
  N at position 1 is an amimo modifier C6 dT
  Nucleotide number 28 is an inverted-orientation (3′3′ linkage) phosphoramidite
nccagtacaaggtgctaaacgtaatggtt

SEQ ID NO: 197 - DNA, 40 bp; single-stranded, linear
taatacgactcactatagggagacaagaataaacgctcaa

SEQ ID NO: 198 - DNA, 24 bp; single-stranded, linear
gcctgttgtgagcctcctgtcgaa

SEQ ID NO: 199 - RNA, 96 nt; single-stranded, linear
  All C′s are 2′-F cytosine
  All U′s are 2′-F uracil
gggagacaagaauaaacgcucaacgaaucaguaaacauaacaccaugaaacauaaauagcacgcgagacgucuucgacaggaggcucacaacaggc

SEQ ID NO: 200 - RNA, 95 nt; single-stranded, linear
  All C′s are 2′-F cytosine
  All U′s are 2′-F uracil
gggagacaagaauaaacgcucaacgaguucacaugggagcaaucuccgaauaaacaacacgckakcgcaaauucgacaggaggcucacaacaggc

SEQ ID NO: 201 - RNA, 96 nt; single-stranded, linear
  All C′s are 2′-F cytosine
  All U′s are 2′-F uracil
gggagacaagaauaaacgcucaacgaccacaauacaaacucguauggaacacgcgagcgacagugacgcauuuucgacaggaggcucacaacaggc

SEQ ID NO: 202 - RNA, 97 nt; single-stranded, linear
  All C′s are 2′-F cytosine
  All U′s are 2′-F uracil
gggagacaagaauaaacgcucaacgucaagccagaauccggaacacgcgagaaaacaaaucaacgaccaaucgauucgacaggaggcucacaaaggc

SEQ ID NO: 203 - RNA, 97 nt; single-stranded, linear
  All C′s are 2′-F cytosine
  All U′s are 2′-F uracil
gggagacaagaauaaacncucaacgaccacaauaaccggaaauccccgcgguuacggaacacgcgaacaugaauucgacaggaggcucacaacaggc

SEQ ID NO: 204 - RNA, 95 nt; single-stranded, linear
  All C′s are 2′-F cytosine
  All U′s are 2′-F uracil
gggagacaagaauaaacgcucaacgaaccacggggaaauccaccaguaacacgcgaggcaaacagacccucuucgacaggaggcucacaacaggc

SEQ ID NO: 205 - RNA, 97 nt; single-stranded, linear
  All C′s are 2′-F cytosine
  All U′s are 2′-F uracil
gggagacaagaauaaacgcucaacgagcaaaaguacucacgggaccaggagaucagcaacacgcgagacgaaauucgacaggaggcucacaacaggc

SEQ ID NO: 206 - RNA, 96 nt; single-stranded, linear
  All C′s are 2′-F cytosine
  All U′s are 2′-F uracil
gggagacaagaauaaacgcucaacgagccaggaacaucgacgucagcaaacgcgagcgcaaccaguaacaccuucgacaggaggcucacaacaggc

SEQ ID NO: 207 - RNA, 94 nt; single-stranded, linear
  All C′s are 2′-F cytosine
  All U′s are 2′-F uracil
gggagacaagaauaaacgcucaacgcaccaggaacaacgagaaccaucaguaaacgcgagcgauugcauguucgacaggaggcucacaacaggc

SEQ ID NO: 208 - RNA, 94 nt; single-stranded, linear
  All C′s are 2′-F cytosine
  All U′s are 2′-F uracil
gggagacaagaauaaacgcucaacgcaccaggaacaacaagaaccaucaguaagcgcgagcgauugcauauucgacaggaggcucacaacaggc

SEQ ID NO: 209 - RNA, 101 nt; single-stranded, linear
  All C′s are 2′-F cytosine
  All U′s are 2′-F uracil
gggagacaagaauaaacgcucaacgagcaaggaacgaauacaaaccaggaaacucagcaacacgcgagcaguaagaauucgacaggaggcucacaacaggc

SEQ ID NO: 210 - DNA (<Unknown>), 97 bp; single-stranded, linear
  All C′s are 2′-F cytosine
  All U′s are 2′-F uracil
gggagacaagaauaaacgcucaacaguucacucaaccggcaccagacuacgaucagcauuggcgagugaacacuucgacaggaggcucacaacaggc

SEQ ID NO: 211 - RNA, 96 nt; single-stranded, linear
  All C′s are 2′-F cytosine
  All U′s are 2′-F uracil
gggagacaagaauaaacgcucaacuggcaacgggauaacaacaaaugucaccagcacuagcgagacggaagguucgacaggaggcucacaacaggc

SEQ ID NO: 212 - RNA, 97 nt; single-stranded, linear
  All C′s are 2′-F cytosine
  All U′s are 2′-F uracil
gggagacaagaauaaacgcucaacgaugagcgugaccgaagcuauaaucaggucgauucaccaagcaaucuuauucgacaggaggcucacaacaggc

SEQ ID NO: 213 - RNA, 95 nt; single-stranded, linear
  All C′s are 2′-F cytosine
  All U′s are 2′-F uracil
gggagacaagaauaaacgcucaaaggaucacacaaacaucggucaauaaauaaguauugauagcggggauauucgacaggaggcucacaacaggc

SEQ ID NO: 214 - RNA, 97 nt; single-stranded, linear
  All C′s are 2′-F cytosine
  All U′s are 2′-F uracil
gggagacaagaauaaacgcucaacaacccaaccaucuagagcuucgaaccaugguauacaagggaacacaaaauucgcggaggcuccaacaggcggc

SEQ ID NO: 215 - RNA, 96 nt; single-stranded, linear
  All C′s are 2′-F cytosine
  All U′s are 2′-F uracil
gggagacaagaauaaacgcucaagcggucagaaacaauagcuggauacauaccgcgcauccgcugggcgauauucgacaggaggcucacaacaggc

SEQ ID NO: 216 - RNA, 97 nt; single-stranded, linear
  All C′s are 2′-F cytosine
  All U′s are 2′-F uracil
gggagacaagaauaaacgcucaaacaagagagucaaaccaagugagaucagagcguuuagcgcggaaagcacauucgacaggaggcucacaacaggc

SEQ ID NO: 217 - RNA, 96 nt; single-stranded, linear
  All C′s are 2′-F cytosine
  All U′s are 2′-F uracil
gggagacaagaauaaacgcucaaacucgacuaguaaucacccuagcauaaaucuccucgagcacagacgauauucgacaggaggcucacaacaggc

SEQ ID NO: 218 - RNA, 94 nt; single-stranded, linear
  All C′s are 2′-F cytosine
  All U′s are 2′-F uracil
gggagacaagaauaaacgcucaaucagcaguaagcgauccuauaaagaucaacuagccaaagaugacuuauucgacaggaggcucacaacaggc

SEQ ID NO: 219 - RNA, 95 nt; single-stranded, linear
  All C′s are 2′-F cytosine
  All U′s are 2′-F uracil
gggagacaagaauaaacgcucaaaaagacguauucgauucgaaacgagaaagacuucaagugagcccgcaguucgacaggaggcucacaacaggc

SEQ ID NO: 220 - RNA, 49 nt; single-stranded, linear
  All C′s are 2′-F cytosine
  All U′s are 2′-F uracil
cucaacgaaucaguaaacauaacaccaugaaacauaaauagcacgcgag

SEQ ID NO: 221 - RNA, 47 nt; single-stranded, linear
  All C′s are 2′-F cytosine
  All U′s are 2′-F uracil
cucaacgaguucacaugggagcaaucuccgaauaaacaacacgcgag

SEQ ID NO: 222 - RNA, 39 nt; single-stranded, linear
  All C′s are 2′-F cytosine
  All U′s are 2′-F uracil
cucaacgaaccacggggaaauccaccaguaacacgcgag

SEQ ID NO: 223 - RNA, 38 nt; single-stranded, linear
  All C′s are 2′-F cytosine
  All U′s are 2′-F uracil
cucaacgagccaggaacaucgacgucagcaaacgcgag

SEQ ID NO: 224 - RNA, 42 nt; single-stranded, linear
  All C′s are 2′-F cytosine
  All U′s are 2′-F uracil
cgcucaacgagccaggaacaucgacgucagcaaacgcgagcg

SEQ ID NO: 225 - RNA, 35 nt; single-stranded, linear
  All C′s are 2′-F cytosine
  All U′s are 2′-F uracil
cucaacgagccaggacuacgaucagcaaacgcgag

SEQ ID NO: 226 - RNA, 42 nt; single-stranded, linear
  All C′s are 2′-F cytosine
  All U′s are 2′-F uracil
cucaacgcaccaggaacaacgagaaccaucaguaaacgcgag

SEQ ID NO: 227 - RNA, 42 nt; single-stranded, linear
  All C′s are 2′-F cytosine
  All U′s are 2′-F uracil
cucaacgcaccaggaacaacaagaaccaucaguaagcgcgag

SEQ ID NO: 228 - RNA, 40 nt; single-stranded, linear
  All C′s are 2′-F cytosine
  All U′s are 2′-F uracil
cacucaaccggcaccagacuacgaucagcauuggcgagug

SEQ ID NO: 229 - RNA, 45 nt; single-stranded, linear
  All C′s are 2′-F cytosine
  All U′s are 2′-F uracil
gaauccggaacacgcgagaaaacaaaucaacgaccaaucgauucg

SEQ ID NO: 230 - RNA, 38 nt; single-stranded, linear; identical to SEQ ID NO: 223
  All C′s are 2′-F cytosine
  All U′s are 2′-F uracil
  7, 9, 14, 21: G are 2′-O-methyl guanine
  8, 15, 18, 22, 27, 31: A are 2′-O-methly adenine

SEQ ID NO: 231 - RNA, 38 nt; single-stranded, linear; identical to SEQ ID NO: 223
  All C′s are 2′-F cytosine
  All U′s are 2′-F uracil
  7, 9, 13, 14, 21, 24, 28: G are 2′-O-methyl-guanine
  8, 15, 18, 22, 27, 30, 31: A are 2′-O-methyl-adenine

SEQ ID NO: 232 - RNA, 38 nt; single-stranded, linear; identical to SEQ ID NO: 223
  All C′s are 2′-F cytosine
  All U′s are 2′-F uracil
  7, 9, 14, 21, 36: G are 2′-O-methyl-guanine
  8, 15, 18, 22, 27, 31, 37: A are 2′-O-methyl-adenine

SEQ ID NO: 233 - RNA, 38 nt; single-stranded, linear; identical to SEQ ID NO: 223
  All C′s are 2′-F cytosine
  All U′s are 2′-F uracil
  7, 9, 13, 14, 21, 24, 28, 36: G are 2′-O-methyl-guanine
  8, 15, 18, 22, 27, 30, 31, 37: A are 2′-O-methyl-adenine

SEQ ID NO: 234 - RNA, 38 nt; single-stranded, linear; identical to SEQ ID NO: 223
  All C′s are 2′-F cytosine
  All U′s are 2′-F uracil
  7, 9, 14: G are 2′-O-methyl-guanine
  8, 15, 18, 27, 31: A are 2′-O-methyl-adenine

SEQ ID NO: 235 - RNA, 38 nt; single-stranded, linear; identical to SEQ ID NO: 223
  All C′s are 2′-F cytosine
  All U′s are 2′-F uracil
  7, 9, 13, 14, 24: G are 2′-O-methyl-guanine
  8, 15, 18, 22, 27, 31: A are 2′-O-methyl-adenine

SEQ ID NO: 236 - RNA, 59 nt; single-stranded, linear
  All C′s are 2′-F cytosine
  All U′s are 2′-F uracil
cucaacgagcaaaaguacucacgggaccaggagaucagcaacacgcgagacgaaauucg

SEQ ID NO: 237 - RNA, 43 nt; single-stranded, linear
  All C′s are 2′-F cytosine
  All U′s are 2′-F uracil
cgcucaacgaccacaauacaaacucguauggaacacgcgagcg

SEQ ID NO: 238 - RNA, 51 nt; single-stranded, linear
  All C′s are 2′-F cytosine
  All U′s are 2′-F uracil
cgcucaacuggcaacgggauaacaacaaaugucaccagcacuagcgagacg

SEQ ID NO: 239 - RNA, 41 nt; single-stranded, linear
  All C′s are 2′-F cytosine
  All U′s are 2′-F uracil
ucacucaaccggcaccagacuacgaucagcauuggcgagug

SEQ ID NO: 240 - RNA, 70 nt; single-stranded, linear
  All C′s are 2′-F cytosine
  All U′s are 2′-F uracil
gggagacaagaauaaacgcucaacgagcaaggaacgaauacaaaccaggaaacucagcaacacgcgagca

SEQ ID NO: 241 - RNA, 51 nt; single-stranded, linear
  All C′s are 2′-F cytosine
  All U′s are 2′-F uracil
cucaacgaccacaauaaccggaaauccccgcgguuacggaacacgcgaaca

SEQ ID NO: 242 - RNA, 69 nt; single-stranded, linear
  All C′s are 2′-F cytosine
  All U′s are 2′-F uracil
agaauaaacgcucaacgaugagcgugaccgaagcuauaaucaggucgauucaccaagcaaucuuauucg

SEQ ID NO: 243 - RNA, 50 nt; single-stranded, linear
  All C′s are 2′-F cytosine
  All U′s are 2′-F uracil
acgcucaaaggaucacacaaacaucggucaauaaauaaguauugauagcg

SEQ ID NO: 244 - RNA, 52 nt; single-stranded, linear
  All C′s are 2′-F cytosine
  All U′s are 2′-F uracil
gcucaagcggucagaaacaauagcuggauacauaccgcgcauccgcugggcg

SEQ ID NO: 245 - RNA, 58 nt; single-stranded, linear
  All C′s are 2′-F cytosine
  All U′s are 2′-F uracil
accaucuagagcuucgaaccaugguauacaagggaacacaaaauucgcggaggcucca

SEQ ID NO: 246 - RNA, 96 nt; single-stranded, linear
  All C′s are 2′-F cytosine
  All U′s are 2′-F uracil
gggagacaagauaaacgcucaaacaagagagucaaaccaagugagaucagagcguuuagcgcggaaagcacauucgacaggaggcucacaacaggc

SEQ ID NO: 247 - RNA, 87 nt; single-stranded, linear
  All C′s are 2′-F cytosine
  All U′s are 2′-F uracil
gggagacaagaauaaacgcucaaaaagacguauucgauucgaaacgagaaagacuucaagugagcccgcaguucgacaggaggcuca

SEQ ID NO: 248 - RNA, 97 nt; single-stranded, linear
  All C′s are 2′-NH2 cytosine
  All U′s are 2′-NH2 uracil
gggagacaagaauaaacgcucaannnnnnnnnnnnnnnnnnnnnnnnnnnnnnnnnnnnnnnnnnnnnnnnnnuucgacaggaggcucacaacaggc

SEQ ID NO: 249 - DNA, 40 bp; single-stranded, linear; identical to SEQ ID NO: 197

SEQ ID NO: 250 - DNA, 24 bp; single-stranded, linear
gcctgttgtgagcctcctgtcgaa

SEQ ID NO: 251 - RNA, 97 nt; single-stranded, linear
  All C′s are 2′-NH2 cytosine
  All U′s are 2′-NH2 uracil
gggagacaagaauaaacgcucaagccccaaacgcaagcgagcauccgcaacagggaagaagacagacgaaugauucgacaggaggcucacaacaggc

SEQ ID NO: 252 - RNA, 97 nt; single-stranded, linear
  All C′s are 2′-NH2 cytosine
  All U′s are 2′-NH2 uracil
gggagacaagaauaaacgcucaagccccaaacgcaagugagcauccgcaacagggaagaagacagacgauugauucgacaggaggcucacaacaggc

SEQ ID NO: 253 - RNA, 98 nt; single-stranded, linear
  All C′s are 2′-NH2 cytosine
  All U′s are 2′-NH2 uracil
gggagacaagaaauaaacncucaagccccaaacgcaagugagcauccgcaacagggaagaagacagaugaaugauucgacaggaggcucacaacaggc

SEQ ID NO: 254 - RNA, 95 nt; single-stranded, linear
  All C′s are 2′-NH2 cytosine
  All U′s are 2′-NH2 uracil
gggagacaagaauaaacncucaagccccaagcaagugagcauccgcaacagggaagaagacagacgagugauucgacaggaggcucacaacaggc

SEQ ID NO: 255 - RNA, 97 nt; single-stranded, linear
  All C′s are 2′-NH2 cytosine
  All U′s are 2′-NH2 uracil
gggagacaagaauaaacncucaagccccaaacgcaagugagcauccgcaacagggaagaagacagacgaaugauucgacaggaggcucacaacaggc

SEQ ID NO: 256 - RNA, 97 nt; single-stranded, linear
  All C′s are 2′-NH2 cytosine
  All U′s are 2′-NH2 uracil
gggagacaagaauaaacgcucaagcaaaaggcguaaauacaccuccgcaacugggaagaagacgcagggacgguucgacaggnggcucacaacaggc

SEQ ID NO: 257 - RNA, 98 nt; single-stranded, linear
  All C′s are 2′-NH2 cytosine
  All U′s are 2′-NH2 uracil
gggagacaagaauaaacgcucaaacagcuacaagugggacaacaggguacagcggagagaaacauccaaacaaguucgacaggaggcucacaacaggc

SEQ ID NO: 258 - RNA, 95 nt; single-stranded, linear
  All C′s are 2′-NH2 cytosine
  All U′s are 2′-NH2 uracil
gggagacaagaauaaacgcucaaaucaacuaaacaacgcagucacgagaacgaccggkcugacuccgaaaguucgacaggaggcucacaacaggc

SEQ ID NO: 259 - RNA, 95 nt; single-stranded, linear
  All C′s are 2′-NH2 cytosine
  All U′s are 2′-NH2 uracil
gggagacaagaauaaacgcucaaacgagagcaccaaggcaacagaugcagaagaagugugcgcgcgcgaaauucgacaggaggcucacaacaggc

SEQ ID NO: 260 - RNA, 98 nt; single-stranded, linear
  All C′s are 2′-NH2 cytosine
  All U′s are 2′-NH2 uracil
gggagacaagaauaaacgcucaauaagacaacgaacagacagaagcgaaaaaggggcgccgcagcaacaacaaauucgacaggaggcucacaacaggc

SEQ ID NO: 261 - RNA, 94 nt; single-stranded, linear
  All C′s are 2′-NH2 cytosine
  All U′s are 2′-NH2 uracil
gggagacaagaauaaacgcucaacguguaccacaacaguuccacggaagcuggaauaggacgcagaggaauucgacaggaggcucacaacaggc

SEQ ID NO: 262 - RNA, 94 nt; single-stranded, linear
  All C′s are 2′-NH2 cytosine
  All U′s are 2′-NH2 uracil
gggagacaagaauaaacgcucaaacaaaauuwuggugggccccgcaacmgggrggragrccguugaaggcuucgacaggaggcucacaacaggc

SEQ ID NO: 263 - RNA, 94 nt; single-stranded, linear
  All C′s are 2′-NH2 cytosine
  All U′s are 2′-NH2 uracil
gggagacaagaauaaacgcucaagaucauaacgagaggagagggagaacuacacgcgcgcgaggaaagaguucgacaggaggcucacaacaggc

SEQ ID NO: 264 - RNA, 89 nt; single-stranded, linear
  All C′s are 2′-NH2 cytosine
  All U′s are 2′-NH2 uracil
gggagacaagaauaaacgcucaaacacaaaucgggcagggacuggguugggcacggcagggcgccuucgacaggaggcucacaacaggc

SEQ ID NO: 265 - RNA, 97 nt; single-stranded, linear
  All C′s are 2′-NH2 cytosine
  All U′s are 2′-NH2 uracil
gggagacaagaauaaacgcucaagugggcucgggccggaugucuacgggugugaagaaaccccuagggcaggguucgacaggaggcucacaacaggc

SEQ ID NO: 266 - DNA (<Unknown>), 95 bp; single-stranded, linear
  All U′s are 2′-NH2 uracil
gggagacaagaauaaacgcucaagaucagcggaacuaagaaauggaaggcuaagcaccgggaucgggagaauucgacaggaggcucacaacaggc

SEQ ID NO: 267 - RNA, 95 nt; single-stranded, linear
  All C′s are 2′-NH2 cytosine
  All U′s are 2′-NH2 uracil
gggagacaagaauaaacgcucaauaacaaagcagcaaaguaccagaggagaguuggcaggguuuaggcagcuucgacaggaggcucacaacaggc

SEQ ID NO: 268 - RNA, 95 nt; single-stranded, linear
  All C′s are 2′-NH2 cytosine
  All U′s are 2′-NH2 uracil
gggagacagaauaaacgcucaaagaccaagggacagcagcggggaaaaacagaucacagcuguaagagggcuucgacaggaggcucacaacaggc

SEQ ID NO: 269 - RNA, 93 nt; single-stranded, linear
  All C′s are 2′-NH2 cytosine
  All U′s are 2′-NH2 uracil
gggagacaagaauaaacgcucaaagucggggauagaaacacacuaagaagugcaucagguaggagauaauucgacaggnggcucacaacaggc

SEQ ID NO: 270 - RNA, 95 nt; single-stranded, linear
  All C′s are 2′-NH2 cytosine
  All U′s are 2′-NH2 uracil
gggagacaagaauaaacgcucaagaguaucacacaaaccggcacggacuaagcagaaggagguacggaagauucgacaggaggcucacaacaggc

SEQ ID NO: 271 - RNA, 94 nt; single-stranded, linear
  All C′s are 2′-NH2 cytosine
  All U′s are 2′-NH2 uracil
gggagacaagaauaaacncucaacgaaauagaaggaacagaagaauggbgawgngggaaauggcaacgaauucgacaggnggcucacaacaggc

SEQ ID NO: 272 - RNA, 97 nt; single-stranded, linear
  All C′s are 2′-NH2 cytosine
  All U′s are 2′-NH2 uracil
gggagacaagaauaaacgcucaaacgagacccuggauacgaggcugagggaaagggagmmmrramcuarrckcuucgacaggaggcucacaacaggc

SEQ ID NO: 273 - RNA, 96 nt; single-stranded, linear
  All C′s are 2′-NH2 cytosine
  All U′s are 2′-NH2 uracil
gggagacaagaauaaacgcucaagaaggauacuuaggacuacgugggaugggaugaaaugggagaacgggaguucgacaggaggcucacaacaggc

SEQ ID NO: 274 - RNA, 96 nt; single-stranded, linear
  All C′s are 2′-NH2 cytosine
  All U′s are 2′-NH2 uracil
gggagacaagaauaaacgcucaaaacgcacaaaguaagggacgggauggaucgcccuaggcuggaagggaacuucgacaggaggcucacaacaggc

SEQ ID NO: 275 - RNA, 96 nt; single-stranded, linear
  All C′s are 2′-NH2 cytosine
  All U′s are 2′-NH2 uracil
gggagacaagaauaaacgcucaaggugaacggcagcaaggcccaaaacguaaggccggaaacnggagagggauucgacaggnggcucacaacaggc

SEQ ID NO: 276 - RNA, 96 nt; single-stranded, linear
  All C′s are 2′-NH2 cytosine
  All U′s are 2′-NH2 uracil
gggagacaagaauaaacgcucaaugauauacacguaagcacugaaccaggcugagauccaucagugcccagguucgacaggaggcucacaacaggc

SEQ ID NO: 277 - RNA, 94 nt; single-stranded, linear; identical to SEQ ID NO: 263
  All C′s are 2′-NH2 cytosine
  All U′s are 2′-NH2 uracil

SEQ ID NO: 278 - RNA, 96 nt; single-stranded, linear
  All C′s are 2′-NH2 cytosine
  All U′s are 2′-NH2 uracil
gggagacaagaauaaacgcucaaucaaguaaggaggaagggucgugacagaaaaacgagcaaaaaacgcgaguucgacaggaggcucacaacaggc

SEQ ID NO: 279 - RNA, 93 nt; single-stranded, linear
  All C′s are 2′-NH2 cytosine
  All U′s are 2′-NH2 uracil (xi)
gggagacaagaauaaacgcucaaaaggugccggguuggagggguagcaagaaauggcuagggcgcasgauucgacaggnggcucacaacaggc

SEQ ID NO: 280 - RNA, 97 nt; single-stranded, linear
  All C′s are 2′-NH2 cytosine
  All U′s are 2′-NH2 uracil
gggagacaagaauaaacgcucaaccaacgcgcaccccgcagcaaacgaaauuggggagacaggugcaagacaguucgacaggaggcucacaacaggc

SEQ ID NO: 281 - RNA, 97 nt; single-stranded, linear
  All C′s are 2′-NH2 cytosine
  All U′s are 2′-NH2 uracil
gggagacaagaauaaackcucaacaaacaauaucggcgcaggaaaacguagaaacgaaamggagcugcgyggauucgacaggaggcucacaacaggc

SEQ ID NO: 282 - RNA, 93 nt; single-stranded, linear
  All C′s are 2′-NH2 cytosine
  All U′s are 2′-NH2 uracil
gggagacaagaauaaacgcucaaugauagcacaguguauaagaaaacgcaacaccgcgcgcggaaagaguucgacaggaggcucacaacaggc

SEQ ID NO: 283 - RNA, 96 nt; single-stranded, linear
  All C′s are 2′-NH2 cytosine
  All U′s are 2′-NH2 uracil
gggagacaagaauaaacgcucaagaucaucgcaguaucggaaucgacccucagugggugacaugcggacaaguucgacaggaggcucacaacaggc

SEQ ID NO: 284 - RNA, 97 nt; single-stranded, linear
  All C′s are 2′-NH2 cytosine
  All U′s are 2′-NH2 uracil
gggagacaagaauaaacgcucaaguaccgggaagggaugaacugggauaugggaacggaggucagaggcacgauucgacaggaggcucacaacaggc

SEQ ID NO: 285 - RNA, 97 nt; single-stranded, linear
  All C′s are 2′-NH2 cytosine
  All U′s are 2′-NH2 uracil
gggagacaagaauaaacgcucaagcaauggaacgcuaggagggaacauaagcagggcgagcggagucgauagcuucgacaggaggcucacaacaggc

SEQ ID NO: 286 - RNA, 97 nt; single-stranded, linear
  All C′s are 2′-NH2 cytosine
  All U′s are 2′-NH2 uracil
gggagacaagaauaaacgcucaaaacagaacugaucggcgcagguugauaaaggggcagcgcgaagaucacaauucgacaggaggcucacaacaggc

SEQ ID NO: 287 - RNA, 97 nt; single-stranded, linear
  All C′s are 2′-NH2 cytosine
  All U′s are 2′-NH2 uracil
gggagacaagaauaaacgcucaagggaaacggaaagggacaaggcgaacagacgagaaguagacggaguaggauucgacaggaggcucacaacaggc

SEQ ID NO: 288 - RNA, 97 nt; single-stranded, linear
  All C′s are 2′-NH2 cytosine
  All U′s are 2′-NH2 uracil
gggagacaagaauaaacgcucaannngaggaagggcacgcaaggaaacaaaacacaaagcagaaguaguaagauucgacaggaggcucacaacaggc

SEQ ID NO: 289 - RNA, 95 nt; single-stranded, linear
  All C′s are 2′-NH2 cytosine
  All U′s are 2′-NH2 uracil
gggagacaagaauaaacgcucaaguacrcagugagcagaagcagagagacuugggaugggaugaaauggkcuucgacaggaggcucacaacaggc

SEQ ID NO: 290 - RNA, 97 nt; single-stranded, linear
  All C′s are 2′-NH2 cytosine
  All U′s are 2′-NH2 uracil
gggagacaagaauaaacncucaaccgacguggacdcgcaucggcauccagaccaggcugnbcngcaccasacguucgacaggaggcucacaacaggc

SEQ ID NO: 291 - RNA, 11 nt; single-stranded, linear
  All C′s are 2′-NH2 cytosine
  All U′s are 2′-NH2 uracil
gggaagaagac

SEQ ID NO: 292 - RNA, 66 nt; single-stranded, linear
  All C′s are 2′-F cytosine
  All U′s are 2′-F uracil
gggaggacgaugcggnnnnnnnnnnnnnnnnnnnnnnnnnnnnnnnnnnncagacgacucgcccga

SEQ ID NO: 293 - RNA, 61 nt; single-stranded, linear
  All C′s are 2′-F cytosine
  All U′s are 2′-F uracil
gggaggacgaugcgggcaaauugcaugcguuuucgagugcuugcucagacgacucgcccga

SEQ ID NO: 294 - RNA, 71 nt; single-stranded, linear
  All C′s are 2′-F cytosine
  All U′s are 2′-F uracil
gggaggacgaugcggugcuuaaacaacgcgugaaucgaguucauccacuccuccucagacgacucgcccga

SEQ ID NO: 295 - RNA, 72 nt; single-stranded, linear
  All C′s are 2′-F cytosine
  All U′s are 2′-F uracil
gggaggacgaugcgguuaauucagucucaaacggugcguuuaucgagccacugaucwgacgacucgcccgaa

SEQ ID NO: 296 - RNA, 71 nt; single-stranded, linear
  All C′s are 2′-F cytosine
  All U′s are 2′-F uracil
gggaggacgaugcggcuuagagcucaaacggugugacuuucaagcccucuaugcccagacgacucgcccga

SEQ ID NO: 297 - RNA, 61 nt; single-stranded, linear
  All C′s are 2′-F cytosine
  All U′s are 2′-F uracil
gggaggacgaugcgguaccucaaauugcguguuuucaagcaguaucagacgacucgcccga

SEQ ID NO: 298 - RNA, 61 nt; single-stranded, linear
  All C′s are 2′-F cytosine
  All U′s are 2′-F uracil
gggaggacgaugcggacccucaaauaacgugucuuucaaguuggucagacgacucgcccga

SEQ ID NO: 299 - RNA, 61 nt; single-stranded, linear
  All C′s are 2′-F cytosine
  All U′s are 2′-F uracil
gggaggacgaugcggacccucaaauagcgugcauuucaagcuggucagacgacucgcccga

SEQ ID NO: 300 - RNA, 61 nt; single-stranded, linear
  All C′s are 2′-F cytosine
  All U′s are 2′-F uracil
gggaagacgaugcggcgcucaaauaaugcguuaaucgaauucgcccagacgacucgcccga

SEQ ID NO: 301 - RNA, 71 nt; single-stranded, linear
  All C′s are 2′-F cytosine
  All U′s are 2′-F uracil
gggaggacgaugcggcaaacaagcucaaaugacguguuuuucaaguccuuguugucagacgacucgcccga

SEQ ID NO: 302 - RNA, 71 nt; single-stranded, linear
  All C′s are 2′-F cytosine
  All U′s are 2′-F uracil
gggaggacgaugcgguaguaagucucaaauguugcguuuuucgaaacacuuacaucagacgacucgcccga

SEQ ID NO: 303 - RNA, 62 nt; single-stranded, linear
  All C′s are 2′-F cytosine
  All U′s are 2′-F uracil
gggaggacgaugcggagacucaaaugguguguuuucaagccucucccagucgacucgcccga

SEQ ID NO: 304 - RNA, 63 nt; single-stranded, linear
  All C′s are 2′-F cytosine
  All U′s are 2′-F uracil
gggaggacgaugcggugcucaaaugaugcguuucucgaauccacccagacgacucgcccgagg

SEQ ID NO: 305 - RNA, 71 nt; single-stranded, linear
  All C′s are 2′-F cytosine
  All U′s are 2′-F uracil
gggaggacgaugcggccaucggucuugggcaacgcguuuucgaguuaccuauggucagacgacucgcccga

SEQ ID NO: 306 - RNA, 70 nt; single-stranded, linear
  All C′s are 2′-F cytosine
  All U′s are 2′-F uracil
gggaggacgaugcggccaucggucuugggcaacgcguuuucgaguuaccuacaucagacgacucgcccga

SEQ ID NO: 307 - RNA, 61 nt; single-stranded, linear
  All C′s are 2′-F cytosine
  All U′s are 2′-F uracil
gggaggacgaugcgggacccuuaggcaacguguuuucaaguuggucagacgacucgcccga

SEQ ID NO: 308 - RNA, 71 nt; single-stranded, linear
  All C′s are 2′-F cytosine
  All U′s are 2′-F uracil
gggaggacgaugcggacguagcucuuaggcaaugcguauuucgaauuagcugugucagacgacucgcccga

SEQ ID NO: 309 - RNA, 71 nt; single-stranded, linear
  All C′s are 2′-F cytosine
  All U′s are 2′-F uracil
gggaggacgaugcggagucuuaggcagcgcguuuucgagcuacuccaucgccagucagacgacucgcccga

SEQ ID NO: 310 - RNA, 71 nt; single-stranded, linear
  All C′s are 2′-F cytosine
  All U′s are 2′-F uracil
gggaagacgaugcggaaugcucuuaggcagcgcguuaaucgagcuagcacauccucagacgacucgcccga

SEQ ID NO: 311 - RNA, 71 nt; single-stranded, linear
  All C′s are 2′-F cytosine
  All U′s are 2′-F uracil
gggaggacgauggggagucuuaggcagcgcguuuucgagcuacuccaucgccagucagacgacucgcccga

SEQ ID NO: 312 - RNA, 71 nt; single-stranded, linear
  All C′s are 2′-F cytosine
  All U′s are 2′-F uracil
gggaggacgaugcgguaaucucuuaggcaucgcguuaaucgagauagaucaccgucagacgacucgcccga

SEQ ID NO: 313 - RNA, 71 nt; single-stranded, linear
  All C′s are 2′-F cytosine
  All U′s are 2′-F uracil
gggaggacgaugcggcaauguchcuuaggccacgcguuaaucgagcgugacugucagacgacucgcccgag

SEQ ID NO: 314 - RNA, 71 nt; single-stranded, linear
  All C′s are 2′-F cytosine
  All U′s are 2′-F uracil
gggaggacgaugcggcauggucuuaggcgacgcguuuauaucgagucaccaugcucagacgacucgcccga

SEQ ID NO: 315 - RNA, 61 nt; single-stranded, linear
  All C′s are 2′-F cytosine
  All U′s are 2′-F uracil
gggaggacgaugcgggaugcuuaggcgccguguuuucaaggccaucagacgacucgcccga

SEQ ID NO: 316 - RNA, 72 nt; single-stranded, linear
  All C′s are 2′-F cytosine
  All U′s are 2′-F uracil
gggaggacgaugcgguaauugucuuaggcgccguguuaucaaggcacaauuucccucagacgacucgcccga

SEQ ID NO: 317 - RNA, 71 nt; single-stranded, linear
  All C′s are 2′-F cytosine
  All U′s are 2′-F uracil
gggaagacgaugcggcuacuagugucuuaggcggaguguuuaucaauccacacaucagacgacucgcccga

SEQ ID NO: 318 - RNA, 61 nt; single-stranded, linear
  All C′s are 2′-F cytosine
  All U′s are 2′-F uracil
gggaggacgaugcggacugacuuaggcugcgcgcacuucgagcaucagacgacucgcccga

SEQ ID NO: 319 - RNA, 70 nt; single-stranded, linear
  All C′s are 2′-F cytosine
  All U′s are 2′-F uracil
gggaggacgaugcgguggugugucuuuggcaccgcguauuuucgagguacacaucagacgacucgcccga

SEQ ID NO: 320 - RNA, 70 nt; single-stranded, linear
  All C′s are 2′-F cytosine
  All U′s are 2′-F uracil
gggaggacgaugcgguggugugucuuuggcaccgcguauucucgagguacacaucagacgacucgcccga

SEQ ID NO: 321 - RNA, 61 nt; single-stranded, linear
  All C′s are 2′-F cytosine
  All U′s are 2′-F uracil
gggaggacgaugcgggcucuucagcaacguguuaucaaguuagcccagacgacucgcccga

SEQ ID NO: 322 - RNA, 71 nt; single-stranded, linear
  All C′s are 2′-F cytosine
  All U′s are 2′-F uracil
gggaggacgaugcggcguaacuucagcgguguguuaaucaagccuuacgccaucucagacgacucgcccga

SEQ ID NO: 323 - RNA, 59 nt; single-stranded, linear
  All C′s are 2′-F cytosine
  All U′s are 2′-F uracil
gaggacgaugcgggcucuuaagcaacguguuaucaaguuagcccagacgacucgcccga

SEQ ID NO: 324 - RNA, 71 nt; single-stranded, linear
  All C′s are 2′-F cytosine
  All U′s are 2′-F uracil
gggaggacgaugcggucucaagcaaugcguuuaucgaauuaccguacgccuccgucagacgacucgcccga

SEQ ID NO: 325 - RNA, 71 nt; single-stranded, linear
  All C′s are 2′-F cytosine
  All U′s are 2′-F uracil
gggaggacgaugcggaaaucucuuaagcagcguguaaaucaagcuagaucuucgucagacgacucgcccga

SEQ ID NO: 326 - RNA, 61 nt; single-stranded, linear
  All C′s are 2′-F cytosine
  All U′s are 2′-F uracil
gggaggacgaugcgguucuuaagcagcgcgucaaucgagcuaacccagacgacucgcccga

SEQ ID NO: 327 - RNA, 62 nt; single-stranded, linear
  All C′s are 2′-F cytosine
  All U′s are 2′-F uracil
gggaggacgaugcggaucuuaagcagcgcgucaaucgagcuaacccagacgacucgcccgag

SEQ ID NO: 328 - RNA, 75 nt; single-stranded, linear
  All C′s are 2′-F cytosine
  All U′s are 2′-F uracil
acagcugaugaccaugauuacgccaagcuuaagcagcgcguuuucgagcucauguuggucagacgacucgcccga

SEQ ID NO: 329 - RNA, 71 nt; single-stranded, linear
  All C′s are 2′-F cytosine
  All U′s are 2′-F uracil
gggaggacgaugcggagggucuuaagcagugugauaaucaaacuacucuccgugucagacgacucgcccga

SEQ ID NO: 330 - RNA, 62 nt; single-stranded, linear
  All C′s are 2′-F cytosine
  All U′s are 2′-F uracil
gggaggacgaugcgggaucuuaagcagugcguuauucgaacuaucccagacgacucgcccga

SEQ ID NO: 331 - RNA, 70 nt; single-stranded, linear
  All C′s are 2′-F cytosine
  All U′s are 2′-F uracil
gggaggacgaugcggugcuauucuuaagcggcguguuuuucaagccaauaucaucagacgacucgcccga

SEQ ID NO: 332 - RNA, 71 nt; single-stranded, linear
  All C′s are 2′-F cytosine
  All U′s are 2′-F uracil
gggaggacgaugcggucuuaagcggcgcgauuuucgagccaccgcauccuccgugcagacgacucgcccga

SEQ ID NO: 333 - RNA, 61 nt; single-stranded, linear
  All C′s are 2′-F cytosine
  All U′s are 2′-F uracil
gggaggacgaugcggccucuuaagcgucguguuuuucaagcuggucagacgacucgcccga

SEQ ID NO: 334 - RNA, 71 nt; single-stranded, linear
  All C′s are 2′-F cytosine
  All U′s are 2′-F uracil
gggaggacgaugcggauaccaccucuuaagcgacgugcauuucaagucagauggucagacgacucgcccga

SEQ ID NO: 335 - RNA, 72 nt; single-stranded, linear
  All C′s are 2′-F cytosine
  All U′s are 2′-F uracil
gggaggacgaugcggugcuauucuuaagcggcguguaaaucaagcuagaucaucgucagacgacucgcccga

SEQ ID NO: 336 - RNA, 71 nt; single-stranded, linear
  All C′s are 2′-F cytosine
  All U′s are 2′-F uracil
gggaggacgaugcggaacgacucuuaagcugugcguuuucgaacaagucguaacucagacgacucgcccga

SEQ ID NO: 337 - RNA, 61 nt; single-stranded, linear
  All C′s are 2′-F cytosine
  All U′s are 2′-F uracil
gggaggacgaugcggcucucauuuwgcgcguaaaucgagcuagcccagacgacucgcccga

SEQ ID NO: 338 - RNA, 71 nt; single-stranded, linear
  All C′s are 2′-F cytosine
  All U′s are 2′-F uracil
gggaggacgaugcggagucwcucuccaccakcgugukuuaaucaagcuanugccucagacgacucgcccga

SEQ ID NO: 339 - RNA, 71 nt; single-stranded, linear
  All C′s are 2′-F cytosine
  All U′s are 2′-F uracil
gggaggacgaugcggucuacggucucucuggcggugcguaaauckaaccagaucgcagacgacucgcccga

SEQ ID NO: 340 - RNA, 71 nt; single-stranded, linear
  All C′s are 2′-F cytosine
  All U′s are 2′-F uracil
gggaggacgaugcggudauuucyuaauchgagcguuuaucuaucumaaukauccucagacgacucgcccga

SEQ ID NO: 341 - RNA, 62 nt; single-stranded, linear
  All C′s are 2′-F cytosine
  All U′s are 2′-F uracil
gggaggacgaugcggaucgcaaumuguwgcguucuckaaacagccucagacgacucgcccga

SEQ ID NO: 342 - RNA, 61 nt; single-stranded, linear
  All C′s are 2′-F cytosine
  All U′s are 2′-F uracil
gggaggacgaugcggugguucuaggcacguguuuucaaguguaaucagacgacucgcccga

SEQ ID NO: 343 - RNA, 62 nt; single-stranded, linear
  All C′s are 2′-F cytosine
  All U′s are 2′-F uracil
gggaggacgaugcggaaacauguguuuucgaaugugcucuccuccccaaacaacycccccaa

SEQ ID NO: 344 - RNA, 70 nt; single-stranded, linear
  All C′s are 2′-F cytosine
  All U′s are 2′-F uracil
gggaggacgaugcggaaggccguguuaaucaaggcugcaauaaaucauccuccccagacgacucgcccga

SEQ ID NO: 345 - RNA, 71 nt; single-stranded, linear
  All C′s are 2′-F cytosine
  All U′s are 2′-F uracil
gggaggacgaugcggaggaucguguucaucaagauugcucguucuuuacugcguucagacgacucgcccga

SEQ ID NO: 346 - RNA, 71 nt; single-stranded, linear
  All C′s are 2′-F cytosine
  All U′s are 2′-F uracil
gggaggacgaugcggucaaagugaagaauggacagcguuuucgaguugcuucacucagacgacucgcccga

SEQ ID NO: 347 - RNA, 71 nt; single-stranded, linear
  All C′s are 2′-F cytosine
  All U′s are 2′-F uracil
gggaggacgaugcggggagaauggccagcguuuaucgaggugcuccguuaaccggcagacgacucgcccga

SEQ ID NO: 348 - RNA, 61 nt; single-stranded, linear
  All C′s are 2′-F cytosine
  All U′s are 2′-F uracil
gggaggacgaugcgggaggaauggacwgcguauaucgaguugccucagacgacucgcccga

SEQ ID NO: 349 - RNA, 61 nt; single-stranded, linear
  All C′s are 2′-F cytosine
  All U′s are 2′-F uracil
gggaggacgaugcggaucgauuucaugcguuuuucgagugacgaucagacgacucgcccga

SEQ ID NO: 350 - RNA, 61 nt; single-stranded, linear
  All C′s are 2′-F cytosine
  All U′s are 2′-F uracil
gggaggacgaugcggagacccuaagmgsguksuuuucaascuggucwgacgacucgcccga

SEQ ID NO: 351 - RNA, 71 nt; single-stranded, linear
  All C′s are 2′-F cytosine
  All U′s are 2′-F uracil
gggaggacgaugcgguuagccuacacucuagguucaguuuucgaaucuuccaccgcwgacgacucgcccga

SEQ ID NO: 352 - RNA, 61 nt; single-stranded, linear
  All C′s are 2′-F cytosine
  All U′s are 2′-F uracil
gggaggacgaugcgguuaggucaaugaucuuaguuuucgauucgucagacgacucgcccga

SEQ ID NO: 353 - RNA, 61 nt; single-stranded, linear
  All C′s are 2′-F cytosine
  All U′s are 2′-F uracil
gggaggacgaugcggacguguguaucraruuuuccgcuguuugugcagacgacucgcccga

SEQ ID NO: 354 - RNA, 71 nt; single-stranded, linear
  All C′s are 2′-F cytosine
  All U′s are 2′-F uracil
gggaggacgaugcggacaggguucuuaggcggaguguucaucaauccaaccaugucagacgacucgcccga

SEQ ID NO: 355 - RNA, 62 nt; single-stranded, linear
  All C′s are 2′-F cytosine
  All U′s are 2′-F uracil
gggaggacgaugcggcgauuuccacaguuugucuuauuccgcauaucagacgacucgcccga

SEQ ID NO: 356 - RNA, 61 nt; single-stranded, linear
  All C′s are 2′-F cytosine
  All U′s are 2′-F uracil
gggaggacgaugcggauayucagcuygugukuuuucdaucuuccccagacgacucgcccga

SEQ ID NO: 357 - RNA, 61 nt; single-stranded, linear
  All C′s are 2′-F cytosine
  All U′s are 2′-F uracil
gggaggacgaugcggcacacguguuuucaagugugcuccugggaucagacgacucgcccga

SEQ ID NO: 358 - RNA, 61 nt; single-stranded, linear
  All C′s are 2′-F cytosine
  All U′s are 2′-F uracil
gggaggacgaugcggcaauguguuucucaaauugcuuucucccuucagacgacucgcccga

SEQ ID NO: 359 - RNA, 71 nt; single-stranded, linear
  All C′s are 2′-F cytosine
  All U′s are 2′-F uracil
gggaggacgaugcggauacuaccgugcgaacacuaagucccgucuguccacuccucagacgacucgcccga

SEQ ID NO: 360 - RNA, 66 nt; single-stranded, linear
  All C′s are 2′-F cytosine
  All U′s are 2′-F uracil
gggaggacgaugcggauacuaugugcguucacuaagucccgucguccccucagacgacucgcccga

SEQ ID NO: 361 - RNA, 71 nt; single-stranded, linear
  All C′s are 2′-F cytosine
  All U′s are 2′-F uracil
gggaggacgaugcggguacuauguacgaucacuaagccccaucacccuucucacucagacnacucgcccga

SEQ ID NO: 362 - RNA, 61 nt; single-stranded, linear
  All C′s are 2′-F cytosine
  All U′s are 2′-F uracil
gggaggacgaugcgguuacuauguacauuuacuaagacccaacgucagacgacucgcccga

SEQ ID NO: 363 - RNA, 72 nt; single-stranded, linear
  All C′s are 2′-F cytosine
  All U′s are 2′-F uracil
gggaggacgaugcgguuwcuauguwcgccuuacuaaguacccgucgacugucccaucagacgacucgcccga

SEQ ID NO: 364 - RNA, 61 nt; single-stranded, linear
  All C′s are 2′-F cytosine
  All U′s are 2′-F uracil
gggaagacgaugcgguguugaucaaugaauguccuccuccuaccccagacgacucgcccga

SEQ ID NO: 365 - RNA, 61 nt; single-stranded, linear
  All C′s are 2′-F cytosine
  All U′s are 2′-F uracil
gggaggacgaugcgguguuugucaaugucaugauuaguuuucccacagacgacucgcccga

SEQ ID NO: 366 - RNA, 64 nt; single-stranded, linear
  All C′s are 2′-F cytosine
  All U′s are 2′-F uracil
gggaggacgaugcggcggucuuaagcagugugucaaucaaacuaucgucagacgacucgcccga

SEQ ID NO: 367 - RNA, 61 nt; single-stranded, linear; identical to SEQ ID NO: 326
  All C′s are 2′-F cytosine
  All U′s are 2′-F uracil

SEQ ID NO: 368 - RNA, 66 nt; single-stranded, linear
  All C′s are 2′-F cytosine
  All U′s are 2′-F uracil
gggaggacgaugcggaaugrcccguuaccawcaaugcgccucduugmccccaaacaacycccccaa

SEQ ID NO: 369 - RNA, 70 nt; single-stranded, linear
  All C′s are 2′-F cytosine
  All U′s are 2′-F uracil
gggaggacgaugcggaauyucgugyuacgcguyyycuauccaaucuaccccmucuccaaucagacgacyc

SEQ ID NO: 370 - RNA, 61 nt; single-stranded, linear
  All C′s are 2′-F cytosine
  All U′s are 2′-F uracil
gggaggacgaugcggcgcuuacaauaauucucccugaguacagcucagacgacucgcccga

SEQ ID NO: 371 - RNA, 71 nt; single-stranded, linear
  All C′s are 2′-F cytosine
  All U′s are 2′-F uracil
gggaggacgaugcggaacuucuuaggcagcgugcuagucaagcuaaguuccaccucagacgacucgcccga

SEQ ID NO: 372 - RNA, 70 nt; single-stranded, linear
  All C′s are 2′-F cytosine
  All U′s are 2′-F uracil
gggaggacgaugcggcacaaucuucggcagcgugcaagaucaagcuauuguugucagacgacucgcccga

SEQ ID NO: 373 - RNA, 61 nt; single-stranded, linear
  All C′s are 2′-F cytosine
  All U′s are 2′-F uracil
gggaggacgaugcggucauuaaccaagauaugcgaaucaccuccucagacgacucgcccga

SEQ ID NO: 374 - RNA, 62 nt; single-stranded, linear
  All C′s are 2′-F cytosine
  All U′s are 2′-F uracil
gggaggacgaugcggucauucucuaaaaaaguauuccguaccuccacagacgacucgcccga

SEQ ID NO: 375 - RNA, 61 nt; single-stranded, linear
  All C′s are 2′-F cytosine
  All U′s are 2′-F uracil
gggaggacgaugcgggugaucuuuuaugcuccucuuguuuccugucagacgacucgcccga

SEQ ID NO: 376 - RNA, 71 nt; single-stranded, linear
  All C′s are 2′-F cytosine
  All U′s are 2′-F uracil
gggaggacnaugcggucuaggcaucgcuauucuuuacugauauaauuacuccccucagacgacucgcccga

SEQ ID NO: 377 - RNA, 61 nt; single-stranded, linear
  All C′s are 2′-F cytosine
  All U′s are 2′-F uracil
gggaggacgaugcggaguwwgcncgguccagucacauccwauccccagacgacucgcccga

SEQ ID NO: 378 - RNA, 62 nt; single-stranded, linear
  All C′s are 2′-F cytosine
  All U′s are 2′-F uracil
gggaggacgaugcggcucucauaukgwguruuyuucmuucsrggcucaaacaayycccccaa

SEQ ID NO: 379 - RNA, 61 nt; single-stranded, linear
  All C′s are 2′-F cytosine
  All U′s are 2′-F uracil
gggaggacgaugcggcuuguuaguuaaacucgagucuccaccccucagacgacucgcccga

SEQ ID NO: 380 - RNA, 62 nt; single-stranded, linear
  All C′s are 2′-F cytosine
  All U′s are 2′-F uracil
gggaggacgaugcggucucuwcuvacvuguruucacauuuucgcyucaaacaacycccccaa

SEQ ID NO: 381 - RNA, 61 nt; single-stranded, linear
  All C′s are 2′-F cytosine
  All U′s are 2′-F uracil
gggaggacgaugcgguuracaaugrsscucrccuucccwgguccucagacgacucgcccga

SEQ ID NO: 382 - RNA, 71 nt; single-stranded, linear
  All C′s are 2′-F cytosine
  All U′s are 2′-F uracil
aggaggacgaugcgguuaucugaarcwugcguaamcuarugusaaasugcaacracraacaacycscccaa

SEQ ID NO: 383 - RNA, 61 nt; single-stranded, linear
  All C′s are 2′-F cytosine
  All U′s are 2′-F uracil
aggaagacgaugcgguucgauuuauuugugucauuguucuuccaucagacgacucgcccga

SEQ ID NO: 384 - RNA, 35 nt; single-stranded, linear
  All C′s are 2′-F cytosine
  All U′s are 2′-F uracil
gugaugacauggauuacgccagacgacucgcccga

SEQ ID NO: 385 - RNA, 16 nt; single-stranded, linear
  All C′s are 2′-F cytosine
  All U′s are 2′-F uracil
ugcguguuuucaagca

SEQ ID NO: 386 - RNA, 23 nt; single-stranded, linear
  All C′s are 2′-F cytosine
  All U′s are 2′-F uracil
cucaaauugcguguuuucaagca

SEQ ID NO: 387 - RNA, 33 nt; single-stranded, linear
  All C′s are 2′-F cytosine
  All U′s are 2′-F uracil
gguaccucaaauugcguguuuucaagcaguauc

SEQ ID NO: 388 - RNA, 33 nt; single-stranded, linear
  All C′s are 2′-F cytosine
  All U′s are 2′-F uracil
ggagucuuaggcagcgcguuuucgagcuacucc

SEQ ID NO: 389 - RNA, 71 nt; single-stranded, linear; identical to SEQ ID NO: 64
  All C′s are 2′-F cytosine
  All U′s are 2′-F uracil

SEQ ID NO: 390 - RNA, 97 nt; single-stranded, linear; identical to SEQ ID NO: 248
  All C′s are 2′-F cytosine
  All U′s are 2′-F uracil

SEQ ID NO: 391 - RNA, 38 nt; single-stranded, linear; identical to SEQ ID NO: 223
  All C′s are 2′-F cytosine
  All U′s are 2′-F uracil
  7, 9, 28: G′s are OMe
  8, 15, 27, 30: A′s are OMe

(19) United States
(12) Patent Application Publication
(10) Pub. No.: US 2009/0118481 A1
(43) Pub. Date: May 7, 2009

(54) High Affinity Nucleic Acid Ligands To Lectins

(75) Inventors: David H. Parma, Boulder, CO (US); Brian Hicke, Foster City, CA (US); Philippe Bridonneau, Lyons (FR); Larry Gold, Boulder, CO (US)

Correspondence Address: SWANSON & BRATSCHUN, L.L.C., 8210 SOUTHPARK TERRACE, LITTLETON, CO, 80120, US

(73) Assignee: GILEAD SCIENCES, INC., Foster City, CA (US)

(21) Appl. No.: 12/133,132

(22) Filed: Jun. 4, 2008

Related U.S. Application Data
(63) Continuation of application No. 10/705,300, filed on Nov. 10, 2003, now abandoned.
Application No. 10/705,300 is a continuation of application No. 10/409,627, filed on Apr. 7, 2003, now Pat. No. 7,399,752.
Application No. 10/409,627 is a continuation of application No. 09/849,928, filed on May 4, 2001, now Pat. No. 6,544,959.
Application No. 09/849,928 is a division of application No. 08/952,793, filed on Nov. 21, 1997, now Pat. No. 6,280,932.
Application No. 08/952,793 is a continuation-in-part of application No. 08/479,724, filed on Jun. 7, 1995, now Pat. No. 5,780,228.
Application No. 08/479,724 is a continuation-in-part of application No. 08/472,256, filed on Jun. 7, 1995, now Pat. No. 6,001,988.
Application No. 08/472,256 is a continuation-in-part of application No. 08/472,255, filed on Jun. 7, 1995, now Pat. No. 5,766,853.
Application No. 08/472,255 is a continuation-in-part of application No. 08/477,829, filed on Jun. 7, 1995, now abandoned.

(51) Int. Cl. C07H 21/00

(52) U.S. Cl. 536/22.1

(57) ABSTRACT

This invention discloses high-affinity oligonucleotide ligands to lectins, specifically nucleic acid ligands having the ability to bind to the lectins, wheat germ agglutinin, L-selectin, E-selectin and P-selectin. Also disclosed are the methods for obtaining such ligands. This invention discloses high-affinity oligonucleotide ligands to lectins, specifically nucleic acid ligands having the ability to bind to the lectins, wheat germ agglutinin, L-selectin, E-selectin and P-selectin. Also disclosed are the methods for obtaining such ligands.

### RELATED APPLICATIONS

This application is a continuation of U.S. Ser. No. 10/705,300, filed Nov. 10, 2003, which is a continuation of U.S. Ser. No. 10/409,627, filed Apr. 7, 2003, which is a continuation of U.S. Ser. No. 09/849,928, filed May 4, 2001, now U.S. Pat. No. 6,544,959, which is a divisional of U.S. Ser. No. 08/952,793, filed Nov. 21, 1997, now U.S. Pat. No. 6,280,932, which is a 35 U.S.C. § 371 national phase of PCT/US96/09455, filed Jun. 5, 1996, which is a continuation-in-part of each of the following: U.S. Ser. No. 08/479,724, filed Jun. 7, 1995, now U.S. Pat. No. 5,780,228; U.S. Ser. No. 08/472,256, filed Jun. 7, 1995, now U.S. Pat. No. 6,001,988; U.S. Ser. No. 08/472,255, filed Jun. 7, 1995, now U.S. Pat. No. 5,766,853; and U.S. Ser. No. 08/477,829, filed Jun. 7, 1995, now abandoned. Each of these applications is hereby incorporated by reference herein in their entirety.

### FIELD OF THE INVENTION

Described herein are methods for identifying and preparing high-affinity nucleic acid ligands to lectins. Lectins are carbohydrate binding proteins. The method utilized herein for identifying such nucleic acid ligands is called SELEX, an acronym for Systematic Evolution of Ligands by EXponential enrichment. Specifically disclosed herein are high-affinity nucleic acid ligands to wheat germ agglutinin (WGA), L-selectin, E-selectin, and P-selectin.

### BACKGROUND OF THE INVENTION

The biological role of lectins (non-enzymatic carbohydrate-binding proteins of non-immune origin; I. J. Goldstein et al., 1980, Nature 285:66) is inextricably linked to that of carbohydrates. One function of carbohydrates is the modification of physical characteristics of glyco-conjugates (i.e., solubility, stability, activity, susceptibility to enzyme or antibody recognition), however, a more interesting and relevant aspect of carbohydrate biology has emerged in recent years; the carbohydrate portions of glyco-conjugates are information rich molecules (N. Sharon and H. L is, 1989, Science 246:227-234; K. Drickamer and M. Taylor, 1993, Annu. Rev. Cell Biol. 9:237-264; A. Varki, 1993, Glycobiol. 3:97-130). Within limits, the binding of carbohydrates by lectins is specific (i.e., there are lectins that bind only galactose or N-acetylgalactose; other lectins bind mannose; still others bind sialic acid and so on; K. Drickamer and M. Taylor, supra). Specificity of binding enables lectins to decode information contained in the carbohydrate portion of glyco-conjugates and thereby mediate many important biological functions.

Numerous mammalian, plant, microbial and viral lectins have been described (I. Ofek and N. Sharon, 1990, Current Topics in Microbiol. and Immunol. 151:91-113; K. Drickamer and M. Taylor, supra; I. J. Goldstein and R. D. Poretz, 1986, in The Lectins, p.p. 33-247; A. Varki, supra). These proteins mediate a diverse array of biological processes which include: trafficking of lysosomal enzymes, clearance of serum proteins, endocytosis, phagocytosis, opsonization, microbial and viral infections, toxin binding, fertilization, immune and inflammatory responses, cell adhesion and migration in development and in pathological conditions such as metastasis. Roles in symbiosis and host defense have been proposed for plant lectins but remain controversial. While the functional role of some lectins is well understood, that of many others is understood poorly or not at all.

The diversity and importance of processes mediated by lectins is illustrated by two well documented mammalian lectins, the asialoglycoprotein receptor and the serum mannose binding protein, and by the viral lectin, influenza virus hemagglutinin. The hepatic asialoglycoprotein receptor specifically binds galactose and N-acetylgalactose and thereby mediates the clearance of serum glycoproteins that present terminal N-acetylgalactose or galactose residues, exposed by the prior removal of a terminal sialic acid. The human mannose-binding protein (MBP) is a serum protein that binds terminal mannose, fucose and N-acetylglucosamine residues. These terminal residues are common on microbes but not mammalian glyco-conjugates. The binding specificity of MBP constitutes a non-immune mechanism for distinguishing self from non-self and mediates host defense through opsonization and complement fixation. Influenza virus hemagglutinin mediates the initial step of infection, attachment to nasal epithelial cells, by binding sialic acid residues of cell-surface receptors.

The diversity of lectin mediated functions provides a vast array of potential therapeutic targets for lectin antagonists. Both lectins that bind endogenous carbohydrates and those that bind exogenous carbohydrates are target candidates. For example, antagonists to the mammalian selecting, a family of endogenous carbohydrate binding lectins, may have therapeutic applications in a variety of leukocyte-mediated disease states. Inhibition of selectin binding to its receptor blocks cellular adhesion and consequently may be useful in treating inflammation, coagulation, transplant rejection, tumor metastasis, rheumatoid arthritis, reperfusion injury, stroke, myocardial infarction, burns, psoriasis, multiple sclerosis, bacterial sepsis, hypovolaemic and traumatic shock, acute lung injury, and ARDS.

The selectins, E-, P- and L-, are three homologous C-type lectins that recognize the tetrasaccharide, sialyl-Lewis^{X} (C. Foxall et al, 1992, J. Cell Biol. 117, 895-902). Selectins mediate the initial adhesion of neutrophils and monocytes to activated vascular endothelium at sites of inflammation (R. S. Cotran et al., 1986, J. Exp. Med. 164, 661-; M. A. Jutila et al., 1989, J. Immunol. 143, 3318-; J. G. Geng et al., 1990, Nature, 757; U. H. Von Adrian et al., 1994, Am. J. Physiol. Heart Circ. Physiol. 263, H1034-H1044). In addition, L-selectin is responsible for the homing of lymphocytes to peripheral and mesenteric lymph nodes (W. M. Gallatin et al., 1983, Nature 304, 30; T. K. Kishimoto et al., 1990, Proc. Natl. Acad. Sci. 87, 2244) and P-selectin mediates the adherence of platelets to neutrophils and monocytes (S-C. Hsu-Lin et al., 1984, J. Biol. Chem. 259, 9121). Selectin antagonists (antibodies and carbohydrates) have been shown to block the extravasation of neutrophils at sites of inflammation (P. Piscueta and F. W. Luscinskas, 1994, Am. J. Pathol. 145, 461-469), to be efficacious in animal models of ischemia/reperfusion (A. S. Weyrich et al., 1993, J. Clin. Invest. 91, 2620-2629; R. K. Winn et al., 1993, J. Clin. Invest. 92, 2042-2047), acute lung injury (M. S. Mulligan et al., 1993, J. Immunol. 151, 6410-6417; A. Seekamp et al., 1994, Am. J. Pathol. 144, 592-598), insulitis/diabetes (X. D. Yang et al., 1993, Proc. Natl. Acad. Sci. 90,10494-10498), meningitis (C. Granet et al., 1994, J. Clin. Invest. 93, 929-936), hemorrhagic shock (R. K. Winn et al., 1994, Am J. Physiol. Heart Circ. Physiol. 267, H2391-H2397) and transplantation. In addition, selectin expression has been documented in models of arthritis (F. Jamar et al., 1995, Radiology 194, 843-850), experimental allergic encephalomyelitis (J. M. Dopp et al., 1994, J. Neuroimmunol. 54, 129-144), cutaneous inflammation (A. Siber et al., 1994, Lab. Invest. 70, 163-170) glomerulonephritis (P. G. Tipping et al., 1994, Kidney Int. 46, 79-88), on leukaemic cells and colon carcinomas (R. M. Lafrenie et al., 1994, Eur. J. Cancer [A] 30A, 2151-2158) and L-selectin receptors have been observed in myelinated regions of the central nervous system (K. Huang et al., 1991, J. Clin. Invest. 88, 1778-1783). These animal model data strongly support the expectation of a therapeutic role for selectin antagonists in a wide variety of disease states in which host tissue damage is neutrophil-mediated.

Other examples of lectins that recognize endogenous carbohydrates are CD22β, CD23, CD44 and sperm lectins (A. Varki, 1993, Glycobiol.3, 97-130; P. M. Wassarman, 1988, Ann. Rev. Biochem. 57, 415-442). CD22β is involved in early stages of B lymphocyte activation; antagonists may modulate the immune response. CD23 is the low affinity IgE receptor; antagonists may modulate the IgE response in allergies and asthma. CD44 binds hyaluronic acid and thereby mediates cell/cell and cell/matrix adhesion; antagonists may modulate the inflammatory response. Sperm lectins are thought to be involved in sperm/egg adhesion and in the acrosomal response; antagonists may be effective contraceptives, either by blocking adhesion or by inducing a premature, spermicidal acrosomal response. Antagonists to lectins that recognize exogenous carbohydrates may have wide application for the prevention of infectious diseases. Many viruses (influenza A, B and C; Sendhi, Newcastle disease, coronavirus, rotavirus, encephalomyelitis virus, enchephalomyocarditis virus, reovirus, paramyxovirus) use lectins on the surface of the viral particle for attachment to cells, a prerequisite for infection; antagonists to these lectins are expected to prevent infection (A. Varki, 1993, Glycobiol.3, 97-130). Similarly colonization/infection strategies of many bacteria utilize cell surface lectins to adhere to mammalian cell surface glyco-conjugates. Antagonists to bacterial cell surface lectins are expected to have therapeutic potential for a wide spectrum of bacterial infections, including: gastric (Helicobacter pylori), urinary tract (E. coli), pulmonary (Klebsiella pneumoniae, 120 Streptococcus pneumoniae, Mycoplasma pneumoniae) and oral (Actinomyces naeslundi and Actinomyces viscosus) colonization/infection (S. N. Abraham, 1994, Bacterial Adhesins, in The Handbook of Immunopharmacology: Adhesion Molecules, C. D. Wegner, ed; B. J. Mann et al., 1991, Proc. Natl. Acad. Sci. 88, 3248-3252). A specific bacterial mediated disease state is Pseudomonas aeruginosa infection, the leading cause of morbidity and mortality in cystic fibrosis patients. The expectation that high affinity antagonists will have efficacy in treating P. aeruginosa infection is based on three observations. First, a bacterial cell surface, GalNAc β1-4Gal binding lectin mediates infection by adherence to asialogangliosides (αGM1 and αGM2) of pulmonary epithelium (L. Imundo et al., 1995, Proc. Natl. Acad. Sci. 92, 3019-3023). Second, in vitro, the binding of P. aeruginosa is competed by the gangliosides' tetrasaccharide moiety, Gal β1-3GalNAc β1-4Gal β1-4Glc. Third, in vivo, instillation of antibodies to Pseudomonas surface antigens can prevent lung and pleural damage (J. F. Pittet et al., 1993, J. Clin. Invest. 92, 1221-1228).

Non-bacterial microbes that utilize lectins to initiate infection include Entamoeba histalytica (a Gal specific lectin that mediates adhesion to intestinal mucosa; W. A. Petri, Jr., 1991, AMS News 57:299-306) and Plasmodium faciparum (a lectin specific for the terminal Neu5Ac(a2-3)Gal of glycophorin A of erthrocytes; P. A. Orlandi et al., 1992, J. Cell Biol. 116:901-909). Antagonists to these lectins are potential therapeutics for dysentery and malaria. Toxins are another class of proteins that recognize exogenous carbohydrates (K-A Karlsson, 1989, Ann. Rev. Biochem. 58:309-350). Toxins are complex, two domain molecules, composed of a functional and a cell recognition/adhesion domain. The adhesion domain is often a lectin (i.e., bacterial toxins: pertussis toxin, cholera toxin, heat labile toxin, verotoxin and tetanus toxin; plant toxins: ricin and abrin). Lectin antagonists are expected to prevent these toxins from binding their target cells and consequently to be useful as antitoxins.

There are still other conditions for which the role of lectins is currently speculative. For example, genetic mutations result in reduced levels of the serum mannose-binding protein (MBP). Infants who have insufficient levels of this lectin suffer from severe infections, but adults do not. The high frequency of mutations in both oriental and Caucasian populations suggests a condition may exist in which low levels of serum mannose-binding protein are advantageous. Rheumatoid arthritis (RA) may be such a condition. The severity of RA is correlated with an increase in IgG antibodies lacking terminal galactose residues on Fc region carbohydrates (A. Young et al., 1991, Arth. Rheum. 34, 1425-1429; I. M. Roitt et al., 1988, J. Autoimm. 1, 499-506). Unlike their normal counterpart, these gal-deficient carbohydrates are substrates for MBP. MBP/IgG immunocomplexes may contribute to host tissue damage through complement activation. Similarly, the eosinophil basic protein is cytotoxic. If the cytotoxicity is mediated by the lectin activity of this protein, then a lectin antagonist may have therapeutic applications in treating eosinophil mediated lung damage. Lectin antagonists may also be useful as imaging agents or diagnostics. For example; E-selectin antagonists may be used to image inflamed endothelium. Similarly antagonists to specific serum lectins, i.e. mannose-binding protein, may also be useful in quantitating protein levels.

Lectins are often complex, multi-domain, multimeric proteins. However, the carbohydrate-binding activity of mammalian lectins is normally the property of a carbohydrate recognition domain or CRD. The CRDs of mammalian lectins fall into three phylogenetically conserved classes: C-type, S-type and P-type (K. Drickamer and M. E. Taylor, 1993, Annu. Rev. Cell Biol. 9, 237-264). C-type lectins require Ca⁺⁺ for ligand binding, are extracellular membrane and soluble proteins and, as a class, bind a variety of carbohydrates. S-type lectins are most active under reducing conditions, occur both intra- and extracellularly, bind β-galactosides and do not require Ca⁺⁺. P-type lectins bind mannose 6-phosphate as their primary ligand. Although lectin specificity is usually expressed in terms of monosaccharides and/or oligosacchrides (i.e., MBP binds mannose, fucose and N-acetylglucosamine), the affinity for monosaccharides is weak. The dissociation constants for monomeric saccharides are typically in the millimolar range (Y. C. Lee, 1992, FASEB J. 6:3193-3200; G. D. Glick et al., 1991, J. Biol. Chem. 266:23660-23669; Y. Nagata and M. M. Burger, 1974, J. Biol. Chem. 249:116-3122).

Co-crystals of MBP complexed with mannose oligomers offer insight into the molecular limitations on affinity and specificity of C-type lectins (W. I. Weis et al., 1992, Nature 360:127-134; K. Drickamer, 1993, Biochem. Soc. Trans. 21:456-459). The 3- and 4-hydroxyl groups of mannose form coordination bonds with bound Ca.⁺⁺ ion #2 and hydrogen bonds with glutamic acid (185 and 193) and asparagine (187 and 206). The limited contacts between the CRD and bound sugar are consistent with its spectrum of monosaccharide binding; N-acetylglucosamine has equatorial 3- and 4-hydroxyls while fucose has similarly configured hydroxyls at the 2 and 3 positions.

The affinity of the mannose-binding protein and other lectins for their natural ligands is greater than that for monosaccharides. Increased specificity and affinity can be accomplished by establishing additional contacts between a protein and its ligand (K. Drickamer, 1993, supra) either by 1) additional contacts with the terminal sugar (i.e., chicken hepatic lectin binds N-acetylglucose amine with greater affinity than mannose or fucose suggesting interaction with the 2-substituent); 2) clustering of CRDs for binding complex oligosaccharides (i.e., the mammalian asialylglycoprotein receptor); 3) interactions with additional saccharide residues (i.e., the lectin domain of selectins appears to interact with two residues of the tetrasaccharide sialyl-Lewis^{X}: with the charged terminal residue, sialic acid, and with the fucose residue; wheat germ agglutinin appears to interact with all three residues of trimers of N-acetylglucosamine); or by 4) contacts with a non-carbohydrate portion of a glyco-protein.

The low affinity of lectins for mono- and oligo-saccharides presents major difficulties in developing high affinity antagonists that may be useful therapeutics. Approaches that have been used to develop antagonists are similar to those that occur in nature: 1) addition or modification of substituents to increase the number of interactions; and 2) multimerization of simple ligands.

The first approach has had limited success. For example, homologues of sialic acid have been analyzed for affinity to influenza virus hemagglutinin (S. J. Watowich et al. 1994, Structure 2:719-731). The dissociation constants of the best analogues are 30 to 300 μM which is only 10 to 100-fold better than the standard monosaccharide. Modifications of carbohydrate ligands to the selectins have also had limited success. In static ELISA competition assays, sialyl-Lewis^{a} and sialyl-Lewis^{X} have IC₅₀s of 220 μM and 750 μM, respectively, for the inhibition of the binding of an E-selectin/IgG chimera to immobilized sialyl-Lewis^{X} (R. M. Nelson et al., 1993, J. Clin. Invest. 91, 1157-1166). The IC₅₀ of a sialyl-Lewis^{a} derivative (addition of an aliphatic aglycone to the GlcNAc and replacement of the N-acetyl with an NH₂ group) improved 10-fold to 21 μM. Similarly, removal of the N-acetyl from sialyl-Lewis^{X} improves inhibition in an assay dependent manner (C. Foxall et al., 1992, J. Cell Biol. 117, 895-902; S. A. DeFrees et al., 1993, J. Am. Chem. Soc. 115, 7549-7550). The second approach, multimerization of simple ligands, can lead to dramatic improvements in affinity for lectins that bind complex carbohydrates (Y. C. Lee, supra). On the other hand, the approach does not show great enhancement for lectins that bind simple oligosaccharides; dimerizing sialyl-Lewis^{X}, a minimal carbohydrate ligand for E-selectin, improves inhibition approximately 5-fold (S. A. DeFrees et al., supra). An alternative approach is to design compounds that are chemically unrelated to the natural ligand. In the static ELISA competition assays inositol polyanions inhibit L- and P-selectin binding with IC₅₀s that range from 1.4 μM to 2.8 mM (O. Cecconi et al., 1994, J. Biol. Chem. 269, 15060-15066). Synthetic oligopeptides, based on selectin amino acid sequences, inhibit neutrophil binding to immobilized P-selectin with IC₅₀s ranging from 50 μM to 1 mM (J-G Geng et al., 1992, J of Biol. Chem. 267, 19846-19853).

Lectins are nearly ideal targets for isolation of antagonists by SELEX technology described below. The reason is that oligonucleotide ligands that are bound to the carbohydrate binding site can be specifically eluted with the relevant sugar(s). Oligonucleotide ligands with affinities that are several orders of magnitude greater than that of the competing sugar can be obtained by the appropriate manipulation of the nucleic acid ligand to competitor ratio. Since the carbohydrate binding site is the active site of a lectin, essentially all ligands isolated by this procedure will be antagonists. In addition, these SELEX ligands will exhibit much greater specificity than monomeric and oligomeric saccharides.

A method for the in vitro evolution of nucleic acid molecules with highly specific binding to target molecules has been developed. This method, Systematic Evolution of Ligands by EXponential enrichment, termed SELEX, is described in U.S. patent application Ser. No. 07/536,428, entitled “Systematic Evolution of Ligands by Exponential Enrichment,” now abandoned, U.S. patent application Ser. No. 07/714,131, filed Jun. 10, 1991, entitled “Nucleic Acid Ligands,” now U.S. Pat. No. 5,475,096, U.S. patent application Ser. No. 07/931,473, filed Aug. 17, 1992, entitled “Methods for Identifying Nucleic Acid Ligands,” now U.S. Pat. No. 5,270,163 (see also PCT/US91/04078), each of which is herein specifically incorporated by reference. Each of these applications, collectively referred to herein as the SELEX patent applications, describes a fundamentally novel method for making a nucleic acid ligand to any desired target molecule.

The SELEX method involves selection from a mixture of candidate oligonucleotides and step-wise iterations of binding, partitioning and amplification, using the same general selection scheme, to achieve virtually any desired criterion of binding affinity and selectivity. Starting from a mixture of nucleic acids, preferably comprising a segment of randomized sequence, the SELEX method includes steps of contacting the mixture with the target under conditions favorable for binding, partitioning unbound nucleic acids from those nucleic acids which have bound specifically to target molecules, dissociating the nucleic acid-target complexes, amplifying the nucleic acids dissociated from the nucleic acid-target complexes to yield a ligand-enriched mixture of nucleic acids, then reiterating the steps of binding, partitioning, dissociating and amplifying through as many cycles as desired to yield highly specific, high affinity nucleic acid ligands to the target molecule.

The basic SELEX method has been modified to achieve a number of specific objectives. For example, U.S. patent application Ser. No. 07/960,093, filed Oct. 14, 1992, entitled “Method for Selecting Nucleic Acids on the Basis of Structure,” describes the use of SELEX in conjunction with gel electrophoresis to select nucleic acid molecules with specific structural characteristics, such as bent DNA. U.S. patent application Ser. No. 08/123,935, filed Sep. 17, 1993, entitled “Photoselection of Nucleic Acid Ligands” describes a SELEX based method for selecting nucleic acid ligands containing photoreactive groups capable of binding and/or photocrosslinking to and/or photoinactivating a target molecule. U.S. patent application Ser. No. 08/134,028, filed Oct. 7, 1993, entitled “High-Affinity Nucleic Acid Ligands That Discriminate Between Theophylline and Caffeine,” describes a method for identifying highly specific nucleic acid ligands able to discriminate between closely related molecules, termed Counter-SELEX. U.S. patent application Ser. No. 08/143,564, filed Oct. 25, 1993, entitled “Systematic Evolution of Ligands by EXponential Enrichment: Solution SELEX,” describes a SELEX-based method which achieves highly efficient partitioning between oligonucleotides having high and low affinity for a target molecule. U.S. patent application Ser. No. 07/964,624, filed Oct. 21, 1992, entitled “Nucleic Acid Ligands to HIV-RT and HIV-1 Rev,” now U.S. Pat. No. 5,496,938, describes methods for obtaining improved nucleic acid ligands after SELEX has been performed. U.S. patent application Ser. No. 08/400,440, filed Mar. 8, 1995, entitled “Systematic Evolution of Ligands by EXponential Enrichment: Chemi-SELEX,” now U.S. Pat. No. 5,705,337, describes methods for covalently linking a ligand to its target.

The SELEX method encompasses the identification of high-affinity nucleic acid ligands containing modified nucleotides conferring improved characteristics on the ligand, such as improved in vivo stability or improved delivery characteristics. Examples of such modifications include chemical substitutions at the ribose and/or phosphate and/or base positions. SELEX-identified nucleic acid ligands containing modified nucleotides are described in U.S. patent application Ser. No. 08/117,991, filed Sep. 8, 1993, entitled “High Affinity Nucleic Acid Ligands Containing Modified Nucleotides,” that describes oligonucleotides containing nucleotide derivatives chemically modified at the 5- and 2′-positions of pyrimidines. U.S. patent application Ser. No. 08/134,028, supra, describes highly specific nucleic acid ligands containing one or more nucleotides modified with 2′-amino (2′-NH₂), 2′-fluoro (2′-F), and/or 2′-O-methyl (2′-OMe). U.S. patent application Ser. No. 08/264,029, filed Jun. 22, 1994, entitled “Novel Method of Preparation of 2′ Modified Pyrimidine Intramolecular Nucleophilic Displacement,” describes novel methods for making 2′-modified nucleosides.

The SELEX method encompasses combining selected oligonucleotides with other selected oligonucleotides as described in U.S. patent application Ser. No. 08/284,063, filed Aug. 2, 1994, entitled “Systematic Evolution of Ligands by Exponential Enrichment: Chimeric SELEX,” now U.S. Pat. No. 5,637,459. The SELEX method also includes combining the selected nucleic acid ligands with non-oligonucleotide functional units and U.S. patent application Ser. No. 08/234,997, filed Apr. 28, 1994, entitled “Systematic Evolution of Ligands by Exponential Enrichment: Blended SELEX,” now U.S. Pat. No. 5,683,867, and U.S. patent application Ser. No. 08/434,465, filed May 4, 1995, entitled “Nucleic Acid Ligand Complexes,” now U.S. Pat. No. 6,011,020. These applications allow the combination of the broad array of shapes and other properties, and the efficient amplification and replication properties, of oligonucleotides with the desirable properties of other molecules. Each of the above described patent applications which describe modifications of the basic SELEX procedure are specifically incorporated by reference herein in their entirety.

The present invention applies the SELEX methodology to obtain nucleic acid ligands to lectin targets. Lectin targets, or lectins, include all the non-enzymatic carbohydrate-binding proteins of non-immune origin, which include, but are not limited to, those described above.

Specifically, high affinity nucleic acid ligands to wheat germ agglutinin, and various selectin proteins have been isolated. For the purposes of the invention the terms wheat germ agglutinin, wheat germ lectin and WGA are used interchangeably. Wheat germ agglutinin (WGA) is widely used for isolation, purification and structural studies of glyco-conjugates. As outlined above, the selectins are important anti-inflammatory targets. Antagonists to the selectins modulate extravasion of leukocytes at sites of inflammation and thereby reduce neutrophil caused host tissue damage. Using the SELEX technology, high affinity antagonists of L-selectin, E-selectin and P-selectin mediated adhesion are isolated.

### BRIEF SUMMARY OF THE INVENTION

The present invention includes methods of identifying and producing nucleic acid ligands to lectins and the nucleic acid ligands so identified and produced. More particularly, nucleic acid ligands are provided that are capable of binding specifically to Wheat Germ Agglutinin (WGA), L-Selectin, E-selectin and P-selectin.

Further included in this invention is a method of identifying nucleic acid ligands and nucleic acid ligand sequences to lectins comprising the steps of (a) preparing a candidate mixture of nucleic acids, (b) partitioning between members of said candidate mixture on the basis of affinity to said lectin, and (c) amplifying the selected molecules to yield a mixture of nucleic acids enriched for nucleic acid sequences with a relatively higher affinity for binding to said lectin.

More specifically, the present invention includes the nucleic acid ligands to lectins identified according to the above-described method, including those ligands to Wheat Germ Agglutinin listed in Table 2, those ligands to L-selectin listed in Tables 8, 12 and 16, and those ligands to P-selectin listed in Tables 19 and 25. Additionally, nucleic acid ligands to E-selectin and serum mannose binding protein are provided. Also included are nucleic acid ligands to lectins that are substantially homologous to any of the given ligands and that have substantially the same ability to bind lectins and antagonize the ability of the lectin to bind carbohydrates. Further included in this invention are nucleic acid ligands to lectins that have substantially the same structural form as the ligands presented herein and that have substantially the same ability to bind lectins and antagonize the ability of the lectin to bind carbohydrates.

The present invention also includes modified nucleotide sequences based on the nucleic acid ligands identified herein and mixtures of the same.

The present invention also includes the use of the nucleic acid ligands in therapeutic, prophylactic and diagnostic applications.

### BRIEF DESCRIPTION OF THE DRAWINGS

FIG. 1 shows consensus hairpin secondary structures for WGA 2′-NH₂ RNA ligands: (a) family 1, (b) family 2 and (c) family 3. Nucleotide sequence is in standard one letter code. Invariant nucleotides are in bold type. Nucleotides derived from fixed sequence are in lower case.

FIG. 2 shows binding curves for the L-selectin SELEX second and ninth round 2′-NH₂ RNA pools to peripheral blood lymphocytes (PBMCs).

FIG. 3 shows binding curves for random 40N7 2′-NH₂ RNA (SEQ ID NO: 64) and the cloned L-selectin ligand, F14.12 (SEQ ID NO: 78), to peripheral blood lymphocytes (PBMC).

FIG. 4 shows the results of a competition experiment in which the binding of 5 nM ³²P-labeled F14.12 (SEQ ID NO: 78) to PBMCs (10⁷/ml) is competed with increasing concentrations of unlabeled F14.12 (SEQ ID NO: 78). RNA Bound equals 100×(net counts bound in the presence of competitor/net counts bound in the absence of competitor).

FIG. 5 shows the results of a competition experiment in which the binding of 5 nM ³²P-labeled F14.12 (SEQ ID NO: 78) to PBMCs (10⁷/ml) is competed with increasing concentrations of the blocking monoclonal anti-L-selectin antibody. DREG-56, or an isotype matched, negative control antibody. RNA Bound equals 100× times.(net counts bound in the presence of competitor/net counts bound in the absence of competitor).

FIG. 6 shows the results of a competitive ELISA assay in which the binding of soluble LS-Rg to immobilized sialyl-Lewisx/BSA conjugates is competed with increasing concentrations of unlabeled F14.12 (SEQ ID NO: 78). Binding of LS-Rg was monitored with an HRP conjugated anti-human IgG antibody. LS-Rg Bound equals 100×(OD₄₅₀ in the presence of competitor)/(OD₄₅₀ in the absence of competitor). The observed OD₄₅₀ was corrected for nonspecific binding by subtracting the OD₄₅₀ in the absence of LS-Rg from the experimental values. In the absence of competitor the OD₄₅₀ was 0.324 and in the absence of LS-Rg 0.052. Binding of LS-Rg requires divalent cations; in the absence of competitor, replacement of Ca⁺⁺/Mg⁺⁺ with 4 mM EDTA reduced the OD₄₅₀ to 0.045.

FIG. 7 shows hairpin secondary structures for representative L-selectin 2′NH₂ RNA ligands: (a) F13.32 (SEQ. ID NO: 67), family I; (b) 6.16 (SEQ. ID NO: 84), family III; and (c) F14.12 (SEQ. ID NO: 78), family II. Nucleotide sequence is in standard one letter code. Invariant nucleotides are in bold type. Nucleotides derived from fixed sequence are in lower case.

FIG. 8 shows a schematic representation of each dimeric and multimeric oligonucleotide complex: (a) dimeric branched oligonucleotide; (b) multivalent streptavidin/bio-oligonucleotide complex (A: streptavidin; B: biotin); (c) dimeric dumbell oligonucleotide; (d) dimeric fork oligonucleotide.

FIG. 9 shows binding curves for the L-selectin SELEX fifteenth round ssDNA pool to PBMCs (10⁷/ml).

FIG. 10 shows the results of a competition experiment in which the binding of 2 nM ³²P-labeled round 15 ssDNA to PBMCs (10⁷/ml) is competed with increasing concentrations of the blocking monoclonal anti-L-selectin antibody, DREG-56, or an isotype matched, negative control antibody. RNA Bound equals 100×(net counts bound in the presence of competitor/net counts bound in the absence of competitor).

FIG. 11 shows L-selectin specific binding of LD201T1 (SEQ ID NO: 185) to human lymphocytes and granulocytes in whole blood a, FITC-LD201T1 binding to lymphocytes is competed by DREG-56, unlabeled LD201T1, and inhibited by EDTA. b, FITC-LD201T1 binding to granulocytes is competed by DREG-56, unlabeled LD201T1, and inhibited by EDTA. All samples were stained with 0.15 mM FITC-LD201T1; thick line: FITC-LD201T1 only; thick dashed line: FITC-LD201T1 with 0.3 mM DREG-56; medium thick line: FITC-LD201T1 with 7 mM unlabeled NX280; thin line: FITC-LD201T1 stained cells, reassayed after addition of 4 mM EDTA; thin dashed line: autofluorescence.

FIG. 12 shows the consensus hairpin secondary structures for family 1 ssDNA ligands to L-selectin. Nucleotide sequence is in standard one letter code. Invariant nucleotides are in bold type. The base pairs at highly variable positions are designated N—N′. To the right of the stem is a matrix showing the number of occurrences of particular base pairs for the position in the stem that is on the same line.

FIG. 13 shows that in vitro pre-treatment of human PBMC with NX288 (SEQ ID NO: 193) inhibits lymphocyte trafficking to SCID mouse PLN. Human PBMC were purified from heparinised blood by a Ficoll-Hypaque gradient, washed twice with HBSS (calcium/magnesium free) and labeled with ⁵¹Cr (Amersham). After labeling, the cells were washed twice with HBSS (containing calcium and magnesium) and 1% bovine serum albumin (Sigma). Female SCID mice (6-12 weeks of age) were injected intravenously with 2×10⁶ cells. The cells were either untreated or mixed with either 13 pmol of antibody (DREG-56 or MEL-14), or 4, 1, or 0.4 nmol of modified oligonucleotide. One hour later the animals were anaesthetized, a blood sample taken and the mice were euthanized. PLN, MLN, Peyer's patches, spleen, liver, lungs, thymus, kidneys and bone marrow were removed and the counts incorporated into the organs determined by a Packard gamma counter. Values shown represent the mean±s.e. of triplicate samples, and are representative of 3 experiments.

FIG. 14 shows that pre-injection of NX288 (SEQ ID NO:193) inhibits human lymphocyte trafficking to SCID mouse PLN and MLN. Human PBMC were purified, labeled, and washed as described above. Cells were prepared as described in FIG. 13. Female SCID mice (6-12 weeks of age) were injected intravenously with 2×10⁶ cells. One to 5 min prior to injecting the cells, the animals were injected with either 15 pmol DREG-56 or 4 nmol modified oligonucleotide. Animals were scarificed 1 hour after injection of cells. Counts incorporated into organs were quantified as described in FIG. 13. Values shown represent the mean±s.e. of triplicate samples, and are representative of 2 experiments.

FIG. 15 shows the consensus hairpin secondary structures for 2′-F RNA ligands to L-selectin. Nucleotide sequence is in standard one letter code. Invariant nucleotides are in bold type. The base pairs at highly variable positions are designated N—N′. To the right of the stem is a matrix showing the number of occurrences of particular base pairs for the position in the stem that is on the same line.

FIG. 16 shows the consensus hairpin secondary structures for 2′-F RNA ligands to P-selectin. Nucleotide sequence is in standard one letter code. Invariant nucleotides are in bold type. The base pairs at highly variable positions are designated N—N′. To the right of the stem is a matrix showing the number of occurrences of particular base pairs for the position in the stem that is on the same line.

### DETAILED DESCRIPTION OF THE INVENTION

This application describes high-affinity nucleic acid ligands to lectins identified through the method known as SELEX. SELEX is described in U.S. patent application Ser. No. 07/536,428, entitled “Systematic Evolution of Ligands by EXponential Enrichment”, now abandoned; U.S. patent application Ser. No. 07/714,131, filed Jun. 10, 1991, entitled “Nucleic Acid Ligands”, now U.S. Pat. No. 5,475,096; U.S. patent application Ser. No. 07/931,473, filed Aug. 17, 1992, entitled “Methods for Identifying Nucleic Acid Ligands”, now U.S. Pat. No. 5,270,163, (see also PCT/US91104078). These applications, each specifically incorporated herein by reference, are collectively called the SELEX patent applications.

In its most basic form, the SELEX process may be defined by the following series of steps:

1) A candidate mixture of nucleic acids of differing sequence is prepared. The candidate mixture generally includes regions of fixed sequences (i.e., each of the members of the candidate mixture contains the same sequences in the same location) and regions of randomized sequences. The fixed sequence regions are selected either: (a) to assist in the amplification steps described below, (b) to mimic a sequence known to bind to the target, or (c) to enhance the concentration of a given structural arrangement of the nucleic acids in the candidate mixture. The randomized sequences can be totally randomized (i.e., the probability of finding a base at any position being one in four) or only partially randomized (e.g., the probability of finding a base at any location can be selected at any level between 0 and 100 percent).

2) The candidate mixture is contacted with the selected target under conditions favorable for binding between the target and members of the candidate mixture. Under these circumstances, the interaction between the target and the nucleic acids of the candidate mixture can be considered as forming nucleic acid-target pairs between the target and those nucleic acids having the strongest affinity for the target.

3) The nucleic acids with the highest affinity for the target are partitioned from those nucleic acids with lesser affinity to the target. Because only an extremely small number of sequences (and possibly only one molecule of nucleic acid) corresponding to the highest affinity nucleic acids exist in the candidate mixture, it is generally desirable to set the partitioning criteria so that a significant amount of the nucleic acids in the candidate mixture (approximately 0.05-50%) are retained during partitioning.

4) Those nucleic acids selected during partitioning as having the relatively higher affinity to the target are then amplified to create a new candidate mixture that is enriched in nucleic acids having a relatively higher affinity for the target.

5) By repeating the partitioning and amplifying steps above, the newly formed candidate mixture contains fewer and fewer unique sequences, and the average degree of affinity of the nucleic acids to the target will generally increase. Taken to its extreme, the SELEX process will yield a candidate mixture containing one or a small number of unique nucleic acids representing those nucleic acids from the original candidate mixture having the highest affinity to the target molecule.

The SELEX patent applications describe and elaborate on this process in great detail. Included are targets that can be used in the process; methods for partitioning nucleic acids within a candidate mixture; and methods for amplifying partitioned nucleic acids to generate enriched candidate mixture. The SELEX patent applications also describe ligands obtained to a number of target species, including both protein targets where the protein is and is not a nucleic acid binding protein.

This invention also includes the ligands as described above, wherein certain chemical modifications are made in order to increase the in vivo stability of the ligand or to enhance or mediate the delivery of the ligand. Examples of such modifications include chemical substitutions at the sugar and/or phosphate and/or base positions of a given nucleic acid sequence. See, e.g., U.S. patent application Ser. No. 08/117,991, filed Sep. 8, 1993, entitled “High Affinity Nucleic Acid Ligands Containing Modified Nucleotides” which is specifically incorporated herein by reference. Additionally, in co-pending and commonly assigned U.S. patent application Ser. No. 07/964,624, filed Oct. 21, 1992 ('624), now U.S. Pat. No. 5,496,938, methods are described for obtaining improved nucleic acid ligands after SELEX has been performed. The '624 application, entitled “Nucleic Acid Ligands to HIV-RT and HIV-1 Rev,” is specifically incorporated herein by reference. Further included in the '624 patent are methods for determining the three-dimensional structures of nucleic acid ligands. Such methods include mathematical modeling and structure modifications of the SELEX-derived ligands, such as chemical modification and nucleotide substitution. Other modifications are known to one of ordinary skill in the art. Such modifications may be made post-SELEX (modification of previously identified unmodified ligands) or by incorporation into the SELEX process. Additionally, the nucleic acid ligands of the invention can be complexed with various other compounds, including but not limited to, lipophilic compounds or non-immunogenic, high molecular weight compounds. Lipophilic compounds include, but are not limited to, cholesterol, dialkyl glycerol, and diacyl glycerol. Non-immunogenic, high molecular weight compounds include, but are not limited to, polyethylene glycol, dextran, albumin and magnetite. The nucleic acid ligands described herein can be complexed with a lipophilic compound (e.g., cholesterol) or attached to or encapsulated in a complex comprised of lipophilic components (e.g., a liposome). The complexed nucleic acid ligands can enhance the cellular uptake of the nucleic acid ligands by a cell for delivery of the nucleic acid ligands to an intracellular target. The complexed nucleic acid ligands can also have enhanced pharmacokinetics and stability. U.S. patent application Ser. No. 08/434,465, filed May 4, 1995, entitled “Nucleic Acid Ligand Complexes,” now U.S. Pat. No. 6,011,020, which is herein incorporated by reference describes a method for preparing a therapeutic or diagnostic complex comprised of a nucleic acid ligand and a lipophilic compound or a non-immunogenic, high molecular weight compound.

The methods described herein and the nucleic acid ligands identified by such methods are useful for both therapeutic and diagnostic purposes. Therapeutic uses include the treatment or prevention of diseases or medical conditions in human patients. Many of the therapeutic uses are described in the background of the invention, particularly, nucleic acid ligands to selectins are useful as anti-inflammatory agents. Antagonists to the selectins modulate extravasion of leukocytes at sites of inflammation and thereby reduce neutrophil caused host tissue damage. Diagnostic utilization may include both in vivo or in vitro diagnostic applications. The SELEX method generally, and the specific adaptations of the SELEX method taught and claimed herein specifically, are particularly suited for diagnostic applications. SELEX identifies nucleic acid ligands that are able to bind targets with high affinity and with surprising specificity. These characteristics are, of course, the desired properties one skilled in the art would seek in a diagnostic ligand.

The nucleic acid ligands of the present invention may be routinely adapted for diagnostic purposes according to any number of techniques employed by those skilled in the art. Diagnostic agents need only be able to allow the user to identify the presence of a given target at a particular locale or concentration. Simply the ability to form binding pairs with the target may be sufficient to trigger a positive signal for diagnostic purposes. Those skilled in the art would also be able to adapt any nucleic acid ligand by procedures known in the art to incorporate a labeling tag in order to track the presence of such ligand. Such a tag could be used in a number of diagnostic procedures. The nucleic acid ligands to lectin, particularly selecting, described herein may specifically be used for identification of the lectin proteins.

SELEX provides high affinity ligands of a target molecule. This represents a singular achievement that is unprecedented in the field of nucleic acids research. The present invention applies the SELEX procedure to lectin targets. Specifically, the present invention describes the identification of nucleic acid ligands to Wheat Germ Agglutinin, and the selecting, specifically, L-selectin, P-selectin and E-selectin. In the Example section below, the experimental parameters used to isolate and identify the nucleic acid ligands to lectins are described.

In order to produce nucleic acids desirable for use as a pharmaceutical, it is preferred that the nucleic acid ligand (1) binds to the target in a manner capable of achieving the desired effect on the target; (2) be as small as possible to obtain the desired effect; (3) be as stable as possible; and (4) be a specific ligand to the chosen target. In most situations, it is preferred that the nucleic acid ligand have the highest possible affinity to the target.

In the present invention, a SELEX experiment was performed in search of nucleic acid ligands with specific high affinity for Wheat Germ Agglutinin from a degenerate library containing 50 random positions (50N). This invention includes the specific nucleic acid ligands to Wheat Germ Agglutinin shown in Table 2 (SEQ ID NOS: 4-55), identified by the methods described in Examples 1 and 2. Specifically, RNA ligands containing 2′-NH₂ modified pyrimidines are provided. The scope of the ligands covered by this invention extends to all nucleic acid ligands of Wheat Germ Agglutinin, modified and unmodified, identified according to the SELEX procedure. More specifically, this invention includes nucleic acid sequences that are substantially homologous to the ligands shown in Table 2. By substantially homologous it is meant a degree of primary sequence homology in excess of 70%, most preferably in excess of 80%. A review of the sequence homologies of the ligands of Wheat Germ Agglutinin shown in Table 2 shows that sequences with little or no primary homology may have substantially the same ability to bind Wheat Germ Agglutinin. For these reasons, this invention also includes nucleic acid ligands that have substantially the same ability to bind Wheat Germ Agglutinin as the nucleic acid ligands shown in Table 2. Substantially the same ability to bind Wheat Germ Agglutinin means that the affinity is within a few orders of magnitude of the affinity of the ligands described herein. It is well within the skill of those of ordinary skill in the art to determine whether a given sequence—substantially homologous to those specifically described herein—has substantially the same ability to bind Wheat Germ Agglutinin.

In the present invention, SELEX experiments were performed in search of nucleic acid ligands with specific high affinity for L-selectin from degenerate libraries containing 30 or 40 random positions (30N or 40N). This invention includes the, specific nucleic acid ligands to L-selectin shown in Tables 8, 12 and 16 (SEQ ID NOS: 67-117, 129-180, 185-196 and 293-388), identified by the methods described in Examples 7, 8, 13, 14, 22 and 23. Specifically, RNA ligands containing 2′-NH₂ or 2′-F pyrimidines and ssDNA ligands are provided. The scope of the ligands covered by this invention extends to all nucleic acid ligands of L-selectin, modified and unmodified, identified according to the SELEX procedure. More specifically, this invention includes nucleic acid sequences that are substantially homologous to the ligands shown in Tables 8, 12 and 16. By substantially homologous it is meant a degree of primary sequence homology in excess of 70%, most preferably in excess of 80%. A review of the sequence homologies of the ligands of L-selectin shown in Tables 8, 12 and 16 shows that sequences with little or no primary homology may have substantially the same ability to bind L-selectin. For these reasons, this invention also includes nucleic acid ligands that have substantially the same ability to bind L-selectin as the nucleic acid ligands shown in Tables 8, 12 and 16. Substantially the same ability to bind L-selectin means that the affinity is within a few orders of magnitude of the affinity of the ligands described herein. It is well within the skill of those of ordinary skill in the art to determine whether a given sequence—substantially homologous to those specifically described herein—has substantially the same ability to bind L-selectin.

In the present invention, SELEX experiments were performed in search of nucleic acid ligands with specific high affinity for P-selectin from degenerate libraries containing 50 random positions (SON). This invention includes the specific nucleic acid ligands to P-selectin shown in Tables 19 and 25 (SEQ ID NOS: 199-247 and 251-290), identified by the methods described in Examples 27, 28, 35 and 36. Specifically, RNA ligands containing 2′-NH₂ and 2′-F pyrimidines are provided. The scope of the ligands covered by this invention extends to all nucleic acid ligands of P-selectin, modified and unmodified, identified according to the SELEX procedure. More specifically, this invention includes nucleic acid sequences that are substantially homologous to the ligands shown in Tables 19 and 25. By substantially homologous it is meant a degree of primary sequence homology in excess of 70%, most preferably in excess of 80%. A review of the sequence homologies of the ligands of P-selectin shown in Tables 19 and 25 shows that sequences with little or no primary homology may have substantially the same ability to bind P-selectin. For these reasons, this invention also includes nucleic acid ligands that have substantially the same ability to bind P-selectin as the nucleic acid ligands shown in Tables 19 and 25. Substantially the same ability to bind P-selectin means that the affinity is within a few orders of magnitude of the affinity of the ligands described herein. It is well within the skill of those of ordinary skill in the art to determine whether a given sequence—substantially homologous to those specifically described herein—has substantially the same ability to bind P-selectin.

In the present invention, a SELEX experiment was performed in search of nucleic acid ligands with specific high affinity for E-selectin from a degenerate library containing 40 random positions (40N). This invention includes specific nucleic acid ligands to E-selectin identified by the methods described in Example 40. The scope of the ligands covered by this invention extends to all nucleic acid ligands of E-selectin, modified and unmodified, identified according to the SELEX procedure.

Additionally, the present invention includes multivalent Complexes comprising the nucleic acid ligands of the invention. The multivalent Complexes increase the binding energy to facilitate better binding affinities through slower off-rates of the nucleic acid ligands. The multivalent Complexes may be useful at lower doses than their monomeric counterparts. In addition, high molecular weight polyethylene glycol was included in some of the Complexes to decrease the in vivo clearance rate of the Complexes. Specifically, nucleic acid ligands to L-selectin were placed in multivalent Complexes.

As described above, because of their ability to selectively bind lectins, the nucleic acid ligands to lectins described herein are useful as pharmaceuticals. This invention, therefore, also includes a method for treating lectin-mediated diseases by administration of a nucleic acid ligand capable of binding to a lectin.

Therapeutic compositions of the nucleic acid ligands may be administered parenterally by injection, although other effective administration forms, such as intraarticular injection, inhalant mists, orally active formulations, transdermal iontophoresis or suppositories, are also envisioned. One preferred carrier is physiological saline solution, but it is contemplated that other pharmaceutically acceptable carriers may also be used. In one preferred embodiment, it is envisioned that the carrier and the ligand constitute a physiologically-compatible, slow release formulation. The primary solvent in such a carrier may be either aqueous or nonaqueous in nature. In addition, the carrier may contain other pharmacologically-acceptable excipients for modifying or maintaining the pH, osmolarity, viscosity, clarity, color, sterility, stability, rate of dissolution, or odor of the formulation. Similarly, the carrier may contain still other pharmacologically-acceptable excipients for modifying or maintaining the stability, rate of dissolution, release, or absorption of the ligand. Such excipients are those substances usually and customarily employed to formulate dosages for parental administration in either unit dose or multi-dose form.

Once the therapeutic composition has been formulated, it may be stored in sterile vials as a solution, suspension, gel, emulsion, solid, or dehydrated or lyophilized powder. Such formulations may be stored either in a ready to use form or requiring reconstitution immediately prior to administration. The manner of administering formulations containing nucleic acid ligands for systemic delivery may be via subcutaneous, intramuscular, intravenous, intranasal or vaginal or rectal suppository.

Well established animal models exist for many of the disease states which are candidates for selectin antagonist therapy. Models available for testing the efficacy of oligonucleotide selectin antagonists include:

1) mouse models for peritoneal inflammation (P. Pizcueta and F. W. Luscinskas, 1994, Am. J. Pathol. 145, 461-469), diabetes (A. C. Hanninen et al., 1992, J. Clin. Invest. 92, 2509-2515), lymphocyte trafficking (L. M. Bradley et al., 1994, J. Exp. Med., 2401-2406), glomerulonephritis (P. G. Tipping et al., 1994, Kidney Int. 46, 79-88), experimental allergic encephalomyelitis (J. M. Dopp et al., 1994, J. Neuroimmunol. 54: 129-144), acute inflammation in human/SCID mouse chimera (H.-C. Yan et al., 1994, J. Immunol. 152, 3053-3063), endotoxin-mediated inflammation (W. E. Sanders et al., 1992, Blood 80, 795-800);

2) rat models for acute lung injury (M. S. Milligan et al., 1994, J. Immunol. 152, 832-840), hind limb ischemia/reperfusion injury (A. Seekamp et al., 1994, Am. J. Pathol 144, 592-598), remote lung injury (A. Seekamp et al., 1994, supra; D. L. Carden et al., 1993, J. Appl. Physiol 75, 2529-2543), neutrophil rolling on mesenteric venules (K. Ley et al., 1993, Blood 82, 1632-1638), myocardial infarction ischemia reperfusion injury (D. Altavilla et al., 1994, Eur. J. Pharmacol. Environ. Toxicol. Pharmacol. 270, 45-51);

3) rabbit models for hemorrhagic shock (R. K. Winn et al., 1994, Am. J. Physiol. Heart Circ. Physiol. 267, H2391-H2397), ear ischemia reperfusion injury (D. Mihelcic et al., 1994, Bollod 84, 2333-2328) neutrophil rolling on mesenteric venules (A. M. Olofsson et al., Blood 84, 2749-2758), experimental meningitis (C. Granert et al., 1994, J. Clin. Invest. 93, 929-936); lung, peritoneal and subcutaneous bacterial infection (S. R. Sharer et al., 1993, J. Immunol. 151, 4982-4988), myocardial ischemia/repefusion (G. Montrucchio et al., 1989, Am. J. Physiol. 256, H1236-H1246), central nervous system ischemic injury (W. M. Clark et al., 1991, Stroke 22, 877-883);

4) cat models for myocardial infarction ischemia reperfusion injury (M. Buerke et al., 1994, J. Pharmacol. Exp. Ther. 271, 134-142);

5) dog models for myocardial infarction ischemia reperfusion injury (D. J. Lefer et al., 1994, Circulation 90, 2390-2401);

6) pig models for arthritis (F. Jamar et al., 1995, Radiology 194, 843-850);

7) rhesus monkey models for cutaneous inflammation (A. Silber et al., Lab. Invest. 70, 163-175);

8) cynomolgus monkey models for renal transplants (S.-L. Wee, 1991, Transplant. Prod. 23, 279-280); and

9) baboon models for dacron grafts (T. Palabrica et al, 1992, Nature 359, 848-851), septic, traumatic and hypovolemic shock (H. Redl et al., 1991, Am. J. Pathol. 139, 461-466).

The nucleic acid ligands to lectins described herein are useful as pharmaceuticals and as diagnostic reagents.

### EXAMPLES

The following examples are illustrative of certain embodiments of the invention and are not to be construed as limiting the present invention in any way. Examples 1-6 describe identification and characterization of 2′-NH₂ RNA ligands to Wheat Germ Agglutinin. Examples 7-12 described identification and characterization of 2′-NH₂ RNA ligands to L-selectin. Examples 13-21 describe identification and characterization of ssDNA ligands to L-selectin. Examples 22-25 describe identification and characterization of 2′-F RNA ligands to L-selectin. Example 26 describes identification of ssDNA ligands to P-selectin. Examples 27-39 describes identification and characterization of 2′-NH₂ and 2′-F RNA ligands to P-selectin. Example 40 describes identification of nucleic acid ligands to E-selectin.

### Example 1

### Nucleic Acid Ligands to Wheat Germ Agglutinin

The experimental procedures outlined in this Example were used to identify and characterize nucleic acid ligands to wheat germ agglutinin (WGA) as described in Examples 2-6.

### Experimental Procedures

### A) Materials

Wheat Germ Lectin (Triticum vulgare) Sepharose 6 MB beads were purchased from Pharmacia Biotech. Wheat Germ Lectin, Wheat Germ Agglutinin, and WGA are used interchangeably herein. Free Wheat Germ Lectin (Triticum vulgare) and all other lectins were obtained from E Y Laboratories; methyl-α-D-mannopyranoside was from Calbiochem and N-acetyl-D-glucosamine, GlcNAc, and the trisaccharide N N′N′-triacetylchitotriose, (GlcNAc)₃, were purchased from Sigma Chemical Co. The 2′-NH₂ modified CTP and UTP were prepared according to Pieken et. al. (1991, Science 253:314-317). DNA oligonucleotides were synthesized by Operon. All other reagents and chemicals were purchased from commercial sources. Unless otherwise indicated, experiments utilized Hanks' Balanced Salt Solutions (HBSS; 1.3 mM CaCl₂, 5.0 mM KCl, 0.3 mM KH₂PO₄, 0.5 mM MgCl₂.6H₂O, 0.4 mM MgSO₄.7H₂O, 138 mM NaCl, 4.0 mM NaHCO₃, 0.3 mM Na₂HPO₄, 5.6 mM D-Glucose; GibcoBRL).

### B) SELEX

The SELEX procedure is described in detail in U.S. Pat. No. 5,270,163 and elsewhere. In the wheat germ agglutinin SELEX experiment, the DNA template for the initial RNA pool contained 50 random nucleotides, flanked by N9 5′ and 3′ fixed regions (50N9) 5′ gggaaaagcgaaucauacacaaga-50N-gcuccgccagagaccaaccgagaa 3′ (SEQ ID NO: 1). All C and U have 2′-NH₂ substituted for 2′-OH for ribose. The primers for the PCR were the following: 5′ Primer 5′ taatacgactcactatagggaaaagcgaatcatacacaaga 3′ (SEQ ID NO: 2) and 3′ Primer 5′ ttctcggttggtctctggcggagc 3′ (SEQ ID NO: 3). The fixed regions of the starting random pool include DNA primer annealing sites for PCR and cDNA synthesis as well as the consensus T7 promoter region to allow in vitro transcription. These single-stranded DNA molecules were converted into double-stranded transcribable templates by PCR amplification. PCR conditions were 50 mM KCl, 10 mM Tris-Cl, pH 8.3, 0.1% TritonX-100, 7.5 mM MgCl₂, 1 mM of each dATP, dCTP, dGTP, and dTTP, and 25 U/ml of Taq DNA polymerase. Transcription reactions contained 5 mM DNA template, 5 units/μl T7 RNA polymerase, 40 mM Tris-Cl (pH 8.0), 12 mM MgCl₂, 5 mM DTT, 1 mM spermidine, 0.002% Triton X-100, 4% PEG 8000, 2 mM each of 2′-OH ATP, 2′-OH GTP, 2′-NH₂ CTP, 2′-NH₂ UTP, and 0.31 mM α-³²P 2′-OH ATP.

The strategy for partitioning WGA/RNA complexes from unbound RNA was 1) to incubate the RNA pool with WGA immobilized on Sepharose beads; 2) to remove unbound RNA by extensive washing; and 3) to specifically elute RNA molecules bound at the carbohydrate binding site by incubating the washed beads in buffer containing high concentrations of (GlcNAc)₃. The SELEX protocol is outlined in Table 1. The WGA density on Wheat Germ Lectin Sepharose 6 MB beads is approximately mg/ml of gel or 116 μM (manufacturer's specifications). After extensive washing in HBSS, the immobilized WGA was incubated with RNA at room temperature for 1 to 2 hours in a 2 ml siliconized column with constant rolling (Table 1). Unbound RNA was removed by extensive washing with HBSS. Bound RNA was eluted as two fractions; first, nonspecifically eluted RNA was removed by incubating and washing with 10 mM methyl-α-D-mannopyranoside in HBSS (Table 1; rounds 1-4) or with HBSS (Table 1; rounds 5-11); second, specifically eluted RNA was removed by incubating and washing with 0.5 to 10 mM (GlcNAc)₃ in HBSS (Table 1). The percentage of input RNA that was specifically eluted is recorded in Table 1.

The specifically eluted fraction was processed for use in the following round. Fractions eluted from immobilized WGA were heated at 90° C. for 5 minutes in 1% SDS, 2% β-mercaptoethanol and extracted with phenol/chloroform. RNA was reverse transcribed into cDNA by AMV reverse transcriptase at 48° C. for 60 min in 50 mM Tris-Cl pH (8.3), 60 mM NaCl, 6 mM Mg(OAc)₂, 10 mM DTT, 100 pmol DNA primer, 0.4 mM each of dNTPs, and 0.4 unit/μl AMV RT. PCR amplification of this cDNA resulted in approximately 500 pmol double-stranded DNA, transcripts of which were used to initiate the next round of SELEX.

### C) Nitrocellulose Filter Binding Assay

As described in SELEX patent applications, a nitrocellulose filter partitioning method was used to determine the affinity of RNA ligands for WGA and for other proteins. Filter discs (nitrocellulose/cellulose acetate mixed matrix, 0.45 μm pore size, Millipore; or pure nitrocellulose, 0.45 μm pore size, Bio-Rad) were placed on a vacuum manifold and washed with 4 ml of HBSS buffer under vacuum. Reaction mixtures, containing ³²P labeled RNA pools and unlabeled WGA, were incubated in HBSS for 10 min at room temperature, filtered, and then immediately washed with 4 ml HBSS. The filters were air-dried and counted in a Beckman LS6500 liquid scintillation counter without fluor.

WGA is a homodimer, molecular weight 43.2 kD, with 4 GlcNAc binding sites per dimer. For affinity calculations, we assume one RNA ligand binding site per monomer (two per dimer). The monomer concentration is defined as 2 times the dimer concentration. The equilibrium dissociation constant, K_{d}, for an RNA pool or specific ligand that binds monophasically is given by the equation

K_{d}=[P_{f}][R_{f}]/[RP]
where,
   [R_{f}]=free RNA concentration
   [P_{f}]=free WGA monomer concentration
   [RP]=concentration of RNA/WGA monomer complexes

K_{d}=dissociation constant

A rearrangement of this equation, in which the fraction of RNA bound at equilibrium is expressed as a function of the total concentration of the reactants, was used to calculate Kds of monophasic binding curves:

q=(P_{T}+R_{T}.+K_{d}−((P_{T}+R_{T}.+K_{d})².−4 P_{T}R_{T})^{1/2})
q=fraction of RNA bound
[P_{T}]=total WGA monomer concentration
[R_{T}]=total RNA concentration
   K_{d}s were determined by least square fitting of the data points using the graphics program Kaleidagraph (Synergy Software, Reading, Pa.).

### D) Cloning and Sequencing

The sixth and eleventh round PCR products were re-amplified with primers which contain a BamH1 or a EcoR1 restriction endonuclease recognition site. Using these restriction sites the DNA sequences were inserted directionally into the pUC18 vector. These recombinant plasmids were transformed into E. coli strain JM109 (Stratagene, La Jolla, Calif.). Plasmid DNA was prepared according to the alkaline hydrolysis method (Zhou et al., 1990 Biotechniques 8:172-173) and about 72 clones were sequenced using the Sequenase protocol (United States Biochemical Corporation, Cleveland, Ohio). The sequences are provided in Table 2.

### E) Competitive Binding Studies

Competitive binding experiments were performed to determine if RNA ligands and (GlcNAc)₃ bind the same site on WGA. A set of reaction mixtures containing α-³²P labeled RNA ligand and unlabeled WGA, each at a fixed concentration (Table 5), was incubated in HBSS for 15 min at room temperature with (GlcNAc)₃. Individual reaction mixtures were then incubated with a (GlcNAc)₃ dilution from a 2-fold dilution series for 15 minutes. The final (GlcNAc)₃ concentrations ranged from 7.8 μM to 8.0 mM (Table 5). The reaction mixtures were filtered, processed and counted as described in “Nitrocellulose Filter Binding Assay,” paragraph D above.

Competition titration experiments were analyzed by the following equation to determine the concentration of free protein [P] as a function of the total concentration of competitor added, [C_{T}]:

0=[P](1+K_{L}[L_{T}]/(1+K_{L}[P])+K_{C}.[C_{T}]/(1+K_{C}[P]))−P_{T}

where L_{T} is the concentration of initial ligand, K_{L} is the binding constant of species L to the protein (assuming 1:1 stiochiometry) and K_{C} is the binding constant of species C to the protein (assuming 1:1 stiochiometry). Since it is difficult to obtain a direct solution for equation 1, iteration to determine values of [P] to a precision of 1×10⁻¹⁵ was used. Using these values of [P], the concentration of protein-ligand complex [PL] as a function of [C_{T}] was determined by the following equation:

[P_{L}]=K_{L}[L_{T}][P](1+K_{L}[P])

Since the experimental data is expressed in terms of % [PL], the calculated concentration of [PL] was normalized by the initial concentration of [PLo] before addition of the competitor. ([PLo] was calculated using the quadratic solution for the standard binding equation for the conditions used. The maximum (M) and minimum (B) % [PL] was allowed to float during the analysis as shown in the following equation.

% [PL]=[PL]/[PLₒ]*(M−B)+B

A non-linear least-squares fitting procedure was used as described by P. R. Bevington (1969) Data Reduction and Error Analysis for the Physical Sciences, McGraw-Hill publishers. The program used was originally written by Stanley J. Gill in MatLab and modified for competition analysis by Stanley C. Gill. The data were fit to equations 1-3 to obtain best fit parameters for K_{C}, M and B as a function of [C_{T}] while leaving K_{L} and P_{T} fixed.

### F) Inhibition of WGA Agglutinating Activity

Agglutination is a readily observed consequence of the interaction of a lectin with cells and requires that individual lectin molecules crosslink two or more cells. Lectin mediated agglutination can be inhibited by sugars with appropriate specificity. Visual assay of the hemagglutinating activity of WGA and the inhibitory activity of RNA ligands, GlcNAc and (GlcNAc)₃ was made in Falcon round bottom 96 well microtiter plates, using sheep erythrocytes. Each well contained 54 μl of erythrocytes (2.5×10⁸ cells/ml) and 54 μl of test solution. To titrate WGA agglutinating activity, each test solution contained a WGA dilution from a 4-fold dilution series. The final WGA concentrations ranged from 0.1 μM to 0.5 μM. For inhibition assays, the test solutions contained 80 nM WGA (monomer) and a dilution from a 4-fold dilution series of the designated inhibitor. Reaction mixtures were incubated at room temperature for 2 hours, after which time no changes were observed in the precipitation patterns of erythrocytes. These experiments were carried out in Gelatin Veronal Buffer (0.15 nM CaCl₂, 141 mM NaCl, 0.5 mM MgCl₂, 0.1% gelatin, 1.8 mM sodium barbital, and 3.1 mM barbituric acid, pH 7.3-7.4; Sigma #G-6514).

In the absence of agglutination, erythrocytes settle as a compact pellet. Agglutinated cells form a more diffuse pellet. Consequently, in visual tests, the diameter of the pellet is diagnostic for agglutination. The inhibition experiments included positive and negative controls for agglutination and appropriate controls to show that the inhibitors alone did not alter the normal precipitation pattern.

### Example 2

### RNA Ligands to WGA

### A. SELEX

The starting RNA library for SELEX, randomized 50N9 (SEQ ID NO: 1), contained approximately 2×10¹⁵ molecules (2 nmol RNA). The SELEX protocol is outlined in Table 1. Binding of randomized RNA to WGA is undetectable at 36 μM WGA monomer. The dissociation constant of this interaction is estimated to be >4 mM.

The percentage of input RNA eluted by (GlcNAc)₃ increased from 0.05% in the first round, to 28.5% in round 5 (Table 1). The bulk K_{d} of round 5 RNA was 600 nM (Table 1). Since an additional increase in specifically eluted RNA was not observed in round 6a (Table 1), round 6 was repeated (Table 1, round 6b) with two modifications to increase the stringency of selection: the volume of gel, and hence the mass of WGA, was reduced ten fold; and RNA was specifically eluted with increasing concentrations of (GlcNAc)3, in stepwise fashion, with only the last eluted RNA processed for the following round. The percentage of specifically eluted RNA increased from 5.7% in round 6b to 21.4% in round 8, with continued improvement in the bulk Kd (260 nM, round 8 RNA, Table 1).

For rounds 9 through 11 the WGA mass was again reduced ten fold to further increase stringency. The K_{d} of round 11 RNA was 68 nM. Sequencing of the bulk starting RNA pool and sixth and eleventh round RNA revealed some nonrandomness in the variable region at the sixth round and increased nonrandomness at round eleven.

To monitor the progress of SELEX, ligands were cloned and sequenced from round 6b and round 11. From each of the two rounds, 36 randomly picked clones were sequenced. Sequences were aligned manually and are shown in Table 2.

### B. RNA Sequences

From the sixth and eleventh rounds, respectively, 27 of 29 and 21 of 35 sequenced ligands were unique. The number before the “.” in the ligand name indicates whether it was cloned from the round 6 or round 11 pool. Only a portion of the entire clone is shown in Table 2 (SEQ ID NOS: 4-55). The entire evolved random region is shown in upper case letters. Any portion of the fixed region is shown in lower case letters. By definition, each clone includes both the evolved sequence and the associated fixed region, unless specifically stated otherwise. A unique sequence is operationally defined as one that differs from all others by three or more nucleotides. In Table 2, ligands sequences are shown in standard single letter code (Cornish-Bowden, 1985 NAR 13: 3021-3030). Sequences that were isolated more than once are indicated by the parenthetical number, (n), following the ligand isolate number. These clones fall into nine sequence families (1-9) and a group of unrelated sequences (Orphans).

The distribution of families from round six to eleven provides a clear illustration of the appearance and disappearance of ligand families in response to increased selective pressure (Table 2). Family 3, predominant (11/29 ligands) in round 6, has nearly disappeared (2/35) by round 11. Similarly, minor families 6 through 9 virtually disappear. In contrast, only one (family 1) of round eleven's predominant families (1, 2, 4 and 5) was detected in round six. The appearance and disappearance of families roughly correlates with their binding affinities.

Alignment (Table 2) defines consensus sequences for families 14 and 6-9 (SEQ ID NOS: 56-63). The consensus sequences of families 1-3 are long (20, 16 and 16, respectively) and very highly conserved. The consensus sequences of families 1 and 2 contain two sequences in common: the trinucleotide TCG and the pentanucleotide ACGAA. A related tetranucleotide, AACG, occurs in family 3. The variation in position of the consensus sequences within the variable regions indicates that the ligands do not require a specific sequence from either the 5′ or 3′ fixed region.

The consensus sequences of family 1 and 2 are flanked by complementary sequences 5 or more nucleotides in length. These complementary sequences are not conserved and the majority include minor discontinuities. Family 3 also exhibits flanking complementary sequences, but these are more variable in length and structure and utilize two nucleotide pairs of conserved sequence.

Confidence in the family 4 consensus sequence (Table 2) is limited by the small number of ligands, the variability of spacing and the high G content. The pentanucleotide, RCTGG, also occurs in families 5 and 8. Ligands of family 5 show other sequence similarities to those of family 4, especially to ligand 11.28.

### C. Affinities

The dissociation constants for representative members of families 1-9 and orphan ligands were determined by nitrocellulose filter binding experiments and are listed in Table 3. These calculations assume one RNA ligand binding site per WGA monomer. At the highest WGA concentration tested (36 μM WGA monomer), binding of random RNA is not observed, indicating a K_{d} at least 100-fold higher than the protein concentration or >4 mM.

The data in Table 3 define several characteristics of ligand binding. First, RNA ligands to WGA bind monophasically. Second, the range of measured dissociation constants is 1.4 nM to 840 nM. Third, the binding for a number of ligands, most of which were sixth round isolates, was less than 5% at the highest WGA concentration tested. The dissociation constants of these ligands are estimated to be greater than 20 μM. Fourth, on average eleventh round isolates have higher affinity than those from the sixth round. Fifth, the SELEX probably was not taken to completion; the best ligand (11.20) (SEQ ID NO: 40) is not the dominant species. Since the SELEX was arbitrarily stopped at the 11th round, it is not clear that 11.20 would be the ultimate winner. Sixth, even though the SELEX was not taken to completion, as expected, RNA ligands were isolated that bind WGA with much greater affinity than do mono- or oligosaccharides (i.e., the affinity of 11.20 is 5×10⁵ greater than that of GlcNAc, Kd=760 μM, and 850 better than that of (GlcNAc)₃, Kd=12 μM; Y. Nagata and M. Burger, 1974, supra). This observation validates the proposition that competitive elution allows the isolation of oligonucleotide ligands with affinities that are several orders of magnitude greater than that of the competing sugar.

In addition these data show that even under conditions of high target density, 116 pmol WGA dimer/μl of beads, it is possible to overcome avidity problems and recover ligands with nanomolar affinities. From the sixth to the eleventh round (Table 2), in response to increased selective pressure as indicated by the improvement in bulk K_{d} (Table 1), sequence families with lower than average affinity (Table 3) are eliminated from the pool.

### Example 3

### Specificity of RNA Ligands to WGA

The affinity of WGA ligands 6.8, 11.20 and 11.24 (SEQ ID NOS: 13, 40, and 19) for GlcNAc binding lectins from Ulex europaeus, Datura stramonium and Canavalia ensiformis were determined by nitrocellulose partitioning. The results of this determination are shown in Table 4. The ligands are highly specific for WGA. For example, the affinity of ligand 11.20 for WGA is 1,500, 8,000 and >15,000 fold greater than it is for the U. europaeus, D. stramonium and C. ensiformis lectins, respectively. The 8,000 fold difference in affinity for ligand 11.20 exhibited by T. vulgare and D. stramonium compares to a 3 to 10 fold difference in their affinity for oligomers of GlcNAc and validates the proposition that competitive elution allows selection of oligonucleotide ligands with much greater specificity than monomeric and oligomeric saccharides (J. F. Crowley et al., 1984, Arch. Biochem. and Biophys. 231:524-533; Y. Nagata and M. Burger, 1974, supra; J-P. Privat et al., FEBS Letters 46:229-232).

### Example 4

### Competitive Binding Studies

If an RNA ligand and a carbohydrate bind a common site, then binding of the RNA ligand is expected to be competitively inhibited by the carbohydrate. Furthermore, if the oligonucleotide ligands bind exclusively to carbohydrate binding sites, inhibition is expected to be complete at high carbohydrate concentrations. In the experiments reported in Table 5, dilutions of unlabeled (GlcNAc)₃, from a 2-fold dilution series, were added to three sets of binding reactions that contained WGA and an α ³²P labeled RNA ligand (6.8, 11.20 or 11.24 (SEQ ID NOS: 13, 40 and 19); [RNA] final=[WGA]final=15 nM). After a 15 minute incubation at room temperature, the reactions were filtered and processed as in standard binding experiments.

Qualitatively, it is clear that RNA ligands bind only to sites at which (GlcNAc)₃ binds, since inhibition is complete at high (GlcNAc)₃ concentrations (Table 5). These data do not rule out the possibility that (GlcNAc)₃ binds one or more sites that are not bound by these RNA ligands.

Quantitatively, these data fit a simple model of competitive inhibition (Table 5) and give estimates of 8.4, 10.9 and 19.4 μM for the Kd of (GlcNAc)₃. These estimates are in good agreement with literature values (12 μM@4 C, Nagata and Burger, 1974, supra; 11 μM@10.8 C, Van Landschoot et al., 1977, Eur. J. Biochem. 79:275-283; 50 μM, M. Monsigny et al., 1979, Eur J. Biochem. 98:39-45). These data confirm the proposition that competitive elution with a specific carbohydrate targets the lectin's carbohydrate binding site.

### Example 5

### Inhibition of WGA Agglutinating Activity

At 0.5 μM, RNA ligands 6.8 and 11.20 (SEQ ID NO: 13 and 40) completely inhibit WGA mediated agglutination of sheep erythrocytes (Table 6). Ligand 11.24 (SEQ ID NO: 19) is not as effective, showing only partial inhibition at 2 μM, the highest concentration tested (Table 6). (GlcNAc)₃ and GlcNAc completely inhibit agglutination at higher concentrations, 8 μM and 800 μM, respectively, (Table 6; Monsigny et al., supra). The inhibition of agglutination verifies the proposition that ligands isolated by this procedure will be antagonists of lectin function. Inhibition also suggests that more than one RNA ligand is bound per WGA dimer, since agglutination is a function of multiple carbohydrate binding sites.

An alternative interpretation for the inhibition of agglutination is that charge repulsion prevents negatively charged WGA/RNA complexes from binding carbohydrates (a necessary condition for agglutination) on negatively charged cell surfaces. This explanation seems unlikely for two reasons. First, negatively charged oligonucleotide ligands selected against an immobilized purified protein are known to bind to the protein when it is presented in the context of a cell surface (see Example 10, L-selectin cell binding). Second, negatively charged (pI=4) succinylated WGA is as effective as native WGA (pI=8.5) in agglutinating erythrocytes (M. Monsigny et al., supra).

### Example 6

### Secondary Structure of High Affinity WGA Ligands

In favorable instances, comparative analysis of aligned sequences allows deduction of secondary structure and structure-function relationships. If the nucleotides at two positions in a sequence covary according to Watson-Crick base pairing rules, then the nucleotides at these positions are apt to be paired. Nonconserved sequences, especially those that vary in length are not apt to be directly involved in function, while highly conserved sequence are likely to be directly involved.

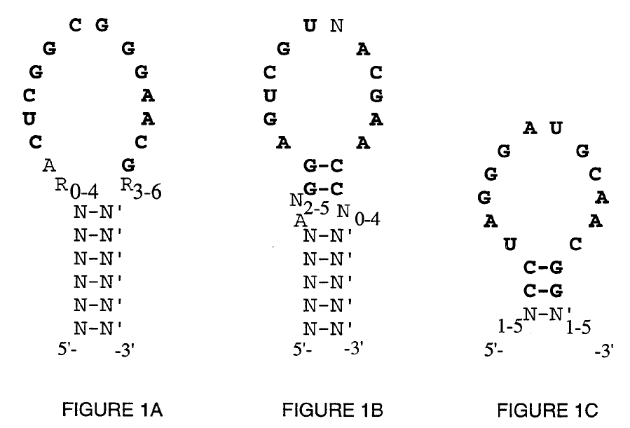

Comparative analyses of both family 1 and 2 sequences each yield a hairpin structure with a large highly conserved loop (FIGS. 1a and 1b). Interactions between loop nucleotides are likely but they are not defined by these data. The stems of individual ligands vary in sequence, length and structure (i.e., a variety of bulges and internal loops are allowed; Table 2). Qualitatively it is clear that the stems are validated by Watson/Crick covariation and that by the rules of comparative analysis the stems are not directly involved in binding WGA. Family 3 can form a similar hairpin in which 2 pairs of conserved nucleotides are utilized in the stem (FIG. 1c).

If it is not possible to fold the ligands of a sequence family into homologous structures, their assignment to a single family is questionable. Both ligand 11.7, the dominant member of family 4, and ligand 11.28 can be folded into two plane G-quartets. However, this assignment is speculative: 1) 11.28 contains five GG dinucleotides and one GGGG tetranucleotide allowing other G-quartets; and 2) ligands 11.2 and 11.33 cannot form G-quartets. On the other hand, all ligands can form a hairpin with the conserved sequence GAGRFTNCRT in the loop. However, the conserved sequence RCTGGC (Table 2) does not have a consistent role in these hairpins.

Multiple G-quartet structures are possible for Family 5. One of these resembles the ligand 11.7 G-quartet. No convincing hairpin structures are possible for ligand 11.20.

### Example 7

### 2′-NH₂ RNA Ligands to Human L-Selectin

The experimental procedures outlined in this Example were used to identify and characterize the 2′-NH₂ RNA ligands to human L-selectin in Examples 8-12.

### Experimental Procedures

### A) Materials

LS-Rg is a chimeric protein in which the extracellular domain of human L-selectin is joined to the Fc domain of a human G2 immunoglobulin (Norgard et al., 1993, PNAS 90:1068-1072). ES-Rg, PS-Rg and CD22β-Rg are analogous constructs of E-selectin, P-selectin and CD22β joined to a human G1 immunoglobulin Fc domain (R. M. Nelson et al., 1993, supra; I. Stamenkovic et al., 1991, Cell 66, 1133-1144). Purified chimera were provided by A. Varki.

Soluble P-selectin was purchased from R&D Systems. Protein A Sepharose 4 Fast Flow beads were purchased from Pharmacia Biotech. Anti-L-selectin monoclonal antibodies: SK11 was obtained from Becton-Dickinson, San Jose, Calif.; DREG-56, an L-selectin specific monoclonal antibody, was purchased from Endogen, Cambridge, Mass. The 2′-NH₂ modified CTP and UTP were prepared according to Pieken et. al. (1991, Science 253:314-317). DNA oligonucleotides were synthesized by Operon. All other reagents and chemicals were purchased from commercial sources. Unless otherwise indicated, experiments utilized HSMC buffer (1 mM CaCl₂, 1 mM MgCl₂, 150 mM NaCl, 20.0 mM HEPES, pH 7.4).

### B) SELEX

The SELEX procedure is described in detail in U.S. Pat. No. 5,270,163 and elsewhere. The nucleotide sequence of the synthetic DNA template for the LS-Rg SELEX was randomized at 40 positions. This variable region was flanked by N7 5′ and 3′ fixed regions (40N7). 40N7 transcript has the sequence 5′ gggaggacgaugcgg-40N-cagacgacucgcccga 3′ (SEQ ID NO: 64). All C and U have 2′-NH₂ substituted for 2′-OH on the ribose. The primers for the PCR were the following:

| | | |
|---|---|---|
| N7 5′ Primer | 5′ taatacgactcactatagggaggacgatgcgg 3′ | (SEQ ID NO: 65) |
| N7 3′ Primer | 5′ tcgggcgagtcgtcctg 3′ | (SEQ ID NO: 66) |

The fixed regions include primer annealing sites for PCR and cDNA synthesis as well as a consensus T7 promoter to allow in vitro transcription. The initial RNA pool was made by first Klenow extending 1 nmol of synthetic single stranded DNA and then transcribing the resulting double stranded molecules with T7 RNA polymerase. Klenow extension conditions: 3.5 nmols primer 5N7, 1.4 nmols 40N7, 1× Klenow Buffer, 0.4 mM each of dATP, dCTP, dGTP and dTTP in a reaction volume of 1 ml.

For subsequent rounds, eluted RNA was the template for AMV reverse transcriptase mediated synthesis of single-stranded cDNA. These single-stranded DNA molecules were converted into double-stranded transcription templates by PCR amplification. PCR conditions were 50 mM KCl, 10 mM Tris-Cl, pH 8.3, 7.5 mM MgCl₂, 1 mM of each dATP, dCTP, dGTP, and dTTP, and 25 U/ml of Taq DNA polymerase. Transcription reactions contained 0.5 mM DNA template, 200 nM T7 RNA polymerase, 80 mM HEPES (pH 8.0), 12 mM MgCl₂, 5 mM DTT, 2 mM spermidine, 2 mM each of 2′-OH ATP, 2′-OH GTP, 2′-NH₂ CTP, 2′-NH₂ UTP, and 250 nM a ³²P 2′-OH ATP.

The strategy for partitioning LS-Rg/RNA complexes from unbound RNA is outlined in Tables 7a and 7b. First, the RNA pool was incubated with LS-Rg immobilized on protein A Sepharose beads in HSMC buffer. Second, the unbound RNA was removed by extensive washing. Third, the RNA molecules bound at the carbohydrate binding site were specifically eluted by incubating the washed beads in HMSC buffer containing 5 mM EDTA in place of divalent cations. The 5 mM elution was followed by a non-specific 50 mM EDTA elution. LS-Rg was coupled to protein A Sepharose beads according to the manufacturer's instructions (Pharmacia Biotech).

The 5 mM EDTA elution is a variation of a specific site elution strategy. Although it is not a priori as specific as elution by carbohydrate competition, it is a general strategy for C-type (calcium dependent binding) lectins and is a practical alternative when the cost and/or concentration of the required carbohydrate competitor is unreasonable (as is the case with sialyl-Lewis^{X}). This scheme is expected to be fairly specific for ligands that form bonds with the lectin's bound Ca⁺⁺ because the low EDTA concentration does not appreciably increase the buffer's ionic strength and the conformation of C-type lectins is only subtly altered in the absence of bound calcium (unpublished observations cited by K. Drickamer, 1993, Biochem. Soc. Trans. 21:456-459).

In the initial SELEX rounds, which were performed at 4° C., the density of immobilized LS-Rg was 16.7 pmols/μl of Protein A Sepharose 4 Fast Flow beads. In later rounds, the density of LS-Rg was reduced (Tables 7a and 7b), as needed, to increase the stringency of selection. At the seventh round, the SELEX was branched and continued in parallel at 4° C. (Table 7a) and at room temperature (Table 7b). Wash and elution buffers were equilibrated to the relevant incubation temperature. Beginning with the fifth round, SELEX was often done at more than one LS-Rg density. In each branch, the eluted material from only one LS-Rg density was carried forward.

Before each round, RNA was batch adsorbed to 100 μl of protein A Sepharose beads for 1 hour in a 2 ml siliconized column. Unbound RNA and RNA eluted with minimal washing (two volumes) were combined and used for SELEX input material. For SELEX, extensively washed, immobilized LS-Rg was batch incubated with pre-adsorbed RNA for 1 to 2 hours in a 2 ml siliconized column with constant rocking. Unbound RNA was removed by extensive batch washing (200 to 500 μl HSMC/wash). Bound RNA was eluted as two fractions; first, bound RNA was eluted by incubating and washing columns with 5 mM EDTA in HSMC without divalent cations; second the remaining elutable RNA was removed by incubating and/or washing with 50 mM EDTA in HSMC without divalents. The percentage of input RNA that was eluted is recorded in Tables 7a and 7b. In every round, an equal volume of protein A Sepharose beads without LS-Rg was treated identically to the SELEX beads to determine background binding. All absorbed, wash and eluted fractions were counted in a Beckman LS6500 scintillation counter in order to monitor each round of SELEX.

The eluted fractions were processed for use in the following round (Tables 7a and 7b). After extracting with phenol/chloroform and precipitating with isopropanol/ethanol (1:1, v/v), the RNA was reverse transcribed into cDNA by AMV reverse transcriptase either 1) at 48° C. for 15 minutes and then 65° C. for 15 minutes or 2) at 37° C. and 48° C. for 15 minutes each, in 50 mM Tris-Cl pH (8.3), 60 mM NaCl, 6 mM Mg(OAc)₂, 10 mM DTT, 100 pmol DNA primer, 0.4 mM each of dNTPs, and 0.4 unit/μl AMV RT. Transcripts of the PCR product were used to initiate the next round of SELEX.

### C) Nitrocellulose Filter Binding Assay

As described in SELEX patent applications, a nitrocellulose filter partitioning method was used to determine the affinity of RNA ligands for LS-Rg and for other proteins. Filter discs (nitrocellulose/cellulose acetate mixed matrix, 0.45 μm pore size, Millipore) were placed on a vacuum manifold and washed with 2 ml of HSMC buffer under vacuum. Reaction mixtures, containing ³²P labeled RNA pools and unlabeled LS-Rg, were incubated in HSMC for 10-20 min at 4° C., room temperature or 37° C., filtered, and then immediately washed with 4 ml HSMC at the same temperature. The filters were air-dried and counted in a Beckman LS6500 liquid scintillation counter without fluor.

LS-Rg is a dimeric protein that is the expression product of a recombinant gene constructed by fusing the DNA sequence that encodes the extracellular domains of human L-selectin to the DNA that encodes a human IgG₂ Fc region. For affinity calculations, we assume one RNA ligand binding site per LS-Rg monomer (two per dimer). The monomer concentration is defined as 2 times the LS-Rg dimer concentration. The equilibrium dissociation constant, K_{d}, for an RNA pool or specific ligand that binds monophasically is given by the equation

K_{d}=[P_{f}][R_{f}]/[RP]
where,
   [R_{f}]=free RNA concentration
   [P_{f}]=free WGA monomer concentration
   [RP]=concentration of RNA/WGA monomer complexes
   K_{d}=dissociation constant
      A rearrangement of this equation, in which the fraction of RNA bound at equilibrium is expressed as a function of the total concentration of the reactants, was used to calculate Kds of monophasic binding curves:

q=(P_{T}+R_{T}.+K_{d}−((P_{T}+R_{T}.+K_{d})².−4 P_{T}R_{T})^{1/2})
q=fraction of RNA bound
[P_{T}]=total WGA monomer concentration
[R_{T}]=total RNA concentration
   Many ligands and evolved RNA pools yield biphasic binding curves. Biphasic binding can be described as the binding of two affinity species that are not in equilibrium. Biphasic binding data were evaluated with the equation

q=2Pₜ+Rₜ+Kd₁+Kd₂−[(Pₜ+X₁R₁+Kd₁)²−4PₜX₁Rₜ]^{1/2}−[(Pₜ+X₂Rₜ+Kd₂)²−4PₜX₂Rₜ]^{1/2},

where X₁ and X₂ are the mole fractions of affinity species R₁ and R₂ and Kd₁ and Kd₂ are the corresponding dissociation constants. Kds were determined by least square fitting of the data points using the graphics program Kaleidagraph (Synergy Software, Reading, Pa.).

### D) Cloning and Sequencing

Sixth, thirteenth (RT) and fourteenth (4° C.) round PCR products were re-amplified with primers which contain either a BamHI or a HinDIII restriction endonuclease recognition site. Using these restriction sites, the DNA sequences were inserted directionally into the pUC9 vector. These recombinant plasmids were transformed into E. coli strain DH5a (Life Technologies, Gaithersburg, Md.). Plasmid DNA was prepared according to the alkaline hydrolysis method (PERFECTprep, 5′-3′, Boulder, Colo.). Approximately 150 clones were sequenced using the Sequenase protocol (Amersham, Arlington Heights, Ill.). The resulting ligand sequences are shown in Table 8.

### E) Cell Binding Studies

The ability of evolved ligand pools and cloned ligands to bind to L-selectin presented in the context of a cell surface was tested in experiments with isolated human peripheral blood mononuclear cells (PBMCs). Whole blood, collected from normal volunteers, was anticoagulated with 5 mM EDTA. Six milliliters of blood were layered on a 6 ml Histopaque gradient in 15 ml polypropylene tube and centrifuged (700 g) at room temperature for 30 minutes. The mononuclear cell layer was collected, diluted in 10 ml of Ca⁺⁺/Mg⁺⁺-free DPBS (DPBS(−); Gibco 14190-029) and centrifuged (225 g) for 10 minutes at room temperature. Cell pellets from two gradients were combined, resuspended in 10 ml of DPBS(−) and recentrifuged as described above. These pellets were resuspended in 100 μl of SMHCK buffer supplemented with 1% BSA. Cells were counted in a hemocytometer, diluted to 2×10⁷ cells/ml in SMHCK/1% BSA and immediately added to binding assays. Cell viability was monitored by trypan blue exclusion.

For cell binding assays, a constant number of cells were titrated with increasing concentrations of radiolabeled ligand. The test ligands were serially diluted in DPBS(−)/1% BSA to 2-times the desired final concentration approximately 10 minutes before use. Equal volumes (25 μl) of each ligand dilution and the cell suspension (2×10⁷ cells/ml) were added to 0.65 ml eppendorf tubes, gently vortexed and incubated on ice for 30 minutes. At 15 minutes the tubes were revortexed. The ligand/PBMC suspension was layered over 50 μl of ice cold phthalate oil (1:1=dinonyl:dibutyl phthalate) and microfuged (14,000 g) for 5 minutes at 4° C. Tubes were frozen in dry ice/ethanol, visible pellets amputated into scintillation vials and counted in Beckman LS6500 scintillation counter as described in Example 7, paragraph C.

The specificity of binding to PBMCs was tested by competition with the L-selectin specific blocking monoclonal antibody, DREG-56, while saturability of binding was tested by competition with unlabeled RNA. Experimental procedure and conditions were like those for PBMC binding experiments, except that the radiolabeled RNA ligand (final concentration 5 nM) was added to serial dilutions of the competitor before mixing with PBMCs.

### F) Inhibition of Selectin Binding to Sialyl-Lewis^{X}

The ability of evolved RNA pools or cloned ligands to inhibit the binding of LS-Rg to sialyl-Lewis^{X} was tested in competitive ELISA assays (C. Foxall et al., 1992, supra). For these assays, the wells of Coming (25801) 96 well microtiter plates were coated with 100 ng of a sialyl-Lewisx/BSA conjugate, air dried overnight, washed with 300 μl of PBS(−) and then blocked with 1% BSA in SHMCK for 60 min at room temperature. RNA ligands were incubated with LS-Rg in SHMCK/1% BSA at room temperature for 15 min. After removal of the blocking solution, 50 μl of LS-Rg (10 nM) or a LS-Rg (10 nM)/RNA ligand mix was added to the coated, blocked wells and incubated at room temperature for 60 minutes. The binding solution was removed, wells were washed with 300 μl of PBS(−) and then probed with HRP conjugated anti-human IgG, at room temperature to quantitate LS-Rg binding. After a 30 minute incubation at room temperature in the dark with OPD peroxidase substrate (Sigma P9187), the extent of LS-Rg binding and percent inhibition was determined from the OD₄₅₀.

### Example 8

### 2′-NH₂ RNA Ligands to Human L-Selectin

### A. SELEX

The starting RNA pool for SELEX, randomized 40N7 (SEQ ID NO: 63), contained approximately 10¹⁵ molecules (1 nmol RNA). The SELEX protocol is outlined in Tables 7a and 7b and Example 7. The dissociation constant of randomized RNA to LS-Rg is estimated to be approximately 10 μM. No difference was observed in the RNA elution profiles with 5 mM EDTA from SELEX and background beads for rounds 1 and 2, while the 50 mM elution produced a 2-3 fold excess over background (Table 7a). The 50 mM eluted RNA from rounds 1 and 2 were amplified for the input material for rounds 2 and 3, respectively. Beginning in round 3, the 5 mM elution from SELEX beads was significantly higher than background and was processed for the next round's input RNA. The percentage of input RNA eluted by 5 mM EDTA increased from 0.5% in the first round to 8.4% in round 5 (Table 7a). An additional increase in specifically eluted RNA from the 10 μM LS-Rg beads was not observed in round 6 (Table 7a). To increase the stringency of selection, the density of immobilized LS-Rg was reduced ten fold in round 5 with further reductions in protein density at later rounds. The affinity of the selected pools rapidly increased and the pools gradually evolved biphasic binding characteristics.

Binding experiments with 6th round RNA revealed that the affinity of the evolving pool for L-selectin was temperature sensitive. Beginning with round 7, the SELEX was branched; one branch was continued at 4° C. (Table 7a) while the other was conducted at room temperature (Table 7b). Bulk sequencing of 6th, 13th (rm temp) and 14th (4° C.) RNA pools revealed noticeable non-randomness at round six and dramatic non-randomness at the later rounds. The 6th round RNA bound monophasically at 4° C. with a dissociation constant of approximately 40 nM, while the 13th and 14th round RNAs bound biphasically with high affinity Kds of approximately 700 pM. The molar fraction of the two pools that bound with high affinity were 24% and 65%, respectively. The binding of all tested pools required divalent cations. In the absence of divalent cations, the Kds of the 13th and 14th round pools increased to 45 nM and 480 nM, respectively (HSMC, minus Ca⁺⁺/Mg⁺⁺, plus 2 mM EDTA).

To monitor the progress of SELEX, ligands were cloned and sequenced from rounds 6, 13 (rm temp) and 14 (4° C.). Sequences were aligned manually and with the aid of a computer program that determines consensus sequences from frequently occurring local alignments.

### B. Sequences

In Table 8, ligand sequences are shown in standard single letter code (Cornish-Bowden, 1985 NAR 13: 3021-3030). The letter/number combination before the “.” in the ligand name indicates whether it was cloned from the round 6, 13 or 14 pools. Only the evolved random region is shown in Table 8. Any portion of the fixed region is shown in lower case letters. By definition, each clone includes both the evolved sequence and the associated fixed region, unless specifically stated otherwise. From the sixth, thirteenth and fourteenth rounds, respectively, 26 of 48, 8 of 24 and 9 of 70 sequenced ligands were unique. A unique sequence is operationally defined as one that differs from all others by three or more nucleotides. Sequences that were isolated more than once, are indicated by the parenthetical number, (n), following the ligand isolate number. These clones fall into thirteen sequence families (I-XIII) and a group of unrelated sequences (Orphans) (SEQ ID NOs: 67-117).

Two families, I and III, are defined by ligands from multiple lineages. Both families occur frequently in round 6, but only one family m ligand was identified in the final rounds. Six families (IV, V, VI, VII, VIII, and possibly II) are each defined by just two lineages which limits confidence in their consensus sequences. Five families (IX through XIII) are defined by a single lineage which precludes determination of consensus sequences.

Ligands from family II dominate the final rounds: 60/70 ligands in round 14 and 9/24 in round 13. Family II is represented by three mutational variations of a single sequence. One explanation for the recovery of a single lineage is that the ligand's information content is extremely high and was therefore represented by a unique species in the starting pool. Family II ligands were not detected in the sixth round which is consistent with a low frequency in the initial population. An alternative explanation is sampling error. Note that a sequence of questionable relationship was detected in the sixth round.

The best defined consensus sequences are those of family I, AUGUGUA (SEQ ID NO: 118), and of family III, AACAUGAAGUA (SEQ ID NO: 120), as shown in Table 8. Family III has two additional, variably spaced sequences, AGUC and ARUUAG, that may be conserved. The tetranucleotide AUGW is found in the consensus sequence of families I, III, and VII and in families II, VIII and IX. If this sequence is significant, it suggests that the conserved sequences of ligands of family VIII are circularly permuted. The sequence AGAA is found in the consensus sequence of families IV and VI and in families X and XIII.

### C. Affinities

The dissociation constants for representative ligands from rounds 13 and 14, including all orphans, were determined by nitrocellulose filter binding experiments are described in Example 7 and the results are listed in Table 9. These calculations assume two RNA ligand binding sites per chimera. The affinity of random RNA cannot be reliably determined but is estimated to be approximately 10 μM.

In general, ligands bind monophasically with dissociation constants ranging from 50 pM to 15 nM at 4° C. Some of the highest affinity ligands bind biphasically. Although ligands of families I, VII, X and orphan F14.70 bind about equally well at 4° C. and room temperature, in general the affinities decrease with increasing temperature. The observed affinities substantiate the proposition that it is possible to isolate oligonucleotide ligands with affinities that are several orders of magnitude greater than that of carbohydrate ligands.

### Example 9

### Specificity of 2′-NH₂ RNA Ligands to L-Selectin

The affinity of L-selectin ligands to ES-Rg, PS-Rg and CD22β-Rg were determined by nitrocellulose partitioning as described in Example 7. As indicated in Table 10, the ligands are highly specific for L-selectin. In general, a ligand's affinity for ES-Rg is 10³-fold lower and that for PS-Rg is about 10⁴-fold less than for LS-Rg. Binding above background is not observed for CD22β-Rg at the highest protein concentration tested (660 nM), indicating that ligands do not bind the Fc domain of the chimeric constructs nor do they have affinity for the sialic acid binding site of an unrelated lectin. The specificity of oligonucleotide ligand binding contrasts sharply with the binding of cognate carbohydrates by the selectins and confirms the proposition that SELEX ligands will have greater specificity than carbohydrate ligands.

### Example 10

### Binding of L-Selectin 2′-NH₂ RNA Ligands to Human PBMCs

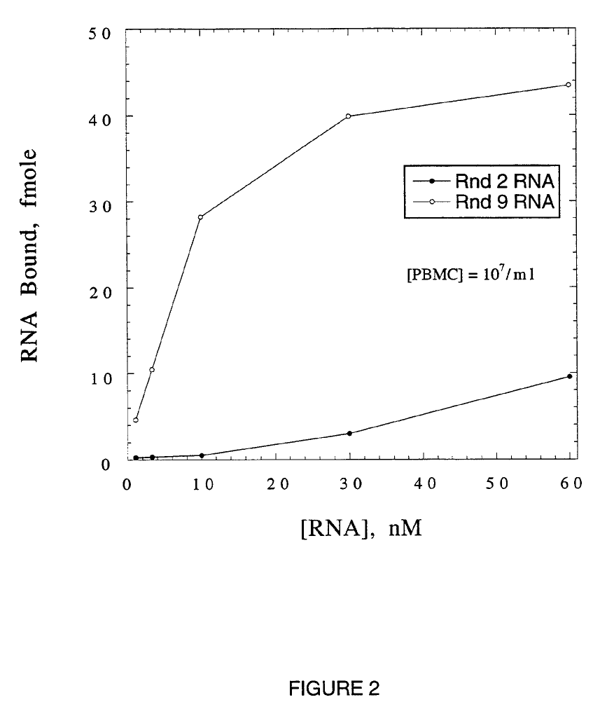

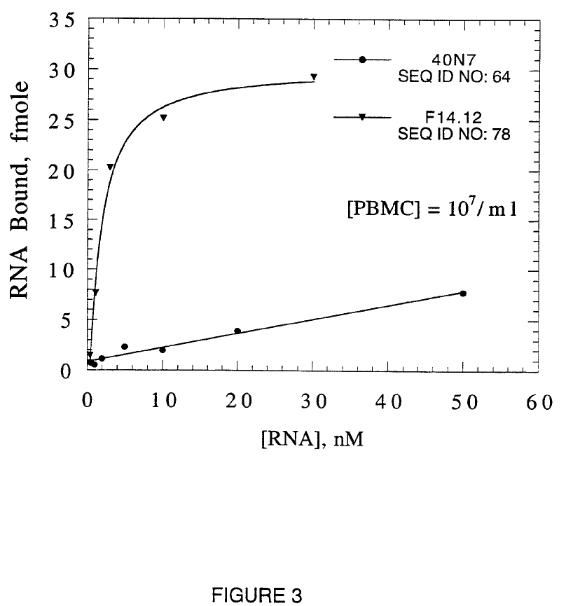

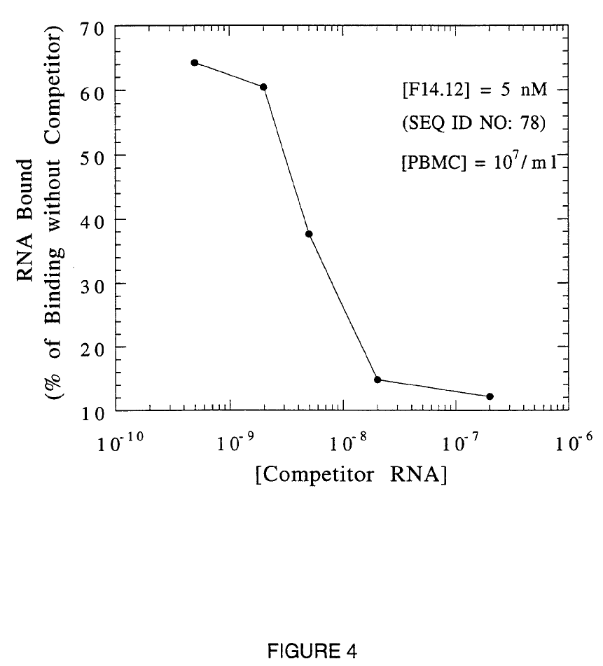

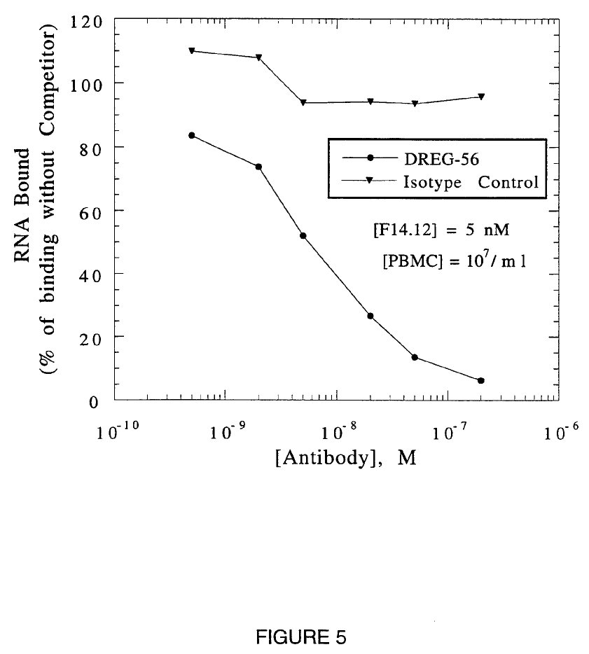

Since the L-selectin ligands were isolated against purified, immobilized protein, it is essential to demonstrate that they bind L-selectin presented in the context of a cell surface. Comparison of 2nd and 9th round RNAs (FIG. 2) shows that the evolved (9th round) ligand pool binds isolated PBMCs with high affinity and, as expected for specific binding, in a saturable fashion. The binding of round 2 RNA appears to be non-saturable as is characteristic of non-specific binding. The cloned ligand, F14.12 (SEQ ID NO: 78), also binds in a saturable fashion with a dissociation constant of 1.3 mM, while random 40N7 (SEQ ID NO: 64) resembles round 2 RNA (FIG. 3). The saturability of binding is confirmed by the data in FIG. 4; >90% of 5 nM ³²P_labeled F14.12 RNA binding is competed by excess cold RNA. Specificity is demonstrated by the results in FIG. 5; binding of 5 nM ³²P-labeled F14.12 RNA is completely competed by the anti-L-selectin blocking monoclonal antibody, DREG-56, but is unaffected by an isotype-matched irrelevant antibody. These data validate the feasibility of using immobilized, purified protein to isolate ligands against a cell surface protein and the binding specificity of F14.12 to L-selectin in the context of a cell surface.

### Example 11

### Inhibition of Binding to Sialyl-Lewis X

Oligonucleotide ligands, eluted by 2-5 mM EDTA, are expected to derive part of their binding energy from contacts with the lectin domain's bound Ca⁺⁺ and consequently, are expected to compete with sialyl-Lewis^{X} for binding. The ability of ligand F14.12 (SEQ ID NO: 78) to inhibit LS-Rg binding to immobilized sialyl-Lewis^{X} was determined by competition ELISA assays. As expected, 4 mM EDTA reduced LS-Rg binding 7.4-fold, while 20 mM round 2 RNA did not inhibit LS-Rg binding. Carbohydrate binding is known to be Ca⁺⁺ dependent; the affinity of round 2 RNA is too low to bind 10 nM LS-Rg (Table 7).

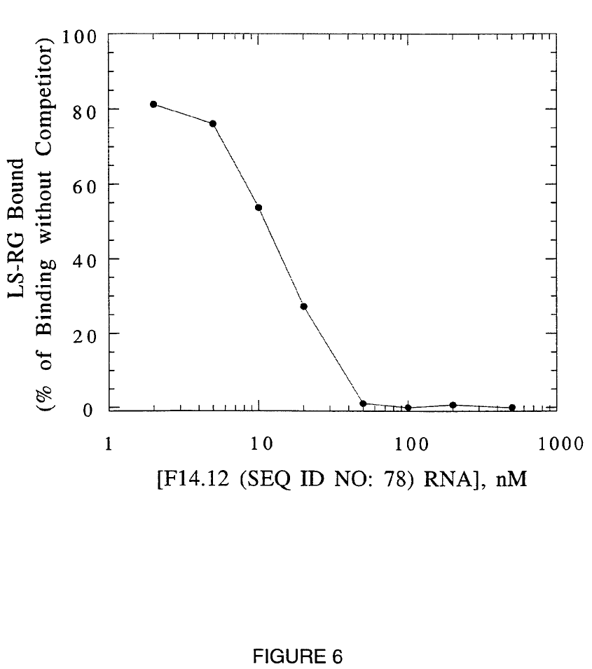

In this assay F14.12 RNA inhibits LS-Rg binding in a concentration dependent manner with an IC₅₀ of about 10 nM (FIG. 6). Complete inhibition is observed at 50 nM F14.12. The observed inhibition is reasonable under the experimental conditions; the Kd of F14.12 at room temperature is about 1 nM (Table 9) and 10 nM LS-Rg is 20 nM binding sites. These data verify that RNA ligands compete with sialyl-Lewis^{X} for LS-Rg binding and support the contention that low concentrations of EDTA specifically elute ligands that bind the lectin domain's carbohydrate binding site.

### Example 12

### Inhibition of Binding to Sialyl-Lewis^{X}

In favorable instances, comparative analysis of aligned sequences allows deduction of secondary structure and structure-function relationships. If the nucleotides at two positions in a sequence covary according to Watson-Crick base pairing rules, then the nucleotides at these positions are apt to be paired. Nonconserved sequences, especially those that vary in length are not apt to be directly involved in function, while highly conserved sequence are likely to be directly involved.

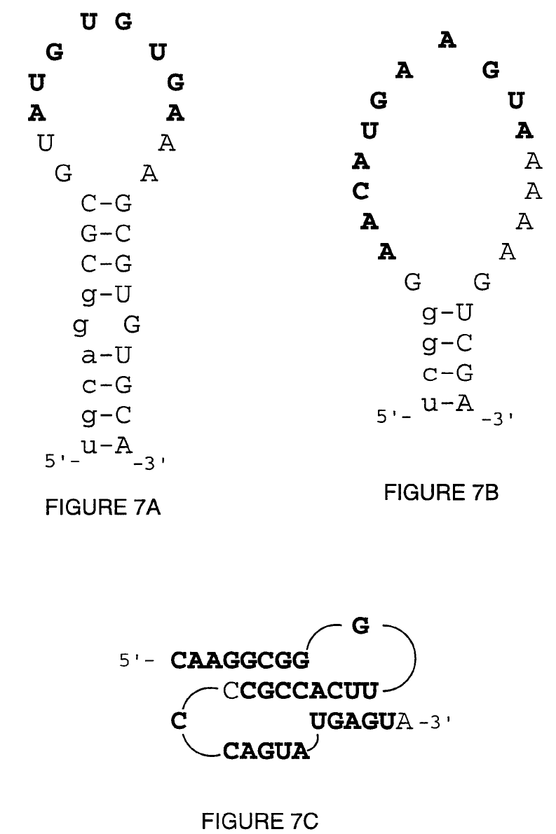

Comparative analysis of the family I alignment suggests a hairpin structure in which the consensus sequence, AUGUGUGA, is contained within a variable size loop (FIG. 7a). The stem sequences are not conserved and may be either 5′ or 3′-fixed or variable sequence. The one ligand that does not form a stem, F14.25 (SEQ ID NO: 73), has a significantly lower affinity than the other characterized ligands (Table 9).

The proposed structure for family III is also a hairpin with the conserved sequence, AACAUGAAGUA, contained within a variable length loop (FIG. 7b). The 5′-half of the stem is 5′-fixed sequence which may account in part for the less highly conserved sequence, AGUC.

Although there is no alignment data for family II, the sequence folds into a pseudoknot (FIG. 7c). Three attractive features of this model are 1) the helices stack on one another, 2) the structure utilizes only variable sequence and 3) the structure is compatible with the major variant sequences.

### Example 13

### ssDNA Ligands to Human L-Selectin

The experimental procedures outlined in this Example were used to identify and characterize ssDNA ligands to human L-selectin as described in Examples 14-21.

### Experimental Procedures

### A) Materials

Unless otherwise indicated, all materials used in the ssDNA SELEX against the L-selectin/IgG2 chimera, LS-Rg, were identical to those of Example 7, paragraph A. The buffer for SELEX experiments was 1 mM CaCl₂, 1 mM MgCl₂, 100 mM NaCl, 10.0 mM HEPES, pH 7.4. The buffer for all binding affinity experiments differed from the above in containing 125 mM NaCl, 5 mM KCl, and 20 nM HEPES, pH 7.4.

### B) SELEX

The SELEX procedure is described in detail in U.S. Pat. No. 5,270,163 and elsewhere. The strategy used for this ssDNA SELEX is essentially identical to that described in Example 7, paragraph B except as noted below. The nucleotide sequence of the synthetic DNA template for the LS-Rg SELEX was randomized at 40 positions. This variable region was flanked by BH 5′ and 3′ fixed regions. The random DNA template was termed 40BH (SEQ ID NO: 126) and had the following sequence: 5′-ctacctacgatctgactagc<40N>sgcttactctcatgtagttcc-3′. The primers for the PCR were the following: 5′ Primer: 5′-ctacctacgatctgactagc-3′ (SEQ ID NO: 127) and 3′ Primer: 5′-ajajaggaactacatgagagtaagc-3′; j=biotin (SEQ ID NO: 128). The fixed regions include primer annealing sites for PCR amplification. The initial DNA pool contained 500 pmols of each of two types of single-stranded DNA: 1) synthetic ssDNA and 2) PCR amplified, ssDNA from 1 nmol of synthetic ssDNA template.

For subsequent rounds, eluted DNA was the template for PCR amplification. PCR conditions were 50 mM KCl, 10 mM Tris-Cl, pH 8.3, 7.5 mM MgCl₂, 1 mM of each DATP, dCTP, dGTP, and dTTP and 25 U/ml of the Stoffel fragment of Taq DNA polymerase. After PCR amplification, double stranded DNAs were end-labeled using γ³²P-ATP. Complementary strands were separated by electrophoresis through an 8% polyacrylamide/7M urea gel. Strand separation results from the molecular weight difference of the strands due to biotintylation of the 3′ PCR primer. In the final rounds, DNA strands were separated prior to end labelling in order to achieve high specific activity. Eluted fractions were processed by ethanol precipitation.

The strategy for partitioning LS-Rg/ssDNA complexes from unbound ssDNA was as described in Example 7, paragraph B, except that 2 mM EDTA was utilized for specific elution. The SELEX strategy is outlined in Table 11.

### C) Nitrocellulose Filter Binding Assay

As described in SELEX patent applications and in Example 7, paragraph C, a nitrocellulose filter partitioning method was used to determine the affinity of ssDNA ligands for LS-Rg and for other proteins. For these experiments a Gibco BRL 96 well manifold was substituted for the 12 well Millipore manifold used in Example 7 and radioactivity was determined with a Fujix BAS100 phosphorimager. Binding data were analyzed as described in Example 7, paragraph C.

### D) Cloning and Sequencing

Thirteenth, fifteenth and seventeenth round PCR products were re-amplified with primers which contain either a BamHI or a HinDIII restriction endonuclease recognition site. Approximately 140 ligands were cloned and sequenced using the procedures described in Example 7, paragraph D. The resulting sequences are shown in Table 12.

### E) Cell Binding Studies

The ability of evolved ligand pools to bind to L-selectin presented in the context of a cell surface was tested in experiments with isolated human peripheral blood mononuclear cells (PBMCs) as described in Example 7, paragraph E

Flow Cytometry

Binding of oligonucleotides to leukocytes was tested in flow cytometry applications. Briefly, peripheral blood mononuclear cells (PBMC) were purified on histoplaque by standard techniques. Cells (500 cells/mL) were incubated with fluorescein labeled oligonucleotide in 0.25 ML SMHCK buffer (140 mM NaCl, 1 mM MgCl₂, 1 mM CaCl₂, 5 mM, KCl, 20 mM HEPES pH 7.4, 8.9 mM NaOH, 0.1% (w/v) BSA, 0.1% (w/v) sodium azide) at room temperature for 15 minutes. Fluorescent staining of cells was quantified on a FACSCaliber fluorescent activated cell sorter (Becton Dickinson, San Jose, Calif.).

To examine the ability of oligonucleotides to bind leukocytes in whole blood, 25 μl aliquots of heparinised whole blood were stained for 30 min at 22° C. with 2 μg Cy5PE labeled anti-CD45 (generous gift of Ken Davis, Becton-Dickinson) and 0.15 μM FITC-LD201T1 (synthesized with a 5′-Fluorescein phosphoramidite by Operon Technologies, Alameda, Calif.; SEQ ID NO: 185). To determine specificity, other samples were stained with FITC-LD201T1 in the presence of 0.3 μM DREG-56 or 7 μM unlabeled LD201T1; or cells were reassayed after addition of 4 mM EDTA. The final concentration of whole blood was at least 70% (v/v). Stained, concentrated whole blood was diluted 1/15 in 140 mM NaCl, 5 mM KCl, 1 mM MgCl₂, 1 mM CaCl₂, 20 mM HEPES pH 7.4, 0.1% bovine serum albumin and 0.1% NaN₃ immediately prior to flow cytometry on a Becton-Dickinson FACS Calibur. Lymphocytes and granulocytes were gated using side scatter and CD45CyPE staining.

### F) Synthesis and Characterization of Multimeric Oligonucleotide Ligands

### Synthesis of Branched Dimeric Oligonucleotide Complexes

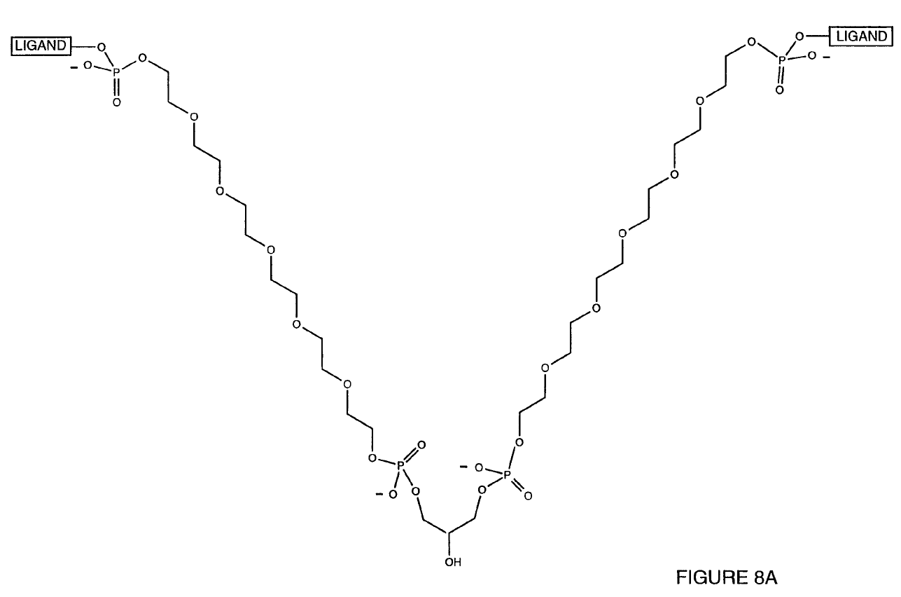

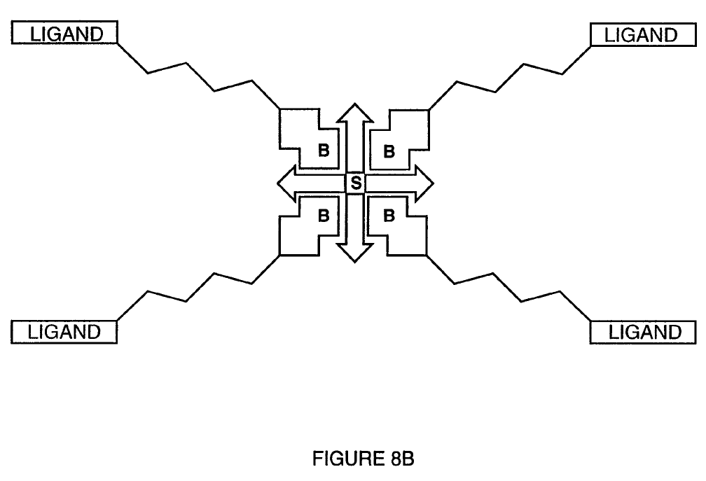

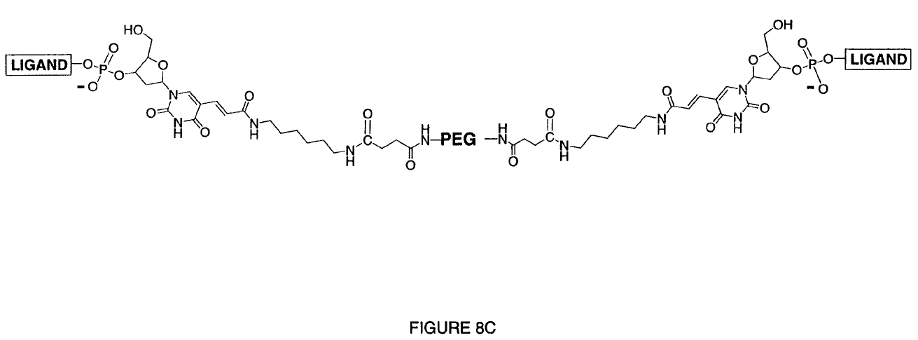

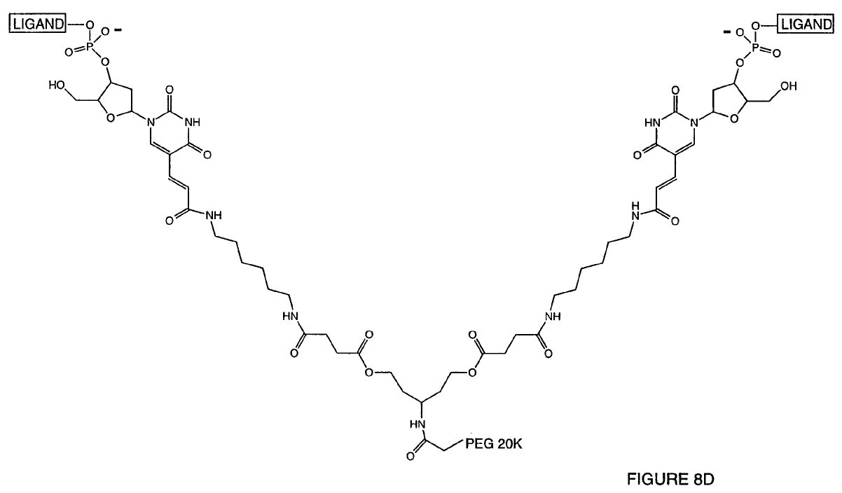

Dimeric oligonucleotides were synthesized by standard solid state processes, with initiation from a 3′-3′ Symmetric Linking CPG (Operon, Alameda, Calif.). Branched complexes contain two copies of a truncated L-selectin DNA ligand, each of which is linked by the 3′ end to the above CPG via a five unit ethylene glycol spacer (FIG. 8A). Each ligand is labeled with a fluorescein phosphoramidite at the 5′ end (Glen Research, Sterling, Va.). Branched dimers were made for 3 truncates of LD201T1 (SEQ ID NO: 142). The truncated ligands used were LD201T4 (SEQ ID NO: 187), LD201T10 (SEQ ID NO: 187) and LD201T1 (SEQ ID NO: 185). Branched dimers were purified by gel electrophoresis.

Synthesis of Multivalent Biotintylated-DNA Ligand/Streptavidin Complexes

Multivalent oligonucleotide complexes were produced by reacting biotintylated DNA ligands with either fluorescein or phycoerythrin labeled streptavidin (SA-FITC, SA-PE, respectively) (FIG. 8B). Streptavidin (SA) is a tetrameric protein, each subunit of which has a biotin binding site. 5′ and 3′ biotintylated DNAs were synthesized by Operon Technologies, Inc (Alameda, Calif.) using BioTEG and BioTEG CPG (Glen Research, Sterling, Va.), respectively. The expected stoichiometry is 2 to 4 DNA molecules per complex. SA/bio-DNA complexes were made for 3 truncates of LD201(SEQ ID NO: 142). The truncated ligands were LD201T4 (SEQ ID NO: 187), LD201T10 (SEQ ID NO: 188) and LD201T1 (SEQ ID NO: 185). The bio-DNA/SA multivalent complexes were generated by incubating biotin modified oligonucleotide (1 mM) and fluorescein labeled streptavidin (0.17 mM) in 150 mM NaCl, 20 mM HEPES pH 7.4 at room temperature for at least 2 hours. Oligonucleotide-streptavidin complexes were used directly from the reaction mixture without additional purification of the Complex from free streptavidin or oligonucleotide.

Synthesis of a Dumbell Dimer Multivalent Complex

A “dumbell” DNA dimer complex was formulated from a homobifunctional N-hydroxysuccinimidyl (or NHS) active ester of polyethelene glycol, PEG 3400 MW, and a 29mer DNA oligonucleotide, NX303 (SEQ ID NO: 196), having a 5′ terminal Amino Modifier C6 dT (Glen Research) and a 3′-3′ terminal phosphodiester linkage (FIG. 8C). NX303 is a truncate of LD201 (SEQ ID NO: 142). The conjugation reaction was in DMSO with 1% TEA with excess equivalents of the DNA ligand to PEG. The PEG conjugates were purified from the free oligonucleotide by reverse phase chromatography. The dimer was then purified from the monomer by anion exchange HPLC. The oligonucleotide was labeled at the 5′ terminus with fluorescein as previously described.

Synthesis of a Fork Dimer Multivalent Complex

To synthesize the fork dimer multivalent complex (FIG. 8D), a glycerol was attached by its 2-position to one terminus of a linear PEG molecule (MW 20 kD) to give the bis alcohol. This was further modified to the bis succinate ester, which was activated to the bis N-hydroxysuccinimidyl active ester. The active ester was conjugated to the primary amine at the 5′ terminus of the truncated DNA nucleic acid ligand NX303 (SEQ ID NO: 196). The conjugation reaction was in DMSO with 1% TEA with excess equivalents of the DNA ligand to PEG. The PEG conjugates were purified away from the free oligonucleotide by reverse phase chromatography. The dimer was then purified away from the monomer by anion exchange HPLC. The oligonucleotide was labeled at the 5′ terminus with fluorescein as previously described.

Characterization of Multimeric Oligonucleotide Ligands

The binding of dimeric and multimeric oligonucleotide complexes to human peripheral blood mononuclear cells was analyzed by flow cytometry as described in Example 13, paragraph D.

### G) Photo-Crosslinking

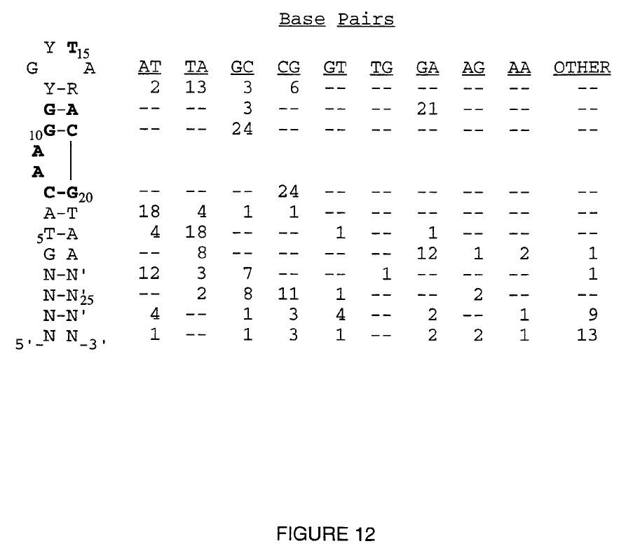

A photo-crosslinking version of DNA ligand LD201T4 (SEQ ID NO: 187) was synthesized by replacing nucleotide T15 (FIG. 12) with 5-bromo-deoxyuracil. 4 nmol of ³²P-labeled DNA was incubated with 4 nmol L-selectin-Rg in 4 ml 1×SHMCK+0.01% human serum albumin (w/v), then irradiated at ambient temperature with 12,500 pulses from an excimer laser at a distance of 50 cm and at 175 mJ/pulse. Protein and DNA were precipitated with 400 μl 3 M sodium acetate and 8.4 ml ethanol followed by incubation at −70 degrees C. Precipitated material was centrifuged, vacuum dried and resuspended in 100 μl 0.1 M Tris pH 8.0, 10 mM CaCl₂. Forty-five fig chymotrypsin were added and after 20 min at 37 degrees C., the material was loaded onto an 8% polyacrylamide/7 M urea/1×TBE gel and electrophoresed until the xylene cyanole had migrated 15 cm. The gel was soaked for 5 min in 1×TBE and then blotted for 30 min at 200 mAmp in 1×TBE onto Immobilon-P (Millipore). The membrane was washed for 2 min in water, air dried, and an autoradiograph taken. A labeled band running slower than the free DNA band, representing a chymotryptic peptide crosslinked to LD201T4, was observed and the autoradiograph was used as a template to excise this band from the membrane. The peptide was sequenced by Edman degradation, and the resulting sequence was LEKTLP_SRSYY. The blank residue corresponds to the crosslinked amino acid, F82 of the lectin domain.

### H) Lymphocyte Trafficking Experiments

Human PBMC were purified from heparinised blood by a Ficoll-Hypaque gradient, washed twice with HBSS (calcium/magnesium free) and labeled with ⁵¹Cr (Amersham). After labeling, the cells were washed twice with HBSS (containing calcium and magnesium) and 1% bovine serum albumin (Sigma). Female SCID mice (6-12 weeks of age) were injected intravenously with 2×10⁶ cells. The cells were either untreated or mixed with either 13 pmol of antibody (DREG-56 or MEL-14), or 4, 1, or 0.4 nmol of modified oligonucleotide (synthesis described below). One hour later the animals were anesthetized, a blood sample taken and the mice were euthanised. PLN, MLN, Peyer's patches, spleen, liver, lungs, thymus, kidneys and bone marrow were removed and the counts incorporated into the organs determined by a Packard gamma counter. In a second protocol, 2×10⁶ human PBMC, purified, labeled, and washed as described above, were injected intravenously into female SCID mice without antibody or oligonucleotide pretreatment. One to 5 min prior to injecting the cells, the animals were injected with either 15 pmol DREG-56 or 4 nmol modified oligonucleotide. Counts incorporated into organs were quantified as described above.

Synthesis of modified nucleotides NX288 (SEQ ID NO: 193) and NX303 (SEQ ID NO: 196) was initiated by coupling to a dT-5′-CE polystyrene support (Glen Research), resulting in a 3′-3′ terminal phosphodiester linkage, and having a 5′ terminal an Amino Modifier C6 dT (Glen Research). Once NX288 and NX303 were synthesized, a 20,000 MW PEG2-NHS ester (Shearwater Polymers, Huntsville, Ala.) was then coupled to the oligonucleotide through the 5′ amine moiety. The molar ratio, PEG:oligo, in the reactions was from 3:1 to 10:1. The reactions were performed in 80:20 (v:v) 100 mM borate buffer pH 8: DMF at 37° C. for one hour.

### I) Inhibition of L-Selectin Binding to Sialyl Lewis^{X}

SLe^{X}-BSA (Oxford GlycoSystems, Oxford, UK) in 1×PBS, without CaCl₂ and MgCl₂,′ (GIBCO/BRL) was immobilized at 100 ng/well onto a microtiter plate by overnight incubation at 22° C. The wells were blocked for 1 h with the assay buffer consisting of 20 mM HEPES, 111 mM NaCl, 1 mM CaCl₂, 1 mM MgCl₂, 5 mM KCl, 8.9 mM NaOH, final pH 8, and 1% globulin-free BSA (Sigma). The reaction mixtures, incubated for 90 min with orbital shaking, contained 5 nM L-Selectin-Rg, a 1:100 dilution of anti-human IgG-peroxidase conjugate (Sigma), and 0-50 nM of competitor in assay buffer. After incubation, the plate was washed with BSA-free assay buffer to remove unbound chimera-antibody complex and incubated for 25 min with O-phenylenediamine dihydrochloride peroxidase substrate (Sigma) by shaking in the dark at 22° C. Absorbance was read at 450 nm on a Bio-Kinetics Reader, Model EL312e (Bio-Tek Instruments, Laguna Hills, Calif.). Values shown represent the mean.+−.s.e from duplicate, or triplicate, samples from one representative experiment.

### Example 14

### ssDNA Ligands to L-Selectin

### A. SELEX

The starting ssDNA pool for SELEX, randomized 40BH (SEQ ID NO: 126), contained approximately 10¹⁵ molecules (1 nmol ssDNA). The dissociation constant of randomized ssDNA to LS-Rg is estimated to be approximately 10 μM. The SELEX protocol is outlined in Table 11.

The initial round of SELEX was performed at 4° C. with an LS-Rg density of 16.7 pmol/μl of protein A Sepharose beads. Subsequent rounds were at room temperature except as noted in Table 11. The 2 mM EDTA elution was omitted from rounds 1-3. The signal to noise ratio of the 50 mM EDTA elution in these three rounds was 50, 12 and 25, respectively (Table 11). These DNAs were amplified for the input materials of rounds 2-4. Beginning with round 4, a 2 mM EDTA elution was added to the protocol. In this and all subsequent rounds, the 2 mM EDTA eluted DNA was amplified for the next round's input material.

To increase the stringency of selection, the density of immobilized LS-Rg was reduced ten fold in round 4 with further reductions in protein as needed to increase the stringency of selectin (Table 11). Under these conditions a rapid increase in the affinity of the selected pools was observed (Tables 11); at 4° C., the dissociation constant of round 7 ssDNA was 60 nM.

Binding experiments with 7th round DNA revealed that the affinity of the evolving pool for L-selectin was weakly temperature sensitive (Kds: 60 nM, 94 nM and 230 nM at 4° C., room temperature and 37° C., respectively). To enhance the selection of ligands that bind at physiological temperature, rounds 8, 13, 16 and 17 were performed at 37° C. Although temperature sensitive, the affinity of round 15 ssDNA was optimal at room temperature (160 pM), with 3-fold higher Kds at 4° C. and 37° C.

Bulk sequencing of DNA pools indicates some non-randomness at round 5 and dramatic non-randomness at round 13. Ligands were cloned and sequenced from rounds 13, 15, and 17. Sequences were aligned manually and with the aid of a NeXstar computer program that determines consensus sequences from frequently occurring local alignments.

### B. Sequences

In Table 12, ligand sequences are shown in standard single letter code (Cornish-Bowden, 1985 NAR 13: 3021-3030). Only the evolved random region is shown in Table 12. Any portion of the fixed region is shown in lower case letters. By definition, each clone includes both the evolved sequence and the associated fixed region, unless specifically stated otherwise A unique sequence is operationally defined as one that differs from all others by three or more nucleotides. Sequences that were isolated more than once are indicated by the parenthetical number, (n), following the ligand isolate number. These clones fall into six families and a group of unrelated sequences or orphans (Table 12) (SEQ ID NOs: 129-180).

Family 1 is defined by ligands from 33 lineages and has a well defined consensus sequence, TACAAGGYGYTAVACGTA (SEQ ID NO: 181). The conservation of the CAAGG and ACG and their 6 nucleotide spacing is nearly absolute (Table 12). The consensus sequence is flanked by variable but complementary sequences that are 3 to 5 nucleotides in length. The statistical dominance of family 1 suggests that the properties of the bulk population are a reflection of those of family 1 ligands. Note that ssDNA family 1 and 2′-NH₂ family I share a common sequence, CAAGGCG and CAAGGYG, respectively.

Family 2 is represented by a single sequence and is related to family 1. The ligand contains the absolutely conserved CAAGG and highly conserved ACG of family 1 although the spacing between the two elements is strikingly different (23 compared to 6 nucleotides).

Families 4-6 are each defined by a small number of ligands which limits confidence in their consensus sequence, while family 7 is defined by a single sequence which precludes determination of a consensus. Family 5 appears to contain two conserved sequences, AGGGT and RCACGAYACA, the positions of which are circularly permuted.

### C. Affinities

The dissociation constants of representative ligands from Table 12 are shown in Table 13. These calculations assume two ssDNA ligand binding sites per chimera. The affinity of random ssDNA cannot be reliably determined but is estimated to be approximately 10 μM.

At room temperature, the dissociation constants range from 43 pM to 1.8 nM which is at least a 5×10³ to 2×10⁵ fold improvement over randomized ssDNA (Table 13). At 37° C., the Kds range from 130 pM to 23 nM. The extent of temperature sensitivity varies from insensitive (ligands LD122 and LD127 (SEQ ID NO: 159 and 162)) to 80-fold (ligand LD112 (SEQ ID NO: 135)). In general, among family 1 ligands the affinity of those from round 15 is greater than that of those from round 13. For the best ligands (LD208, LD227, LD230 and LD233 (SEQ ID NOS: 133, 134, 132, and 146)), the difference in affinity at room temperature and 37° C. is about 4-fold.

The observed affinities of the evolved ssDNA ligand pools reaffirm our proposition that it is possible to isolate oligonucleotide ligands with affinities that are several orders of magnitude greater than that of carbohydrate ligands.

### Example 15

### Specificity of ssDNA Ligands to L-Selectin

The affinity of representative cloned ligands for LS-Rg, ES-Rg, PS-Rg, CD22β-Rg and WGA was determined by nitrocellulose partitioning and the results shown in Table 14. The ligands are highly specific for L-selectin. The affinity for ES-Rg is about 10³-fold lower and that for PS-Rg is about 5×10³-fold less than for LS-Rg. Binding above background is not observed for CD22 β—Rg or for WGA at 0.7 and 1.4 μM protein, respectively, indicating that ligands neither bind the Fc domain of the chimeric constructs nor have affinity for unrelated sialic acid binding sites.

The specificity of oligonucleotide ligand binding contrasts sharply with the binding of cognate carbohydrates by the selectins and reconfirms the proposition that SELEX ligands will have greater specificity than carbohydrate ligands.

### Example 16

### Cell Binding Studies

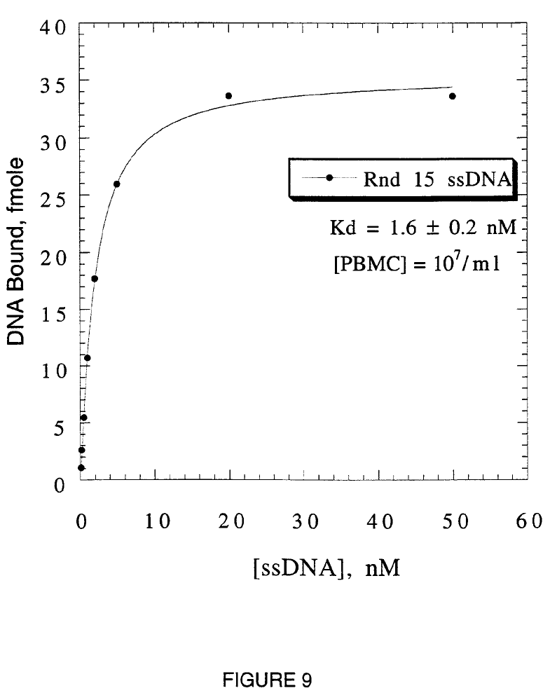

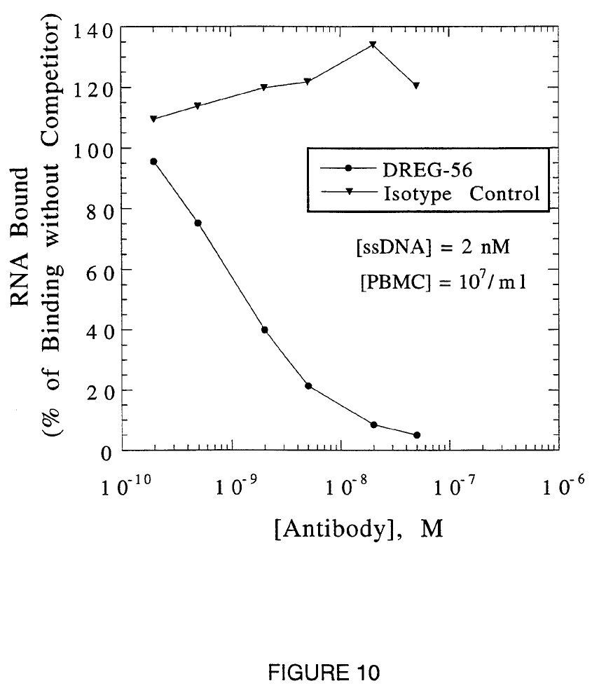

Round 15 ssDNA pool was tested for its ability to bind to L-selectin presented in the context of a peripheral blood mononuclear cell surface as described in Example 13, paragraph E. The evolved pool was tested both for affinity and for specificity by competition with an anti-L-selectin monoclonal antibody. FIG. 9 shows that the round 15 ssDNA pool binds isolated PBMCs with a dissociation constant of approximately 1.6 nM and, as is expected for specific binding, in a saturable fashion. FIG. 10 directly demonstrates specificity of binding; in this experiment, binding of 2 nM ³²P-labeled round 15 ssDNA is completely competed by the anti-L-selectin blocking monoclonal antibody, DREG-56, but is unaffected by an isotype-matched irrelevant antibody. In analogous experiments, LD201T1 (SEQ ID NO: 185) was shown to bind human PBMC with high affinity. Binding was saturable, divalent cation dependent, and blocked by DREG-56.

These data validate the feasibility of using immobilized, purified protein to isolate ligands against a cell surface protein and demonstrate the specific binding of the round 15 ssDNA pool and of ligand LD201T1 to L-selectin in the context of a cell surface.

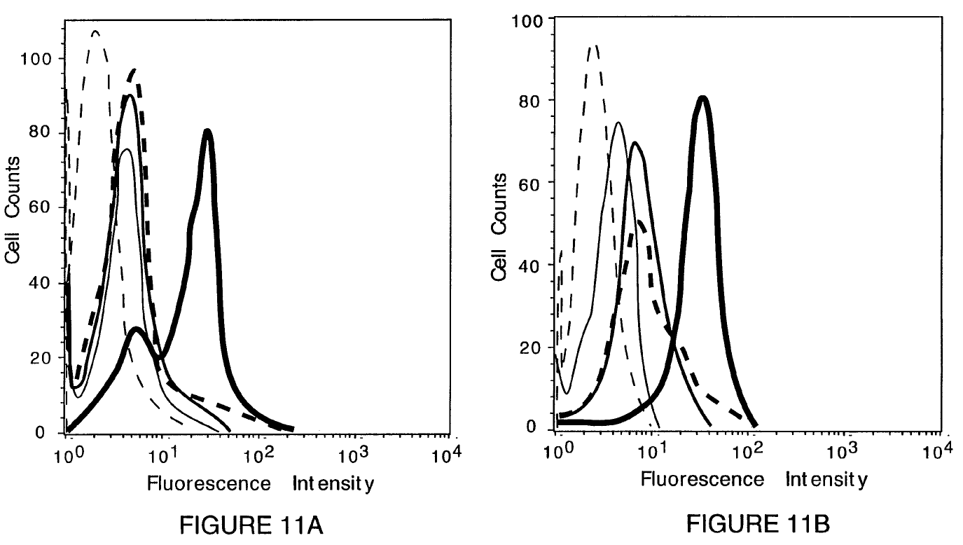

The binding of LD201T1 to leukocytes in whole blood was examined by flow cytometry. Fluorescein isothiocyanate (FITC)-conjugated LD201T1 specifically bind human lymphocytes and neutrophils (FIGS. 11A/B); binding is inhibited by competition with DREG-56, unlabeled LD201, and by the addition of 4 mM EDTA (FIGS. 11A/B). These cell binding studies demonstrate that LD201T1 bind saturably and specifically to human L-selectin on lymphocytes and neutrophils.

### Example 17

### Secondary Structure of High Affinity ssDNA Ligands to L-Selectin

In favorable instances, comparative analysis of aligned sequences allows deduction of secondary structure and structure-function relationships. If the nucleotides at two positions in a sequence covary according to Watson-Crick base pairing rules, then the nucleotides at these positions are apt to be paired. Nonconserved sequences, especially those that vary in length are not apt to be directly involved in function, while highly conserved sequence are likely to be directly involved.

Comparative analysis of 24 sequences from family 1 strongly supports a hairpin secondary structure for these ligands (FIG. 12). In the figure, consensus nucleotides are specified, with invariant nucleotides in bold type. To the right of the stem is a matrix showing the number of occurrences of particular base pairs for the positions in the stem that are on the same line. The deduced structure consists of a GYTA tetraloop, a 3 nucleotide-pair upper stem and a 6 to 7 nucleotide-pair lower stem. The upper and lower stems are separated by an asymmetrical, AA internal loop or “bulge.” “Two of the three base pairs in the upper stem and 6 of 7 in the lower stem are validated by covariation. The two invariant pairs, positions 7/20 and 10/19 are both standard Watson/Crick basepairs. This structure provides a plausible basis for the direct involvement of invariant nucleotides (especially, A8, A9 and T15) in binding the target protein.

The site of oligonucleotide binding on L-selectin can be deduced from a set of competition experiments. DREG56 is an anti-L-selectin, adhesion blocking monoclonal antibody that is known to bind to the lectin domain. Binding of three unrelated ligands, LD201T1 (SEQ ID NO: 185), LD174T1 (SEQ ID NO: 194) and LD196T1 (SEQ ID NO: 195), to LS-Rg was blocked by DREG-56, but not by an isotype-matched control. In cross-competition experiments, LD201 T1, LD174T1, or LD196T1 prevented radio-labeled LD201T1 from binding to LS-Rg, consistent with the premise that the ligands bind the same or overlapping sites. The blocking and competition experiments, taken together with divalent cation-dependence of binding, suggest that all three ligands bind to the lectin domain. This conclusion has been verified for LD201 by photo-crosslinking experiments.

If T15 of LD201T4 (SEQ ID NO: 187; FIG. 12) is replaced with 5-bromo-uracil, the resulting DNA photo-crosslinks at high yield (17%) to LS-Rg following irradiation with an excimer laser as described in Example 13, paragraph G. The high yield of crosslinking indicates a point contact between the protein and T15. Sequencing of the chymotryptic peptide corresponding to this point contact revealed a peptide deriving from the lectin domain; F82 is the crosslinking amino acid. Thus, F82 contacts T15 in a stacking arrangement that permits high yield photo-crosslinking. By analogy to the structure of the highly related E-selectin (Graves et al, Nature 367, 532-538, 1994), F82 is adjacent to the proposed carbohydrate binding site. Thus, this photo-crosslink provides direct evidence that ligand LD201 makes contact with the lectin domain of LS-Rg and provides an explanation for the function of the oligonucleotides in either sterically hindering access to the carbohydrate binding site or in altering the conformation of the lectin domain upon DNA binding.

### Example 18

### L-Selectin ssDNA Ligand Truncate Data

Initial experiments to define the minimal high affinity sequence of family 1 ligands show that more than the 26 nucleotide hairpin (FIG. 12; Table 13) is required. Ligands corresponding to the hairpin, LD201T4 (SEQ ID NO: 187) and LD227T1 (SEQ ID NO: 192) derived from LD201 (SEQ ID NO: 173) and LD227 (SEQ ID NO: 134), respectively, bind with 20-fold and 100-fold lower affinity than their full length progenitors. The affinity of LD201T3 (SEQ ID NO: 186), a 41 nucleotide truncate of ligand LD201, is reduced about 15-fold compared to the full length ligand, while the affinity of the 49-mer LD201T1 (SEQ ID NO: 185) is not significantly altered (Tables 12 and 13).

Additional experiments show that truncates LD201T10 (SEQ ID NO: 188) and LD227X1 (SEQ ID NO: 191) bind with affinities similar to their full length counterparts. Both of these ligands have stems that are extended at the base of the consensus stem. Alterations in the sequence of the added stem have little, if any, effect on binding, suggesting that it is not directly involved in binding.

The added stem is separated from the consensus stem by a single stranded bulge. The two ligands' single stranded bulges differ in length and have unrelated sequences. Furthermore, LD201's bulge is at the 5′-end of the original stem base while that of LD227 is at the 3′-end. Thus, the two ligands do not present an obvious consensus structure. Removal of the loop (LD201) or scrambling or truncating the sequence (LD227) diminishes affinity, suggesting that the bulged sequences may be directly involved in binding. Note that although LD201T3 is longer than LD201T10, it is unable to form the single stranded loop and extended stem because of the position of the truncated ends.

### Example 19

### Inhibition of Binding to Sialyl Lewis^{X}

Sialyl Lewis^{X} is the minimal carbohydrate ligand bound by selectins. The ability of ssDNA ligands to inhibit the binding of L-selectin to Sialyl Lewis^{X} was determined in competition ELISA assays as described in Example 13, paragraph I. LD201T1 (SEQ ID NO: 185), LD174T1 (SEQ ID NO: 194) and LD196T1 (SEQ ID NO: 195) inhibited LS-Rg binding to immobilized SLe^{X} in a dose dependent manner with IC₅₀s of approximately 3 nM. This is a 10⁵-10⁶-fold improvement over the published IC₅₀ values for SLe^{X} in similar plate-binding assays. A scrambled sequence based on LD201T1 showed no activity in this assay. These data verify that DNA ligands compete with sialyl-Lewis^{X} for LS-Rg binding and support the contention that low concentrations of EDTA specifically elute ligands that bind the lectin domain's carbohydrate binding site.

### Example 20

### Inhibition of Lymphocyte Trafficking by L-Selectin ssDNA Ligands

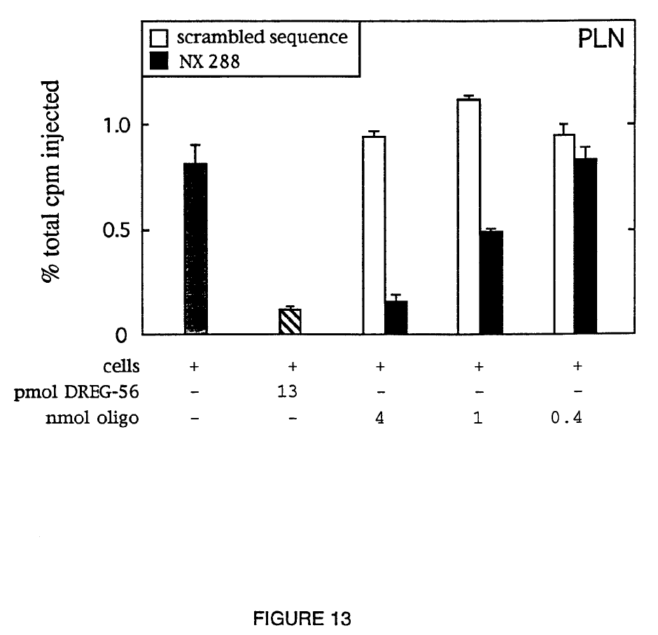

Lymphocyte trafficking to peripheral lymph nodes is exquisitely dependent on L-selectin. Since the ssDNA ligands binds to human but not rodent L-selectin, a xenogeneic lymphocyte trafficking system was established to evaluate in vivo efficacy. Human PBMC, labeled with ⁵¹Cr, were injected intravenously into SCID mice. Cell trafficking was determined 1 hour later. In this system, human cells traffic to peripheral and mesenteric lymph nodes (PLN and MLN). This accumulation is inhibited by DREG-56 (FIG. 13) but not MEL-14, a monoclonal antibody that blocks murine L-selectin-dependent trafficking. In initial experiments cells were incubated with either DREG-56 or 3′ capped and PEG-modified oligonucleotide before injection. NX288 (SEQ ID NO: 193) inhibited trafficking of cells to PLN (FIG. 13) and MLN in a dose-dependent fashion but had no effect on the accumulation of cells in other organs. At the highest dose tested (4 nmol), inhibition by the DNA ligand was comparable to that of DREG-56 (13 pmol), while a scrambled sequence had no significant effect (FIG. 13). The activity of LD174T1 (SEQ ID NO: 194) was similar to that of NX288.

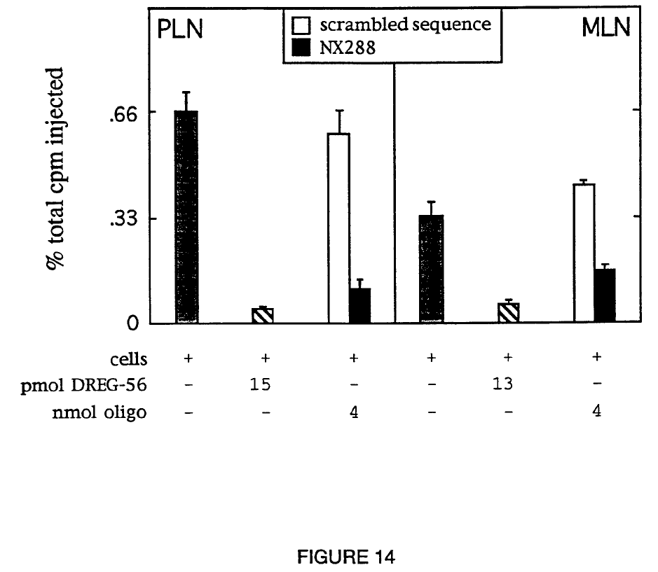

To determine if the modified oligonucleotide was effective when it was not pre-incubated with cells, DREG-56 (13 pmol/mouse) or the modified oligonucleotide (4 nmol/mouse) was injected intravenously into animals and 1-5 min later the radio-labeled human cells were given intravenously. Again, both NX288 (SEQ ID NO: 193) and DREG-56 inhibited trafficking to PLN and MLN while the scrambled sequence had no effect (FIG. 14). Therefore, the modified oligonucleotide did not require pre-incubation with the cells to effectively block trafficking. These experiments demonstrate, in vivo, the efficacy of oligonucleotide ligands in inhibiting a L-selectin dependent process.

### Example 21

### L-Selectin Nucleic Acid Ligand Multimers

Multivalent Complexes were made in which two nucleic acid ligands to L-selectin were conjugated together. Multivalent Complexes of nucleic acid ligands are described in copending U.S. patent application Ser. No. 08/434,465, filed May 4, 1995, entitled “Nucleic Acid Ligand Complexes” which is herein incorporated by reference in its entirety. These multivalent Complexes were intended to increase the binding energy to facilitate better binding affinities through slower off-rates of the nucleic acid ligands. These multivalent Complexes may be useful at lower doses than their monomeric counterparts. In addition, high molecular weight (20 kD) polyethylene gylcol (PEG) was included in some of the Complexes to decrease the in vivo clearance rate of the complexes. Specifically, the nucleic acid ligands incorporated into the Complexes were LD201T1 (SEQ ID NO: 185), LD201T4 (SEQ ID NO: 187), LD201T10 (SEQ ID NO: 188) and NX303 (SEQ ID NO: 196). Multivalent selectin nucleic acid ligand Complexes were produced as described in Example 13, paragraph F.

A variety of monomeric nucleic acid ligands and multivalent Complexes have been examined in flow cytometry. The multivalent Complexes exhibited similar specificity to the monomeric forms, but enhanced affinity as well as improved (i.e., slower) off-rate for human lymphocytes. Titration curves, obtained from incubating fluorescently labeled monomeric FITC-LD201T1 with peripheral blood mononuclear cells (PBMC) purified human lymphocytes, indicated that binding to cells is saturable. Half-saturation fluorescence occurred at 3 nM oligonucleotide. In contrast, the branched dimeric FITC-LD201T1 and bio-LD201T1/SA multivalent Complexes exhibited half-saturation at approximately 0.15 nM, corresponding to an apparent 20-fold increase in affinity. In similar experiments, half saturation of the dumbell and fork dimers of LD201T4 was observed at 0.1 and 0.6 nM, respectively, compared to 20 nM for monomeric LD201T4.

Kinetic competition experiments were performed on monomeric nucleic acid ligands and multivalent Complexes. Kinetic competition experiments were performed with PBMC purified lymphocytes. Cells were stained as described above but used 10 nM oligonucleotide. The off-rate for monomeric, dimeric and multivalent Complexes was determined by addition of 500 nM unlabeled oligonucleotide to cells stained with fluorescently labeled ligand and measurement of the change in the mean fluorescence intensity as a function of time. The dissociation rate of a monomeric LD201T1 from L-selectin expressing human lymphocytes was approximately 0.005 sec-1, corresponding to a half-life of roughly 2.4 minutes. The LD201T1 branched dimer and biotin conjugate multivalent Complexes exhibited apparent off-rates several times slower than that observed for the monomeric ligand and as slow or slower than that observed for the anti-L-selectin blocking antibody DREG56, determined under the same conditions. A multivalent Complex containing a non-binding nucleic acid sequence did not stain cells under identical conditions and did not compete in the off-rate experiments. The off-rate of the LD201T4 dumbell and fork dimers is faster than the LD201T1 branched dimer and is better than all monomers tested. These results confirm the proposition that dimeric and multimeric ligands bind with higher affinities than do monomeric ligands and that the increased affinity results from slower off-rates.

### Example 22

### 2′-F RNA Ligands to Human L-Selectin

The experimental procedures outlined in this Example were used to identify and characterize 2′-F RNA ligands to human L-selectin as described in Examples 23-25.

### Experimental Procedures

### A) Materials

Unless otherwise indicated, all materials used in the 2′-F RNA SELEX against the L-selectin/IgG2 chimera, LS-Rg, were identical to those of Examples 7, paragraph A and 13, paragraph A. SHMCK-140 buffer, used for all SELEX and binding experiments, was 1 mM CaCl₂, 1 mM MgCl₂, 140 mM NaCl, 5 mM KCl, and 20 mM HEPES, pH 7.4. A soluble form of L-selectin, corresponding to the extracellular domains, was purchased from R&D Systems and used for some nitrocellulose filter binding experiments.

### B) SELEX

The SELEX procedure is described in detail in U.S. Pat. No. 5,270,163 and elsewhere. Procedures are essentially identical to those in Examples 7 and 13 except as noted. The variable regions of synthetic DNA templates were randomized at either 30 or 40 positions and were flanked by N7 5′ and 3′ fixed regions producing transcripts 30N7 (SEQ ID NO: 292) and 40N7 (SEQ ID NO: 389). The primers for the PCR were the following:

| | | |
|---|---|---|
| N7 5′ Primer | 5′ taatacgactcactatagggaggacgatgcgg 3′ | (SEQ ID NO: 65) |
| N7 3′ Primer | 5′ tcgggcgagtcgtcctg 3′ | (SEQ ID NO: 66) |

The initial RNA pool was made by first Klenow extending 3 nmol of synthetic single stranded DNA and then transcribing the resulting double stranded molecules with T7 RNA polymerase. Klenow extension conditions: 6 nmols primer 5N7, 3 nmols 30N7 or 40n7, 1× Klenow Buffer, 1.8 mM each of DATP, dCTP, dGTP and dTTP in a reaction volume of 0.5 ml.

For subsequent rounds, eluted RNA was the template for AMV reverse transcriptase mediated synthesis of single-stranded cDNA. These single-stranded DNA molecules were converted into double-stranded transcription templates by PCR amplification. PCR conditions were 50 mM KCl, 10 mM Tris-Cl, pH 8.3, 7.5 mM MgCl₂, 0.2 mM of each dATP, DCTP, dGTP, and dTTP, and 100 U/ml of Taq DNA polymerase. Transcription reactions contained one third of the purified PCR reaction, 200 nM T7 RNA polymerase, 80 mM HEPES (pH 8.0), 12 mM MgCl₂, 5 mM DTT, 2 mM spermidine, 1 mM each of 2′-OH ATP, 2′-OH GTP, 3 mM each of 2′-F CTP, 2′-F UTP, and 250 nM α-³²P 2′-OH ATP. Note that in all transcription reactions 2′-F CTP and 2′-F UTP replaced CTP and UTP.

The strategy for partitioning LS-Rg/RNA complexes from unbound RNA is outlined in Table 15 and is essentially identical to that of Example 7, paragraph B. In the initial SELEX rounds, which were performed at 37° C., the density of immobilized LS-Rg was 10 pmols/μl of Protein A Sepharose 4 Fast Flow beads. LS-Rg was coupled to protein A Sepharose beads according to the manufacturer's instructions (Pharmacia Biotech). In later rounds, the density of LS-Rg was reduced (Table 15), as needed, to increase the stringency of selection. At the seventh round, both SELEXes were branched. One branch was continued as previously described (Example 7, paragraph B). In the second branch of both SELEXes, the RNA pool was pre-annealed to oligonucleotides that are complementary to the 5′ and 3′ fixed sequences. These rounds are termed “counter-selected” rounds. Before each round, RNA was batch adsorbed to 100 μl of protein A Sepharose beads for 15 minutes in a 2 ml siliconized column. Unbound RNA and RNA eluted with minimal washing (two volumes) were combined and used for SELEX input material. For SELEX, extensively washed, immobilized LS-Rg was batch incubated with pre-adsorbed RNA for 1 to 2 hours in a 2 ml column with constant rocking. Unbound RNA was removed by extensive batch washing (500 μl SHMCK 140/wash). In addition, the counter selected rounds were extensively washed with buffer containing 200 nM of both complementary oligos. Bound RNA was eluted as two fractions; first, bound RNA was eluted by incubating and washing columns with 100 μL 5 mM EDTA in SHMCK 140 without divalent cations; second, the remaining elutable RNA was removed by incubating and/or washing with 500 μL 50 mM EDTA in SHMCK 140 without divalents. The percentage of input RNA that was eluted is recorded in Table 22. In every round, an equal volume of protein A Sepharose beads without LS-Rg was treated identically to the SELEX beads to determine background binding. All unadsorbed, wash and eluted fractions were counted in a Beckman LS6500 scintillation counter in order to monitor each round of SELEX.

The 5 mM EDTA eluates were processed for use in the following round (Table 15). After precipitating with isopropanol/ethanol (1:1, v/v), the RNA was reverse transcribed into cDNA by AMV reverse transcriptase either at 48° C. for 15 minutes and then 65° C. for 15 minutes in 50 mM Tris-Cl pH (8.3), 60 mM NaCl, 6 mM Mg(OAc)₂, 10 mM DTT, 200 μmol DNA primer, 0.5 mM each of dNTPs, and 0.4 unit/μL AMV RT. Transcripts of the PCR product were used to initiate the next round of SELEX.

### C) Nitrocellulose Filter Binding Assay

As described in SELEX patent applications, a nitrocellulose filter partitioning method was used to determine the affinity of RNA ligands for LS-Rg and for other proteins. Filter discs (nitrocellulose/cellulose acetate mixed matrix, 0.45 μm pore size, Millipore) were placed on a vacuum manifold and washed with 3 ml of SHMCK 140 buffer under vacuum. Reaction mixtures, containing ³²P labeled RNA pools and unlabeled LS-Rg, were incubated in SHMCK 140 for 10-20 min at 37° C., and then immediately washed with 3 ml SHMCK 140. The filters were air-dried and counted in a Beckman LS6500 liquid scintillation counter without fluor. Alternatively, binding studies employed 96 well micro-titer manifolds essentially as described in Example 13, paragraph E.

### D) Cloning and Sequencing

12th round PCR products were re-amplified with primers which contain either a BamHI or a HinDIII restriction endonuclease recognition site. Using these restriction sites, the DNA sequences were inserted directionally into the pUC9 vector. These recombinant plasmids were transformed into E. coli strain DH5a (Life Technologies, Gaithersburg, Md.). Plasmid DNA was prepared according to the alkaline lysis method (Quiagen, QIAwell, Chattsworth Calif.). Approximately 300 clones were sequenced using the ABI Prism protocol (Perkin Elmer, Foster City, Calif.). Sequences are shown in Table 16.

### E) Cell Binding Studies

Binding of evolved ligands to L-selectin presented in the context of a cell surface was tested by flow cytometry experiments with human lymphocytes. Briefly, peripheral blood mononuclear cells (PBMC) were purified on histoplaque by standard techniques. To evaluate leukocyte binding by unlabeled 2′-F ligands, cells (500 cells/mL) were incubated with fluorescein labeled FITC-LD201T1 (SEQ ID NO: 185) in the presence of increasing concentrations of individual, unlabeled 2′-F ligands in 0.25 mL SMHCK buffer (140 mM NaCl, 1 mM MgCl₂, 1 mM CaCl₂, 5 mM, KCl, 20 mM HEPES pH 7.4, 8.9 mM NaOH, 0.1% (w/v) BSA, 0.1% (w/v) sodium azide) at room temperature for 15 minutes. Fluorescent staining of cells was quantified on a FACSCaliber fluorescent activated cell sorter (Becton Dickinson, San Jose, Calif.). The affinity of the 2′-F competitor was calculated from the fluororescence inhibition curves.

### Example 23

### 2′-F RNA Ligands to L-Selectin

### A. SELEX

The starting RNA pools for SELEX, randomized 30N7 (SEQ ID NO: 292) or 40N7 (SEQ ID NO: 389) contained approximately 10¹⁴ molecules (0.7 nmol RNA). The SELEX protocol is outlined in Table 15 and Example 22. All rounds were selected at 37° C. The dissociation constant of randomized RNA to LS-Rg is estimated to be approximately 10 μM. After six rounds the pool affinities had improved to approximately 300 nM. An aliquot of the RNA recovered from the seventh round was used as the starting material for the first counter-selected rounds. Five rounds of counter-selection and five additional standard rounds were performed in parallel. Thus, a total of twelve rounds were performed in both branches of both SELEXes: 30N7, counter-selected 30N7, 40N7 and counter-selected 40N7. The affinities of each of the 12th round pools ranged from 60 to 400 pM. Ligands were cloned from these pools.

### B. Sequences of 2′-F RNA Ligands to L-Selectin

In Table 16, ligand sequences are shown in standard single letter code (Cornish-Bowden, 1985 NAR 13: 3021-3030). Fixed region sequence is shown in lower case letters. By definition, each clone includes both the evolved sequence and the associated fixed region, unless specifically stated otherwise. A unique sequence is operationally defined as one that differs from all others by three or more nucleotides. Sequences that were isolated more than once are indicated by the parenthetical number, (n), following the ligand isolate number.

The 30N7 and 40N7 SELEX final pools shared a common major sequence family, even though identical sequences from the two SELEXes are rare (Table 16). Most ligands (72 of the 92 unique sequences) from the 30N7 and 40N7 SELEXes contain one of two related sequence motifs, RYGYGUUUUCRAGY or RYGYGUUWWUCRAGY. These motifs define family 1. Within the family there are three subfamilies. Subfamily 1a ligands (53/66) contain an additional sequence motif, CUYARRY, one nucleotide 5′ to the family 1 consensus motifs. Subfamily 1b (9/66 unique sequences) lacks the CUYARRY motif. Subfamily 1c (5/66) is also missing the CUYARRY motif, has an A inserted between the Y and G of consensus YGUU and lacks the consensus GA base pair. The significance of the sequence subfamilies is reflected in the postulated secondary structure of the ligands (Example 25).

A second family, composed of 5 sequences, has a relatively well defined consensus: UACUAN₀₋₁UGURCG . . . UYCACUAAGN₁₋₂CCC (Table 16). Family 3 has a short, unreliable consensus motif (Table 16). In addition, there are approximately 12 orphans or apparently unrelated sequences. Three of the orphan sequences were recovered at least twice (Table 16).

### C. Affinities

The dissociation constants of representative ligands from Table 16 are shown in Table 17. These calculations assume two ligand binding sites per chimera. The affinity of random 2′-F RNA cannot be reliably determined but is estimated to be approximately 10 μM.

The dissociation constants range from 34 μM to 315 nM at 37° C. Binding affinity is not expected to be temperature sensitive since selection was at 37° C. and 2′-F RNA forms thermal stable structures, but binding has not been tested at lower temperatures. For the most part, the extreme differences in affinity may be related to predicted secondary structure (Example 25).

The observed affinities of the evolved 2′-F RNA ligands reaffirm our proposition that it is possible to isolate oligonucleotide ligands with affinities that are several orders of magnitude greater than that of carbohydrate ligands.

### Example 24

### Cell Binding Studies

The ability of full length 2′-F ligands to bind to L-selectin presented in the context of a cell surface was tested by competition-flow cytometry experiments with human peripheral blood lymphocytes. Lymphocytes were stained with 10 nM FITC-conjugated DNA ligand FITC-LD201T1 (SEQ ID NO: 185) in the presence of increasing concentrations of unlabeled 2′-F ligands as described in Example 22, paragraph E. Ligands LF1513 (SEQ ID NO: 321), LF1514 (SEQ ID NO: 297), LF1613 (SEQ ID NO: 331) and LF1618 (SEQ ID NO: 351) inhibited the binding of FITC-LD201T1 in a concentration dependent manner, with complete inhibition observed at competitor concentrations of 10 to 300 nM. These results demonstrate that the 2′-F ligands are capable of binding cell surface L-selectin and suggest that the 2′-F ligands and LD201T1 bind the same or overlapping sites. The affinities of the fluoro ligands, calculated from the competition curves, range from 0.2 to 25 nM. The affinity of two of the ligands for L-selectin on human lymphocytes, LF1613 (Kd=0.2 nM) and LF1514 (Kd=0.8 nM), is significantly better than that of the DNA ligand LD201T1 (Kd=3 nM). The reasonable agreement between the affinities for purified protein and lymphocyte L-selectin suggests that binding to lymphocytes is specific for L-selectin. These data validate the feasibility of using immobilized, purified protein to isolate ligands against a cell surface protein.

### Example 25

### Secondary Structure of High Affinity 2′-F RNA Ligands to L-Selectin

In favorable instances, comparative analysis of aligned sequences allows deduction of secondary structure and structure-function relationships. If the nucleotides at two positions in a sequence covary according to Watson-Crick base pairing rules, then the nucleotides at these positions are apt to be paired. Nonconserved sequences, especially those that vary in length are not apt to be directly involved in function, while highly conserved sequence are likely to be directly involved.

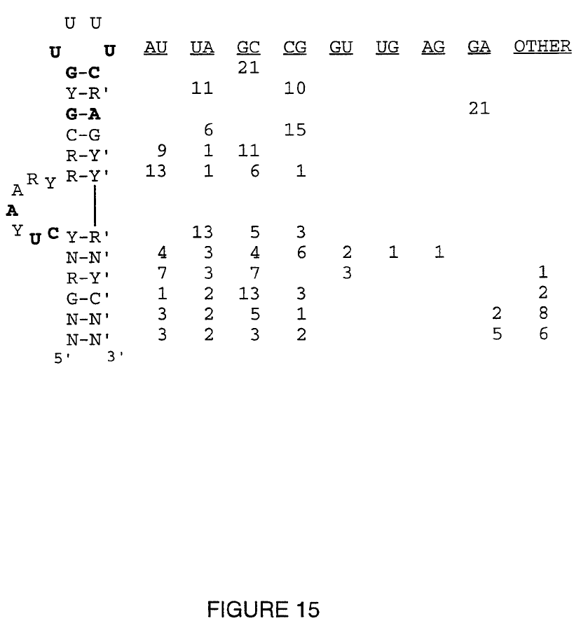

The deduced secondary structure of family 1a ligands from comparative analysis of 21 unique sequences is a hairpin motif (FIG. 15) consisting of a 4 to 7 nucleotide terminal loop, a 6 base upper stem and a lower stem of 4 or more base pairs. The consensus terminal loops are either a UUUU tetraloop or a UUWWU pentaloop. Hexa- and heptaloops are relatively rare. The upper and lower stems are delineated by a 7 nucleotide bulge in the 5′-half of the stem. Four of the six base pairs in the upper stem and all base pairs in the lower stem are supported by Watson-Crick covariation. Of the two invariant base pairs in the upper stem, one is the loop closing GC, while the other is a non-standard GA. The lower stem is most often 4 or 5 base pairs long but can be extended. While the sequence of the upper stem is strongly conserved, that of the lower stem is not, with the possible exception of the YR′ base pair adjacent to the internal bulge. This base pair appears to covary with the 3′ position of the 7 nucleotide bulge in a manner which minimizes the likelihood of extending the upper stem. Both the sequence (CUYARRY) and length (7 nt) of the bulge are highly conserved.

In terms of comparative analysis, the 7 nucleotide bulge, the upper stem and the 5′ and 3′ positions of the terminal loop are most apt to be directly involved in L-selectin binding. Specifically, the 5′ U and 3′ U of the terminal loop, the invariant GC and GA base pairs of the upper stem and the conserved C, U and A of the bulge are the mostly likely candidates. The lower stem, because of its variability in length and sequence, is less likely to be directly involved. The importance of the bulge for binding is supported by the poor affinity of ligand LF1512 (SEQ ID NO: 357; Kd=315 nM); the simplest structure for this ligand is a UUUU tetraloop and a ten base pair, nearly perfect, consensus stem which is missing only the 7 nucleotide bulge.

The deduced secondary structure of family 1b is similar to that of family 1a, except that the upper stem is usually 7 base pairs in length and that the single stranded bulge which does not have a highly conserved consensus is only 4 nucleotide long. This structure may be an acceptable variation of the 1a secondary structure with the upper stem's increased length allowing a shorter bulge; the affinity of ligand LF1511 (SEQ ID NO: 332) is 300 μM.

Although family 1c has a consensus sequence, GUUUUCNR that is related to 1a and 1b, a convincing consensus secondary structure is not evident, perhaps due to insufficient data. The most highly structured member of the family, LF1618 (SEQ ID NO: 351), permits a UUUU tetraloop and “upper” stem of 7 base pairs but has neither a lower stem nor the consensus 7 nucleotide bulge sequence of 1a. The upper stem differs from those of 1a and 1b in that it has an unpaired A adjacent to the loop closing G and does not have the invariant GA base pair of 1a and 1b. The affinity of LF1618 is a modest 10 nM which suggests that family 1c forms a less successful structure.

Predictions of minimal high affinity sequences for family 1 ligands can be made and serve as a partial test of the postulated secondary structure. Truncates which include only the upper stem and terminal loop, LF1514T1 (SEQ ID NO: 385) or these two elements plus the 7 nucleotide bulge sequence, LF1514T2 (SEQ ID NO: 386), are not expected to bind with high affinity. On the other hand, there is a reasonable, but not rigorous, expectation that ligands truncated at the base of the lower consensus stem, LF1514T4 (SEQ ID NO: 387) and LF1807T4 (SEQ ID NO: 388), will bind with high affinity. In side by side comparisons, the affinities of LF1514T1 and LF1514T2 for LS-Rg were reduced at least 100-fold in comparison to full length LD1514 (SEQ ID NO: 297), while the affinity of LF1514T4 was reduced less than two fold and that of LF1807T4 approximately three-fold. The correspondence between the predicted and observed truncate affinities supports the postulated secondary structure.

Since the ssDNA ligand LD201T1 (SEQ ID NO: 185) and the adhesion blocking anti-human L-selectin antibody DREG56 are known to bind to the lectin domain of L-selectin, competition between radio-labeled LF1807 (SEQ ID NO: 309) and either unlabeled DREG56 or unlabeled LD201T1 can serve to determine if the 2′-F ligands also bind the lectin domain of purified LS-Rg. In these experiments, both DREG56 and LD201T1 gave concentration dependent inhibition of LF1807 binding. Complete inhibition was attained with 300 nM Mab and 1 μM LD201T1. The competitors' affinities of LS-Rg, calculated from the competition curves, were in good agreement with their known affinities. These results are consistent with the premise that LF1807, NX280 and DREG56 have the same or overlapping binding sites and consequently it is expected that 2′-F ligands will be antagonists of L-selectin mediated adhesion. These results also reaffirm the proposition that the SELEX protocol, with 5 mM elution of bound oligonucleotides, preferentially elutes ligands bound at or near the lectin domain's bound calcium.

### Example 26

### ssDNA Ligands to Human P-Selectin

PS-Rg is a chimeric protein in which the lectin, EGF, and the first two CRD domains of human P-selectin are joined to the Fc domain of a human G1 immunoglobulin (R. M. Nelson et al., 1993, supra). Purified chimera is provided by A. Varki. Soluble P-selectin is purchased from R&D Systems. Unless otherwise indicated, all materials used in the ssDNA SELEX against the P-selectin/IgG₁ chimera, PS-Rg, are identical to those of Examples 7 and 13.

The SELEX procedure is described in detail in U.S. Pat. No. 5,270,163. The specific strategies and procedures for evolving high affinity ssDNA antagonists to P-selectin are described in Examples 7 and 13.

### Example 27

### 2′-F RNA Ligands to Human P-Selectin

The Experimental procedures outlined in this Example were used to identify 2′-F RNA ligands to human P-selectin as described in Examples 28-34.

### Experimental Procedures

### A) Materials

PS-Rg is a chimeric protein in which the extracellular domain of human P-selectin is joined to the Fc domain of a human G2 immunoglobulin (Norgard et al., 1993, PNAS 90:1068-1072). ES-Rg and CD22 β-Rg are analogous constructs of E-selectin and CD220 joined to a human G1 immunoglobulin Fc domain (R. M. Nelson et al., 1993, supra; I. Stamenkovic et al., 1991, Cell 66, 1133-1144) while LS-Rg has L-selectin joined to an IgG2 Fc domain. Purified chimera were provided by A. Varki. Soluble P-selectin was purchased from R&D Systems. Protein A Sepharose 4 Fast Flow beads were purchased from Pharmacia Biotech. Anti-P-selectin monoclonal antibodies: G1 was obtained from Centocor. The 2′-F modified CTP and UTP were prepared according to Pieken et. al. (1991, Science 253:314-317). DNA oligonucleotides were synthesized by Operon. All other reagents and chemicals were purchased from commercial sources. Unless otherwise indicated, experiments utilized HSMC buffer (1 mM CaCl₂, 1 mM MgCl₂, 150 mM NaCl, 20.0 mM HEPES, pH 7.4).

### B) SELEX

The SELEX procedure is described in detail in U.S. Pat. No. 5,270,163 and elsewhere. The nucleotide sequence of the synthetic DNA template for the PS-Rg SELEX was randomized at 50 positions. This variable region was flanked by N8 5′ and 3′ fixed regions. The transcript 50N8 has the sequence 5′ gggagacaagaauaaacgcucaa-50N-uucgacaggaggcucacaac-aggc 3′ (SEQ ID NO: 390). All C and U have 2′-F substituted for 2′-OH on the ribose. The primers for the PCR were the following:

| | | |
|---|---|---|
| N8 5′ Primer | 5′ taatacgactcactatagggagacaagaataaacgctcaa 3′ | (SEQ ID NO: 197) |
| N8 3′ Primer | 5′ gcctgttgtgagcctcctgtcgaa 3′ | (SEQ ID NO: 198) |

The fixed regions include primer annealing sites for PCR and cDNA synthesis as well as a consensus T7 promoter to allow in vitro transcription. The initial RNA pool was made by first Klenow extending 1 nmol of synthetic single stranded DNA and then transcribing the resulting double stranded molecules with T7 RNA polymerase. Klenow extension conditions: 3.5 nmols primer 5N8, 1.4 nmols 40N8, 1× Klenow Buffer, 0.4 mM each of DATP, dCTP, dGTP and dTTP in a reaction volume of 1 ml.

For subsequent rounds, eluted RNA was the template for AMV reverse transcriptase mediated synthesis of single stranded cDNA. These single-stranded DNA molecules were converted into double-stranded transcription templates by PCR amplification. PCR conditions were 50 mM KCl, 10 mM Tris-Cl, pH 8.3, 7.5 mM MgCl₂, 1 mM of each dATP, dCTP, dGTP, and dTTP, and 25 U/ml of Taq DNA polymerase. Transcription reactions contained 0.5 mM DNA template, 200 nM T7 RNA polymerase, 40 mM Tris-HCl (pH 8.0), 12 mM MgCl₂, 5 mM DTT, 1 mM spermidine, 4% PEG 8000, 1 mM each of 2′-OH ATP and 2′-OH GTP, 3.3 mM each of 2′-F CTP and 2′-F UTP, and 250 nM α-³²P 2′-OH ATP.

The strategy for partitioning PS-Rg/RNA complexes from unbound RNA is essentially identical to the strategy detailed in Example 7 for ligands to L-selectin (Table 18).

In the initial SELEX rounds, which were performed at 37° C., the density of immobilized PS-Rg was 20 pmols/μl of Protein A Sepharose 4 Fast Flow beads. In later rounds, the density of PS-Rg was reduced (Table 18), as needed, to increase the stringency of selection. Beginning with the second round, SELEX was often done at more than one PS-Rg density. At each round, the eluted material from only one PS-Rg density was carried forward.

Before each round, RNA was batch adsorbed to 100 μl of protein A Sepharose beads for 1 hour in a 2 ml siliconized column. Unbound RNA and RNA eluted with minimal washing (two volumes) were combined and used for SELEX input material. For SELEX, extensively washed, immobilized PS-Rg was batch incubated with pre-adsorbed RNA for 0.5 to 1 hours in a 2 ml siliconized column with frequent mixing. Unbound RNA was removed by extensive batch washing (500 μl HSMC/wash). Bound RNA was eluted as two fractions; first, bound RNA was eluted by incubating and washing columns with 5 mM EDTA in HSMC without divalent cations; second, the remaining elutable RNA was removed by incubating and/or washing with 50 mM EDTA in HSMC without divalents. The percentage of input RNA that was eluted is recorded in Table 18. In every round, an equal volume of protein A Sepharose beads without PS-Rg was treated identically to the SELEX beads to determine background binding. All unadsorbed, wash and eluted fractions were counted in a Beckman LS6500 scintillation counter in order to monitor each round of SELEX.

The eluted fractions were processed for use in the following round (Table 18). After precipitating with 300 mM Sodium Acetate pH 7 in ethanol (2.5 volumes), the RNA was resuspended in 80 μl of H₂O* and 40 μl were reverse transcribed into cDNA by AMV reverse transcriptase at 48° C. for 30 minutes, in 50 mM Tris-Cl pH (8.3), 60 mM NaCl, 6 mM Mg(OAc)₂, 10 mM DTT, 200 pmol DNA primer, 0.4 mM each of dNTPs, and 0.4 unit/μl AMV RT. Transcripts of the PCR product were used to initiate the next round of SELEX.

### C) Nitrocellulose Filter Binding Assay

As described in SELEX patent applications, a nitrocellulose filter partitioning method was used to determine the affinity of RNA ligands for PS-Rg and for other proteins. Filter discs (nitrocellulose/cellulose acetate mixed matrix, 0.45 μm pore size, Millipore) were placed on a vacuum manifold and washed with 2 ml of HSMC buffer under vacuum. Reaction mixtures, containing ³²P labeled RNA pools and unlabeled PS-Rg, were incubated in HSMC for 10-20 min at 4° C., room temperature or 37° C., filtered, and then immediately washed with 4 ml HSMC at the same temperature. The filters were air-dried and counted in a Beckman LS6500 liquid scintillation counter without fluor.

PS-Rg is a dimeric protein that is the expression product of a recombinant gene constructed by fusing the DNA sequence that encodes the extracellular domains of human P-selectin to the DNA that encodes a human IgG₁ Fc region. For affinity calculations, one ligand binding site per PS-Rg monomer (two per dimer) were assumed. The monomer concentration is defined as 2 times the PS-Rg dimer concentration. The equilibrium dissociation constant, K_{d}, for an RNA pool or specific ligand is calculated as described in Example 7, paragraph C.

### D) Cloning and Sequencing

Twelfth round PCR products were re-amplified with primers which contain either a BamHI or a HinDIII restriction endonuclease recognition site. Using these restriction sites, the DNA sequences were inserted directionally into the pUC9 vector. These recombinant plasmids were transformed into E. coli strain JM109 (Life Technologies, Gaithersburg, Md.). Plasmid DNA was prepared according to the alkaline hydrolysis method (PERFECTprep, 5′-3′, Boulder, Colo.). Approximately 50 clones were sequenced using the Sequenase protocol (Amersham, Arlington Heights, Ill.). The resulting ligand sequences are shown in Table 19.

### E) Boundary Experiments

The minimal high affinity sequence of individual ligands was determined by boundary experiments (Tuerk et. al. 1990, J. Mol. Biol. 213: 749). Individual RNA ligands, ³²P_labeled at the 5′-end for the 3′ boundary and ³²P-labeled at the 3′-end for the 5′ boundary, are hydrolyzed in 50 mM Na2CO3 pH 9 for 8 minutes at 95° C. The resulting partial hydrolysate contains a population of end-labeled molecules whose hydrolyzed ends correspond to each of the purine positions in the full length molecule. The hydrolysate is incubated with PS-Rg (at concentrations 5-fold above, below and at the measured Kd for the ligand). The RNA concentration is significantly lower than the Kd. The reaction is incubated at room temperature for 30 minutes, filtered, and then immediately washed with 5 ml HSMC at the same temperature. The bound RNA is extracted from the filter and then electrophoresed on an 8% denaturing gel adjacent to hydrolyzed RNA which has not been incubated with PS-Rg. Analysis is as described in Tuerk et. al. 1990, J. Mol. Biol. 213: 749.

### F) 2′-O-Methyl Substitution Experiments

In order to decrease the susceptibility of the 2′-F pyrimidine RNA ligands to nuclease digestion, post-SELEX modification experiments were performed to identify 2′-OH purines that are replaceable with 2′-OMe purines without loss of affinity as described in Green et. al. (1995, J. Mol. Biol. 247: 60-68). Briefly, seven oligonucleotides were synthesized, each with three mixed positions. A mixed position is defined as a 2′-OH purine nucleotide within the RNA which has been synthesized with 2:1 ratio of 2′-OH:2′-OMe. Since the coupling efficiency of 2′-OH phosphoramidites is lower than that of 2′-OMes, the resulting RNA has 25-50% 2′-OH at each mixed position. ³²P end-labeled RNA ligands are then incubated with concentrations of PS-Rg 2-fold above and 2.5-fold below the Kd of the unmodified ligand at room temperature for 30 minutes, filtered, and then immediately washed with 5 ml HSMC at the same temperature. The bound RNA (Selected RNA) is extracted from the filter and then hydrolyzed with 50 mM Na₂CO₃ pH 9 for 8 minutes at 95° C. in parallel with RNA which has not been exposed to binding and filtration (Unselected RNA). The Selected RNA is then electrophoresed on a 20% denaturing gel adjacent to Unselected RNA.

To determine the effect on binding affinity of 2′-OMe substitution at a particular position, the ratio of intensities of the Unselected:Selected bands that correspond to the position in question are calculated. The Unselected:Selected ratio when the position is mixed is compared to the mean ratio for that position from experiments in which the position is not mixed. If the Unselected:Selected ratio of the mixed position is significantly greater than that when the position is not mixed, 2′-OMe may increase affinity. Conversely, if the ratio is significantly less, 2′-OMe may decrease affinity. If the ratios are not significantly different, 2′-OMe substitution has no affect.

### G) Cell Binding Studies

The ability of evolved ligand pools and cloned ligands to bind to P-selectin presented in the context of a cell surface was tested in experiments with human platelet suspensions. Whole blood from normal volunteers was collected in Vacutainer 6457 tubes. Within 5 minutes of collection, 485 g of blood was stimulated with 15 μl Bio/Data THROMBINEX for 5 minutes at room temperature. A 100 μl aliquot of stimulated blood was transferred to 1 ml of BB+ (140 mM NaCl, 20 mM HEPES pH 7.35, 5 mM KCl, 0.01% NaN₃) at 4° C. and spun at 735×g for 5 minutes. This step was repeated and the resulting pellet was re-suspended in 1 ml of BB+(140 mM NaCl, 20 mM HEPES pH 7.35, 5 mM KCl. 0.01% NaN₃, 1 mM CaCl₂, 1 mM MgCl₂) at 4° C.

To detect antigen expression, 15 μl BB+containing FITC conjugated anti-CD61 or PE conjugated anti-CD62 antibody (Becton Dickinson) was incubated for 20-30 minutes at 4° C. with 10 μl of platelet suspension. This was diluted to 200 μl with 4° C. BB+ and analyzed on a Becton Dickinson FACSCaliber using 488 nm excitation and FL1 (530 nm emission) or FL2 (580 rum emission) with the machine live gated on platelets. Between 1000 and 5000 events in this gate were recorded.

To detect oligonucleotide ligand binding, 15 μl BB+containing ligand conjugated to either FITC or biotin was incubated 20-30 minutes at 4° C. with 10 μl platelet suspension. The FITC-ligand incubations were diluted to 200 μl with BB+ and analyzed on a FACSCaliber flow cytometer. The biotinylated-ligand reactions were incubated with streptavidin-phycoerythrin (SA-PE) (Becton Dickinson) for 20 minutes at 4° C., before dilution and analysis. Wash steps with 500 μl BB+ and 700×g spins have been used without compromising the quality of the results.

The specificity of binding to P-selectin (CD62P) expressed on platelets was tested by competition with the P-selectin specific blocking monoclonal antibody, G1. Saturability of binding was tested by self-competition with unlabeled RNA.

### H) Inhibition of Selectin Binding to Sialyl-Lewis^{X}

The ability of evolved RNA pools or cloned ligands to inhibit the binding of PS-Rg to sialyl-Lewis^{X} was tested in competitive ELISA assays (C. Foxall et al., 1992, supra). For these assays, the wells of Corning (25801) 96 well microtiter plates were coated with 100 ng of a sialyl-Lewisx/BSA conjugate, air dried overnight, washed with 300 μl of PBS(−) and then blocked with 1% BSA in HSMC for 60 min at room temperature. RNA ligands were incubated with PS-Rg in HSMC/1% BSA at room temperature for 15 min. After removal of the blocking solution, 50 μl of PS-Rg (10 nM) or a PS-Rg (10 nM)/RNA ligand mix was added to the coated, blocked wells and incubated at room temperature for 60 minutes. The binding solution was removed, wells were washed with 300 μl of PBS(−) and then probed with HRP conjugated anti-human IgG, at room temperature to quantitate PS-Rg binding. After a 30 minute incubation at room temperature in the dark with OPD peroxidase substrate (Sigma P9187), the extent of PS-Rg binding and percent inhibition was determined from the OD₄₅₀.

### Example 28

### 2′-F RNA Ligands to Human P-selectin

### A. SELEX

The starting RNA pool for SELEX, randomized 50N8 (SEQ ID NO: 390), contained approximately 10¹⁵ molecules (1 nmol RNA). The SELEX protocol is outlined in Table 18. The dissociation constant of randomized RNA to PS-Rg is estimated to be approximately 2.5 μM. An eight-fold difference was observed in the RNA elution profiles with 5 mM EDTA from SELEX and background beads for rounds 1 and 2, while the 50 mM elution produced a 30-40 fold excess over background Table 18. For rounds 1 through 3, the 5 mM and 50 mM eluted RNAs were pooled and processed for the next round. Beginning with round 4, only the 5 mM eluate was processed for the following round. To increase the stringency of selection, the density of immobilized PS-Rg was reduced five fold in round 2 and again in round three without greatly reducing the fraction eluted from the column. The density of immobilized PS-Rg was further reduced 1.6-fold in round 4 and remained at this density until round 8, with further reductions in protein density at later rounds. The affinity of the selected pools rapidly increased and the pools gradually evolved biphasic binding characteristics.

Binding experiments with 12th round RNA revealed that the affinity of the evolving pool for P-selectin was not temperature sensitive. Bulk sequencing of 2nd, 6th, 11th and 12th RNA pools revealed noticeable non-randomness by round twelve. The 6th round RNA bound monophasically at 37° C. with a dissociation constant of approximately 85 nM, while the 11th and 12th round RNAs bound biphasically with high affinity Kds of approximately 100 and 20 pM, respectively. The binding of all tested pools required divalent cations. In the absence of divalent cations, the Kds of the 12th round pools increased to >10 nM. (HSMC, minus Ca⁺⁺/Mg⁺⁺, plus 2 mM EDTA). The 12th round pool showed high specificity for PS-Rg with measured Kd's of 1.2 μM and 4.9 μM for ES-Rg and LS-Rg, respectively.

### B. RNA Sequences

In Table 19, ligand sequences are shown in standard single letter code (Cornish-Bowden, 1985 NAR 13: 3021-3030). Fixed region sequence is shown in lower case letters. By definition, each clone includes both the evolved sequence and the associated fixed region, unless specifically stated otherwise. From the twelfth round, 21 of 44 sequenced ligands were unique. A unique sequence is operationally defined as one that differs from all others by three or more nucleotides. Sequences that were isolated more than once, are indicated by the parenthetical number, (n), following the ligand isolate number. These clones fall into five sequence families (1-5) and a group of two unrelated sequences (Orphans) (SEQ ID NOs: 199-219).

Family 1 is defined by 23 ligands from 13 independent lineages. The consensus sequence is composed of two variably spaced sequences, CUCAACGAMC and CGCGAG (Table 19). In 11 of 13 ligands the CUCAA of the consensus is from 5′ fixed sequence which consequently minimizes variability and in turn reduces confidence in interpreting the importance of CUCAA or the paired GAG (see Example 27).

Families 2-5 are each represented by multiple isolates of a single sequence which precludes determination of consensus sequences.

### C. Affinities

The dissociation constants for representative ligands, including all orphans, were determined by nitrocellulose filter binding experiments and are listed in Table 20. These calculations assume two binding sites per chimera. The affinity of random RNA is estimated to be approximately 2.5 μM.

In general, ligands bind monophasically with dissociation constants ranging from 15 pM to 450 pM at 37° C. Some of the highest affinity ligands bind biphasically. Full length ligands of families 14 show no temperature dependence. The observed affinities substantiate the proposition that it is possible to isolate oligonucleotide ligands with affinities that are several orders of magnitude greater than that of carbohydrate ligands.

### Example 29

### Specificity of 2′-F RNA Ligands

The affinity of P-selectin ligands to ES-Rg, LS-Rg and CD22β-Rg were determined by nitrocellulose partitioning. As indicated in Table 20, the ligands are highly specific for P-selectin. In general, a ligand's affinity for ES-Rg and LS-Rg is at least 10⁴-fold lower than for PS-Rg. Binding above background is not observed for CD22β-Rg at the highest protein concentration tested (660 nM), indicating that ligands do not bind the Fc domain of the chimeric constructs nor do they have affinity for the sialic acid binding site of this unrelated lectin. The specificity of oligonucleotide ligand binding contrasts sharply with the binding of cognate carbohydrates by the selectins and confirms the proposition that SELEX ligands will have greater specificity than carbohydrate ligands.

### Example 30

### Inhibition of Binding to sialyl-Lewis^{X}

Oligonucleotide ligands, eluted by 2-5 mM EDTA, are expected to derive part of their binding energy from contacts with the lectin domain's bound Ca⁺⁺ and consequently, are expected to compete with sialyl-Lewis^{X} for binding. In competition assays, the selected oligonucleotide ligands competitively inhibit PS-Rg binding to immobilized sialyl-Lewis^{X} with IC50s ranging from 1 to 4 nM (Table 20). Specifically, ligand PF377 (SEQ ID NO: 206) has an IC50 of approximately 2 nM. Complete inhibition is attained at 10 nM ligand. This result is typical of high affinity ligands and is reasonable under the experimental conditions. The IC50s of ligands whose Kds are much lower than the PS-Rg concentration (10 nM) are limited by the protein concentration and are expected to be approximately one half the PS-Rg concentration. The specificity of competition is demonstrated by the inability of round 2 RNA (Kd.about. 1 μM) to inhibit PS-Rg binding to immobilized sialyl-Lewisx. These data verify that 2′-F RNA ligands are functional antagonists of PS-Rg.

### Example 31

### Secondary Structure of High Affinity Ligands

In favorable instances, comparative analysis of aligned sequences allows deduction of secondary structure and structure-function relationships. If the nucleotides at two positions in a sequence covary according to Watson-Crick base pairing rules, then the nucleotides at these positions are apt to be paired. Nonconserved sequences, especially those that vary in length are not apt to be directly involved in function, while highly conserved sequences are likely to be directly involved.

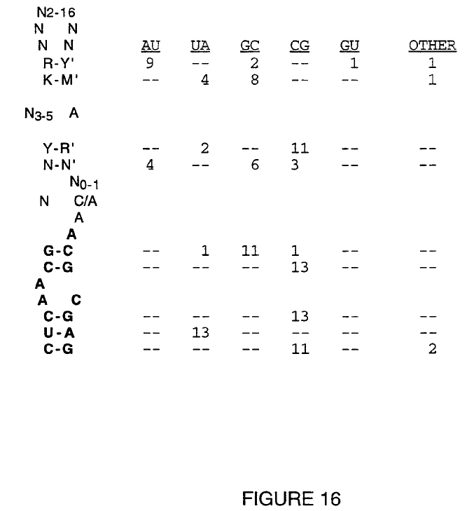

Comparative analysis of the family 1 alignment suggests a hairpin motif, the stem of which contains three asymmetrical internal loops (FIG. 16). In the figure, consensus positions are specified, with invariant nucleotides in bold type. To the right of the stem is a matrix showing the number of occurrences of particular base pairs for the positions in the stem that are on the same line. The matrix shows that 6 of the stem's 9 base pairs are supported by Watson-Crick covariation. Portions of the two consensus motifs, CUC and GAG, form the terminus of the stem. Conclusions regarding a direct role of the terminus in binding are tempered by the use of fixed sequence (11 of 13 ligands) which limits variability. The variability of the loop's sequence and length suggests that it is not directly involved in binding. This conclusion is reinforced by ligand PF422 (SEQ ID NO: 202) which is a circular permutation of the consensus motif. Although the loop that connects the stem's two halves is at the opposite end relative to other ligands, PF422 binds with high (Kd=172 pM; Table 21) affinity.

### Example 32

### Boundary Experiments

Boundary experiments were performed on a number of P-selectin ligands as described in Example 27 and the results are shown in Table 21. The results for family 1 ligands are consistent with their proposed secondary structure. The composite boundary species vary in size from 38-90 nucleotides, but are 40-45 nucleotides in family 1. Affinities of these truncated ligands are shown in Table 22. In general, the truncates lose no more than 10-fold in affinity in comparison to the full length, effectively inhibit the binding of PS-Rg to sialyl-Lewis^{X} and maintain binding specificity for PS-Rg (Table 22). These data validate the boundary method for identifying the minimal high affinity binding element of the RNA ligands.

### Example 33

### Binding of 2′-F RNA Ligands to Human Platelets

Since the P-selectin ligands were isolated against purified protein, their ability to bind P-selectin presented in the context of a cell surface was determined in flow cytometry experiments with activated human platelets. Platelets were gated by side scatter and CD61 expression. CD61 is a constitutively expressed antigen on the surface of both resting and activated platelets. The expression of P-selectin was monitored with anti-CD62P monoclonal antibody (Becton Dickinson). The mean fluorescence intensity of activated platelets, stained with biotintylated-PF377s1 (SEQ ID NO: 223)/SA-PE (Example 27, paragraph G), is 5 times greater than that of similarly stained resting platelets. In titration experiments, half maximal fluorescence occurs at approximately 50 pM PF377s1 (EC50) which is consistent with its equilibrium dissociation constant, 60 pM, for PS-Rg. Binding to platelets is specific by the criterion that it is saturable. Saturability has been demonstrated not only by titration but also by competition with unlabeled PF377s1.

Binding to platelets is P-selectin specific by the criteria that 1) oligonucleotides that do not bind PS-Rg do not bind platelets; 2) that binding of PF377s1 to platelets is divalent cation dependent; and most importantly 3) that binding is inhibited by the anti-P-selectin adhesion blocking monoclonal antibody G1, but not by an isotype control antibody. These data validate the feasibility of using immobilized, purified protein to isolate highly specific ligands against a cell surface P-selectin.

### Example 34

### 2′-O-Methyl Substitution Experiments

2′-OMe purine substitutions were performed on ligand PF377s1 (SEQ ID NO: 223) as described in Example 27 paragraph F and the results are shown in Table 23. The data indicate that 2′-OMe purines at positions 7-9, 15, 27, 28 and 31 enhance binding while substitutions at positions 13, 14, 16, 18, 21 22, 24, and 30 have little or no affect on affinity. Thus it appears that up to 15 positions may be substituted with only slight losses in affinity. In partial confirmation of this expectation, the affinity of 377s1 simultaneously substituted with 2′-OMe purines at 11 positions (PF377M6, SEQ ID NO: 235) is 250 pM (Table 22).

### Example 35

### 2′-NH₂ RNA Ligands to Human P-Selectin

The experimental procedures described in this Example are used in Examples 36-38 to isolate and characterize 2′-NH₂ RNA ligands to human P-selectin.

### Experimental Procedures

### A) Materials

Unless otherwise indicated, all materials used in the 2′-NH₂ RNA SELEX against the P-selectin/IgG₁ chimera, PS-Rg, were identical to those of Example 27. The 2′-NH₂ modified CTP and UTP were prepared according to Pieken et. al. (1991, Science 253:314-317). The buffer for SELEX experiments was 1 mM CaCl₂, 1 mM MgCl₂, 150 mM NaCl, 10.0 mM HEPES, pH 7.4.

### B) SELEX

The SELEX procedure is described in detail in U.S. Pat. No. 5,270,163 and elsewhere. The nucleotide sequence of the synthetic DNA template for the PS-Rg SELEX was randomized at 50 positions. This variable region was flanked by N8 5′ and 3′ fixed regions. The transcript 50N8 has the sequence 5′ gggagacaagaauaaac gcucaa-50N-uucgacaggaggcucacaa-caggc 3′ (SEQ ID NO: 248). All C and U have 2′-NH₂ substituted for 2′-OH on the ribose. The primers for the PCR were the following:

N8 5′ Primer 5′ taatacgactcactatagggagacaagaataaacgctcaa 3′ (SEQ ID NO: 249)

N8 3′ Primer 5′ gcctgttgtgagcctcctgtcgaa 3′ (SEQ ID NO: 250). The procedures used to isolate 2′-NH₂ oligonucleotide ligands to P-selectin are identical to those described 2′-F ligands in Example 27, except that transcription reactions utilized 1 mM each, 2′-NH.sub.2-CTP and 2′—NH₂-UTP, in place of 3.3 mM each 2′-F-CTP and 2′-F-UTP.

### C) Nitrocellulose Filter Binding Assay

As described in SELEX patent applications and in Example 27, paragraph C, a nitrocellulose filter partitioning method was used to determine the affinity of RNA ligands for PS-Rg and for other proteins. Either a Gibco BRL 96 well manifold, as described in Example 23 or a 12 well Millipore manifold (Example 7C) was used for these experiments. Binding data were analyzed as described in Example 7, paragraph C.

### D) Cloning and Sequencing

Twelfth round PCR products were re-amplified with primers which contain either a BamHI or a HinDIII restriction endonuclease recognition site. Approximately 75 ligands were cloned and sequenced using the procedures described in Example 7, paragraph D. The resulting sequences are shown in Table 25.

### E) Cell Binding Studies

The ability of evolved ligand pools to bind to P-selectin presented in the context of a cell surface was tested in flow cytometry experiments with human platelet suspensions as described in Example 7, paragraph E.

### Example 36

### 2′-NH₂ RNA Ligands to Human P-Selectin

### A. SELEX

The starting 2′-NH₂ RNA pool for SELEX, randomized 50N8 (SEQ ID NO: 248), contained approximately 1015 molecules (1 nmol 2′-NH₂ RNA). The dissociation constant of randomized RNA to PS-Rg is estimated to be approximately 6.4 μM. The SELEX protocol is outlined in Table 24.

The initial round of SELEX was performed at 37° C. with an PS-Rg density of 20 pmol/μl of protein A Sepharose beads. Subsequent rounds were all at 37° C. In the first round there was no signal above background for the 5 mM EDTA elution, whereas the 50 mM EDTA elution had a signal 7 fold above background, consequently, the two elutions were combined and processed for the next round. This scheme was continued through round 6. Starting with round seven only the 5 mM eluate was processed for the next round. To increase the stringency of selection, the density of immobilized PS-Rg was reduced ten fold in round 6 with further reductions in protein density at later rounds. Under these conditions a rapid increase in the affinity of the selected pools was observed.

Binding experiments with 12th round RNA revealed that the affinity of the evolving pool for P-selectin was temperature sensitive despite performing the selection at 37° C., (Kds: 13 pM, 91 pM and 390 pM at 4° C., room temperature and 37° C., respectively). Bulk sequencing of RNA pools indicated dramatic non-randomness at round 10 with not many visible changes in round 12. Ligands were cloned and sequenced from round 12.

### B. 2′-NH₂ RNA Sequences

In Table 25, the 2′-NH₂ RNA ligand sequences are shown in standard single letter code (Cornish-Bowden, 1985 NAR 13: 3021-3030) (SEQ ID NOS: 251-290). The evolved random region is shown in upper case letters in Table 25. Any portion of the fixed region is shown in lower case letters. By definition, each clone includes both the evolved sequence and the associated fixed region, unless specifically stated otherwise. From the twelfth round, 40/61 sequenced ligands were unique. A unique sequence is operationally defined as one that differs from all others by three or more nucleotides. Sequences that were isolated more than once are indicated by the parenthetical number, (n), following the ligand isolate number. Ligands from family 1 dominate the final pool containing 16/61 sequences, which are derived from multiple lineages. Families 2 and 3 are represented by slight mutational variations of a single sequence. Sequences labeled as “others” do not have any obvious similarities. Family 1 is characterized by the consensus sequence GGGAAGAAGAC (SEQ ID NO: 291).

### C. Affinities

The dissociation constants of representative ligands are shown in Table 26. These calculations assume two RNA ligand binding sites per chimera. The affinity of random 2′-NH₂ RNA is estimated to be approximately 10 μM.

At 37° C., the dissociation constants range from 60 pM to 50 nM which is at least a 1×10³ to 1×10⁵ fold improvement over randomized 2′-NH₂ RNA (Table 26). There is a marked temperature sensitivity for Clone PA350 (SEQ ID NO: 252) with an increase in affinity of 6 fold at 4° C. (Table 26). The observed affinities of the evolved 2′-NH₂ ligand pools reaffirm our proposition that it is possible to isolate oligonucleotide ligands with affinities that are several orders of magnitude greater than that of carbohydrate ligands.

### Example 37

### Specificity of 2′-NH₂ RNA Ligands to P-Selectin

The affinity of clone PA350 (SEQ ID NO: 252) for LS-Rg and ES-Rg was determined by nitrocellulose partitioning and the results shown in Table 26. The ligands are highly specific for P-selectin. The affinity for ES-Rg is about 600-fold lower and that for LS-Rg is about 5×10⁵-fold less than for PS-Rg. Binding above background is not observed for CD22β-Rg indicating that ligands neither bind the Fc domain of the chimeric constructs nor have affinity for unrelated sialic acid binding sites.

The specificity of oligonucleotide ligand binding contrasts sharply with the binding of cognate carbohydrates by the selectins and reconfirms the proposition that SELEX ligands will have greater specificity than carbohydrate ligands.

### Example 38

### Cell Binding Studies

FITC-labeled ligand PA350 (FITC-350) (SEQ ID NO: 252) was tested for its ability to bind to P-selectin presented in the context of a platelet cell surface by flow cytometry experiments as described in Example 23, paragraph G.

The specificity of FITC-PA350 for binding to P-selectin was tested by competition experiments in which FITC-PA350 and unlabeled blocking monoclonal antibody G1 were simultaneously added to stimulated platelets. G1 effectively competes with FITC-PA350 for binding to platelets, while an isotype matched control has little or no effect which demonstrates that FITC-PA350 specifically binds to P-selectin. The specificity of binding is further verified by the observation that oligonucleotide binding is saturable; binding of 10 nM FITC-PA350 is inhibited by 200 nM unlabeled PA350. In addition, the binding of FITC-PA350 is dependent on divalent cations; at 10 nM FITC-PA350 activated platelets are not stained in excess of autofluorescence in the presence of 5 mM EDTA.

These data validate the feasibility of using immobilized, purified protein to isolate ligands against a cell surface protein and the binding specificity of 2′-NH₂ ligands to P-selectin in the context of a cell surface.

### Example 39

### Inhibition of P-selectin Binding to Sialyl Lewisx

In competition assays, ligands PA341 (SEQ ID NO: 251) and PA350 (SEQ ID NO: 252) competitively inhibit PS-Rg binding to immobilized sialyl-Lewis^{x} with IC50s ranging from 2 to 5 nM (Table 26). This result is typical of high affinity ligands and is reasonable under the experimental conditions. The IC50s of ligands whose Kds are much lower than the PS-Rg concentration (10 nM) are limited by the protein concentration and are expected to be approximately one half the PS-Rg concentration. The specificity of competition is demonstrated by the inability of round 2 RNA (Kd.about. 1 μM) to inhibit PS-Rg binding to immobilized sialyl-Lewis^{x}. These data verify that 2-NH₂ RNA ligands are functional antagonists of P-selectin.

### Example 40

### 2′-NH₂ RNA Ligands to Human E-Selectin

ES-Rg is a chimeric protein in which the extracellular domain of human E-selectin is joined to the Fc domain of a human G1 immunoglobulin (R. M. Nelson et al., 1993, supra). Purified chimera were provided by A. Varki. Unless otherwise indicated, all materials used in this SELEX are similar to those of Examples 7 and 13. The SELEX procedure is described in detail in U.S. Pat. No. 5,270,163 and elsewhere. The rationale and experimental procedures are the same as those described in Examples 7 and 13.

TABLE 1
Wheat Germ Agglutinin Selex

| | Total Protein | Total RNA | Gel Volume | Total Volume | | | Kd |
|---|---|---|---|---|---|---|---|
| Round | (pmole) | (pmole) | (μl) | (μl) | % RNA Eluted | % RNA Amplified | (nM) |
| 1 | 5,800 | 2,020 | 50 | 276 | 0.05 | 0.05 | 6,000,000 |
| 2 | 5,800 | 1,070 | 50 | 276 | 0.12 | 0.12 | |
| 3 | 5,800 | 1,770 | 50 | 280 | 0.21 | 0.21 | |
| 4 | 5,800 | 900 | 50 | 263 | 3 | 3 | |
| 5 | 5,800 | 500 | 50 | 271 | 28.5 | 28.5 | 600 |
| 6a | 5,800 | 1,000 | 50 | 282 | 28.8 | | |
| 6b | 580 | 1,000 | 5 | 237 | 5.7 | 0.18 | 400 |
| 7 | 580 | 940 | 5 | 245 | 12.8 | 0.87 | 320 |
| 8 | 580 | 192 | 5 | 265 | 21.4 | 0.64 | 260 |
| 9 | 58 | 170 | 0.5 | 215 | 3.8 | 0.06 | 130 |
| 10 | 58 | 184 | 0.5 | 210 | 5.2 | 0.12 | 94 |
| 11 | 58 | 180 | 0.5 | 210 | 2.3 | 0.07 | 68 |

Wheat Germ Lectin Sepharose 6 MB, WGA density, approximately 5 mg/ml of gel or 116 μM.
RNA Loading Conditions: Rounds 1-5, 2 hrs @ room temperature on roller; incubation time reduced to 1 hr. for Rounds 6-11.
RNA Elution Conditions: Rounds 1-5, 200 μl of 2 mM (GlcNAc)3, 15 min. @ room temperature on roller; 2 × 200 μl wash with same buffer. Rounds 6: 200 μl of 0.2 mM (GlcNAc)3, incubated as above; washed sequentially with 200 μl of 0.5, 1, 1.5, 2 and 10 mM (GlcNAc)3. Rounds 7-8: 200 μl of 0.2 mM (GlcNAc)3, incubated as in round 6; wash twice with same buffer; washed sequentially with 3 × 200 μl each, of 0.5, 1.0, 1.5, 2.0 and 10 mM (GlcNAc)3. Rounds 9-11: incubated 15 @ room temperature in 200 μl of 1 mM (GlcNAc); washed 2 × with 200 μl of same buffer; incubation and washes repeated with 1.5, 2.0 and 10 mM (GlcNAc).
% RNA Eluted: percentage of input RNA eluted with (GlcNAc)3
% RNA Amplified: percentage of input RNA amplified; Rounds 1-5: entire eluted RNA sample amplified. Rounds 6-11: pooled 2 mM and 10 mM RNA, amplified for subsequent round. Rounds 9-11: 1.5 mM RNA amplified separately.

TABLE 2

Wheat Germ Agglutinin 2′NH₂ RNA Ligands

| | | |
|---|---|---|
| | SEQ | |
| | ID | |
| Ligand | NO. | SEQUENCE |

FAMILY 1

| | | |
|---|---|---|
| 11.8 | 4 | AUGGUUGGCCUGGGCGCAGGCUUCGAAGACUCGGCGGGAA CGGGAAUGgcuccgcc |
| 11.4 | 5 | CAGGCACUG AAAACUCGGCGGGAA CG AAAG UAGUGCCGACUCAGACGCGU |
| (3) | | |
| 11.10 | 6 | AGUCUGGCCAAAGACUCGGCGGGAA CGUAAAACGGCCAGAAUU |
| 11.35 | 7 | GUAGGAGGUUCCAUCACC AGGACUCGGCGGGAA CG GAA GGUGAUGS |
| 11.5 | 8 | ACAAGGAUCGAUGGCGAGCCGGGGAGG GCUCGGCGGGAA CG AAA UCUgcuccgcc |
| 11.26 | 9 | UUGGGCAGGCAGAGCGAGACCGGGGGCUCGGCGGGAA CG GAACAGGAAUcgcuccgcc |
| 11.19 | 10 | AAGGGAUGGGAUUGGGACGAGCGGCC AAGACUCGGCGGGAA CG AAG GGUcgcuccgcc |
| 11.15 | 11 | aaucauacac aagaCUCGGCGGGAA CG AAA GUGUCAUGGUAGCAAGUCCAAUGGUGGACUCUc |
| 11.34 | 12 | aaucauacac aagaCUCGGCGGGAA CGUGAA GUGGGUAGGUAGCUGAAGACGGUCUGGGCGCCA |
| 6.8 | 13 | AAGGGAUGGGAUUGGCACGAGCGGCC AAGACUCGGCGGGAA CG AAG GGUCCgcuccgcc |
| 6.9 | 14 | aaucauacaca agaCUCGGCGGGAA CG AAG UGUGUGAGUAACGAUCACUUGGUACUAAAAGCCC |
| 6.23 | 15 | aaucauacac aagaCUCGGCGGGAAUCG AAA GUGUACUGAAUUAGAACGGUGGGCCUGCUCAUCGU |
| 6.26 | 16 | aaucauacaca agaCUCGGCGGGAAUCGUAA UGUGGAUGAUAGCACGAUGGCAGYAGUAGUCGGACCGC |
| 6.14 | 17 | aaucauacacaagaCAGCGGCGG AGUC A GUGAAAGCGUGGGGGGYGCGGGAGGUCUACCCUGAC |
| CON- | 56 | AAGACUCGGCGGGAA CG AAA |
| SEN- | | |
| SUS: | | |

FAMILY 2

| | | |
|---|---|---|
| 11.12 | 18 | CGGCUGUGUGUGGU AGCGUCAUAGUAGGAGUCGUCACGAACCAA GGCgcuccgcc |
| 11.24 | 19 | CGGCUGU GUGGUGUUGGAGCGUCAUAGUAGGAGUCGUCACGAACCAA GGCgcuccgcc |
| (2) | | |
| 11.27 | 20 | CGAUGCGAGGCAAGAA AUGGAGUCGUUACGAACCC UCUUGCAGUGCGCGc |
| (2) | | |
| 11.32 | 21 | CGUGCGGAGCAAAUAGGGGAUC AUGGAGUCGU ACGAACCGUUAUCGCcgcuccgcc |
| 11.6 | 22 | CUGGGGAGCAGGAUAUGAGAUGUGCGGGGCA AUGGAGUCGUGACGAACC gcuccgcc |
| CON- | 57 | GGAGUCGUGACGAACC |
| SEN- | | |
| SUS: | | |

FAMILY 3

| | | |
|---|---|---|
| 11.13 | 23 | GUCCGCCCCCAGGGAUGCAACGGGGUGGCUCUAAAAGGCUUGGCUAA |
| 11.23 | 24 | GAGAAUGAGCAUGGCCGGGGCAGGAAGUGGGUGGCAACGGAGGCCA |
| 6.3 | 25 | GAUACAGCGCGGGUCUAAAGACCUUGCCCCUAGG AUGCAACGGGGUGCGUCCGCC |
| 6.7 | 26 | UGAAGGGUGGUAAGAGAGAGUCUGAGCUCGUCCUAGGGAUGCAACGGCACGUCCGCC |
| 6.20 | 27 | CAAACCUGCAGUCGCGCGGUGAAACCUAGGGUUGCAACGGUACAUCGCUGUCGUCCGCC |
| 6.34 | 28 | GUGGACUGGAAUCUUCGAGGACAGGAACGUUCCUAGGGAUGCAACGGACCGUCCGCC |
| 6.35 | 29 | GUGUACCAAUGGAGGCAAUGCUGCGGGAAUGGAGGCCUAGGGAUGCAAC |
| 6.5 | 30 | GUCCCUAGGGAUGCAACGGGCAGCAUUCGCAUAGGAGUAAUCGGAGGUC |
| 6.16 | 31 | GCCUAGGGAUGCAACGGCGAAUGGAUAGCGAUGUCGUGGACAGCCAGGU |
| 6.19 | 32 | AUCGAACCUAGGGAUGCAACGGUGAAGGUUGUGAGGAUUCGCCAUUAGGC |
| 6.21 | 33 | GCUAGGGAUGCCGCAGAAUGGUCGCGGAUGUAAUAGGUGAAGAUUGUUGC |
| 6.25 | 34 | GGACCUAGGGAUGCAACGGUCCGACCUUGAUGCGCGGGUGUCCAAGCUAC |
| 6.33 | 35 | AAGGGAGGAGCUAGAGAGGGAAAGGUUACUACGCGCCAGAAUAGGAUGU |
| CON- | 58 | CCUAGGGAUCCAACGG |
| SEN- | | |
| SUS: | | |

FAMILY 4

| | | |
|---|---|---|
| 11.2 | 36 | CCAACGUA CAUCGCGAGCUGGUG GAGAGUUCAUGA GGGUGUUACGGGGU |
| 11.33 | 37 | CCCAACGUGUCAUCGCGAGCUGGCG GAGAGUUCAUGA GGGU UACGGGU |
| 11.28 | 38 | GUUGGUGCGAGCUGGGGCGGCGA GAAGGUAGGCGGUCCGAGUGUU CGAAU |
| 11.7 | 39 | aCUGGCAAGRAGUGCGUGAGGGUACGUUAG GGGUGUU UGGGCCGAUCGCAU |
| (4) | | |
| CON- | 59 | RCUGG GAGRGU GGGUGUU |
| SEN- | | |
| SUS: | | |

FAMILY 5

| | | |
|---|---|---|
| 11.20 | 40 | UUGGUCGUACUGGACAGAGCCGUGGUAGAGGGAUUGGGACAAAGUGUCA |
| (5) | | |

FAMILY 6

| | | |
|---|---|---|
| 6.15 | 41 | UGUGAGAAAGUGGCCAACUUUAGGACGUCGGUGGACUGYGCGGGUAGGCUC |
| 6.28 | 42 | CAGGCAGAUGUGUCUGAGUUCGUCGGAGUA GACGUCGGUGGAC GCGGAAC |
| CON- | 60 | UGUGNNNNAGUNNNNNNNNNUA GACGUCGGUGGACNNNGCGG |
| SEN- | | |
| SUS: | | |

FAMILY 7

| | | |
|---|---|---|
| 6.24 | 43 | UGUGAUUAGGCAGUUGCAGCCGCC GU GCGGAGACGU GA CUCGAG GAUUC |
| 6.27 | 44 | UGCCGGUGGAAAGGCGGGUAGGU GA CCCGAG GAUUCCUACCAAGCCAU |
| 11.3 | 45 | GAGGUGRA UGGGAGAGUGGAGCCCGGGUGACUCGAGGAUUCCCGU |
| CON- | 61 | GGGNNNGU GA CYCGRG GAYUC |
| SEN- | | |
| SUS: | | |

FAMILY 8

| | | |
|---|---|---|
| 6.2 | 46 | GUCAUGCUGUGGCUGAACAUACUGGUGAAAGUUCAGUAGGGUGGAUACAgcuccgcc |
| 6.6 | 47 | CCGGGGAUGGUGAGUCGGGCAGUGUGACCGAACUGGUGCCCGCUCACAgcucc |
| (2) | | |
| CON- | 62 | UGANCNNACUGGUGNNNGNGNAG |
| SEN- | | |
| SUS: | | |

FAMILY 9

| | | |
|---|---|---|
| 6.11 | 48 | ACACUAACCAGGUCUCU GAACGCGGGAC GGAGGUG UGGGCGAGGUGGAA |
| 6.13 | 49 | CCGUCUCCCGAGAACCAGGCAGAGGACGUGCUGAAGGAGCUG CAUCUAGAA |
| 6.17 | 50 | CCGUCUCC GAGAACCAGGCAGAGGAGGUGCUGAAGGRGCUGGCAUCUACAA |
| CON- | 63 | GUCUCY GAACNNGGNA GGANGUGNUG GAGNUG |
| SEN- | | |
| SUS: | | |

ORPHANS

| | | |
|---|---|---|
| 6.1 | 51 | CCCGCACAUAAUGUAGGGAACAAUGUUAUGGCGGAAUUGAUAACCGGU |
| 6.4 | 52 | CGAUGUUAGCGCCUCCGGGAGAGGUUAGGGUCGUGCGGNAAGAGUGAGGU |
| 6.18 | 53 | GGUACGGGCGAGACGAGAUGGACUUAUAGGUCGAUGAACGGGUAGCAGCUC |
| 11.30 | 54 | CGGUUGCUGAACAGAACGUGAGUCUUGGUGAGUCGCACAGAUUGUCCU |
| 11.29 | 55 | ACUGAGUAAGGUCUGGCGUGGCAUUAGGUUAGUGGGAGGCUUGGAGUAGc |

TABLE 3
Dissociation Constants of RNA Ligands to WGA

| | | |
|---|---|---|
| Ligand | SEQ ID NO: | Kd |

Family 1

| | | | |
|---|---|---|---|
| 11.8 | 4 | 9.2 | nM |
| 11.4 | 5 | 32 | nM |
| 11.35 | 7 | 90 | nM |
| 11.5 | 8 | 44 | nM |
| 11.26 | 9 | 38 | nM |
| 11.19 | 10 | 22 | nM |
| 11.15 | 11 | 54 | nM |
| 11.34 | 12 | 92 | nM |
| 6.8 | 13 | 11 | nM |
| 6.9 | 14 | 396 | nM |
| 6.23 | 15 | 824 | nM |

| | | |
|---|---|---|
| 6.14 | 17 | <5% |

Family 2

| | | | |
|---|---|---|---|
| 11.12 | 18 | 15.2 | nM |
| 11.24 | 19 | 19.4 | nM |
| 11.27 | 20 | 30 | nM |
| 11.32 | 21 | 274 | nM |
| 11.6 | 22 | 702 | nM |

Family 3

| 11.13 | 23 | <5% | |
|---|---|---|---|
| 11.23 | 24 | <5% | |
| 6.3 | 25 | 120 | nM |
| 6.2 | 27 | <5% | |
| 6.34 | 28 | <5% | |
| 6.35 | 29 | <5% | |
| 6.5 | 30 | 678 | nM |
| 6.16 | 31 | <5% | |
| 6.19 | 32 | 74 | nM |

Family 4

| 11.2 | 36 | 62 | nM |
|---|---|---|---|
| 11.33 | 37 | <5% | |
| 11.28 | 38 | 9.2 | nM |
| 11.7 | 39 | 16 | nM |

Family 5

| | | | |
|---|---|---|---|
| 11.2 | 40 | 1.4 | nM |

Family 7

| | | | |
|---|---|---|---|
| 6.27 | 44 | 56 | nM |
| 11.3 | 45 | 410 | nM |

Family 8

| | | |
|---|---|---|
| 6.6 | 47 | <5% |

Family 9

| | | |
|---|---|---|
| 6.11 | 48 | <5% |

Orphans

| | | | |
|---|---|---|---|
| 11.3 | 54 | 56 | nM |
| 11.29 | 55 | 32 | nM |

The Kds of ligands that show <5% binding at 1 μM WGA is estimated to be >20 μM.

TABLE 4
Specificity of RNA Ligands to WGA

| | Kds for N-acetyl-glucosamine | | |
|---|---|---|---|
| | Binding Lectins | | |
| | Ligand 6.8 | Ligand 11.20 | Ligand 11.24 |
| | (SEQ ID | (SEQ ID | (SEQ ID |
| LECTIN | NO: 13) | NO: 40) | NO: 19) |
| Triticum vulgare (WGA) | 11.4 nM | 1.4 nM | 19.2 nM |
| Canavalia ensiformis (Con A)** | <5%* | <5%* | <5%* |
| Datura stramonium | <5%* | 11.2 μM | <5%* |
| Ulex europaeus (UEA-II) | 4.4 μM | 2.2 μM | <5%* |

*Less than 5% binding at 1 μM protein; estimated Kd >20 μM
**succinylated Con A

TABLE 5
INHIBITION OF RNA LIGAND BINDING
TO WHEAT GERM AGGULTININ

| Ligand | SEQ ID NO: | Competitor | IC₅₀ (μM) | Max Inhib | K_{c} (μM) |
|---|---|---|---|---|---|
| 6.8 | 13 | (GlcNAc)₃ | 95 | >95% | 10.9 |
| 11.20 | 40 | (GlcNAc)₃ | 120 | >95% | 8.4 |
| 11.24 | 19 | (GlcNAc)₃ | 120 | >95% | 19.4 |

K_{c} is the dissociation constant of (GlcNAc)₃ calculated from these data, assuming competitive inhibition and two RNA ligand binding sites per dimer.

TABLE 6
INHIBITION OF WGA MEDIATED AGGLUTINATION
OF SHEEP ERYTHROCYTES

| | Inhibitory Concentration (μM) | | |
|---|---|---|---|
| Inhibitor | SEQ ID NO: | Complete | Partial |
| 6.8 | 13 | 0.5 | 0.12 |
| 11.20 | 40 | 0.5 | 0.12 |
| 11.24 | 19 | * | 2 |
| (GlcNAc)₃ | | 8 | 2 |
| GlcNAc | | 780 | 200 |

* Complete inhibition of agglutination by ligand 11.24 was not observed in this experiment.

TABLE 7a
L-Selectin 2′NH₂-RNA SELEX at 4° C.

| | | | | | | % 5 mM | % 50 mM | |
|---|---|---|---|---|---|---|---|---|
| | Total | Total | | | | EDTA | EDTA | |
| SELEX | RNA | Protein | RNA:LS- | Bead | Total | Eluted | Eluted | |
| Round # | pmoles | pmoles | Rg Ratio | Volume | Volume | RNA | RNA | Kd (nM) |
| Rnd 0 | | | | | | | | 10,000 |
| Rnd 1 | 1060 | 167.0 | 6.3 | 10 μL | ~100 μL | 0.498 | 0.301 | |
| Rnd 2 | 962 | 167.0 | 5.8 | 10 μL | ~100 μL | 0.306 | 0.114 | |
| Rnd 3 | 509 | 167.0 | 3.0 | 10 μL | ~100 μL | 1.480 | 0.713 | |
| Rnd 4 | 407 | 167.0 | 2.4 | 10 μL | ~100 μL | 5.010 | 1.596 | 434 |
| Rnd 5 | 429 | 167.0 | 2.6 | 10 μL | ~100 μL | 8.357 | 7.047 | |
| | 439 | 16.7 | 26.3 | 10 μL | ~100 μL | 0.984 | 0.492 | 133 |
| Rnd 6 | 452 | 167.0 | 2.7 | 10 μL | ~100 μL | 7.409 | 6.579 | |
| | 46 | 16.7 | 2.8 | 10 μL | ~100 μL | 3.468 | 1.312 | 37 |
| Rnd 7 | 43 | 16.7 | 2.6 | 10 μL | ~100 μL | 8.679 | 2.430 | |
| | 44 | 16.7 | 2.6 | 10 μL | ~100 μL | 7.539 | 2.358 | |
| | 22 | 4.2 | 5.2 | 10 μL | ~100 μL | 2.748 | 1.298 | |
| Rnd 8 | 43 | 16.7 | 2.6 | 10 μL | ~100 μL | 8.139 | 1.393 | 33 |
| | 23 | 4.2 | 5.5 | 10 μL | ~100 μL | 2.754 | 0.516 | |
| Rnd 9 | 23 | 4.2 | 5.5 | 10 μL | ~100 μL | 4.352 | 0.761 | |
| Rnd 10 | 21 | 4.2 | 5.0 | 10 μL | ~100 μL | 6.820 | 1.123 | 13 |
| | 23 | 8.4 | 2.7 | 50 μL | ~150 μL | 14.756 | 1.934 | |
| Rnd 11 | 30 | 10.5 | 2.9 | 250 μL | ~500 μL | 0.707 | 0.033 | |
| Rnd 12 | 12 | 10.5 | 1.1 | 250 μL | ~500 μL | 3.283 | 0.137 | |
| Rnd 13 | 7 | 1 | 7 | 250 μL | ~500 μL | 4.188 | 0.136 | 0.3 |
| Rnd 14 | 9 | 1 | 9 | 250 μL | ~500 μL | 4.817 | 0.438 | 0.7 |

L-Selectin Rg was immobilized on Protein A Sepharose 4 Fast Flow. Protein A density is approximately 6 mg/ml drained gel (143 μM).
RNA Loading Conditions: All selections were carried out in the cold room. The RNA used in each selection was first incubated for 30 minutes with 100 μL Protein A Sepharose in the cold room on a roller. Only RNA which flowed through this column was used on the LS-Rg selection column. The RNA was incubated on the selection column for 90 minutes on a roller before being washed extensively with binding buffer (20 mM HEPES pH7.4 150 mM NaCl, 1 mM MgCl₂, 1 mM CaCl₂.)
RNA Elution Conditions: RNA was eluted by incubating the extensively-washed columns in 100 μL of HEPES buffered EDTA (pH7.4) for 30 minutes on a roller followed by three 100 μL HEPES buffered EDTA washes.

TABLE 7b
L-Selectin 2′NH₂-RNA SELEX at Room Temperature

| | | | | | | % 5 mM | % 50 mM | |
|---|---|---|---|---|---|---|---|---|
| | Total | Total | | | | EDTA | EDTA | |
| SELEX | RNA | Protein | RNA:LS- | Bead | Total | Eluted | Eluted | |
| Round # | pmoles | pmoles | Rg Ratio | Volume | Volume | RNA | RNA | Kd (nM) |
| Rnd 7 | 43 | 10.0 | 4.3 | 10 μL | ~100 μL | 1.205 | 0.463 | |
| Rnd 8 | 35 | 10 | 3.5 | 10 μL | ~100 μL | 6.642 | 0.401 | |
| | 35 | 10 | 3.5 | 10 μL | ~100 μL | 5.540 | 0.391 | |
| Rnd 9 | 24 | 2.5 | 9.6 | 10 μL | ~100 μL | 1.473 | 0.383 | 13 |
| Rnd 10 | 30 | 6.3 | 4.9 | 250 μL | ~500 μL | 0.707 | 0.033 | |
| Rnd 11 | 12 | 6.3 | 1.9 | 250 μL | ~500 μL | 3.283 | 0.134 | |
| Rnd 12 | 6 | 0.6 | 9.4 | 250 μL | ~500 μL | 0.877 | 0.109 | 0.3 |
| Rnd 13 | 1 | 0.6 | 1.4 | 250 μL | ~500 μL | 5.496 | 0.739 | 0.7 |

L-Selectin Rg was immobilized on Protein A Sepharose 4 Fast Flow. Protein A density is approximately 6 mg/ml drained gel (143 μM).
RNA Loading Conditions: Selections were carried out at room temperature. The RNA used in each selection was first incubated for 30 minutes with 100 μL Protein A Sepharose at room temp. Only RNA which flowed through this column was used on the LS-Rg selection column. The RNA was incubated on the selection column for 90 minutes on a roller before being washed extensively with binding buffer (20 mM HEPES pH7.4 150 mM NaCl, 1 mM MgCl₂, 1 mM CaCl₂.)
RNA Elution Conditions: RNA was eluted by incubating the extensively-washed columns in 100 μL of HEPES buffered EDTA (pH7.4) for 30 minutes on a roller followed by three 100 μL HEPES buffered EDTA washes.

TABLE 8

L-Selectin 2′NH₂ RNA LIGANDS

| | | |
|---|---|---|
| Ligand | SEQ ID NO. | Sequences |

Family I

| | | |
|---|---|---|
| F13.32(5) | 67 | CGCGUAUGUGUGAAAGCGUGUGCACGGAGGCGU-CUACAAU |
| 6.60(2) | 68 | GGCAUUGUGUGAAUAGCUGAUCCCACAGGUAACAACAGCA |
| 6.50(3) | 69 | UAAUGUGUGAAUCAAGCAGUCUGAAUAGAUUAGACAAAAU |
| 6.79 | 70 | AUGUGUGAGUAGCUGAGCGCCCGAGUAUGAWACCUGACUA |
| F14.9 | 71 | AAACCUUGAUGUGUGAUAGAGCAUCCCCCAGGCGACGUAC |
| F14.21 | 72 | UUGAGAUGUGUGAGUACAAGCUCAAAAUCCCGUUGGAGG |
| F14.25 | 73 | UAGAGGUAGUAUGUGUGGGAGAUGAAAAUACUGUGGAAAG |
| F13.48(2) | 74 | AAAGUUAUCAGUCCGUAUAUCAAGGUCGACAUGUGUGAAU |
| 6.71 | 75 | CACGAAAAACCCGAAUUGGGUCGCCCAUAAGGAUGUGUGA |
| 6.28 | 76 | GUAAAGAGAUCCUAAUGGCUCGCUAGAUGUGAUGUGAAAC |
| CONSENSUS: | 118 | AUGUGUGA |

Family II

| | | |
|---|---|---|
| F14.20(26) | 77 | UAACAA CAAUCAAGGCGGGUUCACCGCCCCAGUAUGAGUG |
| F14.12(22) | 78 | UAACAA CAAUCAAGGCGGGUUYACCGCCCCAGUAUGAGUA |
| F14.11(12) | 79 | UAACAA CAAUCAAGGCGGGUUYACCGCUCCAGUAUGAGUA |
| F13.45(9) | 80 | UAACAA CAAUCAAGGCGGGUUCACCGCCCCAGUAUGAGUG |
| 6.80 | 81 | ACCAAGCAAUCUAU GGUCGAACGCUACA CAUGAAUGACGUc |
| CONSENSUS: | 119 | CAA CAAUC AUGAGUR |

Family III

| | | |
|---|---|---|
| 6.17 | 82 | GAACAUGAAGUAAUCAAAGUCGUACC AAUAUACAGGAAGC |
| 6.49 | 83 | GAACAUGAAGUAAGAC CGUCAC AAUUCGAAUGAUUGAAUA |
| 6.16 | 84 | GAACAUGAAGUAAAA AGUCGACG AAUUAGCUGUAACCAAAA |
| 6.37 | 85 | GAACAUGAAGUAAA AGUCUG AGUUAGUAAAUUACAGUGAU |
| 6.78 | 86 | GAACUUGAAGUUGA ANUCGCUAA GGUUAUGGAUUCAAGAUU |
| 6.26 | 87 | AACAUGAAGUAAUA AGUC GACGUAAUUAGCUGUAACUAAA |
| 6.40 | 88 | AACAUGAAGUAAA AGUCUG AGUUAGAAAUUACAAGUGAU- |
| F13.57 | 89 | UAACAUAAAGUAGCG CGUCUGUGAGAGGAAGUGCCUGGAU |
| CONSENSUS: | 120 | AACAUGAAGUA AGUC ARUUAG |

Family IV

| | | |
|---|---|---|
| 6.58 | 90 | AUAGAACCGCAAGGAUAACCUCGACCGUGGUCAACUGAGA |
| 6.69 | 91 | UAAGAACCGCUAGCGCACGAUCAAACAAAGAGAAACAAA- |
| CONSENSUS: | 121 | AGAACCGCWAG |

Family V

| | | |
|---|---|---|
| 6.59 | 92 | UUCUCUCCAAGAACYGAGCGAAUAAACSACCGGASUCACA |
| F13.55 | 93 | UGUCUCUCCUGACUUUUAUUCUUAGUUCGAGCUGUCCUGG |
| CONSENSUS: | 122 | UCUCUCC |

Family VI

| | | |
|---|---|---|
| F14.27 | 94 | CCGUACAUGGUAARCCU CGAAGGAUUCCCGGGAUGAUCCC |
| F14.53 | 95 | UCCCAGAGUCCCGUGAUGCGAAGAAUCCAUUAGUACCAGA |
| CONSENSUS: | 123 | CGAAGAAUYC |

Family VII

| | | |
|---|---|---|
| F13.42 | 96 | GAUGUAAAUGACAAAUGAACCUCGAAAGAUUGCACACUC |
| F13.51 | 97 | AUGUAAAUCUAGGCAGAAACGUAGGGCAUCCACCGCAACGA |
| CONSENSUS: | 124 | AUGUAAAU |

Family VIII

| | | |
|---|---|---|
| 6.33(11) | 98 | AUAACCCAAGCAGCNUCGAGAAAGAGCUCCAUAGAUGAU- |
| 6.41 | 99 | CAAAGCACGCGUAUGGCAUGAAACUGGCANCCCAAGUAAG |
| CONSENSUS: | 125 | AACCCAAG |

Family IX

| | | |
|---|---|---|
| F13.46(4) | 100 | CAAAAGGUUGACGUAGCGAAGCUCUCAAAAUGGUCAUGAC |

Family X

| | | |
|---|---|---|
| F14.2 | 101 | AAGUGAAGCUAAAGCGGAGGG CCAUUCAGUUUCNCACCA |
| F14.13(2) | 102 | AAGUGAAGCUAAAGSGGAGGG CCACUCAGAAACGCACCA |

Family XI

| | | |
|---|---|---|
| 6.72(2) | 103 | CACCGCUAAGCAGUGGCAUAGCCCAGUAACCUGUAAGAGA |
| 6.42 | 104 | CAC-GCUAAGCAGUGGCAUAGC---GWAACCUGUAAGAGA |

Family XII

| | | |
|---|---|---|
| 6.30(5) | 105 | AGAUUACCAUAACCGCGUAGUCGAAGACAUAUAGUAGCGA |

Family XIII

| | | |
|---|---|---|
| 6.52(2) | 106 | ACUCGGGUAGAACGCGACUUGCCACCACUCCCAUAAAGAC |

Orphans

| | | |
|---|---|---|
| 6.14 | 107 | UCAGAACUCUGCCGCUGUAGACAAAGAGGAGCUUAGCGAA |
| 6.36 | 108 | AAUGAGCAUCGAGAGAGCGCGAACUCAUCGAGCGUACUAA |
| 6.41 | 119 | CAAAGCACGCGUAUGGCAUGAAACUGGCANCCCAAGUAAG |
| 6.44 | 110 | GAUGCAGCAACCUGAAAACGGCGUCCACAGGUAAUAACAG |
| 6.70 | 111 | AAACUCGCUACAAACACCCAAUCCUAGAACGUUAUGGAGA |
| 6.76 | 112 | CUAGCAUAGCCACCGGAACAGACAGAUACGAGCACGAUCA |
| 6.89 | 113 | GAUUCGGAGUACUGAAAAACAACCCUCAAAAGUGCAUAGG |
| 6.81 | 114 | GUCCAGGACGGACCGCAGCUGUGAUACAAUCGACUUACAC |
| 6.70 | 115 | AAACUCGCUACAAACACCCAAUCCUAGAACGUUAUGGAGA |
| F13.59 | 116 | CGGCCCUUAUCGGAGGUCUGCGCCACUAAUUACAUCCAC |
| F14.70 | 117 | UCCAGAGCGUGAAGAUCAACGUCCCGGNGUCGAAGA |

TABLE 9
Dissociation Constants of 2′ NH₂ RNA
Ligands to L-Selectin*

| | | | |
|---|---|---|---|
| Ligand | SEQ ID NO: | 4° C. | Rm Temp |

Family I

| | | | | | |
|---|---|---|---|---|---|
| F13.32 | 67 | 15.7 | nM | 14.9 | nM |
| F13.48 | 74 | 15.9 | nM | 9.2 | nM |
| F14.9 | 71 | 8.2 | nM | 15.4 | nM |
| F14.21 | 72 | 2.3 | nM | 15.9 | nM |
| F14.25 | 73 | 1300 | nM | | |

Family II

| F14.12 | 78 | 5.8 | pM | 1.7 | nM |
|---|---|---|---|---|---|
| | | (0.68) | | (0.62) | |
| | | 16.2 | nM | 94 | nM |
| F14.20 | 77 | 58 | pM | 1.0 | nM |

| (0.68) | | (0.28) | |
|---|---|---|---|
| 60 | nM | 48 | nM |

Family III

| | | | | | |
|---|---|---|---|---|---|
| F13.57 | 89 | 3.0 | nM | 75 | nM |

Family V

| | | | | | |
|---|---|---|---|---|---|
| F13.55 | 93 | 62 | pM | 1.5 | nM |

Family VI

| | | | | | |
|---|---|---|---|---|---|
| F14.53 | 95 | 97 | pM | 142 | nM |

(0.65)

| | | 14.5 | nM |
|---|---|---|---|
| F14.27 | 94 | 145 | nM |

Family VII

| | | | | | |
|---|---|---|---|---|---|
| F13.42 | 96 | 2.0 | nM | 5.5 | nM |
| F13.51 | 97 | 8.8 | nM | 18 | nM |

Family X

| | | | | | |
|---|---|---|---|---|---|
| F14.2 | 101 | 1.8 | nM | 7.2 | nM |
| F14.13 | 102 | 1.3 | nM | | |

(0.74)

| | |
|---|---|
| 270 | nM |

Orphans

| F13.59 | 116 | <5% | | <5% | |
|---|---|---|---|---|---|
| F14.70 | 117 | 2.0 | nM | 7.8 | nM |

| (0.75) | | (0.58) | |
|---|---|---|---|
| 254 | nM | 265 | nM |

*Kds of monophasic binding ligands are indicated by a single number; the high affinity K_{d} (ie., K_{d1}), the mole fraction binding with K_{d1}, and the low affinity K_{d} (ie., K_{d2}) are presented for biphasic binding ligands.

TABLE 10
Specificity of 2′ NH₂ RNA Ligands to L-Selectin*

| | | | | | |
|---|---|---|---|---|---|
| Ligand | SEQ ID NO: | LS-Rg | ES-Rg | PS-Rg | CD22-Rg |

Family I

| | | | | | |
|---|---|---|---|---|---|
| F13.32 | 67 | 15.7 nM | <5% | 17 μM | <5% |
| F13.48 | 74 | 15.9 nM | <5% | 720 nM | <5% |
| F14.9 | 71 | 8.2 nM | <5% | | <5% |
| F14.21 | 72 | 2.3 nM | 2.6 μM | | <5% |
| F14.25 | 73 | 1300 nM | | | |

Family II

| | | | | | |
|---|---|---|---|---|---|
| F14.12 | 78 | 60 pM | 47 nM | 910 nM | <5% |
| F14.20 | 77 | 58 pM | 70 nM | | <5% |
| | | (0.68) | | | |
| | | 60 nM | | | |

Family III

| | | | | |
|---|---|---|---|---|
| F13.57 | 89 | 3.0 nM | 2.7 μM | <5% |

Family V

| | | | | | |
|---|---|---|---|---|---|
| F13.55 | 93 | 62 pM | 49 nM | 5.8 μM | <5% |

Family VI

| | | | | | |
|---|---|---|---|---|---|
| F14.53 | 95 | 97 pM | 355 nM | 5.2 μM | <5% |
| | | (0.65) | | | |
| | | 14.5 nM | | | |

Family VII

| | | | | |
|---|---|---|---|---|
| F13.42 | 96 | 2.0 nM | 4.4 μM | <5% |
| F13.51 | 97 | 8.8 nM | 2.0 μM | |

Family X

| | | | | | |
|---|---|---|---|---|---|
| F14.2 | 101 | 1.8 nM | 1.9 μM | 450 nM | <5% |

Orphans

| | | | | |
|---|---|---|---|---|
| F13.59 | 116 | <5% | <5% | <5% |
| F14.70 | 117 | 2.0 nM | 5.9 μM | <5% |
| | | (0.75) | | |
| | | 254 nM | | |

*Dissociation constants were determined at 4° C. in HSMC buffer. When <5% binding was observed at the highest protein concentration, the Kd is estimated to be >20 μM.

TABLE 11
L-SELECTIN ssDNA SELEX

| | | Total | Total | | | | | | | |
|---|---|---|---|---|---|---|---|---|---|---|
| | | DNA | Prot. | | | | % Eluted | % Eluted | Kd, nM | signal:bkgd |
| Round | Temp. | pmol | pmol | DNA:Protein | Bead Vol. | Total Vol. | 2 mM EDTA | 50 mM EDTA | 4 degrees | 2 mM |
| Rnd 0 | | | | | | | | | 10,000 | |
| Rnd 1 | 4 | 930 | 167 | 5.6 | 10 μL | ~100 μL | n/a | 5.5 | | 50 |
| Rnd 2 | 25 | 400 | 167 | 2.4 | 10 μL | ~100 μL | n/a | 2.19 | | 12 |
| Rnd 3 | 25 | 460 | 167 | 2.8 | 10 μL | ~100 μL | n/a | 2.55 | | 25 |
| Rnd 4 | 25 | 100 | 16.7 | 6 | 10 μL | ~100 μL | 0.35 | 0.29 | | 1.3 |
| Rnd 5 | 25 | 100 | 16.7 | 6 | 10 μL | ~100 μL | 0.23 | 0.08 | 967 | 3 |
| Rnd 6 | 25 | 1000 | 16.7 | 60 | 10 μL | ~100 μL | 1.42 | 0.38 | | 4 |
| Rnd 7 | 25 | 100 | 16.7 | 6 | 10 μL | ~100 μL | 6.9 | 0.93 | 60 | 18 |
| Rnd 8 | 37 | 100 | 16.7 | 6 | 10 μL | ~100 μL | 1.9 | 0.31 | | 9 |
| Rnd 9 | 25 | 10 | 1.67 | 6 | 10 μL | ~100 μL | 0.5 | 0.16 | 2.1 | 1.6 |
| Rnd10 | 25 | 10 | 1.67 | 6 | 10 μL | ~100 μL | 2.2 | 0.57 | | 5 |
| Rnd 11 | 25 | 2.5 | 0.42 | 6 | 10 μL | ~100 μL | 0.37 | 0.07 | 1.3 @ 25° C. | 8 |
| Rnd 12 | 25 | 2.5 | 0.42 | 6 | 10 μL | ~100 μL | 0.86 | 0.13 | | 11 |
| Rnd 13 | 37 | 2.5 | 0.42 | 6 | 10 μL | ~100 μL | 0.7 | 0.35 | 0.44 @ 25° C. | 5 |
| Rnd 14 | 25 | 5 | 0.84 | 6 | 50 μL | ~100 μL | 2.8 | 0.76 | | 4 |
| Rnd 15 | 25 | 1.25 | 0.21 | 6 | 50 μL | ~100 μL | 1.7 | 0.5 | 0.16 @ 25° C. | 7 |

Binding Buffer, Rounds 1-9 10 mM HEPES, pH at room temp w/NaOH to 7.4 100 mM NaCl 1 mM MgCl2 1 mM CaCl2 5 mM KCl
Elution Buffers: replace divalent cations with EDTA

TABLE 12

L-Selectin ssDNA Ligands

| | | |
|---|---|---|
| Ligand | SEQ ID NO | SEQUENCE |

Family 1

| | | |
|---|---|---|
| LD204(3) | 129 | GGAACACGTGAGGTTTAC AAGGCACTCGAC GTAAACACTT |
| LD145 | 130 | CCCCGAAGAACATTTTAC AAGGTGCTAAAC GTAAAATCAG |
| LD183(2) | 131 | GGCATCCCTGAGTCATTAC AAGGTTCTTAAC GTAATGTAC |
| LD230(2) | 132 | TGCACACCTGAGGGTTAC AAGGCGCTAGAC GTAACCTCTC |
| LD208(7) | 133 | CACGTTTC AAGGGGTTACAC GAAACGATTCACTCCTTGGC |
| LD227(5) | 134 | CGGACATGAGCGTTAC AAGGTGCTAAAC GTAACGTACTT |
| LD112 | 135 | CGCATCCACATAGTTC AAGGGGCTACAC GAAATATTGCA |
| LD137 | 136 | TACCCCTTGgGCCTCATAGAC AAGGTCTTAAAC GTTAGC |
| LD179(2) | 137 | CACATGCCTGACGCGGTAC AAGGCCTGC AC GTAACGTTG |
| LD182 | 138 | TAGTGCTCCACGTATTC AAGGTGCTAAAC GAAGACGGCCT |
| LD190 | 139 | AGCGATGC AAGGGGCTACAC GCAACGATTTAGATGCTCT |
| LD193(2) | 140 | CCAGGAGCACAGTAC AAGGTGTTAAAC GTAATGTCTGGT |
| LD199 | 141 | ACCACACCTGGGCGGTAC AAGGAGTTATCC GTAACGTGT |
| LD201(2) | 142 | CAAGGTAACCAGTAC AAGGTGCTAAAC GTAATGGCTTCG |
| LD203 | 143 | ACCCCCGACCCGAGTAC AAGGCATTCGAC GTAATCTGGT |
| LD207 | 144 | CAGTAC AAGGTGTTAAAC GTAATGCCGATCGAGTTGTAT |
| LD216 | 145 | ACAACGAGTAC AAGGAGATAGAC GTAATCGGCGCAGGTATC |
| LD233(5) | 146 | CACGACAGAGAAC AAGGCGTTAGAC GTTATCCGACCACG |
| LD191 | 147 | AGGGAGAAC AAGGTGCTAAAC GTTTATCTACACTTCACCT |
| LD128(3) | 148 | AGGACC AAGGTGTTAAAC GGCTCCCCTGGCTATGCCTCTT |
| LD111(2) | 149 | gcTACAC AAGGTGCTAAAC GTAGAGCCAGATCGGATCTGAGC |
| LD139 | 150 | GGAC AAGGCACTCGAC GTAGTTTATAACTCCCTCCGGgCC |
| LD237 | 151 | gcTACAC AAGGGGCCAAAC GGAGAGCCAGACGCGGATCTGACA |
| LD173 | 152 | CGGCTATAC NNGGTGCTAAAC GCAGAGACTCGATCAACA |
| LD209 | 153 | GAGTAGCC AAGGCGTTAGAC GGAGGGGGAATGGAAGCTTG |
| LD221 | 154 | GAGTAGCC AAGGCGTTAGAC GGAGGGGGAATGG |
| LD108 | 155 | GAGTAGCC AAGGCGTTAGAC GGAGGGGGAATGTGAGCACA |
| LD141 | 156 | TAGCTCCACACAC AASSCGCRGCAC ATAGGGGATATCTGG |
| LD539 | 175 | CGGCAGGGCACTAAC AAGGTGTTAAAC GTTACGGATGCC |
| LD547 | 176 | TGCACACCGGCCCACCCGGAC AAGGCGCTAGAC GAAATGACTCTGTTCTG |
| LD516 | 177 | GACGAAGAGGCC AAGGTGATAACC GGAGTTTCCGTCCGC |
| LD543 | 178 | AAGGACTTAGCTATCC AAGGCACTCGAC GAAGAGCCCGA |
| LD545 | 179 | ATGCCCAGTTC AAGGTTCTGACC GAAATGACTCTGTTCTG |
| Truncates | | |
| LD201T1 | 185 | tagcCAAGGTAACCAGTAC AAGGTGCTAAAC GTAATGGCTTCGgcttac |
| LD201T3 | 186 | GTAACCAGTAC AAGGTGCTAAAC GTAATGGCTTCGgcttac |
| LD201T4 | 187 | CCAGTAC AAGGTGCTAAAC GTAATGG |
| LD201T10 | 188 | CGCGGTAACCAGTAC AAGGTGCTAAAC GTAATGGCGCG |
| LD201T12 | 189 | GCGGTAACCAGTAC AAGGTGCTAAAC GTAATGGCGC |
| LD227t5 | 190 | ACATGAGCGTTAC AAGGTGCTAAAC GTAACGTACTTgcttactctcatgt |
| LD227x1 | 191 | cgcGCGTTAC AAGGTGCTAAAC GTAACGTACTTgcttactcgcg |
| LD227t1 | 192 | GCGTTAC AAGGTGCTAAAC GTAACGT |
| NX288 | 193 | dtₐtagcCAAGGTAACCAGTAC AAGGTGCTAAAC GTAATGGCTTCGgcttact[3′3′]t |
| NX303 | 196 | dtₐCCAGTAC AAGGTGCTAAAC GTAATGGt[3′3′]t |
| Consensus: | 181 | TAC AAGGYGYTAVAC GTA |

Family 2

| | | |
|---|---|---|
| LD181(3) | 157 | CAT CAAGGACTTTGCCCGAAACCCTAGGTTCACG TGTGGG |

Family 4

| | | |
|---|---|---|
| LD174(2) | 158 | CATTCACCATGGCCCCTTCCTACGTATGTTCTGCGGGTG |
| LD122 | 159 | GCAACGTGGCCCCGTT TAGCTCATTTGACCGTTCCATCCG |
| LD239 | 160 | CCACAGACAATCGCAGTCCCCGTG TAGCTCTGGGTGTCT |
| LD533 | 180 | GCAGCGTGGCCCTGTT TAGCTCATTTGACCGTTCCATCCG |
| Truncates | | |
| LD174t1 | 194 | tagcCATTCACCATGGCCCCTTCCTACGTATGTTCTGCGGGTGgctta |
| Consensus: | 182 | GGCCCCGT |

Family 5

| | | |
|---|---|---|
| LD109 | 161 | CCACCGTGATGCACGATACATGAGGGTGTGTCAGCGCAT |
| LD127 | 162 | CGAGGTAGTCGTTATAGGGTGCGCACGACACACAGCGGTRG |
| Consensus: | 183 | RCACGAYACA |

Family 6

| | | |
|---|---|---|
| LD196 | 163 | TGGCGGTACGGGCCGTGCACCCACTTACCTGGGAAGTGA |
| LD229 | 164 | CTCTGCTTACCTCATGTAGTTCCAAGCTTGGCGTAATCATG |
| Truncate | | |
| LD196t1 | 195 | agcTGGCGGTACGGGCCGTGCACCCACTTACCTGGGAAGTGAgctta |
| Consensus: | 184 | CTTACCT |

Family 7

| | | |
|---|---|---|
| LD206(2) | 165 | AGCGTTGT ACGGGGTTACAC ACAACGATTTAGATGCTCT |

Orphans

| | | |
|---|---|---|
| LD214 | 166 | TGATGCGACTTTAGTCGAACGTTACTGGGGCTCAGAGGACA |
| LD102 | 167 | CGAGGATCTGATACTTATTGAACATAMCCGCACNCAGGCTT |
| LD530 | 168 | CGATCGTGTGTCATGCTACCTACGATCTGACTA |
| LD504 | 169 | GCACACAAGTCAAGCATGCGACCTTCAACCATCGACCCGA |
| LD509 | 170 | ATGCCAGTGCAGGCTTCCATCCATCAGTCTGACANNNNNN |
| LD523 | 171 | CACTTCGGCTCTACTCCACCTCGGTCCTCCACTCCACAG- |
| LD527 | 172 | CGCTAACTGACCCTCGATCCCCCCAAGCCATCCTCATCGC |
| LD541 | 173 | ATCTGACTAGCTCGGCGAGAGTACCCGCTCATGGCTTCGGCGAATGCCCT |
| LD548 | 174 | TCCTGAGACGTTACAATAGGCTGCGGTACTGCAACGTGGA |

TABLE 13
Dissociation Constants of ssDNA Ligands
to L-Selectin

| | | | |
|---|---|---|---|
| | | Room | |
| Ligand | SEQ ID NO: | Temperature | 37° C. |

Family 1

| | | | | | |
|---|---|---|---|---|---|
| LD111 | 149 | 330 | pM | 11.8 | nM |
| LD128 | 148 | 310 | pM | 1.8 | nM |
| LD108 | 155 | 160 | pM | 8.5 | nM |
| LD112 | 135 | 300 | pM | 23.2 | nM |
| LD137 | 136 | 520 | pM | 0.65 | nM |
| LD139 | 150 | 210 | pM | 6.8 | nM |
| LD145 | 130 | 920 | pM | 8.8 | nM |
| LD179 | 137 | 180 | pM | 590 | pM |
| LD182 | 138 | 130 | pM | 2.0 | nM |
| LD183 | 131 | 170 | pM | 1.0 | nM |
| LD193 | 140 | 88 | pM | 970 | pM |
| LD201 | 142 | 110 | pM | 1.2 | nM |
| LD204 | 129 | 100 | pM | 3.7 | nM |
| LD208 | 155 | 110 | pM | 380 | pM |
| LD227 | 134 | 43 | pM | 160 | pM |
| LD230 | 132 | 57 | pM | 260 | pM |
| LD233 | 146 | 110 | nM | 380 | pM |

Family 2

| | | | | | |
|---|---|---|---|---|---|
| LD181 | 157 | 84 | pM | 1.8 | nM |

Family 4

| | | | | | |
|---|---|---|---|---|---|
| LD122 | 159 | 1.8 | nM | 2.1 | nM |
| LD174 | 158 | 43 | pM | 370 | pM |
| LD239 | 160 | 170 | pM | 1.6 | nM |

Family 5

| | | | | | |
|---|---|---|---|---|---|
| LD109 | 161 | 190 | pM | 9.6 | nM |
| LD127 | 162 | 1.0 | nM | 890 | pM |

Family 6

| | | | | | |
|---|---|---|---|---|---|
| LD196 | 163 | 130 | pM | 3.4 | nM |

Family 7

| | | | | | |
|---|---|---|---|---|---|
| LD206 | 165 | 330 | pM | 6.0 | nM |

Orphans

| LD102 | 167 | not | | 7.9 | nM |
|---|---|---|---|---|---|
| | | determined | | | |
| LD214 | 166 | 660 | pM | 8.4 | nM |
| Round 15 Pool | | 160 | pM | 660 | pM |
| LD201T1* | | | | 4.8 | nM |
| LD201T3* | | | | 43 | nM |

*LD201T1 and LD201T3 were made by solid state synthesis; the Kd of the synthetic full length LD201 control was 3.8 nM while that of enzymatically synthesized LD201 was 1.8 nM.

TABLE 14
Specificities of ssDNA Ligands to L-Selectin*

| | | | | |
|---|---|---|---|---|
| Ligand | SEQ ID NO: | LS-Rg | ES-Rg | PS-Rg |

Family 1

| | | | | | | | |
|---|---|---|---|---|---|---|---|
| LD111 | 149 | 1.1 | nM | 1.2 | μM | 840 | nM |
| LD201 | 142 | 110 | nM | 37 | nM | 1.0 | μM |
| LD204 | 129 | 450 | pM | 1.5 | μM | 2.9 | μM |
| LD227 | 134 | 64 | pM | 33 | nM | 560 | nM |
| LD230 | 132 | 44 | pM | 19 | nM | 600 | nM |
| LD233 | 146 | 120 | pM | 39 | nM | 420 | nM |

Family 2

| | | | | | | | |
|---|---|---|---|---|---|---|---|
| LD181 | 157 | 200 | pM | 37 | nM | 1.6 | μM |

Family 4

| | | | | | | | |
|---|---|---|---|---|---|---|---|
| LD122 | 159 | 340 | pM | 400 | nM | 420 | nM |
| LD174 | 158 | 46 | pM | 28 | nM | 380 | nM |

Family 5

| | | | | | | | |
|---|---|---|---|---|---|---|---|
| LD127 | 162 | 250 | pM | 1.3 | μM | 780 | nM |

Family 6

| | | | | | | | |
|---|---|---|---|---|---|---|---|
| LD196 | 163 | 220 | pM | 50 | nM | 3.4 | μM |

Family 7

| | | | | | | | |
|---|---|---|---|---|---|---|---|
| LD206 | 165 | 120 | pM | 100 | nM | 600 | nM |

*Kds were determined at room temperature. In assays with 700 nM CD22 β-Rg and 1.4 μM WGA less than 1% and 3% binding, respectively, was observed for all ligands suggesting that the dissociation constants are greater than 100 μM for these proteins.

TABLE 15
Summary of Selection Conditions and Results from
2′F RNA Human L-selectin SELEXes

| | | | | | | |
|---|---|---|---|---|---|---|
| | Total | Total | Temp, | % Bound | % 5 mM | |
| SELEX | RNA | Protein | Time, | LS-Rg | EDTA | |
| Round | pmoles | pmoles | Vol. | Sites | Eluted | Kd(nM) |

EDTA
Signal/
Bkgnd

30n7 2′Fluro SELEX

| | | | | | | | |
|---|---|---|---|---|---|---|---|
| 1 | 630 | 100 | 37° C. 15′ 10 μl | 0.7 | 0.1 | 20 | |
| 2 | 656 | 100 | 37° C. 15′ 10 μl | 2.8 | 0.4 | 24 | |
| 3 | 608 | 100 | 37° C. 15′ 10 μl | 11.6 | 1.9 | 68 | 10000 |
| 4 | 193 | 20 | 37° C. 15′ 10 μl | 7.4 | 0.8 | 24 | |
| 5 | 193 | 20 | 37° C. 15′ 10 μl | 19.7 | 2.1 | 17 | 850 |
| 6 | 86 | 10 | 37° C. 15′ 10 μl | 15.7 | 1.9 | 8 | 360 |
| 7 | 17 | 2 | 37° C. 15′ 10 μl | 12.1 | 1.4 | 3 | |
| 8 | 17 | 2 | 37° C. 15′ 10 μl | 55.1 | 6.6 | 2 | |
| 9 | 19 | 2 | 37° C. 15′ 10 μl | 40.1 | 4.2 | 4 | |
| 10 | 18 | 2 | 37° C. 15′ 10 μl | 28.4 | 3.3 | 3 | 3 |
| 11 | 103 | 12.5 | 37° C. 15′ 50 μl | 647.7 | 8.3 | 65 | |
| 11 | 27 | 2.5 | 37° C. 15′ 50 μl | 63.1 | 5.9 | 3 | 0.5 |
| 12 | 89 | 5 | 37° C. 15′ 50 μl | 53.2 | 3.0 | 7 | |
| 12 | 79 | 5 | 37° C. 15′ 50 μl | 54.8 | 3.5 | 65 | 0.4 |

Signal/
Bkgnd
EDTA

40n7 2′Fluro SELEX

| | | | | | | | |
|---|---|---|---|---|---|---|---|
| 1 | 677 | 100 | 37° C. 15′ 10 μl | 1.8 | 0.3 | 31 | |
| 2 | 659 | 100 | 37° C. 15′ 10 μl | 5.8 | 0.9 | 19 | |
| 3 | 499 | 100 | 37° C. 15′ 10 μl | 9.6 | 1.9 | 25 | 10000 |
| 4 | 187 | 20 | 37° C. 15′ 10 μl | 4.3 | 0.5 | 7 | |
| 5 | 179 | 20 | 37° C. 15′ 10 μl | 19.7 | 2.2 | 8 | 1024 |
| 6 | 89 | 10 | 37° C. 15′ 10 μl | 17.7 | 2.0 | 12 | 240 |
| 7 | 19 | 2 | 37° C. 15′ 10 μl | 17.3 | 1.8 | 2 | |
| 8 | 17 | 2 | 37° C. 15′ 10 μl | 78.9 | 10.4 | 5 | |
| 9 | 19 | 2 | 37° C. 15′ 10 μl | 36.5 | 4.1 | 3 | |
| 10 | 18 | 2 | 37° C. 15′ 10 μl | 14.1 | 2.3 | 2 | 0.9 |
| 11 | 99 | 12.5 | 37° C. 15′ 50 μl | 60.3 | 7.7 | 16 | |
| 11 | 22 | 2.5 | 37° C. 15′ 50 μl | 90.1 | 10.4 | 18 | 0.3 |
| 12 | 89 | 5 | 37° C. 15′ 50 μl | 53.2 | 3.0 | 7 | |
| 12 | 92 | 5 | 37° C. 15′ 50 μl | 92.2 | 5.0 | 80 | 0.1 |

EDTA
Signal/
Bkgnd

30n7 Primer Competition Counter-SELEX

| | | | | | | | |
|---|---|---|---|---|---|---|---|
| 1 | 168 | 20 | 37° C. 15′ 100 μl | 2.1 | 0.25 | 6 | |
| 2 | 189 | 20 | 37° C. 15′ 100 μl | 15.4 | 1.62 | 119 | |
| 3 | 185 | 20 | 37° C. 15′ 100 μl | 9.2 | 0.99 | 66 | 2 |
| 4 | 95 | 5 | 37° C. 15′ 100 μl | 44.0 | 2.33 | 6 | 0.3 |
| 5 | 100 | 5 | 37° C. 15′ 100 μl | 29.0 | 1.43 | 43 | |
| 5 | 104 | 5 | 37° C. 15′ 100 μl | 36.0 | 1.70 | 24 | 0.4 |

40n7 Primer Competition Counter-SELEX

| | | | | | | | |
|---|---|---|---|---|---|---|---|
| 1 | 155 | 20 | 37° C. 15′ 100 μl | 1.9 | 0.25 | 5 | |
| 2 | 184 | 20 | 37° C. 15′ 100 μl | 26.8 | 2.92 | 172 | |
| 3 | 117 | 20 | 37° C. 15′ 100 μl | 12.9 | 2.21 | 78 | 2 |
| 4 | 93 | 5 | 37° C. 15′ 100 μl | 46.0 | 2.43 | 3 | 0.2 |
| 5 | 93 | 5 | 37° C. 15′ 100 μl | 37.0 | 2.00 | 52 | |
| 5 | 94 | 5 | 37° C. 15′ 100 μl | 42.0 | 2.25 | 15 | 0.06 |

TABLE 16

L-selectin 2′F Ligands Sequences

| | | |
|---|---|---|
| | | SEQ |
| | | ID |
| Ligand | Sequence | NO. |

Family 1a

| | | |
|---|---|---|
| LF1518 | gggaggacgau gcggG CAAAUUG CAUGCG UU-UU-- CGAGUG CUUGC UcagacGacucgcccga | 293 |
| LF1817 | gggaggacgaugc ggUG CUUAAAC AACGCG UGAAU-- CGAGUU CAUC CACUCCUCCU cagacgacucgcccga | 294 |
| LF1813 | gggaggacgaugcggUUAAU UCAGU CUCAAAC GGUGCG UUUAU-- CGAGCC ACUGA UcwgacgacucgcccgaA | 295 |
| LF1822 | gggaggacgaugcggCU UAGAG CUCAAAC GGUGUG ACUUU-- CAAGCC CUCUA UGCCcagacgacucgcccga | 296 |
| LF1514 | gggaggacgaugc ggUAC CUCAAAU UGCGUG UU-UU-- CAAGCA GUAUc agacgacucgcccga | 297 |
| LF1529 | gggaggacgaugcg gACC CUCAAAU AACGUG UCUUU-- CAAGUU GGUc agacgacucgcccga | 298 |
| LF1527 | gggaggacgaugcg gACC CUCAAAU AGCGUG CAUUU-- CAAGCU GGUc agacgacucgcccga | 299 |
| (2) | | |
| LF1536 | gggaAgacgaugc ggCG CUCAAAU AAUGCG UUAAU-- CGAAUU CGCC cagacgacucgcccga | 300 |
| (2) | | |
| LF1614 | gggaggacgaugcggCA AACAAG CUCAAAU GACGUG UUUUU-- CAAGUC CUUGUU GUcagacgacucgcccga | 301 |
| LF1625 | gggaggacgaugcggUA GUAAGU CUCAAAU GUUGCG UUUUU-- CGAAAC ACUUAC AUcaGacgacucgcccga | 302 |
| LF1728 | gggaggacgaugc ggAGA CUCAAAU GGUGUG UU-UU-- CAAGCC UCUCC cagUcgacucgcccga | 303 |
| LF1729 | gggaggacgaugc ggUG CUCAAAU GAUGCG UUUCU-- CGAAUC CACC cAgacgacucgcccga GG | 304 |
| LF1815 | gggaggacgaugc ggCCAUCGGU CUUGGGC AACGCG UU-UU-- CGAGUU ACCUAUGGUc agacgacucgcccga | 305 |
| LF1834 | gggaggacgaugcggCCAUC GGU CUUGGGC AACGCG UU-UU-- CGAGUU aCC UACAUcagacgacucgcccga | 306 |
| LF1508 | gggaggacgaugcg gGACC CUUAGGC AACGUG UU-UU-- CAAGUU GGUc agacgacucgcccga | 307 |
| LF1828 | gggaggacgaugcgg ACGUAGCU CUUAGGC AAUGCG UAUUU-- CGAAUU AGCUGUGU cagacgacucgcccga | 308 |
| LF1807 | gggaggacgaugc ggAGU CUUAGGC AGCGCG UU-UU-- CGAGCU ACUCC AUCGCCAGUcagacgacucgcccga | 309 |
| LF1825 | gggaAgacgaugcgg AAUGCU CUUAGGC AGCGCG UUAAU-- CGAGCU AGCACAUCCUcagacgacucgcccga | 310 |
| LF1855 | gggaggacgaugG ggAGU CUUAGGC AGCGCG UU-UU-- CGAGCU ACUCC AUCGCCAGUcagacgacucgcccga | 311 |
| LF1811 | gggaggacgaugcgg UAAUCU CUUAGGC AUCGCG UUAAU-- CGAGAU AGAUCACCGU cagacgacucgcccga | 312 |
| LF1626 | gggaggacgaugcgg CAAUGUCh CUUAGGC CACGCG UUAAU-- CGAGCG UGACUGU cagacgacucgcccgag | 313 |
| LF1808 | gggaggacgaugc ggCAUGGU CUUAGGC GACGCG UUUAUAU CGAGUC ACCAUGCU cagacgacucgcccga | 314 |
| (3) | | |
| LF1719 | gggaggacgaugcgg GAUG CUUAGGC GCCGUG UU-UU-- CAAGGC CAUc agacgacucgcccga | 315 |
| (2)* | | |
| LF1619 | gggaggacgaugcggu AAUUGU CUUAGGC GCCGUG UU-AU-- CAAGGC ACAAUU UCCCUcagacgacucgcccga | 316 |
| LF1620 | gggaagacgaugcggCUACUA GUGU CUUAGGC GGAGUG UUUAU-- CAAUCC ACAC aUcagacgacucgcccga | 317 |
| LF1756 | gggaggacgaugcggA CUGA CUUAGGC UGCGCG CACUU-- CGAGCA UcaG acgacucgcccga | 318 |
| LF1629 | gggaggacgaugcgg UGGUGUGU CUUUGGC ACCGCG UAUUUU- CGAGGU ACACAUca gacgacucgcccga | 319 |
| (2) | | |
| LF1821 | gggaggacgaugcggUG GUGUGU CUUUGGC ACCGCG UA-UU-- CUCGAG GUACAC AUcagacgacucgcccga | 320 |
| LF1513 | gggaggacgaugcg gGCU CUUCAGC AACGUG UU-AU-- CAAGUU AGCCc agacgacucgcccga | 321 |
| LF1615 | gggaggacgaugc ggCGUAA CUUCAGC GGUGUG UUAAU-- CAAGCC UUACGCC AUCUcagacgacucgcccga | 322 |
| LF1521 | gaggacgaugc ggGCU CUUAAGC AACGUG UU-AU-- CAAGUU AGCCc agacgacucgcccga | 323 |
| (2) | | |
| LF1651 | gggaggacga ugcggU CUCAAGC aAUGCG UUUAU-- CGAAUU ACCGUA CGCCUCCGUcagacgacucgcccga | 324 |
| LF1830 | gggaggacgaugcggAA AUCU CUUAAGC AGCGUG UAAAU-- CAAGCU AGAU CUUCGUcAgacgacucgcccga | 325 |
| LF1523 | gggaggacgaugc ggUU CUUAAGC AGCGCG UCAAU-- CGAGCU AACC cagacgacucgcccga | 326 |
| (2)* | | |
| LF1708 | gggaggacgaugc ggAU CUUAAGC AGCGCG UCAAU-- CGAGCU AACC cagacgacucgcccgag | 327 |
| ** | | |
| LF1851 | ACAGCUGAUGACCAUGAUUACGCCAAG CUUAAGC AGCGCG UU-UU-- CGAGCU CAUGUUGGUcagacgacucgcccga | 328 |
| LF1610 | gggaggac gaugcggAGGGU CUUAAGC AGUGUG AUAAU-- CAAACU ACUCUCCGUGUc agacgacucgcccga | 329 |
| (3)** | | |
| LF1712 | gggaggacgaugc ggGAU CUUAAGC AGUGCG UUAUU-- CGAACU AUCCc agacgacucgcccga | 330 |
| LF1613 | gggaggacgaugcggUGC UAUU CUUAAGC GGCGUG UUUUU-- CAAGCC AAUA UCAUcagacgacucgcccga | 331 |
| (3) | | |
| LF1735 | gggaggac gaugcggU CUUAAGC GGCGCG AUUUU-- CGAGCC ACCGCAUCCUC CGUGcaGacgacucgcccga | 332 |
| LF1731 | gggaggacgaugcg gCCU CUUAAGC GUCGUG UUUUU-- CAAGCU GGUc agacgacucgcccga | 333 |
| LF1853 | ggga ggacgaugcggAUACCACCU CUUAAGC GACGUG CAUUU-- CAAGUC AGAUGGucagacgacUcgcccga | 334 |
| LF1816 | gggaggacgAugcggUGCUA UU CUUAAGC GGCGUG UAAAU-- CAAGCU AG AUCAUCGUcagacgacucgcccga | 335 |
| LF1622 | gggaggacgaugcggA ACGACU CUUAAGC UGUGCG UU-UU-- CGAACA AGUCGU AACUcagacgacucgcccga | 336 |
| (3)* | | |
| LF1725 | gggaggacgaugc ggCU CUCAUUU wGCGCG UAAAU-- CGAGCU AGCC cagacgacucgcccga | 337 |
| LF1632 | gggaggacgaugcggAG UCwCU CUCcacC AkCGUG UkUUAAU CAAGCU AnUG CCucagacGacucgcccga | 338 |
| LF1856 | gggaggacGaugcggUCUAC GGUCU CUCUGGC GGUGCG UAAAU-- CkAACC AGAUCG cagacgacucgcccga | 339 |
| LF1631 | gggaggacgaugc ggUdAUUU CyUAAUC hGAGCG UUUAU-- CUAUCU mAAUkAUC CUcagacgacucgcccga | 340 |
| LF1730 | gggaggacgaugc ggaU CgCAAUmU GUwGCG UU-CU-- CkAAAC AGCC Ucagacgacucgcccga | 341 |
| LF1852 | gggaggacgaugc ggAACUU CUUAGGC AGCGUG CUAGU-- CAAGCU AAGUUCC ACCUcagacgacucgcccga | 371 |
| LF1653 | gggaggacgaugcggC ACAAU CUUCGGC AGCGUG CAAGAU- CAAGCU AUUGU UGUcagacgacucgcccga | 372 |
| LF1554 | gggaggacgaugc ggCGGU CUUAAGC AGUGUG UCAAU-- CAAACU AUCGUc agacgacucgcccga | 366 |
| LF1722 | gggaggacgaugc ggUU CUUAAGC AGCGCG UCAAU-- CGAGCU AACC cagacgacucgcccga | 367 |

Truncates

| | | |
|---|---|---|
| LF1514 | UGCGUG UU-UU-- CAAGCA | 385 |
| T1 | | |
| LF1514 | CUCAAAU UGCGUG UU-UU-- CAAGCA | 386 |
| T2 | | |
| LF1514 | ggUAC CUCAAAU UGCGUG UU-UU-- CAAGCA GUAUc | 387 |
| T4 | | |
| LF1807 | ggAGU CUUAGGC AGCGCG UU-UU-- CGAGCU ACUCC | 388 |
| T5 | | |

Family 1b

| | | |
|---|---|---|
| LF1511 | gggaggacgaugcgg UGGUU CUAG GCACGUG UU-UU-- CAAGUGU AAUca gacgacucgcccga | 342 |
| (4) | | |
| LF1753 | gggaggac gaugc ggAA ACAUGUG UU-UU-- CGAAUGU gCUC UCCUCCCCAAACAACyCCCCCAA | 343 |
| LF1524 | gggaggacg augc ggAA GGCCGUG UUAAU-- CAAGGCU GCAAU AAAUCAUCCUCCC cagacgacucgcc | 344 |
| | cga | |
| LF1810 | g ggaggacgaugc ggAG GAUCGUG UUCAU-- CAAGAUU GCUCGUUCUUU ACUGCGUUcagacgacucgcc | 345 |
| | cga | |
| LF1621 | gggaggacgaugcggUCAA AGUGAAG AAUG GACaGCG UU-UU-- CGAGUU GCUUCACU cagacGacucgcccga | 346 |
| (2)* | | |
| LF1826 | gggaggacgaugcgg GGAG AAUG GCCAGCG UUUAU-- CGAGGU GCUCCGUUAACCGG cAgacgacucgcccga | 347 |
| (2)* | | |
| LF1713 | gggaggacgaugcgg GAGG AAUG GACwGCG UAUAU-- CGAGUUG CCUc agacgacucgcccga | 348 |
| LF1520 | gggaggacgaugcg gAUCG AUU UCAUGCG UUUUU-- CGAGUGA CGAUc agacgacucgcccga | 349 |
| LF1552 | gggaggacgaugcggA GACc CUA AGmGsG UksUUUU CAAsCU GGUc wgacgacucgcccga | 350 |

Family 1c

| | | |
|---|---|---|
| LF1618 | gggaggacgaugcgg UUAGCCUACACUCUAGGUUCAG UU-UU-- CGAAUCUUCCACCG cWgacgacucgcccga | 351 |
| (2) | | |
| LF1528 | gggaggacgaugcgg UUAGGUCAAUGAUCUUAG UU-UU-- CGAUUCGU cagacgacucgcccga | 352 |
| (3) | | |
| LF1718 | gggaggacga ugcggA CGUGUG UAUCrAr UU-UU-- CCGCUG UUUGUG cagacgacucgcccga | 353 |
| LF1623 | gggaggacGaugcgg ACAGGGUUCUUAG GCGGAG UG-UU-- CAUCAA UCCAACCAUGU cagacgacucgcccga | 354 |
| LF1557 | gggaggacgaugcgg CGAUUUCCAC AGUUUG UCUUAUU CCGCAU AU cagacgacucgcccga | 355 |

Family 1 (Unclassified)

| | | |
|---|---|---|
| LF1707 | gggaggacgaugcgg AUAyUCAgCUyGUGUk UU-UU-- CdAUCUUCCC cagacgacucgcccga | 356 |
| LF1512 | gggaggacgaugc ggCACACGUG UU-UU-- CAAGUGUGCU CCUGGGAU cagacgacucgcccga | 357 |
| LF1535 | gggaggacgaugc ggCAAUGUG UUUCU-- CAAAUUGCU UUCUCCCUU cagacgacucgcccga | 358 |
| (2) | | |
| LF1711 | gggaAgacg augcggUG UUGAU-- CAAUG AAUGUCCUCCUCCUACCC cagacgacucgcccga | 364 |
| LF1517 | gggaggacgau gcggUG UUUGU-- CAAUGU CAUGAUUAGUUUUCCCA cagacgacucgcccga | 365 |

Family 2

| | | |
|---|---|---|
| LF1627 | gggaggacgaugc ggAUACUACCGUGCG AACaCUAAG UCCCGUCUGUCCACUCCU cagacgacucgcccga | 359 |
| (2) | | |
| LF1724 | gggaggacgaugc ggAUaCUA-UGUGCG UUCACUAAG UCCCGUC-GUCCCCU cagacgacucgcccga | 360 |
| (2)* | | |
| LF1652 | gggaggacgaugc ggGUACUA UGUACG AUCaCUAAG CCCCAUCACCCUUCUCACU cagacnacucgcccga | 361 |
| (2) | | |
| LF1519 | gggaggacgaugc ggUUACUA UGUACA UUUACUAAG ACCCAACGU cagacgacucgcccga | 362 |
| LF1608 | gggaggacgaugc ggUUwCUA UGUwCGCCUUACUAAGUACCCGUCGACUGUCCCAU cagacgacucgcccga | 363 |

Family 3

| | | |
|---|---|---|
| LF1710 | gggaggacgaugcgg AAUGrCCCGUUACCAwCAAUGCGCCUCdUUGmCCCCAAACAACyCCCCCAA | 368 |
| LF1829 | gacgaugcgg AAUyUCGUGyUAcGCGUyyyCUAUCCAAUCUACCCCmUCUCCAAU cagacgacyc----- | 369 |
| LF1509 | gggaggacgaugcgg CGCUUACAAUAAUUCUCCCUGAGUACAGCucag acgacucgcccga | 370 |

Orphans

| | | |
|---|---|---|
| LF1507 | gggaggacgaugcgg UCAUUAACCAAGAUAUGCGAAUCACCUCCU cagacgacucgcccga | 373 |
| LF1516 | gggaggacgaugcgg UCAUUCUCUAAAAAAGUAUUCCGUACCUCCa cagacgacucgcccga | 374 |
| (2) | | |
| LF1530 | gggaggacgaugcgg GUGAUCUUUUAUGCUCCUCUUGUUUCCUGU cagacgacucgcccga | 375 |
| (2)* | | |
| LF1835 | gggaggacnaugcgg UCUAGGCaUCGCUAUUCUUUACUGAUAUAAUUACUCCCCU cagacgacucgcccga | 376 |
| (4*) | | |
| mon- | gggaggacgaugcgg AGUWWGCNCGGUCCAGUCACAUCCwAUCCC cagacGacucgcccga | 377 |
| ster | | |
| LF1522 | gggaggacgAugcgg CUCUCAUAUkGwGUrUUyUUCmUUCsrGGCUCAAACAAyyCCCCCAA | 378 |
| LF1727 | gggaggacgaugcgg CUUGUUAGUUAAACUCGAGUCUCCACCCCU cagacgacucgcccga | 379 |
| LF1510 | gggaggacgaugcgg UCUCUwCUvACvUGUrUUCACAUUUUCGCyUCAAACAACyCCCCCAA | 380 |
| LF1715 | gggaggacgaugcgg UUrACAAUGrssCUCrCCUUCCCwGGUCCU cagacgacucgcccga | 381 |
| LF1809 | AggaggacGaugcgg UUAUCUGAArCwUGCGUAAmCUArUGUsAAAsUGCAACrA cRaacaacYcScccaa | 382 |
| LF1533 | Aggaagacgaugcgg UUCGAUUUAUUUGUGUCAUUGUUCUUCCAU cagacgacucgcccga | 383 |
| LF1720 | --------------- -----------GUGAUGACAUGGAUUACGC cagacgacucgcccga | 384 |

TABLE 17
2′ Fluoro L-selectin SELEXes:
Full Length Transcribed Ligands:
Protein and Lymphocyte Binding Affinity

| | | L-selectin# | Lymphocytes# # |
|---|---|---|---|
| LIGAND | SEQ ID NO | Kd (nM) | Kd (nM) |
| LF1508 | 307 | 0.5 | |
| LF1511 | 342 | 0.48 | |
| LF1512 | 357 | 315 | |
| LF1513 | 321 | 0.16 | 4 |
| LF1514 | 297 | 0.13 | 0.8 |
| LF1516 | 374 | 1.3* | |
| LF1518 | 293 | 0.42 | |
| LF1520 | 339 | 0.5* | |
| LF1521 | 323 | 0.25* | |
| LF1523 | 326 | 0.25 | |
| LF1524 | 344 | 2.1* | |
| LF1527 | 299 | 0.32 | |
| LF1528 | 352 | —* | |
| LF1529 | 298 | 0.6 | |
| LF1535 | 358 | —* | |
| LF1536 | 300 | 0.22* | |
| LF1610 | 329 | 0.53 | |
| LF1613 | 331 | 0.034 | 0.2 |
| LF1614 | 301 | 0.17 | |
| LF1615 | 322 | 0.32 | |
| LF1618 | 351 | 9.6 | 25 |
| LF1707 | 356 | 0.16* | |
| LF1708 | 327 | 70 | |
| LF1712 | 330 | 0.065* | |
| LF1713 | 338 | 0.22* | |
| LF1718 | 353 | 6.4* | |
| LF1807 | 309 | 0.034 | |
| LF1808 | 314 | 0.6 | |
| LF1810 | 345 | 8.1* | |
| LF1811 | 312 | 0.19 | |
| LF1815 | 305 | 0.18* | |
| LF1816 | 335 | —* | |
| LF1817 | 294 | 2.3* | |
| 40N7 | | — | |
| NX280 | | 1.6 | 3 |

#Nitrocellulose filter partitioning @ 37° C.;
*designate soluble L-selectin, others LS-Rg;
— indicates binding was undetectable
##Flow cytometry competition @ room temperature;

TABLE 18
P-SELECTIN 2′F RNA SELEX

| | | | | | % RNA | Signal to | % RNA | Signal to | | |
|---|---|---|---|---|---|---|---|---|---|---|
| | | | | | eluted | Noise- | eluted | Noise- | % | |
| SELEX | RNA Load | PS-Rg | Bead | Total | 5 mM | 5 mM | 50 mM | 50 mM | Retained | |
| Round # | (pmol) | (pmol) | Volume | Volume | EDTA | EDTA | EDTA | EDTA | on column | Kd (nM) |
| Rnd 1 | 320 | 200 | 10 μl | 125 μl | 1.4 | 8 | 8.3 | 40 | 0.7 | 2500 |
| Rnd 2 | 510 | 100 | 10 μl | 125 μl | 1.8 | 9 | 3.5 | 30 | 0.6 | |
| | 200 | 40 | 10 μl | 125 μl | 1.7 | 5 | 2.6 | 12 | 0.3 | |
| Rnd 3 | 200 | 40 | 10 μl | 125 μl | 2.3 | 15 | 3.0 | 13 | 0.1 | |
| | 40 | 8 | 10 μl | 125 μl | 1.3 | 4 | 0.8 | 8 | 0.3 | 1200 |
| Rnd 4 | 25 | 5 | 10 μl | 125 μl | 1.2 | 3 | 0.6 | 3 | 0.7 | |
| Rnd 5 | 25 | 5 | 10 μl | 125 μl | 0.9 | 3 | 0.15 | 1.5 | 0.3 | 280-900 |
| Rnd 6 | 25 | 5 | 10 μl | 125 μl | 0.8 | 2 | 0.0 | 1 | 0.4 | 85 |
| Rnd 7 | 50 | 5 | 10 μl | 125 μl | 4.0 | 8 | 1.0 | 4.3 | 0.5 | 13 |
| Rnd 8 | 50 | 5 | 10 μl | 125 μl | 4.6 | 16 | 0.4 | 6.7 | 0.3 | 5 |
| | 10 | 1 | 10 μl | 125 μl | 4.5 | 6 | 0.2 | 2.3 | 1.4 | 5 |
| Rnd 9 | 10 | 1 | 10 μl | 125 μl | 5.3 | 28 | 0.05 | 1.5 | 1.2 | |
| | 10 | 1 | 100 μl | 250 μl | 2.8 | 6 | 0.3 | 2 | 0.8 | |
| Rnd 10 | 5 | 0.5 | 10 μl | 500 μl | 5.6 | 20 | 0.2 | 5 | 1.2 | |
| Rnd 11 | 5 | 1 | 250 μl | 500 μl | 10 | 11 | 0.4 | 2 | 2.5 | 0.1-2 |
| | 1 | 0.2 | 10 μl | 500 μl | 14.2 | 15 | 0.6 | 3 | 13 | |
| Rnd 12 | 1 | 0.1 | 250 μl | 500 μl | 4.5 | 4 | 0.8 | 2 | 4.7 | 0.02-20 |
| Rnd 13 | 0.1 | 0.01 | 250 μl | 500 μl | 2.6 | 2 | ND | ND | 3.6 | |

TABLE 19

P-Selectin 2′-F RNA Ligands

| | | |
|---|---|---|
| | | SEQ |
| | | ID |
| Ligand | Sequence | NO. |

Family 1

| | | |
|---|---|---|
| PF373 | gggagacaagaauaaacgcucaaCGAAUCAGUAAACAUAACACCAUGAAACAUAAAUAGCACGCGAGACGUCuucgacaggaggcucacaa | 199 |
| (6) | caggc | |
| PF424 | gggagacaagaauaaacgcucaaCGAGUUCACAUGGGAGCAAUCUCCGAAUAAACAACACGCKAKCGCAAAuucgacaggaggcucacaac | 200 |
| | aggc | |
| PF412 | gggagacaagaauaaacgcucaaCGACCACAAUACAAACUCGUAUGGAACACGCGAGCGACAGUGACGCAUUuucgacaggaggcucacaa | 201 |
| | caggc | |
| PF422 | gggagacaagaauaaacgcucaaCGUCAAGCCAGAAUCCGGAACACGCGAGAAAACAAAUCAACGACCAAUCGAuucgacaggaggcucac | 202 |
| | aaaggc | |
| PF426 | gggagacaagaauaaacNcucaaCGACCACAAUAACCGGAAAUCCCCGCGGUUACGGAACACGCGAACAUGAAuucgacaggaggcucaca | 203 |
| | acaggc | |
| PF398 | gggagacaagaauaaacgcucaaCGAACCACGGGGAAAUCCACCAGUAACACGCGAGGCAAACAGACCCUCuucgacaggaggcucacaac | 204 |
| | aggc | |
| PF380 | gggagacaagaauaaacgcucaaCGAGCAAAAGUACUCA CGGGACCAGGAGAUCAGCAACACGCGAGACGAAAuucgacaggaggcucac | 205 |
| (2) | aacaggc | |
| PF377 | gggagacaagaauaaacgcucaaCGAGCCAGGAACAUCGACGUCAGCAAACGCGAGCGCAACCAGUAACACCuucgacaggaggcucacaa | 206 |
| (2) | caggc | |
| PF387 | gggagacaagaauaaacgcucaaCGCACCAGGAACAACGAGAACCAUCAGUAAACGCGAGCGAUUGCAUGuucgacaggaggcucacaaca | 207 |
| (2) | ggc | |
| PF383 | gggagacaagaauaaacgcucaaCGCACCAGGAACAACAAGAACCAUCAGUAAGCGCGAGCGAUUGCAUAuucgacaggaggcucacaaca | 208 |
| | ggc | |
| PF395 | gggagacaagaauaaacgcucaaCGAGCAAGGAACGAAUACAAACCAGGAAACUCAGCAACACGCGAGCAGUAAGAAuucgacaggaggcu | 209 |
| | cacaacaggc | |
| PF416 | gggagacaagaauaaacgcucaaCAGUUCACUCAACCGGCACCAGACUACGAUCAGCAUUGGCGAGUGAACACuucgacaggaggcucaca | 210 |
| (2) | acaggc | |
| PF388 | gggagacaagaauaaacgcucaaCUGGCAACGGGAUAACAACAAAUGU CACCAGCACUAGCGAGACGGAAGGuucgacaggaggcucaca | 211 |
| (2) | acaggc | |

Family 1 Truncates

| | | |
|---|---|---|
| PF373s1 | CUCAACGAAUCAGUAAACAUAACACCAUGAAACAUAAAUAGCACGCGAG | 220 |
| PF424s1 | CUCAACGAGUUCACAUGGGAGCAAUCUCCGAAUAAACAACACGCGAG | 221 |
| PF3981 | CUCAACGAACCACGGGGAAAUCCACCAGUAACACGCGAG | 222 |
| PF377s1 | CUCAACGAGCCAGGAACAUCGACGUCAGCAAACGCGAG | 223 |
| PF377s2 | CGCUCAACGAGCCAGGAACAUCGACGUCAGCAAACGCGAGCG | 224 |
| PF377L1 | CUCAACGAGCCAGGACUACGAUCAGCAAACGCGAG | 225 |
| PF387s1 | CUCAACGCACCAGGAACAACGAGAACCAUCAGUAAACGCGAG | 226 |
| PF383s1 | CUCAACGCACCAGGAACAACAAGAACCAUCAGUAAGCGCGAG | 227 |
| PF416s2 | CACUCAACCGGCACCAGACUACGAUCAGCAUUGGCGAGUG | 228 |
| PF422s1 | GAAUCCGGAACACGCGAGAAAACAAAUCAACGACCAAUCGAUUCG | 229 |

2′-O-Methyl Substituted Truncates

| | | |
|---|---|---|
| PF377M1 | CUCAACGAGCCAGGAACAUCGACGUCAGCAAACGCGAG | 230 |
| PF3772 | CUCAACGAGCCAGGAACAUCGACGUCAGCAAACGCGAG | 231 |
| PF377M3 | CUCAACGAGCCAGGAACAUCGACGUCAGCAAACGCGAG | 232 |
| PF377M4 | CUCAACGAGCCAGGAACAUCGACGUCAGCAAACGCGAG | 233 |
| PF377M5 | CUCAACGAGCCAGGAACAUCGACGUCAGCAAACGCGAG | 234 |
| PF377M6 | CUCAACGAGCCAGGAACAUCGACCUCAGCAAACGCGAG | 235 |

Family 2

| | | |
|---|---|---|
| PF378 | gggagacaagaauaaacgcucaaCGAUGAGCGUGACCGAAGCUAUAAUCAGGUCGAUUCACCAAGCAAUCUUAuucgacaggaggcucaca | 212 |
| (8) | acaggc | |

Family 3

| | | |
|---|---|---|
| PF381 | gggagacaagaauaaacgcucaaAGGAUCACACAAACAUCGGUCAAUAAAUAAGUAUUGAUAGCGGGGAUAuucgacaggaggcucacaac | 213 |
| (5) | aggc | |

Family 4

| | | |
|---|---|---|
| PF411 | gggagacaagaauaaacgcucaaCAACCCAACCAUCUAGAGCUUCGAACCAUGGUAUACAAGGGAACACAAAAuucgcggaggcuccaaca | 214 |
| (2) | ggcggc | |

Family 5

| | | |
|---|---|---|
| PF396 | gggagacaagaauaaacgcucaaGCGGUCAGAAACAAUAGCUGGAUACAUACCGCGCAUCCGCUGGGCGAUAuucgacaggaggcucacaa | 215 |
| (2) | caggc | |

Orphans

| | | |
|---|---|---|
| PF386 | gggagacaagaauaaacgcucaaACAAGAGAGUCAAACCAAGUGAGAUCAGAGCGUUUAGCGCGGAAAGCACAuucgacaggaggcucaca | 216 |
| | acaggc | |
| PF382 | gggagacaagaauaaacgcucaaACUCGACUAGUAAUCACCCUAGCAUAAAUCUCCUCGAGCACAGACGAUAuucgacaggaggcucacaa | 217 |
| | caggc | |
| PF404 | gggagacaagaauaaacgcucaaUCAGCAGUAAGCGAUCCUAUAAAGAUCAACUAGCCAAAGAUGACUUAuuucgacaggaggcucacaac | 218 |
| | aggc | |
| PF417 | gggagacaagaauaaacgcucaaAAAGACGUAUUCGAUUCGAAACGAGAAAGACUUCAAGUGAGCCCGCAGuucgacaggaggcucacaac | 219 |
| | aggc | |

TABLE 20
Dissociation Constants and Specificity of 2′F RNA
Ligands to P-Selectin

| | Kd | S LeX | Kd | Kd | | SEQ ID |
|---|---|---|---|---|---|---|
| Ligand | (PS-Rg) | (IC50) | (ES-Rg) | (LS-Rg) | Tm(° C.) | NO. |
| PF373 | 49.5 pM | | >3 μM | >3 μM | | 199 |
| PF377 | 18.5 pM | 3 nM | 2.3 μM | >3 μM | 53° C. | 206 |
| PF378 | 51.5 pM | | | | | 212 |
| PF380 | 74.5 pM | 4 nM | | | | 205 |
| PF381 | 16.5 pM | 1 nM | | | | 213 |
| PF386 | 45.5 pM | | | | | 216 |
| PF387 | 16 pM | | | | | 207 |
| PF388 | 90 pM | | | | | 211 |
| PF395 | 26 pM | | | | | 209 |
| PF396 | 24 pM | | | | | 215 |
| PF398 | 46 pM | | | | | 204 |
| PF404 | 47.5 pM | | | | | 218 |
| PF411 | 13 pM | 2 nM | | | | 214 |
| PF412 | 450 pM | | | | | 201 |
| PF416 | 63 pM | | | | | 210 |
| PF417 | 69 pM | | | | | 219 |
| PF422 | 172 pM | 3 nM | | | | 202 |
| PF424 | 36.5 pM | | | | | 200 |

TABLE 21

Boundary Results for 2′F RNA Ligands to P-Selectin

| | | |
|---|---|---|
| | | SEQ |
| Kd | | ID |
| (pM) | Clone# | NO. |

FAMILY 1

| | | | |
|---|---|---|---|
| 56 | PF373s1 | cucaaCGAAUCAG UA AACAUAACACCAUGAAACA UAAAUAGCACGCGAG | 220 |
| 178 | PF424s1 | cucaaCGAGUUCACAUG GGAGCAAUCUCCGAA UAAACAACACGCGAG | 221 |
| 63 | PF398s1 | cucaaCGAACCAC GG GGAAAUCCA CCAGUAACACGCGAG | 222 |
| ND | PF380s1 | cucaaCGAGCAAAAGUACUCACGGGACCAGGAGA UCAGCAACACGCGAG ACGAAAuucg | 236 |
| 50 | PF377s1 | cucaaCGAGCCAG GA ACAUCGACG UCAGCAAA CGCGAG CG | 223 |
| 50 | PF377s2 | cg cucaaCGAGCCAG GA ACAUCGACG UCAGCAAA CGCGAG CG | 224 |
| | PF412 | cg cucaaCGACCACAA UA CAAACUCG UAUGGAACACGCGAG CG | 237 |
| 63 | PF387s1 | cg cucaaCGCACCAG GA ACAACGAGAACCA UCAGUAAA CGCGAG CG | 226 |
| 10000 | PF383s1 | acg cucaaCGCACCAG GA ACAACAAGAACCA UCAGUAAG CGCGAG CG | 227 |
| | PF388 | cg cucaaCUGGCAAC GG GAUAACAACAAAUGUCA CCAGCACU AGCGAG ACG | 238 |
| 150 | PF416s1 | UCA CUCAACCGGCACCA GA CUACGA UCAGCAUU GGCGAG UG | 239 |
| | PF395 | gggagacaagaauaaacg cucaaCGAGCAAG GA ACGAAUACAAACCAGGAAACUCAGCAACACGCGAG CA | 240 |
| | PF426 | cucaaCGACCACAA UA ACCGGAAAUCCCCGCGGU UACGGAACACGCGAA CA | 241 |
| 1000 | PF422s1 | AUCAACGACCAAUC GA uucg3′ 5′GAAUCCGGAACACGCGAG AAAACAA | 229 |

FAMILY 2

| | | |
|---|---|---|
| PF378 | agaauaaacgcucaaCGAUGAGCGUGACCGAAGCUAUAAUCAGGUCGAUUCACCAAGCAAUCUUAuucg | 242 |

FAMILY 3

| | | |
|---|---|---|
| PF381 | acgcucaaAGGAUCACACAAACAUCGGUCAAUAAAUAAGUAUUGAUAGCG | 243 |

FAMILY 4

| | | |
|---|---|---|
| PF396 | gcucaaGCGGUCAGAAACAAUAGCUGGAUACAUACCGCGCAUCCGCUGGGCG | 244 |

FAMILY 5

| | | |
|---|---|---|
| PF411 | ACCAUCUAGAGCUUCGAACCAUGGUAUACAAGGGAACACAAAAuucgcggaggcucca | 245 |

ORPHANS

| | | |
|---|---|---|
| PF386 | gggagacaaga- | 246 |
| | uaaacgcucaaACAAGAGAGUCAAACCAAGUGAGAUCAGAGCGUUUAGCGCGGAAAGCACAuucgacaggaggcucacaacaggc | |
| PF417 | gggagacaagaauaaacgcucaaAAAGACGUAUUCGAUUCGAAACGAGAAAGAC UUCAAGUGAGCCCGCAGuucgacaggaggc | 247 |
| | uca | |

TABLE 22
Dissociation Constants and Specificity of Truncated
2′F RNA Ligands to P-Selectin

| | Kd | S LeX | Kd | Kd | Tm | # | SEQ ID |
|---|---|---|---|---|---|---|---|
| Ligand | (PS-Rg) | (IC50) | (ES-Rg) | (LS-Rg) | (° C.) | Bases | NO. |
| PF373s1 | 56 pM | 3 nM | >3 μM | >3 μM | | | 220 |
| PF377s1 | 60 pM | 2 nM | >3 μM | >3 μM | 59° C. | 38 | 223 |
| PF377s2 | 45 pM | 4 nM | | | | 42 | 224 |
| PF383s1 | 10000 pM | 25 nM | | | | 46 | 227 |
| PF387s1 | 63 pM | 2 nM | >3 μM | >3 μM | | 46 | 226 |
| PF398s1 | 178 pM | 2 nM | >3 μM | >3 μM | | 39 | 222 |
| PF416s2 | 150 pM | 3 nM | | | | 42 | 228 |
| PF422s1 | 1000 pM | 8 nM | >3 μM | >3 μM | | 44 | 229 |
| PF377s1B | 65 pM | 3 nM | >3 μM | >3 μM | | 38 | 223 |
| PF377s1B:SA | 30 pM | | | | | 38 | 223 |
| PF377s1F | 60 pM | 3 nM | | | | 38 | 223 |
| PF377s1- | 125 pM | 2 nM | | | | 41 | 223 |
| 5′NH2 | | | | | | | |
| PF377L1 | 220 pM | 4 nM | >3 μM | >3 μM | | 35 | 225 |
| PF377t3 | 30 pM | 2 nM | | | | 59 | 223 |
| PF377M1 | 120 pM | | >3 μM | | | 38 | 230 |
| PF377M2 | 1700 pM | | | | | 38 | 231 |
| PF377M3 | 900 pM | 10 nM | >3 μM | | | 38 | 232 |
| PF377M4 | 1700 pM | | | | | 38 | 233 |
| PF377M5 | 60 pM | 2 nM | >3 μM | | | 38 | 234 |
| PF377M6 | 250 pM | | | | | 38 | 235 |

TABLE 23
2′OMe Substitution of 2′F RNA
Ligands to P-Selectin

| Purine | Unmixed | | Mixed | Mixed | Predicted | Actual |
|---|---|---|---|---|---|---|
| Position | Ratio | Std. Dev. | 40 pM | 200 pM | Pref. | Pref. |
| 4 | 1.07 | 0.12 | 0.3 | 0.4 | 2′-OH | untested |
| 5 | 1.00 | 1.00 | 0.4 | 0.7 | 2′-OH | untested |
| 7 | 1.00 | 0.13 | 1.2 | 1.5 | 2′-O-Me | 2′-O-Me |
| 8 | 1.00 | 0.20 | 2.3 | 1.3 | 2′-O-Me | 2′-O-Me |
| 12 | 0.83 | 0.12 | 0.4 | 0.5 | 2′-OH | untested |
| 13 | 0.90 | 0.17 | 0.8 | 0.8 | neutral | 2′-O-Me |
| 14 | 0.73 | 0.15 | 0.8 | 0.9 | neutral | 2′-O-Me |
| 15 | 0.63 | 0.15 | 0.8 | 1.3 | 2′-O-Me | 2′-O-Me |
| 16 | 0.67 | 0.10 | 0.5 | 0.7 | neutral | untested |
| 18 | 0.60 | 0.10 | 0.7 | 0.7 | neutral | 2′-O-Me |
| 21 | 0.87 | 0.30 | 0.5 | 0.7 | neutral | 2′-O-Me |
| 22 | 0.72 | 0.16 | 0.7 | 0.8 | neutral | 2′-O-Me |
| 24 | 0.70 | 0.16 | 0.6 | 0.8 | neutral | 2′-O-Me |
| 27 | 0.83 | 0.12 | 1.3 | 1.5 | 2′-O-Me | 2′-O-Me |
| 28 | 0.69 | 0.09 | 0.6 | 1.0 | 2′-O-Me | ? |
| 30 | 0.90 | 0.00 | 0.8 | 1.0 | neutral | ? |
| 31 | 0.92 | 0.16 | 1.2 | 1.5 | 2′-O-Me | 2′-O-Me |
| 32 | 1.10 | 0.06 | 0.5 | 0.8 | 2′-OH | untested |
| 34 | 0.93 | 0.06 | 0.7 | 0.9 | 2′-OH | untested |

TABLE 24
P-Selectin 2′NH₂ RNA SELEX

| | | | | | % RNA | Signal to | % RNA | Signal to | | |
|---|---|---|---|---|---|---|---|---|---|---|
| | | | | | eluted | Noise- | eluted | Noise- | % | |
| SELEX | RNA Load | PS-Rg | Bead | Total | 5 mM | 5 mM | 50 mM | 50 mM | Retained | |
| Round # | (pmol) | (pmol) | Volume | Volume | EDTA | EDTA | EDTA | EDTA | on column | Kd (nM) |
| Rnd 1 | 330 | 200 | 10 μl | 125 μl | 0.0 | 1 | 1.3 | 6.5 | 0.2 | 6350 |
| Rnd 2 | 300 | 100 | 10 μl | 100 μl | 0.8 | 8 | 0.3 | 2.7 | 0.6 | |
| Rnd 3 | 550 | 100 | 10 μl | 125 μl | 0.6 | 21 | 0.2 | 8 | 0.1 | 1900 |
| Rnd 4 | 500 | 100 | 10 μl | 125 μl | 1.0 | 33 | 0.8 | 10 | 0.4 | |
| Rnd 5 | 365 | 100 | 10 μl | 125 μl | 1.5 | 30 | 1.6 | 32 | 0.4 | 470 |
| Rnd 6 | 500 | 50 | 10 μl | 125 μl | 1.9 | 22 | 0.9 | 17 | 0.3 | |
| | 50 | 5 | 10 μl | 125 μl | 1.1 | 5 | 0.4 | 2.3 | 1.2 | 103 |
| Rnd 7 | 50 | 5 | 10 μl | 125 μl | 1.8 | 7 | 0.05 | 1.8 | 0.6 | 31 |
| Rnd 8 | 50 | 5 | 10 μl | 125 μl | 3.6 | 7 | 0.0 | <1 | 0.6 | |
| Rnd 9 | 10 | 1 | 10 μl | 125 μl | 3.3 | 5 | 0.1 | 2 | 1.2 | |
| Rnd 10 | 1 | 0.2 | 10 μl | 500 μl | 2.5 | 3 | 0.0 | <1 | 0.3 | 0.2-6 |
| Rnd 11 | 1 | 0.1 | 10 μl | 500 μl | 2.0 | 2 | 0.0 | <1 | 5.0 | |
| | 1 | 0.1 | 250 μl | 500 μl | 1.5 | 2 | 0.0 | <1 | 12.0 | |
| Rnd 12 | 1 | 0.1 | 10 μl | 500 μl | 4.1 | 5 | 0.2 | 2 | 3.2 | |
| | 1 | 0.1 | 250 μl | 500 μl | 3.1 | 2 | 0.2 | 1 | 14.0 | |

TABLE 25

P-Selectin 2′NH₂ RNA Ligands

| | | |
|---|---|---|
| | | SEQ |
| | | ID |
| Ligand | Sequence | NO. |

family 1

| | | |
|---|---|---|
| PA341 (7) | gggagacaagaauaaacgcucaaGCCCCAAACGCAAGCGAGCAUCCGCAACAGGGAAGAAGACAGACGAAUGAuucgacaggaggcucac | 251 |
| | aacaggc | |
| PA350 | gggagacaagaauaaacgcucaaGCCCCAAACCCAAGUGAGCAUCCGCAACAGGGAAGAAGACAGACGAUUGAuucgacaggaggcucac | 252 |
| | aacaggc | |
| PA466 | gggagacaagaaauaaacncucaaGCCCCAAACGCAAGUGAGCAUCCGCAACAGGGAAGAAGACAGAUGAAUGAuucgacaggaggcuca | 253 |
| | caacaggc | |
| PA473 | gggagacaagaauaaacncucaaGCCCCAA GCAAGUGAGCAUCCGCAACAGGGAAGAAGACAGACGAGUGAuucgacaggaggcuca | 254 |
| | caacaggc | |
| PA477 (3) | gggagacaagaauaaacncucaaGCCCCAAaCGCAAGUG AGCAUCCGCAACAGGGAAGAAGACAGACGAAUGAuucgacaggaggcuca | 255 |
| | caacaggc | |
| PA328 (3) | gggagacaagaauaaacgcucaaGCAAAAGGCGUAAAUACACC UCCGCAACUGGGAAGAAGACGCAGGGACGGuucgacaggNggcuca | 256 |
| | caacaggc | |

family 2

| | | |
|---|---|---|
| PA337 (6) | gggagacaagaauaaacgcucaaACAGCUACAAGUGGGACAACAGGGUACAGCGGAGAGAAACAUCCAAACAAGuucgacaggaggcuca | 257 |
| | caacaggc | |

family 3

| | | |
|---|---|---|
| PA448 (7) | gggagacaagaauaaacgcucaaAUCAACUAAACAACGCAGUCACGAGAACGACCGGKCUGACUCCGAAAG uucgacaggaggcuca | 258 |
| | caacaggc | |

others

| | | |
|---|---|---|
| PA325 | gggagacaagaauaaacgcucaaACGAGAGCACCAAGGCAACAGAUGCAGAAGAAGUGUGCGCGCGCGAAA uucgacaggaggcuca | 259 |
| | caacaggc | |
| PA327 | gggagacaagaauaaacgcucaaUAAGACAACGAACAGACAGAAGCGAAAAAGGGGCGCCGCAGCAACAACAAAuucgacaggaggcuca | 260 |
| | caacaggc | |
| PA446 | gggagacaagaauaaacgcucaaCGUGUACCACAACAGUUCCACG GAAGCUGGAAUAGGACCCAGAGGAA uucgacaggaggcuca | 261 |
| | caacaggc | |
| PA313 | gggagacaagaauaaacgcucaaACAAAAUUWUGUUGGGCCCCGcAACMGGGRGGRAGRCCGUUGAAGGC uucgacaggaggcuca | 262 |
| | caacaggc | |
| PA336 | gggagacaagaauaaacgcucaaGAUCAUAACGAGAGGAGAGGGAGAACUACACGCGCGCGAGGAAAGAG uucgacaggaggcuca | 263 |
| | caacaggc | |
| PA301 | gggagacaagaauaaacgcucaaACACAAAUCGGGCAGGGACUGGGUUGGGCACGGCAGGGCGCC uucgacaggaggcuca | 264 |
| | caacaggc | |
| PA305 | gggagacaagaauaaacgcucaaGUGGGCUCGGGCCGGAUGUCUACGGGUGUGAAGAAACCCCUAGGGCAGGG uucgacaggaggcuca | 265 |
| | caacaggc | |
| PA309 | gggagacaagaauaaacgcucaaGAUCAGCGGAACUAAGAAAUGGAAGGCUAAGCACCGGGAUCGGGAGAA uucgacaggaggcuca | 266 |
| | caacaggc | |
| PA315 | gggagacaagaauaaacgcucaaUAACAAAGCAGCAAAGUACCAGAGGAGAGUUGGCAGGGUUUAGGCAGC uucgacaggaggcuca | 267 |
| | caacaggc | |
| PA318 | gggagaca-gaauaaacgcucaaAGACCAAGGGACAGCAGCGGGGAAAAACAGAUCACAGCUGUAAGAGGGC uucgacaggaggcuca | 268 |
| | caacaggc | |
| PA319 | gggagacaagaauaaacgcucaaAGUCGGGGAUAGAAACACACUAAGAAGUGCAUCAGGUAGGAGAUAA uucgacaggnggcuca | 269 |
| | caacaggc | |
| PA320 | gggagacaagaauaaacgcucaaGAGUAUCACACAAACCGGCACGGACUAAGCAGAAGGAGGUACGGAAGA uucgacaggaggcuca | 270 |
| | caacaggc | |
| PA321 | gggagacaagaauaaacNcucaaCGAAAUAGAAGGAACAGAAGAAUGGBGAWGNGGGAAAUgGCAACGAA uucgacaggnggcuca | 271 |
| | caacaggc | |
| PA324 | gggagacaagaauaaacgcucaaACGAGACCCUGGAUACGAGGCUGAGGGAAAGGGAGMMMRRAMCUARRCKC uucgacaggaggcuca | 272 |
| | caacaggc | |
| PA329 | gggagacaagaauaaacgcucaaGAAGGAUACUUAGGACUACGUGGGAUGGGAUGAAAUGGGAGAACGGGAG uucgacaggaggcuca | 273 |
| | caacaggc | |
| PA330 | gggagacaagaauaaacgcucaaAACGCACAAAGUAAGGGACGGGAUGGAUCGCCCUAGGCUGGAAGGGAAC uucgacaggaggcuca | 274 |
| | caacaggc | |
| PA332 | gggagacaagaauaaacgcucaaGGUGAACGGCAGCAAGGCCCAAAACGUAAGGCCGGAAACNGGAGAGGGA uucgacaggnggcuca | 275 |
| | caacaggc | |
| PA335 | gggagacaagaauaaacgcucaaUGAUAUACACGUAAGCACUGAACCAGGCUGAGAUCCAUCAGUGCCCAGG uucgacaggaggcuca | 276 |
| | caacaggc | |
| PA336 | gggagacaagaauaaacgcucaaGAUCAUAACGAGAGGAGAGGGAGAACUACACGCGCGCGAGGAAAGAG uucgacaggaggcuca | 277 |
| | caacaggc | |
| PA338 | gggagacaagaauaaacgcucaaUCAAGUAAGGAGGAAGGGUCGUGACAGAAAAACGAGCAAAAAACGCGAG uucgacaggaggcuca | 278 |
| | caacaggc | |
| PA339 | gggagacaagaauaaacgcucaaAAGGUGCCGGGUUUGGAGGGGUAGCAAGAAAUGGCUAGGCGCASGA uucgacaggnggcuca | 279 |
| | caacaggc | |
| PA342 | gggagacaagaauaaacgcucaaCCAACGCGCACCCCGCAGCAAACGAAAUUGGGGAGACAGGUGCAAGACAG uucgacaggaggcuca | 280 |
| | caacaggc | |
| PA349 | gggagacaagaauaaackcucaaCAAACAAUAUCGGCGCAGGAAAACGUAGAAACGAAAMGGAGCUGCGYGGA uucgacaggaggcuca | 281 |
| | caacaggc | |
| PA351 | gggagacaagaauaaacgcucaaUGAUAGCACAGUGUAUAAGAAAACGCAACACCGCGCGCGGAAAGAG uucgacaggaggcuca | 282 |
| | caacaggc | |
| PA352 | gggagacaagaauaaacgcucaaGAUCAUCGCAGUAUCGGAAUCGACCCUCAGUGGGUGACAUGCGGACAAG uucgacaggaggcuca | 283 |
| | caacaggc | |
| PA353 | gggagacaagaauaaacgcucaaGUACCGGGAAGGGAUGAACUGGGAUAUGGGAACGGAGGUCAGAGGCACGA uucgacaggaggcuca | 284 |
| | caacaggc | |
| PA354 | gggagacaagaauaaacgcucaaGCAAUGGAACGCUAGGAGGGAACAUAAGCAGGGCGAGCGGAGUCGAUAGC uucgacaggaggcuca | 285 |
| | caacaggc | |
| PA447 | gggagacaagaauaaacgcucaaAACAGAACUGAUCGGCGCAGGUUGAUAAAGGGGCAGCGCGAAGAUCACAA uucgacaggaggcuca | 286 |
| | caacaggc | |
| PA463 | gggagacaagaauaaacgcucaaGGGAAACGGAAAGGGACAAGGCGAACAGACGAGAAGUAGACGGAGUAGGA uucgacaggaggcuca | 287 |
| | caacaggc | |
| PA465 | gggagacaagaauaaacgcucaaNNNGAGGAAGGGCACGCAAGGAAACAAAACACAAAGCAGAAGUAGUAAGA uucgacaggaggcuca | 288 |
| | caacaggc | |
| PA467 | gggagacaagaauaaacgcucaaGUACRCAGAGAGCAGAAGCAGAGAGACUUGGGAUGGGAUGAAAUGGKC uucgacaggaggcuca | 289 |
| | caacaggc | |
| PA479 | gggagacaagaauaaacNcucaaCCGACGUGGACDCGCAUCGGCAUCCAGACCAGGCUGNBCNGCACCASACG uucgacaggaggcuca | 290 |
| | caacaggc | |

TABLE 26
Dissociation Constants and Specificity of
2′NH2 RNA Ligands to P-Selectin

| | Kd | | Kd | SLeX | Kd | Kd | SEQ ID |
|---|---|---|---|---|---|---|---|
| Ligand | (PS-Rg) | | (4° C.) | (IC50) | (ES-Rg) | (LS-Rg) | NO. |
| PA301 | 2.5 | nM | | | | | 264 |
| PA305 | 0.21 | pM | | | | | 265 |
| PA309 | 0.656 | pM | | | | | 266 |
| PA315 | 5 | nM | | | | | 267 |
| PA318 | 2 | nM | | | | | 268 |
| PA319 | 11 | nM | | | | | 269 |
| PA320 | 4.5 | nM | | | | | 270 |
| PA321 | 8 | nM | | | | | 271 |
| PA325 | >10 | nM | | | | | 259 |
| PA327 | 13.5 | nM | | | | | 260 |
| PA328 | 3 | nM | | | | | 256 |
| PA329 | 4 | nM | | | | | 273 |
| PA330 | 0.237 | nM | | | | | 274 |
| PA335 | 10.5 | nM | | | | | 276 |
| PA336 | 15 | nM | | | | | 277 |
| PA337 | 4.5 | nM | | | | | 257 |
| PA338 | 57 | nM | | | | | 278 |
| PA339 | 13.5 | nM | | | | | 279 |
| PA341 | 0.44 | nM | | 3 nM | | | 251 |
| PA342 | 4 | nM | | | | | 280 |
| PA350 | 0.06 | nM | 0.01 nM | 2 nM | 375 nM | >3 μM | 252 |
| PA351 | 2 | nM | | | | | 282 |
| PA352 | 6 | nM | | | | | 283 |
| PA353 | 9 | nM | | | | | 284 |
| PA354 | 5 | nM | | | | | 285 |
| PA447 | 50 | nM | | | | | 286 |
| PA448 | 5 | nM | | | | | 258 |
| PA463 | 8 | nM | | | | | 287 |
| PA465 | >50 | nM | | | | | 288 |
| PA466 | 0.43 | nM | | | | | 253 |
| PA467 | 24 | nM | | | | | 289 |
| PA473 | 0.36 | nM | | | | | 254 |
| PA477 | 0.57 | nM | | | | | 255 |

1-64. (canceled)

1. An aptamer that binds to P-selectin wherein said aptamer comprises SEQ ID NO: 223.

2. The aptamer of claim 1, wherein said aptamer is conjugated to a polyethylene glycol moiety.

3. The aptamer of claim 2, wherein said polyethylene glycol moiety is conjugated to the 5′ end of said aptamer.

4. The aptamer of claim 3, wherein said aptamer further comprises an inverted deoxythymidine at the 3′ end of said aptamer.

5. The aptamer of claim 1, wherein at least one of said guanines within said aptamer is 2′-O-methyl (2′-OMe) and at least one of said adenines within said aptamer is 2′-O-methyl (2′-OMe).

6. The aptamer of claim 5, wherein said aptamer is conjugated to a polyethylene glycol moiety.

7. The aptamer of claim 6, wherein said polyethylene glycol moiety is conjugated to the 5′ end of said aptamer.

8. The aptamer of claim 7, wherein said aptamer further comprises an inverted deoxythymidine at the 3′ end of said aptamer.

9. An aptamer that binds to P-selectin wherein said aptamer comprises SEQ ID NO: 223, wherein SEQ ID NO: 223 further comprises 2′-OMe purines at positions 7, 8, 9, 13, 14, 15, 18, 21, 22, 24, 27, 28 and 31.

10. The aptamer of claim 9, wherein said aptamer is conjugated to a polyethylene glycol moiety.

11. The aptamer of claim 10, wherein said polyethylene glycol moiety is conjugated to the 5′ end of said aptamer.

12. The aptamer of claim 11, wherein said aptamer further comprises an inverted deoxythymidine at the 3′ end of said aptamer.

13. An aptamer that binds to P-selectin wherein said aptamer comprises: PEG40K-nh-fC-fU-fC-rA-rA-fC-mG-mA-mG-fC-fC-rA-mG-mG-mA-rA-fC-mA-fU-fC-mG-mA-fC-mG-fU-fC-mA-mG-fC-rA-mA-rA-fC-rG-fC-rG-rA-rG-idT (SEQ ID NO: 392)
wherein “PEG40K” is a 40 KDa polyethylene glycol moiety, “nh” is an amine linker and “idT” is an inverted deoxythymidine, “f” is fluoro, “m” is methoxy, and “r” is ribo.

14. The aptamer of claim 13, wherein said amine linker is a hexylamine linker.
